(19) United States
(12) Patent Application Publication
(10) Pub. No.: US 2021/0220480 A1
(43) Pub. Date: Jul. 22, 2021

(54) Cancer Treatment And Imaging Methods Using Thermotherapy And Drug Delivery

(71) Applicant: Gholam A. Peyman, Sun City, AZ (US)

(72) Inventor: Gholam A. Peyman, Sun City, AZ (US)

(21) Appl. No.: 17/220,878

(22) Filed: Apr. 1, 2021

Related U.S. Application Data
(63) Continuation-in-part of application No. 16/988,697, filed on Aug. 9, 2020.
Application No. 16/988,697 is a continuation-in-part of application No. 16/843,831, filed on Apr. 8, 2020.
Application No. 16/843,831 is a continuation-in-part of application No. 16/004,401, filed on Jun. 10, 2018.
Application No. 16/004,401 is a continuation-in-part of application No. 15/853,821, filed on Dec. 24, 2017, now Pat. No. 10,300,121.
Application No. 15/853,821 is a continuation-in-part of application No. 15/143,981, filed on May 2, 2016, now Pat. No. 9,849,092.
Application No. 15/143,981 is a continuation-in-part of application No. 14/976,321, filed on Dec. 21, 2015, now Pat. No. 10,136,820.
Application No. 16/843,831 is a continuation-in-part of application No. PCT/US2018/054880, filed on Oct. 8, 2018.
(60) Provisional application No. 63/004,256, filed on Apr. 2, 2020.
(60) Provisional application No. 62/885,173, filed on Aug. 9, 2019.
(60) Provisional application No. 63/004,256, filed on Apr. 2, 2020.
(60) Provisional application No. 62/614,456, filed on Jan. 7, 2018.
(60) Provisional application No. 62/569,592, filed on Oct. 8, 2017.
(60) Provisional application No. 62/577,485, filed on Oct. 26, 2017.
(60) Provisional application No. 62/614,456, filed on Jan. 7, 2018.
(60) Provisional application No. 62/720,258, filed on Aug. 21, 2018.
(60) Provisional application No. 62/569,592, filed on Oct. 8, 2017.
(60) Provisional application No. 62/577,485, filed on Oct. 26, 2017.

(51) Int. Cl. A61K 47/68; A61K 41/00; A61K 9/127; A61K 9/51; A61K 47/69; A61K 31/155; A61K 45/06; A61M 1/36; A61M 1/34; A61K 49/00; A61N 5/10; A61K 35/28; A61K 9/00; A61N 1/32; A61P 35/00; A61B 5/00

(52) U.S. Cl. CPC A61K 47/6851; A61N 2005/1098; A61K 9/127; A61K 9/51; A61K 47/6931; A61K 31/155; A61K 45/06; A61M 1/36; A61M 1/3496; A61K 49/0043; A61K 49/006; A61K 9/5115; A61N 5/10; A61K 35/28; A61K 9/0043; A61N 1/32; A61K 41/0028; A61P 35/00; A61K 9/0009; A61B 5/0093; A61K 41/0033

(57) ABSTRACT

Cancer treatment and imaging methods using thermotherapy and drug delivery are disclosed herein. In one embodiment, the method comprises the steps of administering a plurality of antibody or aptamer-conjugated nanoparticles, liposomes, and/or micelles containing a medication and/or gene to a patient in need thereof so as to target a tumor in the patient, at least some of the antibody or aptamer-conjugated nanoparticles, liposomes, and/or micelles attaching to surface antigens of tumor cells of the tumor so as to form a tumor cell/nanoparticle/liposome/micelle complex; and heating the antibody or aptamer-conjugated nanoparticles, liposomes, and/or micelles using an energy source so as to raise the temperature of the tumor cell/nanoparticle complex, micelle complex, and/or liposome complex, thereby releasing one or more medications from the antibody or aptamer-conjugated nanoparticles, liposomes, and/or micelles, and damaging one or more tumor cell membranes at the tumor site.

### CROSS-REFERENCE TO RELATED APPLICATIONS

This patent application claims priority to U.S. Provisional Patent Application No. 63/004,256, entitled “Cancer Treatment Methods Using Thermotherapy And Drug Delivery”, filed on Apr. 2, 2020, and is a continuation-in-part of U.S. patent application Ser. No. 16/988,697, entitled “Cancer Treatment Methods Using Thermotherapy And/Or Enhanced Immunotherapy”, filed on Aug. 9, 2020, which claims priority to U.S. Provisional Patent Application No. 62/885,173, entitled “Cancer Treatment Methods Using Thermotherapy and/or Enhanced Immunotherapy”, filed on Aug. 9, 2019, and U.S. Provisional Patent Application No. 63/004,256, entitled “Cancer Treatment Methods Using Thermotherapy And Drug Delivery”, filed on Apr. 2, 2020, and Ser. No. 16/988,697 is a continuation-in-part of U.S. patent application Ser. No. 16/843,831, entitled “Cancer Treatment Methods Using Thermotherapy And/Or Enhanced Immunotherapy”, filed on Apr. 8, 2020, and Ser. No. 16/843,831 is a continuation-in-part of U.S. patent application Ser. No. 16/004,401, entitled “Early Cancer Detection And Enhanced Immmunotherapy”, filed Jun. 10, 2018, which claims priority to U.S. Provisional patent Application No. 62/614,456, entitled “Cancer Treatment Methods Using Thermotherapy and/or Enhanced Immunotherapy”, filed on Jan. 7, 2018, and Ser. No. 16/004,401 is a continuation-in-part of application Ser. No. 15/853,821, entitled “Early Cancer Detection And Enhanced Immunotherapy”, filed Dec. 24, 2017, now U.S. Pat. No. 10,300,121, which claims priority to U.S. Provisional Patent Application No. 62/569,592, entitled “Cancer Treatment Methods Using Thermotherapy and/or Enhanced Immunotherapy”, filed on Oct. 8, 2017, and to U.S. Provisional Patent Application No. 62/577,485, entitled “Cancer Treatment Methods Using Thermotherapy and/or Enhanced Immunotherapy”, filed on Oct. 26, 2017, and Ser. No. 15/853,821 is a continuation-in-part of application Ser. No. 15/143,981, entitled “Early Cancer Detection And Enhanced Immunotherapy”, filed May 2, 2016, now U.S. Pat. No. 9,849,092, which is a continuation-in-part of application Ser. No. 14/976,321, entitled “Method to Visualize Very Early Stage Neoplasm or Other Lesions”, filed Dec. 21, 2015, now U.S. Pat. No. 10,136,820, the disclosure of each of which is hereby incorporated by reference as if set forth in their entirety herein.

U.S. patent application Ser. No. 16/843,831 also is a continuation-in-part of International Patent Application Ser. No. PCT/US2018/054880, entitled “Cancer Treatment Methods Using Thermotherapy and/or Enhanced Immunotherapy”, filed Oct. 8, 2018, which claims priority to U.S. Provisional Patent Application No. 62/569,592, entitled “Cancer Treatment Methods Using Thermotherapy and/or Enhanced Immunotherapy”, filed on Oct. 8, 2017, U.S. Provisional Application No. 62/577,485, entitled “Cancer Treatment Methods Using Thermotherapy and/or Enhanced Immunotherapy”, filed on Oct. 26, 2017, U.S. Provisional Application No. 62/614,456, entitled “Cancer Treatment Methods Using Thermotherapy and/or Enhanced Immunotherapy”, filed on Jan. 7, 2018, and U.S. Provisional Patent Application No. 62/720,258, entitled “Cancer Treatment Methods Using Thermotherapy and/or Enhanced Immunotherapy”, filed on Aug. 21, 2018; the disclosure of each of which is hereby incorporated by reference as if set forth in their entirety herein.

### STATEMENT REGARDING FEDERALLY SPONSORED RESEARCH OR DEVELOPMENT

Not Applicable.

### NAMES OF THE PARTIES TO A JOINT RESEARCH AGREEMENT

Not Applicable.

### INCORPORATION BY REFERENCE OF MATERIAL SUBMITTED ON A COMPACT DISK

Not Applicable.

### BACKGROUND OF THE INVENTION

### 1. Field of the Invention

The invention generally relates to cancer treatment methods using thermotherapy and/or enhanced immunotherapy. More particularly, the invention relates a treatment modality for the treatment of a cancerous tumor in the body or a benign compressive tumor in the brain, such as benign tumor fibroma, schwannoma, neurofibroma, meningioma, leiomyoma, etc., or a tumor in any other part of the body using thermotherapy and drug delivery. The invention further relates to cancer treatment methods using nanoparticles and photoacoustics, ultrasound, laser, and/or an alternating magnetic field for thermotherapy and drug delivery. The invention still further relates to Wnt-inhibitors and other cellular pathway inhibitors in combination with immunothermotherapy or immuneradiothermotherapy.

### 2. Background

Wnt signaling is involved in control of stem cell proliferation. Wnt mutation causes developmental defects in many disease processes including cancer.

On the cell surface membrane, Wnt proteins bind to receptors of the Frizzled and LRP protein families causing accumulation of beta-catenin in the cytoplasm and its translocation in the nucleus forms a complex with transcriptional cofactor (TCF) to activate the transcription of Wnt targeted genes.

The Wnt pathway is considered canonical when it is dependent on beta-catenin or non-canonical when it is independent, Wnt signaling is involved in cell fate specification, cell proliferation, cell migration, and insulin sensitivity.

Wnt signaling is activated in many cancer stem cells and metastatic cells influencing the immune response to the cancer. Increased Wnt signaling releases a compound from the cancer cell by which the cancer cell evades recognition by the T-lymphocytes. Thus, increased Wnt signaling predicts a poor prognosis in cancer.

Furthermore, the cancer cell membrane produces the extracellular vesicles or exosomes that contain Wnt ligands. When these exosomes are picked up by neighboring cells, the Wnt ligands become liberated in the cytoplasm causing accumulation of β-catenin and translocated in the nucleus of the host cell, thereby influencing the Wnt related genes in the normal host cells. This mechanism is used by cancer cells when the tumor metastasizes to prepare a niche for the growth of the circulating tumor cells.

Wnt activation is reported in adenomatous polyposis coli, Cholangiocarcinoma, Leukemia, leukemia initiating cell, Acute Myeloid leukemia, Chorionic lymphocytic leukemia, acute lymphoblastic leukemia, Melanoma, breast cancer, prostate cancer, glioblastoma, ovarian cancer, esophageal cancer, cervical cancer, etc.

Wnt inhibitors are compounds, such as FH535, IWP-2, PNU-74654, IWR-lendo, IWR-exo, Demethoxycurcumin, CCTO36477, KY02111, WAY-316606, SFRP, IWP, LGK974, C59, Ant1.4Br/Ant 1.4C1, ivermectin, niclosamide, apicularen and bafilomycin, XAV939, XAV939, G007-LK and G244-LM, NSC668036, SB-216763, gemtuzumab, etc.

Glycogen Synthase Kinase-3 (GSK-3) is a serine/threonine protein kinase, which plays a key role in Wnt/β-catenin signaling during embryonic development, inflammation and cancer. Inhibition of GSK-3 inhibits Wnt pathway in cancer.

RHO associated protein Kinase (Rock) is a kinase belonging to the family of serine-threonine Kinase involved in regulating the shape and the cytoskeleton of the cells, it is an important regulator of cell migration, stimulates PTEN phosphatase activity, leading to uncontrolled cell division in cancer. Rock is active in inflammatory processes, cancer, Parkinson's disease, diabetes and many neurodegenerative diseases, such as Alzheimer's disease, and in the production and stiffening of collagen in tumors, such as pancreatic cancer. Therefore, Rock inhibitors or in combination with PDGF inhibitors inhibit inflammatory processes, reduce TGF-β formation, block cell migration, and inhibit metastatic spread of the tumors.

There are a number of Rock inhibitors available however they have not been used in combination with functionalized nanoparticles to reduce the inflammation during immune therapy or thermoimmune therapy. The following compounds are readily available and some have been approved by the FDA: potent ROCK inhibitor; orally bioavailable Fasudil hydrochloride, Inhibitor of cyclic nucleotide dependent- and Rho-kinases GSK 269962, Potent and selective ROCK inhibitor GSK 429286, Selective Rho-kinase (ROCK) inhibitor H1152 dihydrochloride, Selective Rho-kinase (ROCK) inhibitor Glycyl H 1152 dihydrochloride, Selective Rho-kinase (ROCK) inhibitor; more selective analogue of H1152, Cell-permeable, selective Rho-kinase inhibitor OXA 06 dihydrochloride, potent ROCK inhibitor PKI1447 dihydrochloride, potent and selective ROCK inhibitor; antitumor SB 772077B, potent Rho-kinase inhibitor; vasodilator SR 3677 dihydrochloride, potent, selective Rho-kinase (ROCK) inhibitorTC-57001, potent and highly selective ROCK inhibitor; orally active Y-27632 dihydrochloride.

The ras oncogenes and the proteins (Ras) encoded genes have had an important roles in the study of human cancer affecting >50% of colon and >90% of pancreatic cancers.

Ras proteins, are membrane-bound GTP-binding proteins that serve as molecular switches in mitogenic signal transduction. Prenylation of Ras proteins by intermediates of the isoprenoid analogs of Farnesyl Diphosphate inhibits Farnesyl transferase and Ras activation. Other relevant pathways influencing the activation of Ras are PK1 and PK8 and P13K, RAF/MEK/ERK, PI3K/AKT/mTOR and RalA/B that can be inhibited by gemcitabine, wortmannin, LY294002 and inhibition of Cyclin-dependent kinase 5 (CDK5) and tipifarnib alone or in combination with Gemcitabine or rapamycin and AZD8055 (a TORKi) everolimus preventing Ras-Ral signaling.

In a simple form, cancer therapy needs a robust immune response of one's body to be successful and prevent recurrences.

Antineoplastic medication kills tumor cells, inhibiting their metabolism and cell pathways. The dead tumor cells are lysed by their enzymatic processes that exists inside the tumor cells or the enzymes that are present in any tissue releasing the tumor antigens. The tumor antigen induces an immune response by being picked up by the dendritic cell in the lymph nodes. The dendritic cells present the antigen to Cytotoxic T-Lymphocytes. The cytotoxic T-lymphocytes migrate thorough the circulation, recognize the tumor cells and kill them.

Similarly, the check point inhibitors prevent the tumor cells from escaping recognition by the cytotoxic T-lymphocytes, macrophages and killer cells leukocytes, etc.

Neoplastic medications, attack cells in an indiscriminating manner, killing the tumor cells, normal cells, and weaken the immune response. The checkpoint inhibitors, though extremely beneficial, attack the checkpoint inhibitors of the normal cells and induce autoimmunity.

Nervous system structures include the neurons or nerve cells, and meninges tissue that surround and protect the brain and spinal cord. The bony skull surrounding the brain and the vertebra surround the spinal cord. The cerebrospinal fluid (CSF) is the fluid that surrounds, protects, and cushions the brain and spinal cord. The CSF provides nutrition to the brain and spinal cord. The brain and spinal cord make up the central nervous system (CNS).

Cancer or benign tumors can affect almost any organ, e.g., the brain and the spinal cord, etc. The brain tumors affect mostly the cerebrum, cerebellum, and the brain stem of the brain, thus creating the symptoms of visual disturbance, hearing loss, loss of balance, seizure, numbness of the extremities, facial paralysis, headache, memory loss, and speech changes, or affect the sense of smell, or cause vomiting and nausea. The tumors affecting the spinal cord are associated with back pain. The brain tumor can be malignant or benign. The benign tumors grow gradually and have distinct borders, but damage the adjacent vital brain or spinal cord structures. The malignant brain tumors grow fast, but seldom metastasize. The most common malignant brain tumors are gliomas classified grade I-IV depending on their degree of the malignancy, such as gliomas with their subtypes of pilocytic Astrocytoma (grade I), Diffuse Astrocytoma (grade II), Anaplastic Astrocytoma (grade III), Glioblastoma Multiforme (grade IV), Oligodendroglioma (grade II) Anaplastic Oligodendroglioma (grade III), Ependymoma (grade II), Anaplastic Ependyma (grade III), etc. The tumor can however originate in the other parts of the body, such as the breast, skin melanoma, lung, gastrointestinal tract, or kidney. The most common benign tumors of the brain are memingiomas, schwannomas, pituatory adenomas, hemangiblastomas, craniopharyngiomas, and papillomas of the choroid plexus of the brain. The spinal cord tumors can either grow intramedullary from the spinal cord glial cells, such as astocytomas and ependymomas, or extramedullary from the dura layer covering the spinal cord and the nerve roots, such as a schwannoma, but mostly the lesions are metastatic from other parts of the body. The most common spinal cord tumors are osteomas or osteoblastoma, Ewing sarcomas, osteosarcomas, eosinophilic granulomas aneurism, mesenchymal chondrosarcomas, fibrous dysplasia, angiomas and angiosarcomas, and fibromas. Other genetic diseases causing benign lesions in the brain and spinal cord are neurofibromatosis and Van Hipple-Lindau disease. The symptoms are back pain or lower back, stiff neck, paralysis, loss of sensation or by compressing the blood vessels and the nerves or bones. The diagnosis of the brain and spinal cord tumors is done by X-ray, CT/CAT scan, MRI, or bone scan, Technectium-99, etc. The therapy is mostly is done by brain surgery by opening the skull and exposing the tumor which often is imprecise, since the borders of the tumors are not well-defined; shunt-implants to drain the excessive intracranial fluid and reduce the pressure, radiation is effective partially to the malignant tumors, but not the benign lesion, these include also proton beam, helium neon beam, or gamma knife radiation, which are mostly useful for very early stage lesions, and anticancer medications administered systemically, by intravenous administration, or orally.

Measurement of the body temperature is done presently either non-invasively using an MRI system or invasively by inserting a temperature sensitive fiber optic or a thermocouple inside the tissue. The MRI systems are bulky and non-transportable, and the fiber optics, such as Luxtron, and electrodes, such as thermocouples, are useful for very limited areas (e.g., 1-2 mm near the probe) and once placed inside the tissue they cannot be repeatedly moved from one spot to the next one. The non-invasive concept of thermometry is desirable, but seldom used in treatment of cancer or a benign compressive tumor, such as a benign tumor fibroma, schwannoma, neurofibroma, meningioma, or leiomyoma, which by their growth can damage or compromise vital organs.

Therefore, what is needed are cancer or benign compressive tumor treatment methods using controlled localized thermotherapy and/or enhanced immunotherapy that are able to effectively target and treat tumors, while having minimal deleterious effects on the healthy cells of the patient.

### BRIEF SUMMARY OF EMBODIMENTS OF THE INVENTION

Accordingly, the present invention is directed to cancer treatment methods using thermotherapy and/or enhanced immunotherapy that substantially obviate one or more problems resulting from the limitations and deficiencies of the related art.

In accordance with one or more embodiments of the present invention, there is provided a cancer treatment method using controlled localized thermotherapy. The method comprises the steps of: (i) administering a plurality of nanoparticles to a patient in need thereof so as to target a tumor in the patient, the administered nanoparticles being coated with an antitumor antibody and a thermosensitive polymer, and the administered nanoparticles containing quenched fluorescein or doxorubicin, liposomes, fluorescent dextrans, polymer micelles, or nanoparticles carrying a dye or another dye indicator, such as bubble liposomes containing air pocket or nanoemulsion of PFC carrying fluorescein, a gene and a drug in the thermosensitive polymer coating (e.g., poly(N-isopropylacrylamide); (ii) heating the nanoparticles with an energy source to a temperature of about 41° C. to about 43° C. so as to damage one or more tumor cell membranes at a treatment site of the tumor and melt the thermosensitive polymer coating of the nanoparticles, thereby releasing the fluorescein or doxorubicin into the circulation of the patient; and (iii) radiating a nail bed of the patient or any other part of the body with ultraviolet radiation so that the released fluorescein or doxorubicin fluoresces, thus indicating that the temperature of about 41° C. to about 43° C. has been achieved at the treatment site.

In a further embodiment of the present invention, the nanoparticles comprise perfluorocarbon liquid (PFCL) nanoparticles, and the method further comprising the steps of: (iv) additionally heating the nanoparticles with the energy source to a temperature of about 56° C. so as to reach the boiling point of the PFCL nanoparticles, thereby creating a detectable cavitation sound; and (v) recording the detectable cavitation sound with an ultrasonic receiver or microphone so that a control signal is capable being sent to the energy source to indicate that the heating of target tissue in the patient is to be stopped, thereby preventing thermal damage to surrounding healthy cells.

In yet a further embodiment, the antitumor antibody of the nanoparticles is in the form of a monoclonal antibody.

In still a further embodiment, the thermosensitive polymer coating of the nanoparticles further comprises at least one medication configured to be released when the thermosensitive polymer is melted, the medication present in an amount to provide the patient a dose lower than a conventional dose of the medication due to the one or more tumor cell membranes being previously compromised by the thermotherapy of the method.

In yet a further embodiment, the thermosensitive polymer coating of the pluralities of the nanoparticles further comprises checkpoint inhibitors alone or in combination with Rock inhibitors configured to be released when the thermosensitive polymer is melted, the checkpoint inhibitors being used in a localized immunotherapy treatment procedure targeting the cells of the tumor without causing damage to the healthy cells of the patient, the Rock inhibitor reducing TGF-β production after therapy and the subsequent scar formation.

In still a further embodiment, the energy source heats the nanoparticles using focused ultrasonic energy in a thermal mode.

In yet a further embodiment, the nanoparticles are selected from a group consisting of iron oxide gold nanoparticles, gold graphene oxide nanoparticles, gold nanoparticles, silicone nanoparticles, carbon nanoparticles, magnetic nanoparticles, gold nanorods, gold nanoshells, gold nanocages, iron oxide nanotubes, gold nanotubes, carbon nanotubes, and combinations thereof.

In still a further embodiment, the nanoparticles are further conjugated with cell penetrating peptides (CPPs) or activatable cell-penetrating peptides (ACPPs) or Vitamin E so to enhance cell penetration into the cells of the tumor.

In yet a further embodiment, the released fluorescein fluoresces as green light passing through a blue absorbing filter, the blue absorbing filter being operatively connected to a photomultiplier via a fiber optic, the photomultiplier converting the green light into an electrical signal that is delivered to a processor, the processor configured to control the energy source at the desired temperature of about 41° C. to about 43° C. for a predetermined period of time.

In accordance with one or more other embodiments of the present invention, there is provided a cancer treatment method comprising administering to a patient having an early stage tumor a combination of thermotherapy and immunotherapy, where thermotherapy comprises systemically administering tumor-antibody-coated nanoparticles coated with a thermosensitive polymer, and a Wnt inhibitor being conjugated with the thermosensitive polymer coating of the nanoparticles, the thermotherapy further comprises heating the tumor-antibody-coated nanoparticles using an energy source at the site of the tumor so as to melt the thermosensitive polymer coating of the nanoparticles and release the Wnt inhibitor to inhibit the Wnt/β-catenin pathway in the cells of the tumor, wherein the heating of the nanoparticles further damages one or more tumor cell membranes and releases antigenic material in vivo that activates and stimulates an immunogenic response of the patient at the site of the tumor; and immunotherapy comprises systemically administering the patient's natural killer (NK) cells/dendritic cells pre-sensitized in vitro to the tumor.

In a further embodiment of the present invention, the thermosensitive polymer coating of the nanoparticles further comprises at least one rho-kinase inhibitor configured to be released when the thermosensitive polymer is melted, the rho-kinase inhibitor acting as an anti-inflammatory agent to prevent a cytokine storm resulting from the immunotherapy, and to inhibit Wnt activation in the cells of the tumor.

In yet a further embodiment, the method further comprises the step of removing cytokines after the immunotherapy by electrophoresis, plasmapheresis, plasma exchange, or ARF6 inhibition so to prevent a cytokine storm, or oral Rho kinase inhibitors.

In still a further embodiment, the thermosensitive polymer coating of the nanoparticles further comprises metformin, buformin, or phenformin alone or with C. chinensis Franch polysaccharides (CCP) so as to inhibit glucose metabolism in the cells of the tumor, and may be provided in combination with a glutaminase inhibitor, such as Calithera, thus inhibiting glutamine uptake for the tumor to which the tumor cells are addicted.

In yet a further embodiment, the thermosensitive polymer coating of the antibody conjugated nanoparticles further comprises ivermectin or niclosamide configured to be released when the thermosensitive polymer is melted, the ivermectin or niclosamide inhibiting the Wnt/β-catenin pathway in the cells of the tumor.

In still a further embodiment, the thermosensitive polymer coating of the antibody conjugated nanoparticles further comprises metformin, buformin, or phenformin, and additionally comprises syrosingopine so as to inhibit glucose metabolism in the cells of the tumor.

In yet a further embodiment, the thermosensitive polymer coating of the antibody conjugated nanoparticles further comprises metformin, buformin, or phenformin, and additionally comprises syrosingopine so as to inhibit glucose metabolism in the cells of the tumor, and may be provided in combination with a glutaminase inhibitor, such as Calithera, thus inhibiting glutamine uptake for the tumor to which the tumor cells are addicted.

In still a further embodiment, the thermosensitive polymer coating of the antibody conjugated nanoparticles further comprises an inhibitory gene(s) and a CRISPR/cas9 complex or Cas9 ribonucleoproteins (RNPs) to stimulate or modify tumor genes at the site of the tumor upon release from the thermosensitive polymer coating of the nanoparticles at a temperature of about 41° C. to about 43° C.

In yet a further embodiment, gene modification is done using antibody coated nanoparticles conjugated with CRISPR/cas9 or Cas9 RNPs mediated homology-independent targeted integration (HITI) or homology directed repair (HDR) to modify the mutated gene of the patients CAR-T-cells prior to their culture in vitro with the tumor antigen and with or without viral-like particle (VLP) and growing them to be administered to the patient.

In yet a further embodiment, antibody coated nanoparticles conjugated with CRISPR/cas9 or Cas9 RNPs mediated homology-independent targeted integration (HITI) or homology directed repair (HDR) are used to modify the genes of the natural killer cells obtained from the patient to eliminate or replace the mutated genes in these cells prior to culturing them with tumor antigens and VLP in vitro and growing them to be administered to the patient as needed.

In one embodiment, genetically modified CAR-T cells and/or natural killer cells are grown with tumor antigens, VLPs, and immune stimulators such as IL-2, etc. conjugated with antibody coated nanoparticles, then readministered along with Rock inhibitors or anti-integrins, GSK-inhibitors, and/or Wnt inhibitors to the patient after high intensity focused ultrasound (HIFU), or preferably low intensity focused ultrasound (LIFU), therapy of the entire tumor volume from inside the tumor to outside the tumor, the cellular immune therapy is administered in the tumor directly or intra-arterially and/or intravenously or subsequently used as vaccine for repeated therapy or therapy of the metastatic disease or potential recurrences.

In accordance with yet one or more other embodiments of the present invention, there is provided a cancer treatment and imaging method using compressive ultrasound comprising the steps of: (i) administering a plurality of antibody conjugated nanoparticles or liposomes to a patient in need thereof so as to target a tumor in the patient, the administered nanoparticles being coated with an antitumor antibody and a thermosensitive polymer, and the administered nanoparticles containing medication and/or gene and quenched fluorescein or doxorubicin in the thermosensitive polymer coating, at least some of the nanoparticles attaching to surface antigens of tumor cells of the tumor so as to form a tumor cell/nanoparticle complex; (ii) exciting the nanoparticles in a first compressive non-thermal mode (i.e., a LIFU mode) using a ultrasound generally less than 1 or 300 kHz and less than a one Watt power source that generates a focused compressive ultrasonic wave so as to peel off the thermosensitive polymer coating of the nanoparticles by the focused vibrational force of the ultrasonic wave, thereby releasing fluorescein or doxorubicin from the liposomes or nanoparticles into the circulation of the patient and the medication and/or gene at the tumor site; and (iii) imaging a body region of the patient so as to detect the fluorescein released into the circulation of the patient.

In a further embodiment of the present invention, in the first compressive non-thermal mode (i.e., a Low Intensity Focused Ultrasound (LIFU) mode), the focused ultrasonic wave generated by the ultrasound source has a frequency between about 1 kilohertz and about 100 kilohertz and has a power less than 1-3 W/cm² which is accompanied with none to minimal thermal energy of 38-43 degrees C. and it is easy to control since the temperature rise occurs more slowly than with HIFU, where the tumor cells are affected by the therapy while leaving the normal cells intact.

In yet a further embodiment, the method further comprises the steps of (i) heating the nanoparticles in a second thermal mode using the high intensity focused ultrasound (HIFU) source at a frequency of >1 MHz to 50 MHz and using a power of >1 Watt/cm² to 50 Watts/cm² or more as High Intensity Focused Ultrasound (HIFU) that raises the temperature of the tumor cell/nanoparticle complex to a temperature of about 41° C. to about 43° C. or higher so as to damage one or more tumor cell membranes at the tumor site and melt the thermosensitive polymer or lipid coating of the nanoparticles or liposomes, thereby releasing the fluorescein or doxorubicin into the circulation of the patient and the medication and/or gene at the tumor site; and (iii) alternating the heating of the nanoparticles in the second thermal mode with the focused compressive ultrasonic wave in the first compressive non-thermal mode (i.e., the LIFU mode) under the control of a processor controlling the thermal energy intensity and duration of the ultrasound source.

In one embodiment, the LIFU is used to focus inside a tumor (e.g., a brain tumor) to damage the core of the tumor and disrupt its blood brain barrier so that medication, etc. can reach the core of the tumor first, then gradually expand the coverage area until the peripheral borders of the tumor are reached so that the LIFU covers the entire tumor from inside the tumor to outside, thus contributing to increased access of the medications inside the tumor.

In one embodiment, a patient with a metastatic malignancy is treated initially with the antibody coated nanoparticles and/or liposomes conjugated with medications and immune stimulators, such as VLP, IL-2, IL-15, IL-17, IL-6, IL-1β, TNF-α, or toll-like receptors TLR 7 and TLR 8, to attach to the circulating exosomes and extracellular vesicles and circulating tumor cells to be destroyed by the body's cellular immune system and followed with the treatment of the main tumor as a second stage at the same session or another session of therapy to stimulate the immune response initially followed with the destruction of the main tumor. In one embodiment, the treatment is repeated in a scheduled fashion of bi-monthly therapy until the tumor or metastatic disease disappears by image analysis and liquid biopsy of the blood.

In one embodiment, the nanoparticles can be liposomes, micelles, or metallic nanoparticles having a lipid coating or another thermosensitive polymer coating or the nanoparticles can be liposomes which are loaded with metallic or non-metallic nanoparticles in their cavities.

In one embodiment, the heating source is an alternating magnetic field in low frequency range of 10-1000 Hertz or higher in which the rotation of the magnetic nanoparticles can release the polymeric coating conjugated with medication just by mechanical rotation, such as when one liposome is filled with a magnetic nanoparticle(s), or the medication can be conjugated with the nanoparticles and separated by the rotation of the nanoparticles induced by the alternating magnetic field.

In another embodiment, the alternating magnet has high frequencies of 10 kilohertz and more, up to 1 megahertz and more so that the thermal energy is proportional to the frequency of the magnetic field and the mechanical rotation of the nanoparticles, thereby generating thermal energy of 40 degrees C. to 60 degrees C. and more to denature the proteins.

In one embodiment, the thermal energy is created by a microwave or radiofrequency wave to increase the temperature of the nanoparticles to which the antibody coated nanoparticles are attached.

In still a further embodiment, in the second thermal mode, the ultrasonic wave generated by the ultrasound source has a frequency between about 150 kilohertz and about 300 kilohertz but a power of >1 W-50 W/cm2.

In yet a further embodiment, the nanoparticles are coated with one or more antibodies, and the antibody coated nanoparticles contain a medication and the medication is selected from the group consisting of Wnt inhibitors, Rock inhibitors, metformin, buformin, syrosingopine, phenformin, and the medication may provided in combination with a glutaminase inhibitor, such as Calithera, thus inhibiting glutamine uptake for the tumor to which the tumor cells are addicted. The medications may also include anti-VEGFs, such as Bevacizumab, Ranibizumab, Aflibercept, Avastin, anti-platelet derived growth factor (PDGF) agents, and/or notch pathway inhibitors, checkpoint inhibitors, and combinations thereof.

In one embodiment, one uses piezoelectric nanoparticles to generate a sound wave from an electric pulse (e.g., a pulse from a battery) from the exposed nanoparticles, such as in a telephone receiver, yet one can also use piezoelectric nanoparticles to expose the antibody conjugated nanoparticles to an ultrasonic pulse which is absorbed by the piezoelectric nanoparticles, such as boron nitride nanotubes, and convert the sound wave to an electric pulse. These principles allow one to image a tumor using an external electric pulse and antibody coated piezoelectric nanoparticles to create a sound wave inside the body to be imaged by an ultrasound transducer or apply the external ultrasound to antibody coated piezoelectric nanoparticles, such as boron nitride nanotube-filled liposomes, and deliver medications, etc. to treat a tumor non-invasively inside the body by depolarizing the tumor cell membranes by the generated electrons from the piezoelectric nanoparticles and make the cell membrane permeable to the medication (e.g., anti-cancer medication, genes, etc.) by the internally generated electrical pulses.

In one embodiment, the antibody coated piezoelectric nanoparticles or piezoelectric quantum dots or boron nitride nanotubes or carbon nanotube-filled liposomes may be administered intravenously, intra-arterially, topically, or locally by injecting them submucosally and/or spraying a nasal mucosa to travel through olfactory nerves or trigeminal nerves to the brain, piezoelectric nanoparticles can be rendered biocompatible by coating with them with zirconate titanate, perovskite-based oxides, alone or at low concentrations combined with samarium, barium titanate, polyvinylidene fluoride (PVDF), perovskites, piezoelectric antibody conjugated nanoparticles, nanoparticles coated with chitosan, PEG, biotin, streptavidin, radionuclides, CPPs, ACCPs, or Vitamin E, etc. for cell penetration to deliver drugs or genes, etc.

In accordance with still one or more other embodiments of the present invention, there is provided a cancer treatment and imaging method comprising the steps of: (i) systemically administering intravenously antibody coated piezoelectric or pyroelectric nanoparticles, and/or boron nitride nanotubes or carbon nanotubes, to a patient in need thereof so as to target a tumor in the patient, the piezoelectric or pyroelectric nanoparticles being coated with a thermosensitive polymer, and a medication being conjugated with the thermosensitive polymer coating of the piezoelectric or pyroelectric nanoparticles, the piezoelectric or pyroelectric nanoparticles travel through the body attaching to surface antigens of tumor cells of the tumor so as to form a tumor cell/nanoparticle complex; (ii) applying a pulsed electrical current to the piezoelectric or pyroelectric nanoparticles using an electrical source at the site of the tumor so as to create an electroacoustic sound from the piezoelectric or pyroelectric nanoparticles, and/or the boron nitride nanotubes or carbon nanotubes; (iii) recording the electroacoustic sound generated by the piezoelectric or pyroelectric nanoparticles, and/or the boron nitride nanotubes or carbon nanotubes, using a transducer to convert the electroacoustic sound to an electrical signal; and (iv) amplifying and transmitting the electrical signal to a processor so as is done with an ultrasonic imaging system, that a 1-dimensional, 2-dimensional, or 3-dimensional image of the tumor structure is able to be generated from the piezoelectric nanoparticles/tumor cells to produce an electroacoustic computed tomogram.

In another embodiment, the electroacoustic system can be used to scan the entire body, by systemically administering, or intravenously administering antibody coated piezoelectric or pyroelectric nanoparticles, boron nitride nanotubes, or carbon nanotubes to a patient in need thereof so as to target a tumor in the patient, metastatic lesion circulating tumor cells and the exosomes, the piezoelectric or pyroelectric nanoparticles being coated with a thermosensitive polymer, and a medication being conjugated with the thermosensitive polymer coating/CPP or vitamin E of the piezoelectric or pyroelectric nanoparticles, the piezoelectric or pyroelectric nanoparticles travel through the body attaching to surface antigens of tumor cells of the tumor so as to form a tumor cell/nanoparticle complex. A pulsed electrical current is applied from moveable electrodes placed stepwise from the top of head to the bottom of feet of the patient to the piezoelectric nanoparticles, boron nitride nanotubes, carbon nanotubes, or pyroelectric nanoparticles attached to the tumor cells while using an electrical pulse at the site so as to create an electroacoustic sound from the piezoelectric or pyroelectric nanoparticles attached to the tumor cells indicating the presence of the tumor in that area, reconstructing a full-body electroacoustic tomogram of the patient.

In one embodiment, the electrical pulse can vary from 1-30 pulse/sec (30 Hz) to 1 kHz, to 1 MHz or more or the electrical pulse can be longer than one second to induce various electrical responses in the tumor to which the antibody coated piezoelectric nanoparticles are attached.

In one embodiment, the lesion discovered by the electroacoustic tomogram is treated with focused ultrasound and antibody coated piezoelectric nanoparticles conjugated with either stimulating genes or inhibitory genes or genes are replaced using CRISPR-cas9 mediated Homology-Independent Targeted Integration (HITI) or Homology Directed Repair (HDR).

In another embodiment, the genetic composition of the tumor can be modified using electroacoustic tomography and administration of nanoparticle conjugates gene(s) using CRISPR/cas9 mediated Homology-Independent Targeted Integration (HITI) or Homology Directed Repair (HDR).

In one embodiment, a genetic disease is being treated, such as Alzheimer's disease, retinal degeneration, Parkinson's disease, muscular dystrophy, diabetes, or lung disease and other genetic diseases of the skin, kidney, or spinal cord, etc.

In a further embodiment of the present invention, the method further comprises the step of (v) increasing the permeability of one or more tumor cell membranes of the tumor using the pulsed ultrasound, thereby initiating an electric pulse from the piezoelectric nanoparticles (e.g. quartz or perovskites, coated with zirconate titanate, perovskite-based, oxides, barium titanate, polyvinylidene fluoride (PVDF)), piezoelectric nanoparticles, nanoparticles coated with chitosan, PEG, biotin, streptavidin, radionuclides, CPPs, ACCPs or Vitamin E to depolarize the tumor cells attached to the nanoparticles, and minimally facilitating the entry of the medication into the depolarized tumor cells of the tumor.

In a further embodiment of the present invention, the method further comprises the step of (v) increasing the permeability of one or more tumor cell membranes of the tumor using the pulsed focused ultrasound, thereby initiating an electric pulse from the piezoelectric nanoparticles (e.g., boron nitride nanotubes or carbon nanotubes, quartz or perovskites, conjugated with a medication, CPP, gene or VLP exposed to the ultrasound to depolarize the tumor cells while increasing the temperature of the tissue with the focused ultrasonic waves and simultaneously measuring the tissue temperature with second harmonic wave backscattered ultrasound generated from the heated tissue recorded with a transducer, located on the patient's skin, and connected to an imaging system, recording the tissue temperature and enhancing penetration of piezoelectric nanoparticles inside the cell that release the medication, etc. to damage the tumor cells. This imaging unit, is in turn connected via a software to the initial focused ultrasound producing unit, controlling the intensity of the pulsed ultrasound keeping it at <10 kHz intensity and the power at <1 W/cm² power to peel off and release medication from the nanoparticles (e.g., from antibody coated quartz or perovskites nanoparticles conjugated with medication, similar to a commercially available ultrasonic watch or instrument cleaner removing the dirt and cleaning from the instrument), and to simultaneously depolarize the tumor cells attached to the piezoelectric nanoparticles, such as boron nitride nanotubes or carbon nanotubes, facilitating the entry of the medication into the depolarized tumor cells membrane and as needed to heat them under the control of power of the focused ultrasound connected to the thermal imaging system via a processor.

In yet a further embodiment, the method further comprises the step of (v) heating the piezoelectric or pyroelectric nanoparticles, such as boron nitride nanotubes or carbon nanotubes, using a ultrasound source operating in a thermal mode as high intensity focused ultrasound (HIFU) so as to raise the temperature of the tumor cell/nanoparticle complex controllably to a temperature of about 41° C. to about 43° C., thereby damaging one or more tumor cell membranes at the tumor site and melting the thermosensitive polymer coating of the nanoparticles to release the medication at the tumor site.

In still a further embodiment, in a patient (e.g., with a thyroid tumor), the electrical source comprises a battery device with anode/cathode leads positioned on a first side of the body (e.g., neck) of the patient and a cathode being located on a second side of the neck of the patient, the pulsed electrical current passing through the neck of the patient from the anode to the cathode of the battery device, and where the pulsed electrical current passes through a tumor pretreated with intravenous or intra-arterial injection of the tumor supplying artery with antibody coated piezoelectric nanoparticles conjugated with medication attached to the tumor cells, where an electrical pulse creates an ultrasonic wave from the piezoelectric nanoparticles that can be recorded by an ultrasonic transducer located on the skin, imaged and localize the tumor precisely, then the lesion is treated non-invasively with a focused ultrasound beam through the skin and simultaneously heat up the tissue to the temperature of 39-40 degrees C. to damage the tumor cells with thermal energy and depolarize the tumor cell membranes by converting the sound waves to an electric pulse to depolarize the tumor cells exposed to the ultrasound, making the tumor cells permeable to the released medication/gene, etc.

In accordance with still one or more other embodiments of the present invention, there is provided a cancer treatment method using focused ultrasound comprising the steps of: (i) administering a plurality of piezoelectric or pyroelectric nanoparticles to a patient in need thereof so as to target a tumor in the patient, the administered piezoelectric or pyroelectric nanoparticles being coated with an antitumor antibody and a thermosensitive polymer, and the administered piezoelectric or pyroelectric nanoparticles containing medication, a gene, a checkpoint inhibitor, and/or viral-like particles (VLP), 1L-2, ACPP, a toxin, TLR 7/8 to stimulate cellular immune response and quenched fluorescein or doxorubicin in the thermosensitive polymer coating, at least some of the piezoelectric or pyroelectric nanoparticles attaching to surface antigens of tumor cells of the tumor so as to form a tumor cell/nanoparticle complex; and (ii) stimulating the piezoelectric or pyroelectric nanoparticles in a thermal or non-thermal mode (LIFU) using a focused ultrasound source that generates a focused ultrasonic wave so as to produce an electrical current from the piezoelectric or pyroelectric nanoparticles that paralyses cells of the tumor, thus permitting piezoelectric or pyroelectric nanoparticles with the antitumor antibody coating to enter the cytoplasm of the tumor cells and release the medication, gene, checkpoint inhibitor, and/or VLP, etc. inside the tumor cells when the medication, gene, checkpoint inhibitor, and/or VLP, CD40, TLR3, or TLR7 is released from the thermosensitive polymer coating of the piezoelectric or pyroelectric nanoparticles upon the heating of the nanoparticles to a temperature of about 41° C. to about 43° C.

In a further embodiment of the present invention, the antibody coated piezoelectric or pyroelectric nanoparticles are further conjugated with cell penetrating peptides (CPPs) or activatable cell-penetrating peptides (ACPPs) so to enhance cell penetration into the cells of the tumor prior to treatment to release the medication inside the tumor cells during non-thermal therapy with focused ultrasound at pulses of <100 kHz and power of <1 W/cm².

In yet a further embodiment, the nanoparticles are coated with one or more antibodies, and the antibody coated nanoparticles contain medication and the medication is selected from the group consisting of Wnt inhibitors, Rock inhibitors, GSK inhibitors, metformin, buformin, syrosingopine, phenformin, or in combination with a glutaminase inhibitor, such as Calithera, thus inhibiting glutamine uptake for the tumor to which the tumor cells are addicted, anti-VEGFs, such as Bevacizumab, Ranibizumab, Aflibercept, Avastin, and anti-platelet derived growth factor (PDGF) agents and/or notch pathway inhibitors, and checkpoint inhibitors, and combinations thereof further conjugated with cell penetrating peptides (CPPs) or activatable cell-penetrating peptides (ACPPs) so as to enhance cell penetration into the cells of the tumor prior to treatment to release the medication inside the tumor cells during non-thermal therapy with focused ultrasound at pulses or <100 KHz and power of <1 W/cm².

In accordance with yet one or more other embodiments of the present invention, there is provided a cancer treatment method comprising the steps of: (i) growing genetically modified CAR-T cells and/or natural killer cells in vitro with tumor antigens, viral-like particles (VLPs), and/or immune stimulators conjugated with tumor-antibody-coated nanoparticles for providing cellular immune therapy to a patient; and (ii) administering the cellular immune therapy together with checkpoint inhibitors, Rock inhibitors, anti-integrins, GSK-inhibitors, and/or Wnt inhibitors to the patient after ultrasound therapy of an entire tumor volume from inside the tumor to outside the tumor. The cellular immune therapy is administered in the tumor directly, intra-arterially in the patient, and/or intravenously in the patient, and/or the cellular immune therapy is subsequently used as vaccine for repeated therapy or therapy of a metastatic disease or potential recurrences of the metastatic disease.

In a further embodiment of the present invention, the immune stimulators conjugated with tumor-antibody-coated nanoparticles are selected from the group consisting of bee venom, scorpion venom, viral-like particles (VLPs), IL-2, TLR 7, and combinations thereof.

In yet a further embodiment, the ultrasound therapy comprises high intensity focused ultrasound (HIFU) therapy and/or low intensity focused ultrasound (LIFU) therapy.

It is to be understood that the foregoing general description and the following detailed description of the present invention are merely exemplary and explanatory in nature. As such, the foregoing general description and the following detailed description of the invention should not be construed to limit the scope of the appended claims in any sense.

### BRIEF DESCRIPTION OF THE DRAWINGS

The invention will now be described, by way of example, with reference to the accompanying drawings, in which:

FIG. 1 illustrates a schematic diagram of a cancer treatment and evaluation system, according to an embodiment of the invention;

FIG. 2 illustrates a schematic diagram of a cancer treatment and imaging system, according to another embodiment of the invention;

FIG. 3 illustrates a schematic diagram of a cancer treatment system, according to yet another embodiment of the invention, wherein a thyroid tumor is being treated;

FIG. 4 illustrates a schematic diagram of a cancer treatment and imaging system, according to still another embodiment of the invention, wherein a thyroid tumor is being treated and imaged; and

FIG. 5 illustrates a schematic diagram of a cancer treatment and imaging system, according to yet another embodiment of the invention, wherein a thyroid tumor is being treated and imaged.

Throughout the figures, the same elements are always denoted using the same reference characters so that, as a general rule, they will only be described once.

### DETAILED DESCRIPTION OF EMBODIMENTS OF THE INVENTION

Herein, in one embodiment, the controlled localized thermotherapy, damages the tumor cells releasing their antigens augmenting the immune response without weakening the natural humoral or cellular response to the tumor cells.

In one embodiment, the use of controlled thermotherapy at 41-43 degrees C. at the tumor site using antibody coated nanoparticles weakens the tumor cells that preferentially are sensitive to heat compared to the normal cells and makes them susceptible to antitumor medication and/or immunotherapy reducing their ability to become chemo-resistant.

In one embodiment, the use of controlled thermotherapy at 41-43 degrees C. at the tumor site using antibody coated nanoparticles weakens the tumor cells that preferentially are sensitive to heat compared to the normal cells and makes them less likely to change their genetic composition to become less resistant to heat since the thermal effect at generally about 1 MHz to 50 MHz or 100 MHz and one Watt or more of HIFU is instantaneous when the temperature reaches these levels, or is maintained at the temperatures of these levels (41-43 C) for a period of 5-10 minutes under the control of thermal delivery system and a processor.

In one embodiment, the nanoparticles absorb preferentially either electromagnetic radiation or ultrasound, or respond to an alternating magnetic field, or a combination of them that can be heated to a desired degree of temperature under the control of a processor that records the change of the temperature in the body for a period of time, or increases it to higher detectable level of 56 C degrees, and imaged with a thermoacoustic imaging system for control of the temperature using backscattered ultrasound waves via a transducer.

In one embodiment, the alternating magnetic field is generated by applying alternating current to a magnet that creates the magnetic field that reverses itself and thereby activating the magnetic nanoparticles that lay in the magnetic field, thus creating an automatic real-time feedback control system, and a processor is measured the alternating magnetic field's intensity and the temperature created at the site of the magnetic nanoparticles induced by the change of its magnetic field and the tumor to which the nanoparticles are attached, and the alternating magnetic field heats up the antibody coated magnetic particles/tumor, or if the nanoparticles are incorporated inside liposomes, breaks up the liposome's wall releasing its contents, such as dye or medication or in combination, at about 40-41 degrees C. temperature in the circulation.

In one embodiment, the antibody coated nanoparticles are chosen to last in the tumor cells and the treatment can be repeated locally (e.g., with breast cancer, ovarian cancer, cancer of brain tumors, lung cancer, prostate cancer) as desired without overheating the body, and the tumor can be imaged post treatment to examine the effect of the therapy on the tumor.

In one embodiment, the pluralities of antibody coated nanoparticles, liposomes, for example, formed from magnetic and paramagnetic fullerene, carry thermosensitive polymers, such as chitosan hyaluronic acids which melts at a 41-43 degree C. temperature and can release antineoplastic medication, gene modifying RNAi, CRISPR cas 9, etc., or gene modification is done through CRISPR cas9 or Cas9/gRNA ribonucleoprotein complexes (Cas9 RNPs) mediated Homology-Independent Targeted Integration (HITI) or Homology Directed Repair (HDR). The pluralities of nanoparticles may also carry and release checkpoint inhibitors and/or Rock inhibitors from the thermosensitive polymer coating so as to attack the tumor cells with multiple modalities, and obtain post treatment biomarkers from the circulation for creating an appropriate vaccine of each patient's tumor for the future administration of the vaccine intravenously, subcutaneously or inside the tumor combined with antibody coated nanoparticles/checkpoint inhibitors, such as PD-1, Jagged 1 inhibitor 15D11, etc., Rock inhibitors, such as Fasudil, anti-VEGFs and/or a Wnt inhibitor, such as niclosamide, to reactivate the immune cells to attack the tumor cells with multiple modalities as needed in any cancer therapy, and the Rock inhibitor, or in combination with anti-VEGFs, such as Bevacizumab, Ranibizumab, Aflibercept, Avastin, and/or PDGF inhibitors, and/or notch pathway inhibitors, thus reducing TGF-β production after therapy and the subsequent scar formation.

In one embodiment, vaccination is done every week for a month, and then every three months for the first year, then every six months and beyond to enhance immunity of the patient and prevent recurrences.

In one embodiment, the pluralities of antibody coated nanoparticles or liposomes carry with their thermosensitive polymeric coating one or multiple medications, such as cobimetinib, an MEK1 and MEK2 inhibitor, BRAF and MEK inhibitors, sorafenib, vemurafenib, dabrafenib, pembrolizumab, a check point inhibitor, ipilimumab, nivolumab, olaparib, niraparib, pazopanib, dacarbazine, temozolomide, imatinib, carmustine, cisplatin, carboplatin, and paclitaxel, or a combination thereof for treatment of brain cancers, ovarian cancer, melanoma, lung cancer, in combination with controlled thermotherapy using an internal or external energy source, such as LIFU or HIFU or alternating magnetic field, etc. at a temperature of 41-43 C degree for 1-10 minutes as predetermined by the physician under the control of a processor.

In one embodiment, the pluralities of antibody coated nanoparticles including piezoelectric nanoparticles or bubble liposomes carrying fluorescein/doxorubicin which contain air pockets or perfluorocarbon (PFC) nanoemulsions carry with their thermosensitive polymeric coating one or multiple antineoplastic medications, check point inhibitors etc. are used for treatment of cancer using a combination of focused ultrasound LIFU or HIFU, or alternating magnetic field (or electromagnetic radiation, non-invasively at a temperature 41-43 degrees C., and then imaged with a thermoacoustic imaging system for control of the temperature under the control of a processor controlling thermal energy, along with low-intensity (1-3 V/cm), low frequency 1-50-kHz or intermediate frequency 100-300 kHz non-thermal as LIFU and more than 1 MHz to create thermal energy as LIFU achieving thermotherapy, electroacoustic imaging using an array of ultrasound transducers, focused ultrasound, or a moving transducer, etc. that can move and record the produced sounds from the piezoelectric nanoparticles transmitted to a processor or computer to 2-3D electroacoustic images or live video or reconstructed by a computer software, such as in focused ultrasound, and dielectric effect on the cellular components of the tumors, such as brain tumors, lung cancer, ovarian cancer, breast cancer, prostate cancer, and other cancers, which can be repeated numerous times post therapy to eliminate the cancer.

In one embodiment, one can diagnose and image an early stage cancer in a genetically predisposed person, using a combination of cancer antibodies attached to a pluralities of nanoparticles with activatable cell-penetrating peptides (ACPPs), injected systemically intravenously or in the cerebrospinal fluid to travel to the suspected tumor location and attach to the tumor receptors, applying external thermal energy to the suspected area or body, create a photoacoustic or thermoacoustic sound that can be recorded by a transducer indicating thermal expansion of the nanoparticle and the degree of the temperature imaged, and the location indicating the presence of a lesion.

In one embodiment, the antibody coated pluralities of nanoparticles or liposomes with activatable cell-penetrating peptides (ACPPs) are selected from magnetic, paramagnetic, non-magnetic sphere, rod, nanocarbon, nanowire, nanorod, nanowire, magnetic nanoshells, nanocages, gold nanoshells or silica-gold nanoshells, silica iron oxide nanoshells, gold coated ferric oxide, quantum dots, magnetic and paramagnetic fullerene, encapsulate ferromagnetic nanoclusters, dendrimers, micelles of perfluorocarbon liquids, liposomes, liposomes in combination with the nanoparticle, nanoshells carrying quenched fluorescein or doxorubicin, fluorescent dextrans/dye or liposomes or polymer micelles or nanoparticles usually poly(N-isopropyl acrylamide carrying a dye indicator in thermosensitive polymers that are administered to a patient to seek the potential tumor cells and attach to them exposed to the external energy source to release the fluorescein in the circulation at 41-43 degrees C. indicating the temperature at the tumor or nanoparticle site attached to the tumor.

In one embodiment, the energy delivery system is a focused ultrasound which heats up as HIFU, the tissue located at the focal point of the focused ultrasound using generally about a frequency of 2 MHz to 50 MHz or 100 MHz and more than one Watt and an antibody coated ultrasound contrast agent, such as gold nanoparticles (GNPs), gold nanorods (GNRs), gold nanoshells (GNS), graphene oxides (GOs), polypyrrole (PPy) nanocapsules, or nanoshells nanocage magnetic, gold, silicone, carbon, piezoelectric nanotubes, perfluorocarbon (PFC) nanoemulsions, the mixture of magnetic nanoparticles, piezoelectric nanotransducers, boron nitride nanotubes, perovskites, and quenched fluorescein or doxorubicin, fluorescent dextrans, or another dye indicator with the thermosensitive polymer. The temperature is measured non-invasively by the estimation of the temperature depending on acoustic harmonic or backscattered ultrasound from 26-46 C degree generated by nonlinear ultrasound wave.

In one embodiment, the thermal energy delivery system is a focused ultrasound which is capable of either producing non-thermal focused ultrasound at a single frequency of, for example, <100 kHz and less than one Watt of power, in a non-thermal mode LIFU by regulating its duration by the software of the system to deliver focused ultrasonic pulses of a few microsecond duration or repeated in an interval that permits dissipation of thermal energy before the second pulse arrives while the same system can deliver focused ultrasound, for example, at <1 MHz at a power of less than one Watt.

In one embodiment, only the non-thermal mode, low intensity focused ultrasound (LIFU), such as ultrasound with a frequency of generally about >300 kHz and less, and than less one Watt, is used with pluralities of antibody coated ultrasound contrast agents, or liposome-filled nanoparticles such as gold nanoparticles (GNPs), gold nanorods (GNRs), gold nanoshells (GNS), graphene oxides (GOs), polypyrrole (PPy) nanocapsules, or nanoshells, nanocages; magnetic, gold, silicone, carbon nanotubes, perfluorocarbon (PFC) nanoemulsions, piezoelectric nanotubes, or piezoelectric boron nitride nanotubes, nanogel, liposome and releases antineoplastic medication/dye, a gene, and a polymeric coating conjugated with quenched fluorescein or another dye or indicator, such as polymeric materials, such as poly(γ-2-(2-(2-methoxyethoxy)-ethoxy)ethoxy-ε-caprolactone)-b-poly(γ-octyloxy-ε-caprolactone) and a dye, have demonstrated significant transition temperatures, allowing improved drug release at low hyperthermia (<40° C.), and the release is recognized by the presence of the fluorescein in the circulation in the body or under the nail bed or any other part of the body.

In one embodiment, only the thermal mode of the focused ultrasound is used with pluralities of antibody coated ultrasound contrast agents, such as gold nanoparticles (GNPs), liposome-filled nanoparticles, gold nanorods (GNRs), gold nanoshells (GNS), graphene oxides (GOs), polypyrrole (PPy) nanocapsules, or nanoshells, nanocages; magnetic, gold, silicone, carbon nanotubes, perfluorocarbon (PFC) nanoemulsions, piezoelectric nanotubes, or piezoelectric boron nitride nanotubes, nanogels, liposome and releases an antineoplastic medication/dye, a gene, and a polymeric coating conjugated with quenched fluorescein or doxorubicin or another dye or indicator, such as polymeric materials, such as poly(γ-2-(2-(2-methoxyethoxy)-ethoxy)ethoxy-ε-caprolactone)-b-poly(γ-octyloxy-ε-caprolactone) and a dye, have demonstrated significant transition temperatures, allowing improved drug release at low hyperthermia (40 to 43° C.), measured by acoustic backscattered ultrasound and the release is recognized by the presence of the fluorescein in the circulation in the body or under the nail bed or any other part of the body

In one embodiment, the non-thermal mode LIFU and thermal mode of the focused ultrasound or high intensity focused ultrasound (HIFU) is used sequentially with pluralities of antibody coated ultrasound contrast agents, liposome-filled nanoparticles, such as gold nanoparticles (GNPs), gold nanorods (GNRs), gold nanoshells (GNS), graphene oxides (GOs), polypyrrole (PPy) nanocapsules, or nanoshells, nanocages, magnetic, gold, silicone, carbon nanotubes, perfluorocarbon (PFC) nanoemulsions, piezoelectric nanotubes, or piezoelectric boron nitride nanotubes, nanogels, liposome and releases antineoplastic medication/dye, a gene, and a polymeric coating conjugated with quenched fluorescein another dye or indicator, such as polymeric materials, such as poly(γ-2-(2-(2-methoxyethoxy)-ethoxy)ethoxy-ε-caprolactone)-b-poly(γ-octyloxy-ε-caprolactone) and a dye, have demonstrated significant transition temperatures, allowing improved drug release at low hyperthermia (<40 to 43° C.), and the release is recognized by the presence of the fluorescein in the circulation in the body or under the nail bed or any other part of the body.

In one embodiment, the non-thermal mode and thermal mode of the focused ultrasound is used sequentially or simultaneously with another source of energy radiation or x-ray at lower doses than normally recommended to achieve a complementary effect on the tumors using pluralities of antibody coated ultrasound contrast agents, liposomes filled with nanoparticles such as gold nanoparticles (GNPs), gold nanorods (GNRs), gold nanoshells (GNS), graphene oxides (GOs), polypyrrole (PPy) nanocapsules, or nanoshells, nanocages; magnetic, gold, silicone, carbon nanotubes, perfluorocarbon (PFC) nanoemulsions, piezoelectric nanotubes, or piezoelectric boron nitride nanotubes, nanogels, liposome and releases antineoplastic medication/dye, a gene, and a polymeric coating conjugated with quenched fluorescein another dye or indicator, such as polymeric materials, such as poly(γ-2-(2-(2-methoxyethoxy)-ethoxy)ethoxy-ε-caprolactone)-b-poly(γ-octyloxy-ε-caprolactone) and a dye, have demonstrated significant transition temperatures, allowing improved drug release, from antibody coated nanoparticles carrying Rock inhibitors, or a combination of anti-VEGF or PDGF inhibitors, notch pathway inhibitors, Wnt inhibitors to reduce TGF-β production after therapy and the subsequent scar formation and antibody coated nanoparticles/checkpoint inhibitors and VLP to prevent the localized or circulating tumor cells and their exosomes carrying PD-L1 from disguising themselves, thus being recognized by the T-cells, which together with killer cells phagocytose them, and at low hyperthermia (<40 to 43° C.), and the release is recognized by the presence of the fluorescein in the circulation in the body or under the nail bed or any other part of the body.

In one embodiment, the thermal energy delivery system is a focused ultrasound which vibrates the tissue located at the focal point of the focused compressive ultrasound (e.g., 1-40 kHz frequency) using an antibody coated ultrasound contrast agent, lipid nanoparticles, or liposomes filled with nanoparticles such as gold nanoparticles (GNPs), gold nanorods (GNRs), gold nanoshells (GNS), graphene oxides (GOs), polypyrrole (PPy) nanocapsules, or nanoshells, nanocages; magnetic, gold, silicone, carbon nanotubes, perfluorocarbon (PFC) nanoemulsions, piezoelectric nanotubes, or piezoelectric boron nitride nanotubes, and releases antineoplastic medication, a gene, and a polymeric coating conjugated with quenched fluorescein another dye or indicator, such as polymeric materials, such as poly(γ-2-(2-(2-methoxyethoxy)-ethoxy)ethoxy-ε-caprolactone)-b-poly(γ-octyloxy-ε-caprolactone) and a dye, have demonstrated significant transition temperatures, allowing improved drug release at low hyperthermia (40° C.), and the release is recognized by the presence of the fluorescein in the circulation in the body or under the nail bed or any other part of the body.

In one embodiment, the energy delivery system is a focused ultrasound in a compressive non-thermal mode LIFU (e.g., at 1-100 kHz and <1 W to 2-4 W), which is visualized in the tissue by ultrasonic imaging only, the top of the cone-shaped image of the tissue is seen where the tip of the focused cone exerts the compressive or pressure force without producing significant thermal effect, or in combination with another imaging modality such as MRI or CT-scan or PET-scan that defines the focal point of the focused compressive ultrasound in relationship with the other body's structures, using an antibody coated ultrasound contrast agent such as gold nanoparticles (GNPs), gold nanorods (GNRs), gold nanoshells (GNS), graphene oxides (GOs), polypyrrole (PPy) nanocapsules, or nanoshells nanocages, magnetic, gold, silicone, carbon nanotubes, piezoelectric nanotubes, the mixture of magnetic nanoparticles, perfluorocarbon (PFC) nanoemulsions, piezoelectric nanotransducers, boron nitride nanotubes, lipid nanoparticles, or liposomes filled with nanoparticles or quenched fluorescein, fluorescent dextrans or another dye or indicator with chitosan, liposomes, or polymer micelles or nanoparticles usually poly(N-isopropyl acrylamide carrying a dye, or bubble liposomes carrying fluorescein which contain air pockets or nanoemulsions of PFC measuring the release of medication non-invasively by the release of fluorescein in the circulation.

In one embodiment, the energy delivery system is a focused ultrasound in a thermal mode which is visualized in the tissue by ultrasonic imaging only by seeing the top of the cone shaped image of the tissue where the tip of the focused cone produces a thermal effect seen as an increase in thermal coagulation and whitening of the tissue effect or in combination with another imaging modality such as MRI that defines the focal point of the focused compressive or pressure mode ultrasound LIFU (e.g., at 1-100 kHz) in relationship with the other body's structures, using an antibody coated ultrasound contrast agent, such as gold nanoparticles (GNPs), magnetic or paramagnetic nanoparticles, gold, silicone, carbon nanotubes perfluorocarbon (PFC) nanoemulsions, piezoelectric particles or nanotubes, the mixture of magnetic nanoparticles, piezoelectric nanotransducers, boron nitride nanotubes, lipid nanoparticles, or liposomes filled with nanoparticles, or quenched fluorescein, fluorescent dextrans or another dye or indicator with chitosan, liposomes, or polymer micelles or nanoparticles usually poly(N-isopropyl acrylamide) carrying a dye or polymeric materials, such as poly(γ-2-(2-(2-methoxyethoxy)-ethoxy)ethoxy-ε-caprolactone)-b-poly(γ-octyloxy-ε-caprolactone)7, which have demonstrated significant transition temperatures, allowing improved drug release at low hyperthermia (e.g., at 40° C.), gold nanorods (GNRs), gold nanoshells (GNS), graphene oxides (GOs), polypyrrole (PPy) nanocapsules, or nanoshells, nanocages, with perfluorocarbon hexane, sealed nanotubes with a non-toxic gas such as SF6, that expands with thermal energy of either focused thermal ultrasound, or high intensity focused ultrasound (HIFU), or low intensity focused ultrasound (LIFU), electromagnetic radiation, RF, microwave, or an alternating magnetic field in case of magnetic nanotubes etc., measuring the release of medication non-invasively at temperature of 41-43 degrees C. seen by the release of fluorescein in the circulation or ultrasound backscattered energy.

In one embodiment, the patient is exposed to external or internal thermal energy to heat the antibody coated nanoparticles using laser, electromagnetic radiation, visible or infrared light, microwave, radiofrequency, a focused ultrasound, or an alternating magnetic field to heat and damage the tumor cells and increase tumor biomarker in the circulation after the thermotherapy having an important diagnostic value (i.e. indicating presence of a tumor by increased biomarkers) and therapeutic value for vaccine production after harvesting the circulating biomarkers and vaccine production for the future management of the tumor recurrences in the patient to reactivate the immune response and kill the tumor cells.

The standard ultrasonic transducer produces a divergent wave of ultrasound such that its tissue penetration depends on its frequency and the tissue property. The higher the frequency of the ultrasound, the lower is its transmission into the tissue. However, the ultrasound wave can be made to focus at a certain distance from its transducer head by using a concave ultrasonic transducer using a single array or multiple arrays.

In one embodiment, the physical effects of the focused ultrasonic waves depend on the power and intensity of the ultrasound wave. At a low power of about less than one Watt/cm² (e.g., low power 0.3-1 Watts), and frequencies of 1-140 KHz, 140 KHz-1 MHz or higher, the focused ultrasound has a more compressive, and none, to minimal significant thermal effect (i.e., non-thermal or compressive mode LIFU), while the high power of 1-100 Watts/cm², etc. and frequencies of 40 KHz-3 MHz or more show an increased thermal effect (i.e., thermal mode HIFU), and those with a high power of 3-50W or more and frequencies of 0.5-50 MHz or higher, produce thermal coagulative/ablative properties (i.e., in a coagulative and ablative mode).

In one embodiment, the second or third harmonic ultrasound back scatter is measured indicating the temperature at the focal point of the focused ultrasound in the tissue.

In one embodiment, the focused ultrasound can be imaged simultaneously with a non-focused ultrasound in order to image the lesion.

In one embodiment, the energy source heats the antibody coated nanoparticles conjugated with medication, etc. using a focused low power compressive or pressure mode ultrasound, and the nanoparticles with cell penetrating agents or activatable cell penetrating agents and quenched fluorescein or another dye or indicator with polymeric materials, such as poly(γ-2-(2-(2-methoxyethoxy)-ethoxy)ethoxy-ε-caprolactone)-b-poly(γ-octyloxy-ε-caprolactone) and a dye have demonstrated significant transition temperatures, allowing improved drug release at low hyperthermia (40° C.), are injected in the patient's circulation, lymphatic vessels, inside a body cavity, and the focused ultrasound is applied in the compressive low power non-thermal mode 1-40 KHz, where the dye is merely peeled off the nanoparticles or the polymeric carrier by its focused vibrational force. In one embodiment, the energy source, which uses focused compressive or pressure mode ultrasound with low frequencies 1-140 kHz, and <0.3 W is used with antibody coated nanoparticles that are conjugated with a medication, a gene, etc., and contain at least one radioactive agent conjugated with cell penetrating agents or activatable cell penetrating agents and quenched fluorescein, fluorescent dextrans, liposomes filled with fluorescein or another dye or indicator, or bubble liposomes carrying fluorescein which contain air pockets or nanoemulsions of PFC. The nanoparticles are injected in the patient's circulation, lymphatic vessels, inside a body cavity, or cerebrospinal fluid, and the focused low power ultrasound is applied in the compressive or pressure mode (i.e., a non-thermal mode) where the dye is merely peeled off the nanoparticles by the focused vibrational force of the ultrasound while the tissue is imaged by the ultrasound or computer-assisted ultrasound imaging.

In one embodiment, the antibody coated nanoparticles are injected in the body, and are exposed to thermal energy, such as electromagnetic radiation. The nanoparticles absorb the energy and expand producing a sound wave that is known as photoacoustic sound which is recorded by a transducer and converted to an electrical signal transmitted to a processor, and then converted to 1D, 2D, or 3D computerized images or one can create a dual system imaging using the ultrasound imaging combined with photoacoustic imaging inside the tissue.

In one embodiment, antibody coated piezoelectric nanoparticles pyroelectric nanoparticles, or perfluorocarbon (PFC) nanoemulsions are injected inside the body, and the nanoparticles attach to the surface antigen of the normal cells or tumor cells and, when the nanoparticles are exposed to a pulse of electrical current, a sound is created by piezoelectric nanoparticles inside the body (called an electroacoustic sound) that can be recorded with a transducer, using an array of the ultrasound receivers or a single moving transducer that can move and record the produces sounds from the piezoelectric nanoparticle transmitted to a processor or computer to 2-3D electroacoustic images or live video, and then the signal is amplified and forwarded to a processor in order to be converted to a 1D, 2D, or 3D image as an electroacoustic computed tomogram of the structure.

In one embodiment, antibody coated piezoelectric nanoparticles pyroelectric nanoparticles, or perfluorocarbon (PFC) nanoemulsions are injected inside the body, and the nanoparticles attach to the surface antigen of the normal cells or tumor cells and, when exposed to a pulse of electrical current, a sound is created by piezoelectric nanoparticles inside the body (call an electroacoustic sound) that can be recorded with a transducer, and then the signal is amplified and forwarded to a processor in order to be converted to a 1D, 2D, or 3D image as an electroacoustic computed tomogram of the structure using a multi-view Hilbert transformation method to recover the unipolar initial pressure for full-ring electroacoustic computed tomography.

In one embodiment, the antibody coated perfluorocarbon (PFC) nanoemulsions, or piezoelectric or pyroelectric nanoparticles are coated with thermosensitive polymers, such as chitosan, liposomes, liposomes filled with fluorescein or polymer micelles or nanoparticles usually poly(N-isopropylacrylamide) carrying a dye quenched with fluorescein, fluorescent dextrans or another dye or indicator, which is able to be released when the nanoparticles are exposed to electromagnetic radiation, microwave, radiofrequency, ultrasound, or an alternating magnetic field to raise the temperature of piezoelectric or pyroelectric nanoparticles to 41 to 43 degrees C. The piezoelectric or pyroelectric nanoparticles are injected inside the body, and attach to the surface antigens of normal cells or the surface antigens of tumor cells and, when exposed to a pulse of electrical current with an adjustable signal frequency and voltage, an acoustic response is produced by the electrical stimulation of piezoelectric nanoparticles inside the body (called an electroacoustic sound) that can be recorded using an array of the ultrasound receivers or a moving transducer that can move and record the sounds from the piezoelectric nanoparticles transmitted to a processor or computer to 2-3D electroacoustic images or live video or the signal is amplified and forwarded to a processor in order to be converted to a 1D, 2D, or 3D image as an electroacoustic computed tomogram, where the system is equipped with a high frequency linear transducer array for mapping the microvascular network of, e.g., the brain, lung, breast, genitourinary tract, digestive tract, and studying its physical and hemodynamic activities in the organ.

In one embodiment, thermal and electrical energy is used to influence the permeability of the tumor cell membrane so as to utilize the effect or thermotherapy to enhance the effect of chemotherapy or immune therapy, gene therapy, gene modification using CRISPR-cas9 or Cas9 RNPs in benign and malignant cells, such as brain and spinal cord tumors, breast cancer, lung cancer, prostate and ovarian cancer and melanoma, glioblastoma, retinoblastoma, meduloblastoma, gastrointestinal and genitourinary tumor of sarcoma, etc. Nanoparticles are used having pyroelectric or piezoelectric characteristics, and the antibody coated nanoparticle tube complex is imaged with an electroacoustic computed tomography imaging system.

In one embodiment, the antibody coated pluralities of gold, piezoelectric, iron oxide, nanocage, nanotube, nanoshell, magnetic, paramagnetic, pyroelectric, perfluorocarbon (PFC) nanoemulsions, and piezoelectric nanoparticles are coated with thermosensitive polymers, such as chitosan, liposomes, or polymer micelles or nanoparticles usually poly(N-isopropyl acrylamide) carrying a dye or bubble liposomes carrying fluorescein which contain air pockets or nanoemulsions of PFC and conjugated with a medication and/or gene, with cell penetrating agents or activatable cell penetrating agents, and with quenched fluorescein or another dye or indicator, then injected in the patient's circulation, lymphatic vessels, inside a body cavity, cerebrospinal fluid or inside the tumor, etc. When the nanoparticles are exposed to electromagnetic radiation, microwaves, radiofrequency, ultrasound, or an alternating magnetic field the temperature of the piezoelectric or pyroelectric nanoparticles and the tumor cell complex is raised to 41-43 degrees C. to release the quenched fluorescein or another dye or indicator. When injected into the body, the piezoelectric or pyroelectric nanoparticles attach to the surface antigens of normal cells or the surface antigens of tumor cells and, when exposed to a pulse of electrical current with an adjustable signal frequency and voltage, an acoustic response is produced by electrical stimulation of the piezoelectric or pyroelectric nanoparticles inside the body (i.e., called an electroacoustic sound) that can be recorded by an array of transducers or a single moving transducer, and then the signal is amplified and forwarded to a processor in order to be converted to a 1D, 2D, or 3D or live real time video of a stable lesion or pulsating lesion as an electroacoustic computed tomogram using the movement of the piezoelectric nanoparticles to be evaluated by the Doppler of the blood vessels for blood flow measurement.

In one embodiment, focused compressive ultrasound at low power, 1-40 kHz is used with pluralities of antibody coated nanoparticles conjugated with a medication and/or gene, and conjugated with cell penetrating agents or activatable cell penetrating agents and quenched fluorescein liposomes, liposomes filled with fluorescein or, or polymer micelles or nanoparticles usually poly(N-isopropyl acrylamide) carrying a dye or an indicator injected in the patient's circulation, lymphatic vessels, inside a body cavity, cerebrospinal fluid, etc. where the dye, gene, and/or medication is merely peeled off the nanoparticles by the focused vibrational force of the focused low power ultrasound in a non-thermal mode (LIFU), and this minimally-invasive procedure can be repeated many times as needed without damaging the normal tissue for localized controlled gene or drug delivery in a precise location using the ultrasonic imaging technology.

In one embodiment, the energy source vibrates the pluralities of antibody/medication coated ferroelectric nanoparticles, piezoelectric nanotubes, or pyroelectric nanoparticles conjugated with a medication and/or gene, and quenched fluorescein, liposomes filled with fluorescein or polymer micelles or nanoparticles usually poly(N-isopropyl acrylamide) carrying another dye or indicator etc., or bubble liposomes carrying fluorescein which contain air pockets or nanoemulsions of PFC using pulses of electrical current with an adjustable signal frequency and voltage that create an acoustic response by the electrically stimulated ferroelectric nanoparticles, piezoelectric nanotubes, or pyroelectric nanoparticles inside the body, which are recorded by one or multiple transducers located in different parts of the body. The signal is then amplified and forwarded to a processor in order to be converted to a 1D, 2D, or 3D image by electroacoustic computed tomography. The lesion or lesions to which the piezoelectric nanoparticles and other nanoparticles are attached are recognized by electroacoustic computed tomography, and are radiated with pulses of non-thermal, low intensity focused ultrasound (LIFU), thereby releasing the dye, medication, gene etc. by vibrational force of ultrasound in a non-thermal mode under observation of the electroacoustic imaging, not only indicating the presence of tumor cells using universal back-projection (UBP) and time reversal algorithm, etc., but also the lesion is treated simultaneously with the medication/gene and imaged both by the ultrasound and electroacoustic imaging. The piezoelectric or pyroelectric nanoparticle, or nanotube/cell complex, which releases the medication, is verified by the release of fluorescein in the circulation.

In one embodiment, the non-thermal focused high power ultrasound energy source vibrates the antibody/medication coated magnetic nanoparticles conjugated with a medication and/or gene, and quenched fluorescein or another dye or indicator etc. using an alternating magnetic field without producing a significant thermal response, thus releasing the dye, medication, gene, etc. by vibrational force created in the magnetic nanoparticle, and the release of dye/medication is verified by the release of the fluorescein in the circulation.

In one embodiment, the antibody/medication coated pluralities of gold, iron oxide, nanocage, nanotube, nanoshell, magnetic, paramagnetic, pyroelectric and piezoelectric nanoparticles are coated with thermosensitive polymers, such as chitosan, liposomes, or polymer micelles or nanoparticles usually poly(N-isopropyl acrylamide) carrying a dye quenched with fluorescein or another dye or indicator etc. and conjugated with a medication, and with cell penetrating agents or an activatable cell penetrating agent and quenched fluorescein, and are injected in the patient's circulation, lymphatic vessels, inside a body cavity, cerebrospinal fluid, etc. so as to be attached to the surface antigen of normal cells or of tumor cells, and then the piezoelectric nanoparticle are exposed to a pulse of electrical current thereby creating an electroacoustic sound from the piezoelectric nanoparticles that can be recorded with one or multiple transducers. Then, the signal is amplified and forwarded to a processor so as to be converted to a 1D, 2D, or 3D image as an electroacoustic computed tomogram. Focused high power ultrasound energy is used in a thermal mode, as high intensity focused ultrasound HIFU generally about or 2 MHz to 50 MHz or 100 MHz and one Watt or more, to raise the temperature of nanoparticles/tumor cells complex to 41-43 degrees C. or more, thus releasing the fluorescein and the medication, which is alternated with focused low power ultrasound in a compressive mode 1-140 kHz and <1 W power or more under the control of a processor controlling the thermal energy intensity and duration of the ultrasound, while imaging the lesion with the ultrasound and electroacoustic computerized tomography or in combination with another imaging modality such as MRI or CT-scan or PET-scan that defines the focal point of the focused low power compressive ultrasound in relationship with the other body's structures.

In one embodiment, the antibody coated pluralities of gold, iron oxide, nanocage, nanotube, nanoshell, magnetic, paramagnetic, pyroelectric and piezoelectric nanoparticles are coated thermosensitive polymers, such as chitosan, liposomes, or polymer micelles or nanoparticles usually poly(N-isopropyl acrylamide) carrying a dye quenched with fluorescein or another dye or indicator etc., and conjugated with a medication and/or gene, and conjugated with cell penetrating agents or activatable cell penetrating agents and quenched fluorescein injected in the patient's circulation, lymphatic vessels, inside a body cavity, cerebrospinal fluid, etc. so as to be attached to the surface antigen of normal cells or of tumor cells, and the antibody conjugated piezoelectric nanoparticles are exposed to pulses of electrical current with an adjustable signal frequency and voltage, thereby creating an electroacoustic sound from the piezoelectric nanoparticles that can be recorded with one or multiple array or a single moving transducers. The signal is then amplified and forwarded to a processor to be converted to a 1D, 2D stereoscopic image, or 3D image or video as an electroacoustic computed tomogram, using focused high power ultrasound energy in a thermal mode simultaneously to raise the temperature of nanoparticles/tumor cells complex to 41-43 degrees C., or more thereby releasing the fluorescein and the medication using focused ultrasound energy in a LIFU or a HIFU thermal mode under the control of a processor controlling the thermal energy intensity and duration of the ultrasound while imaging the lesion with the ultrasound electroacoustic computed tomography or using harmonic back scatter ultrasonic waves that can be seen simultaneously during the thermal heating of the tissue or chance in the back scatter energy.

In one embodiment, a robotic arm can be used to heat the tissue or the tumor at the focal point of the focused ultrasound, thus producing a 2-3D image of the ultrasound, while the thermal energy is controlled automatically by the software construing the thermal or temperature of the image to the ultrasonic system to generate more or less energy while the focal point under observation of 2-3 D imaging system heats up the entire tumor with a 50 micron by 50 micron sized focal point releasing the medication, gene, etc. from the antibody coated nanoparticles, the medication being injected in the circulation through the tumor.

In one embodiment, non-thermal low power ultrasound (LIFU) and therapy is combined with MRI, thereby eliminating cavity formation of the high power high-intensity focused ultrasound (HIFU) and potential scar and bubble formation with HIFU in the path of the ultrasound, and reducing the time of the ultrasound surgery with HIFU and its complications.

In one embodiment, the antibody coated pluralities of gold, iron oxide, silica, nanocage, nanotube, nanoshell, magnetic, paramagnetic, pyroelectric, and piezoelectric nanoparticles are coated with thermosensitive polymers, such as chitosan, liposomes, or polymer micelles or solid lipid nanoparticles or nanoparticles usually poly(N-isopropyl acrylamide) carrying a dye quenched with fluorescein or another dye or indicator etc. and conjugated with a medication and/or gene, and conjugated with Wnt inhibitors or rock inhibitors with cell penetrating agents or activatable cell penetrating agents and quenched fluorescein injected in the patient's circulation, lymphatic vessels, inside a body cavity, cerebrospinal fluid, etc. so as to be attached to the surface antigen of normal cells or of tumor cells, and to expose the piezoelectric nanoparticles to pulses of electrical current with adjustable signal frequency and voltage thereby creating an electroacoustic sound from the piezoelectric nanoparticles that can be recorded with one or multiple transducers. The signal is then amplified and forwarded to a processor so as to be converted to a 1D, 2D, or 3D image or video as an electroacoustic computed tomogram, using simultaneously either low intensity focused ultrasound (LIFU) or focused high power ultrasound energy in a thermal mode (HIFU) to raise the temperature of nanoparticles/tumor cells complex to 41-43 degrees C., or more thereby releasing the fluorescein and the medication using focused ultrasound energy in the thermal mode under the control of a processor controlling the thermal energy intensity and duration of the ultrasound, while imaging the lesion with the ultrasound and electroacoustic computerized tomography or videography.

In one embodiment, the antibody coated pluralities of gold, piezoelectric, iron oxide, nanocage, nanotube, nanoshell, magnetic, paramagnetic, pyroelectric and piezoelectric nanoparticles are coated with thermosensitive polymers, such as chitosan, liposomes, or polymer micelles or nanoparticles usually poly(N-isopropyl acrylamide) carrying a dye quenched with fluorescein or another dye or indicator etc. or bubble liposomes carrying fluorescein which contain air pockets or nanoemulsions of PFC conjugated with a medication, and conjugated with cell penetrating agents or activatable cell penetrating agents and quenched fluorescein, and injected in the patient's circulation, lymphatic vessels, inside a body cavity, cerebrospinal fluid, etc. such that the dye is released when the nanoparticles are exposed to low power focused ultrasound energy in a non-thermal compressive mode (LIFU) at frequency of 1-140 kHz and a power of 0.1 Watt to vibrate the piezoelectric, pyroelectric nanoparticles/tumor cells complex that are injected inside the body and are attached to the surface antigen of normal cells or of tumor cells, and when exposed to pulses of electrical current with an adjustable signal frequency and voltage, an electroacoustic sound is created inside the body from the piezoelectric nanoparticles that can be recorded with a transducer. The signal is then amplified and forwarded to a processor to be converted to a 1D, 2D, or 3D image as an electroacoustic computed tomogram and ultrasound image of the structure, and the dye/medication is released under observation of the lesion as video-electroacoustic imaging.

In one embodiment, antibody/medication coated gold, iron oxide, nanocage, nanotube, nanoshell, magnetic, paramagnetic, pyroelectric and piezoelectric nanoparticles are conjugated with a medication and checkpoint inhibitors and immune stimulators such as VLP, TLR3, TLR7, TLR8 or CD40 and quenched fluorescein or another dye or indicator, etc. so that the medications are released using high power focused ultrasound energy in a thermal mode alternating with focused ultrasound in a non-thermal compressive mode at 1-140 kHz to one MHz or more, and less than 1 Watt power (LIFU) while antibody coated nanoparticles/checkpoint inhibitors and VLP attach to the localized or circulating tumor cells and their exosomes, which are carrying a checkpoint protein, such as PD-L1 (to disguise themselves), and the tumor cells are recognized by the T-cells, which together with killer cells phagocytose them, and enhance the immune response to the tumor and its exosomes and the circulating cells. The T-cells destroy the tumor cells, circulating tumor cells, and their exosomes as a result of the VLPs and checkpoint inhibitors that are attached to the tumor cells, circulating tumor cells, and their exosomes. The VLPs make the tumor cells, circulating tumor cells, and their exosomes visible to the T-cells, which destroy them.

In one embodiment, antibody/medication coated gold, iron oxide, nanocage, nanotube, nanoshell, magnetic, paramagnetic, pyroelectric and piezoelectric nanoparticles that contain at least one radioactive alpha, or beta emitter conjugated with medication and checkpoint inhibitors, VLP, TLR3, TLR7, TLR8 or CD40, etc. and quenched fluorescein or another dye or indicator, etc. are exposed to pulses of electrical current with an adjustable signal frequency and voltage so that a electroacoustic sound is created inside the body from the piezoelectric nanoparticles that can be recorded with a transducer. The signal is then amplified and forwarded to a processor so as to be converted to a 1D, 2D, or 3D image as an electroacoustic computed tomogram and ultrasound to release the dye/medication, gene using focused ultrasound energy in a thermal mode at a temperature of 41-43 degrees C. alternating with focused ultrasound in a compressive mode at low power 1-140 or more kHz under the control of a processor controlling the thermal energy intensity and duration of the ultrasound while imaging the lesion with the ultrasound by a software connecting the thermal energy delivery to the temperature imaging system, while antibody coated nanoparticles/checkpoint inhibitors and VLP attach to the localized or circulating tumor cells and their exosomes, which carry PD-L1 (to disguise themselves), and the tumor cells are recognized by the T-cells, which together with killer cells phagocytose them, and enhance the immune response to the tumor and its exosomes and the circulating cells and eliminate them.

In one embodiment, antibody/medication coated gold, iron oxide, nanocage, nanotube, nanoshell, magnetic, paramagnetic, pyroelectric and piezoelectric nanoparticles are conjugated with medication and checkpoint inhibitors, VLP, TLR3, TLR7, TLR8, or CD40, etc. and quenched fluorescein to be released at a temperature of 41-43 degrees C. using focused ultrasound energy in a thermal mode alternating with focused low power ultrasound in a compressive mode at 1-140 kHz or more as many times as needed after initial treatment to damage the remaining tumor cells or recurrences with simultaneous vaccination while imaging the lesion with the ultrasound/electroacoustic tomography and initiate an immune response to the tumor cells, circulating cells and their exosomes.

In one embodiment, the energy source heats the antibody/medication coated pyroelectric or piezoelectric nanoparticles conjugated with medication and checkpoint inhibitors, VLP etc and quenched fluorescein or another dye or indicator, etc. to be released at temperature of 41-43 degrees C. for the desired time using focused ultrasonic energy in a thermal mode under the control of a processor controlling the intensity of the focused ultrasound for a desired duration, while antibody coated nanoparticles/checkpoint inhibitors and VLP, TLR3, TLR7, TLR8 or CD40 attach to the localized or circulating tumor cells and their exosomes, which are carrying a checkpoint protein, such as PD-L1 (to disguise themselves), and the tumor cells are recognized by the T-cells, which together with killer cells phagocytose them, and enhance the immune response to the tumor and its exosomes and the circulating cells.

In one embodiment, the focused high power ultrasound energy source at 0.5-3 MHz heats the antibody/medication coated gold, iron oxide, nanocage, nanotube, nanoshell, solid lipid nanoparticles, piezoelectric nanoparticles using focused ultrasound so as to heat the tumor cell nanoparticle complex conjugated with quenched fluorescein or another dye or indicator etc., and VLP, CD40 or TLR3, TLR7 and apply a pulse of electric current to induce a sound from the piezoelectric nanoparticles, image the lesion and to release fluorescein and medication at 41-43 degrees C., while maintaining the intensity, duration of the ultrasound energy or reducing its intensity to a desired level, and for a desired time, obtaining blood samples to evaluate increased biomarkers in the circulation and produce a vaccine with viral-like particles (VLPs) and TLR3, TLR7, TLR8 or CD40 and administering them with checkpoint inhibitors, such as PD-1, PD-11, CTLA-4, Jagged 1 inhibitor 15D11, etc. and Rock inhibitors, such as Fasudil, or Wnt inhibitors, such as niclosamide for future use, as needed (e.g. every six months or once a year) while antibody coated nanoparticles/checkpoint inhibitors and VLP, TLR3, TLR7, TLR8 or CD40 attach to the potentially localized or residual tumor or circulating tumor cells and their exosomes, which are carrying a checkpoint protein, such as PD-L1 (to disguise themselves), and the tumor cells are recognized by the T-cells, which together with killer cells phagocytose them, and enhance the immune response to the tumor and its exosomes and the circulating cells.

In one embodiment, the focused high power ultrasound energy source heats the antibody/medication coated gold, iron oxide, nanocage, nanotube, nanoshell, or piezoelectric nanoparticles using focused ultrasound so as to heat the tumor cell nanoparticle complex conjugated with quenched fluorescein or another dye or indicator etc., and VLP, CD40 or TLR3, TLR7 and a pulse of electric current is applied to induce a sound from the piezoelectric nanoparticles, image the lesion and to release fluorescein and medication at 41-43 degrees C., while maintaining the intensity, duration of the ultrasound energy or reducing its intensity to a desired level, and for a desired time, obtaining blood samples to evaluate increased biomarkers in the circulation and produce a vaccine with viral-like particles (VLPs), or oncolytic viruses, T-Vec and administering them with thermotherapy and releasing them at the temperature or 41-43 degrees C. that also damages or kills the VLP and oncolytic viruses such as T-Vec while leaving their antigenic foreign proteins in the tumor site to enhance an immune response, while releasing checkpoint inhibitors, such as PD-1, PD-11, CTLA-4, Jagged 1 inhibitor 15D11, while antibody coated nanoparticles/checkpoint inhibitors and VLP, CD40 or TLR3, TLR7 attach to the localized or circulating tumor cells and their exosomes, which are carrying a checkpoint protein, such as PD-L1 (to disguise themselves), and the tumor cells are recognized by the T-cells, which together with killer cells phagocytose them, and enhance the immune response to the tumor and its exosomes and the circulating cells, etc. and Rock inhibitors, such as Fasudil, in combination with anti-VEGF, Notch pathway inhibitors, PDGF inhibitors, or Wnt inhibitors, such as niclosamide for future use, as needed (e.g. every six months or once a year) reducing TGF-β production after therapy and the subsequent scar formation.

In one embodiment, antibody coated iron oxide, nanocage, nanotube, nano shell, magnetic, paramagnetic, pyroelectric and piezoelectric nanoparticles are conjugated with medication, checkpoint inhibitors, and quenched fluorescein or bubble liposomes carrying fluorescein which contain air pockets or nanoemulsions of PFC to be released using focused ultrasonic in a compressive mode at 1-40 kHz where the medications are from the group consisting of Wnt inhibitors, Rock inhibitors, metformin, anti-VEGFs, such as Bevacizumab, Ranibizumab, Aflibercept, Avastin, and anti-platelet derived growth factor (PDGF) agents, and notch pathway inhibitors, Doxorubicin, cyclophosphamide, antibiotics, anti-inflammatory, mammalian target of rapamycin, etc.

In one embodiment, glucose metabolism activates the Ras signaling that regulates the cell proliferation, along or independently from the Wnt activation and glucose triggers activation of MEK and ERK and inhibition of glucose metabolism at the tumor cell level with metformin, etc. delivered with antibody coated nanoparticles of iron oxide, nanocage, nanotube, nanoshell, magnetic, paramagnetic, pyroelectric and piezoelectric nanoparticles conjugated with a medication selected from the group consisting of Wnt inhibitors, Rock inhibitors, metformin, buformin, and syrosingopine, phenformin, or in combination with a glutaminase inhibitor, such as Calithera, inhibiting glutamine uptake for the tumor to which the tumor cells are addicted, anti-VEGFs, such as Bevacizumab, Ranibizumab, Aflibercept, Avastin, and anti-platelet derived growth factor (PDGF) agents and notch pathway inhibitors, and combination of checkpoint inhibitors, VLPs, IL-2, or IL-15, IL-6 IL, IL-15, IL-17 or polymeric antibody coated nanoparticles carrying IL-2 and IL-21 or anti-CD3 (OKT3) antibody, IFN-γ or granzyme β, related apoptosis-inducing ligand (TRAIL), or the Fas ligand or bee toxins, etc., enzymes such as Matrix metalloproteinases as immune stimulators and quenched fluorescein or another dye or indicator, etc. are released using focused ultrasonic energy in a compressive mode or a thermal mode at 50-150-kHz where the medications/dye are released affecting the membranes of the cancer cells and their metabolism more significantly than the normal surrounding normal cells while affecting the membranes of the cancer cells and their metabolism more significantly than the normal surrounding normal cells and release of VLP, CD40 or TLR3, TLR7, etc. as immune stimulators and checkpoint inhibitors enhance the immune response to the tumor locally and also their invisible metastatic lesions and their exosomes carrying the same tumor receptors and PD-L1 on their membranes.

In one embodiment, glucose metabolism activates the Ras signaling that regulates the cell proliferation, along or independently from the Wnt activation and glucose triggers activation of MEK and ERK and inhibition of glucose metabolism at the tumor cell level with metformin, etc. delivered with antibody coated nanoparticles of iron oxide, nanocage, nanotube, nanoshell, magnetic, paramagnetic, pyroelectric. nanobubbles, or microbubbles and piezoelectric nanoparticles conjugated with a medication selected from the group consisting of Wnt inhibitors, Rock inhibitors, metformin, buformin, or in combination with a glutaminase inhibitor, such as Calithera, inhibiting glutamine uptake for the tumor to which the tumor cells are addicted, or cysteine protease inhibitors such as stefins and syrosingopine, phenformin, anti-VEGFs, and the notch pathway inhibitors, and checkpoint inhibitors, VLP, IL-2, or IL-15, or anti-CD3 (OKT3) antibody, IFN-γ or granzyme β, related apoptosis-inducing ligand (TRAIL), or the Fas ligand, or bee toxins, etc., enzymes such as Matrix metalloproteinases as immune stimulators and quenched fluorescein or another dye or indicator, etc. are released using focused ultrasonic energy in a compressive mode at 10-50 kHz where the medications/dye are released affecting the membranes of the cancer cells and their metabolism more significantly than the normal surrounding normal cells and release of VLP, CD40 or TLR3, TLR7, etc. as stimulators and checkpoint inhibitors enhance the immune response to the tumor locally and also their invisible metastatic lesions, circulating tumor cells and their exosomes carrying the same tumor receptors and PD-L1 on their membranes.

In one embodiment, the thermosensitive polymer coating of the antibody conjugated nanoparticles further comprises buformin or phenformin so as to inhibit glucose metabolism in the cells of the tumor, and inhibit glucose metabolism at the tumor cell level with buformin or phenformin etc. delivered with antibody coated nanoparticles of iron oxide, nanocage, nanotube, nanoshell, magnetic, paramagnetic, pyroelectric and/or piezoelectric nanoparticles conjugated with medication selected from the group consisting of Wnt inhibitors, Rock inhibitors, buformin, phenformin, cysteine protease inhibitors, such as stefins, anti-VEGFs such as Bevacizumab, Ranibizumab, Aflibercept, Avastin, and anti-platelet derived growth factor (PDGF) agents, anti-integrins and notch pathway inhibitors, and checkpoint inhibitors, and quenched fluorescein or another dye or indicator, etc. to be released using focused ultrasonic energy in a compressive mode or a thermal mode (e.g., at 50-150-kHz) where the medications/dye are released affecting the cell membranes of the cancer cells and their metabolism more significantly than the normal surrounding normal cells.

In one embodiment, antibody/medication coated gold, iron oxide, nanocage, nanotube, nanoshell, magnetic, paramagnetic, pyroelectric, and piezoelectric nanoparticles are conjugated with medication and checkpoint inhibitors, VLP, or immune stimulators and quenched fluorescein or doxorubicin or another dye or indicator, etc. to be released using focused ultrasound in a thermal mode alternating with focused compressive ultrasound in a non-thermal mode.

In one embodiment, the functionalized piezoelectric nanoparticles are injected in a patient suspected of a malignancy under a pulse of electrical current creating an electroacoustic sound from the piezoelectric nanoparticles that can be recorded with one or multiple transducers. The signal is then amplified and forwarded to a processor so as to be converted to a 1D, 2D, or 3D image as an electroacoustic computed tomogram verifying the presence or absence of a suspected tumor or to differentiate a benign lesion from a malignant lesion to which the pluralities of nanoparticles are attached and produce the electroacoustic computerized tomography.

In one embodiment, the antibody piezoelectric nanoparticles are injected to reveal the existent of a lesion or tumor under a pulse of electrical current that produces an electroacoustic sound wave that can be captured and amplified, and converted to a signal producing an image as an electroacoustic computed image.

In one embodiment, the antibody/medication piezoelectric nanoparticles, pyroelectric, or perovskites nanoparticles are re-injected to reveal the existent of a lesion or tumor after the tumor has been treated previously under pulses of electrical current with an adjustable signal frequency and voltage that produces an electroacoustic sound wave from the piezoelectric nanoparticles or pyroelectric or perovskites that can be captured, amplified, and converted to a signal producing an image or as an electroacoustic computed tomogram indicating either persistence of the tumor cells, recurrences, or metastatic lesion or the tumor while electric pulses of 30-100 Hz and more paralyses the tumor cells by depolarizing the membrane potential and making their cell membrane accessible to medication.

In one embodiment, the antibody piezoelectric nanoparticles, pyroelectric or perovskites, Boron tubes, etc., iron oxide nanoparticles or bubble liposomes carrying fluorescein which contain air pockets or nanoemulsions of perfluorocarbon (PFCL) liquid are conjugated with monoclonal antibodies, checkpoint inhibitors, VLP, and IL-2, or IL-15, or polymeric antibody coated nanoparticles carrying IL-2 and IL-21 or anti-CD3 (OKT3) antibody, IL-6, IFN-γ or granzyme β, related apoptosis-inducing ligand (TRAIL), or the Fas ligand, etc., Wnt inhibitors, Rock inhibitors, genes, or gene inhibitors, and are injected to reveal the existent of a lesion or tumor after the tumor has been treated previously under pulses of electrical current with an adjustable signal frequency and voltage that produces an electroacoustic sound wave from the piezoelectric nanoparticles or pyroelectric or perovskites that can be captured, amplified, and converted to a signal producing an image or as an electroacoustic computed tomogram indicating either persistence of the tumor cells, recurrences, or a metastatic lesion or tumor, and simultaneously treated with non-thermal or thermal focused low power ultrasound, electromagnetic radiation, or an alternating magnetic field, etc. to damage the tumor cells thermally, while antibody coated nanoparticles/checkpoint inhibitors and VLP attach to the localized or circulating tumor cells and their exosomes, which are carrying a checkpoint protein, such as PD-L1 (to disguise themselves), and the tumor cells are recognized by the T-cells, which together with killer cells phagocytose them, and enhance the immune response to the tumor and its exosomes and the circulating cells, and the Rock inhibitor or in combination with anti-VEGF, or PDGF inhibitors, thus reducing TGF-β production after therapy and the subsequent scar formation.

In one embodiment, the antibody coated pluralities of gold, iron oxide, silica, nanocage, nanotube, nanoshell, magnetic, paramagnetic, pyroelectric and piezoelectric nanoparticles are coated with thermosensitive polymers, such as chitosan, liposomes, or polymer micelles or nanoparticles usually poly(N-isopropyl acrylamide) carry a dye quenched with fluorescein or another dye or indicator, etc. conjugated with a medication and/or gene, and conjugated with Wnt inhibitors or rock inhibitors and with cell penetrating agents or activatable cell penetrating agents and quenched fluorescein or another dye or indicator etc. injected in the patient's circulation, lymphatic vessels, inside a body cavity, cerebrospinal fluid, etc. so as to be attached to the surface antigen of normal cells or of tumor cells, and the piezoelectric nanoparticles are exposed to a pulse of electrical current, thereby creating an electroacoustic sound from the piezoelectric nanoparticles that can be recorded with a transducer. The transducer signal is then amplified and forwarded to a processor so as to be converted to a 1D, 2D, or 3D image using focused low power ultrasound energy in a non-thermal mode, thus releasing the fluorescein and the medication and/or gene etc. using the focused non-thermal mode to strip away the dye, medications, and/or gene under the control of a processor controlling the thermal ultrasonic intensity and duration of the ultrasound, while imaging the lesion with the ultrasound along with electroacoustic 2-D or 3-D images as an electroacoustic computed tomogram or in combination with another imaging modality such as MRI or CT-scan or PET-scan that defines the focal point of the focused compressive ultrasound in relationship with the other body's structures.

In one embodiment, iron oxide, nanocage, nanotube, nanoshell, magnetic, paramagnetic, pyroelectric and piezoelectric antibody/medication coated nanoparticles contain at least one radioactive agent conjugated with medication and checkpoint inhibitors Botulinum toxin, VLP, etc. Rock inhibitors, such as Fasudil, netarsudil, and quenched fluorescein or another dye or indicator, etc. so as to be released using focused ultrasonic in thermal mode alternating with a non-thermal focused compressive low power ultrasound mode and the repeated ultrasound pulses generate an electric pulse from the piezoelectric nanoparticles that damages the tumor cell by depolarizing their cell membrane while antibody coated nanoparticles/checkpoint inhibitors and VLP attach to the localized or circulating tumor cells and their exosomes, which are carrying a checkpoint protein, such as PD-L1 (to disguise themselves), and the tumor cells are recognized by the T-cells, which together with killer cells phagocytose them, and enhance the immune response to the tumor and its exosomes and the circulating cells.

In one embodiment, iron oxide, nanocage, nanotube, nanoshell, magnetic, paramagnetic, pyroelectric and piezoelectric antibody coated nanoparticles are conjugated with a Wnt inhibitor, Rock inhibitors, such as Fasudil, etc., antineoplastic medication, checkpoint inhibitors, immune stimulators, and quenched fluorescein or another dye or indicator, etc. to be released using focused ultrasonic in as thermal mode (e.g., at a frequency of 50-kHz-1 MHz) alternating with a focused compressive low power ultrasound mode and the repeated ultrasound pulses generate an electric pulse from the piezoelectric nanoparticles that damages the tumor cell by depolarizing their cell membrane and initiate an immune response to the tumor cells, circulating cells, and their exosomes.

In one embodiment, iron oxide, nanocage, nanotube, nanoshell, magnetic, paramagnetic, pyroelectric, and piezoelectric antibody/medication coated nanoparticles are conjugated with a Rock inhibitor, such as Fasudil, and its derivatives etc., antineoplastic medication, checkpoint inhibitors, immune stimulators, e.g., VLP, and quenched fluorescein or another dye or indicator, etc. to be released using high power focused ultrasound in a thermal mode alternating with a low power focused compressive ultrasound mode and initiate an immune response to the tumor cells, circulating cells, and their exosomes.

In one embodiment, iron oxide, nanocage, nanotube, nanoshell, magnetic, paramagnetic, pyroelectric, and/or piezoelectric antibody coated nanoparticles are conjugated with a rock inhibitor, antineoplastic medication, RNAi, siRNA, and checkpoint inhibitors, Rock inhibitors, such as Fasudil, and its derivatives, etc., and quenched fluorescein or another dye or indicator, etc., and are exposed to pulses of electrical current with an adjustable signal frequency and voltage so that an electroacoustic sound is created inside the body from the piezoelectric nanoparticles that can be recorded with a transducer. The transducer signal is then amplified and forwarded to a processor in order to be converted to a 1D, 2D, or 3D image as an electroacoustic computed tomogram, and to release the medication using focused high power ultrasound in a thermal mode (e.g., at a frequency of 50-1 MHz) alternating with a focused compressive low power ultrasound mode and the repeated low power ultrasound pulses generate an electric pulse from the piezoelectric nanoparticles that damages the tumor cell by depolarizing their cell membrane.

In one embodiment, pluralities of iron oxide, gold, nanocage, nanotube, nanoshell, magnetic, paramagnetic, pyroelectric, and piezoelectric antibody coated nanoparticles are conjugated with rock inhibitors, an antineoplastic medication, genes, CRISPR-cas9, or Cas9 RNPs, checkpoint inhibitors, Rock inhibitors such as Fasudil, its derivatives, etc. and quenched fluorescein or another dye or indicator, etc., and then exposed to pulses of electrical current with an adjustable signal frequency and voltage so that a electroacoustic sound is created inside the body from the piezoelectric nanoparticles that can be recorded with a transducer. The transducer signal is amplified and forwarded to a processor so as to be converted to a 1D, 2D, or 3D image as an electroacoustic computed tomogram, and the gene, CRISPR-cas9, or medication is alone or in combination, released using focused low power compressive ultrasound to strip the coatings off the nanoparticles by vibrational forces of the non-thermal mode LIFU of ultrasound and to modify the gene using CRISPR-cas9 or Cas9 RNPs mediated Homology-Independent Targeted Integration (HITI) or Homology Directed Repair (HDR).

In one embodiment, iron oxide, nanocage, nanotube, nanoshell, magnetic, paramagnetic, pyroelectric, and piezoelectric antibody coated nanoparticles conjugated with a rock inhibitor, antineoplastic medication, CRISPR-cas9, or Cas9 RNPs, a corrective gene, and/or quenched fluorescein or another dye or indicator, etc. are exposed to pulses of electrical current with an adjustable signal frequency and voltage so that an electroacoustic sound is created inside the body from the piezoelectric nanoparticles that can be recorded with a transducer. The transducer signal is then amplified and forwarded to a processor to be converted to a 1D, 2D, or 3D image as an electroacoustic computed tomogram, and the nanoparticle coating is released using focused low power ultrasound in a compressive ultrasound mode so as to modify the gene using CRISPR-cas9 mediated Homology-Independent Targeted Integration (HITI) or Homology Directed Repair (HDR) while imaging the lesion.

In one embodiment, pluralities of nanoparticles, iron oxide, nanocage, nanotube, nanoshell, magnetic, paramagnetic, pyroelectric, and/or piezoelectric antibody coated nanoparticles are conjugated with a rock inhibitor, antineoplastic medication, CRISPR-cas9 or Cas9 RNPs with a gene, and with cell penetrating agents or activatable cell penetrating agents, and quenched fluorescein or another dye or indicator, etc. to be released using focused low power ultrasound in a compressive mode to modify the defective gene using CRISPR cas9 or Cas9 RNP mediated Homology-Independent Targeted Integration (HITI) or Homology Directed Repair (HDR) under observation of an electroacoustic computed tomogram and the repeated low power ultrasound pulses generate an electric pulse from the piezoelectric nanoparticles that damages the tumor cell by depolarizing their cell membrane end enhance gene entry to the cell.

In one embodiment, the transducer of a focused low power, compressive, or thermal high power ultrasound is positioned on the surface of the body to aim the focal point of the ultrasound at specific part of the lesion now made visible by electroacoustic imaging technology, such as its center or the borders of the lesion, or the focal point can move so that the entire lesion is treated with low power, compressive or low thermal focused ultrasound providing thermal energy to heat antibody/medication coated nanoparticles and its thermosensitive polymers carrying a dye or gene, checkpoint inhibitors, such as Fasudil, its derivatives and its derivatives etc., VLP or oncolytic viruses, such as T-Vec, monoclonal antibodies, IL-2, or IL-15, IL-17 or anti-CD3 (OKT3) antibody, IL-6, IL-1β, IL-17, bee toxins, immune stimulators along with checkpoint inhibitors, medication are released and damage already heated tumor cells at a temperature of 41-43 degrees C. and release dye and medication from the nanoparticles indicating the temperature of 41-43 degrees C. has been achieved which can be controlled by the processor or computer controlling energy delivery, under direct imaging the tumor, or the lesion or modify the genetic component of the tissue with appropriate genes and CRISPR-cas 9 mediated Homology-Independent Targeted Integration (HITI) or Homology Directed Repair (HDR) or enhance immune therapy by the dead VLP or oncolytic viruses, such as T-Vec and release other immune stimulators and checkpoint inhibitors to enhance the immune response to the tumor locally and also their invisible metastatic lesions and initiate an immune response to the tumor cells, circulating cells and their circulating exosomes and eliminate them.

In one embodiment, the transducer of a low power focused compressive non-thermal, or high power thermal ultrasound is positioned on the surface of the body to aim the focal point of the ultrasound at a specific part of the lesion now made visible by electroacoustic imaging technology such as its center or the borders of the lesion or the focal point of the none-thermal focused ultrasound connected to a movable arm can be moved on the body surface under the control of a processor or computer that show on it monitor both the location of the lesion and the images of the focused low power ultrasound simultaneously and a software controls the areas that are being treated in 3-D manner or as a live video so that the entire lesion is treated with compressive or low thermal focused, low power ultrasound providing minimal thermal energy to heat antibody/medication coated nanoparticles and its polymeric coating carrying a dye, medication or gene, checkpoint inhibitors, Rock inhibitors, such as Fasudil, etc., VLP or oncolytic viruses, such as T-Vec, or monoclonal antibodies, bee or scorpion toxin, metalloproteinases as immune stimulators, antineoplastic medication are released and damage already heated tumor cells at temperature or 41-43 degrees C. and release dye and medication from the nanoparticles indicating the temperature of 41-43 degrees C. has been achieved which can be controlled by the processor or computer controlling energy delivery, under direct imaging the tumor, lesion or modify the genetic component of the tissue with appropriate gene and CRISPR-cas 9 mediated Homology-Independent Targeted Integration (HITI) or Homology Directed Repair (HDR) and induce an immune response by released of VLP, etc. as stimulators and checkpoint inhibitors enhance the immune response to the tumor locally and also their invisible metastatic lesions and initiate an immune response to the tumor cells, circulating cells and their exosomes.

In one embodiment, the antibody coated nanoparticles, nanospheres, liposomes, solid nanoparticles, nanowires, nanorods, nanocages, nanoshells, magnetic, ferromagnetic, piezoelectric, pyroelectric, nanotubes, zinc oxide, barium titanate, iron oxide, gold, gold silica, strontium titanate, perovskites, and polyvinylidene fluoride, boron nitride nanotubes or a combination thereof are coated with a thermosensitive polymer, such as chitosan, polysaccharides, (e.g., IFN-γ or granzyme β, related apoptosis-inducing ligand (TRAIL), or the Fas ligand, to be released using focused non-thermal ultrasound (LIFU) in a compressive mode to modify the defective gene using CRISPR-cas9 or Cas9 RNP mediated Homology-Independent Targeted Integration (HITI) or Homology Directed Repair (HDR) under observation by an electroacoustic computed tomogram and) and induce an immune response by released of VLP, etc. as stimulators and checkpoint inhibitors enhance the immune response to the tumor locally and also their invisible metastatic lesions elsewhere and initiate an immune response to the tumor cells, circulating cells, and their exosomes and eliminate them.

In one embodiment, the nanoparticles are selected from a group consisting of iron oxide gold nanoparticles, gold graphene oxide nanoparticles, gold nanoparticles, silicone nanoparticles, carbon nanoparticles, magnetic nanoparticles, gold nanorods, gold nanotubes, gold nanoshells, gold nanocages, iron oxide, gold, boron nitride, and carbon nanotubes and combinations thereof that are under observation by an electroacoustic computed tomogram.

In one embodiment, the nanoparticles are selected from a group consisting of iron oxide gold nanoparticles, gold graphene oxide nanoparticles, gold nanoparticles, silicone nanoparticles, liposomes, solid lipid nanoparticles, carbon nanoparticles, magnetic nanoparticles, gold nanorods, gold nanotubes, gold nanoshells, gold nanocages, iron oxide, gold, boron nitride, and carbon nanotubes and combinations thereof conjugated with the photosensitizer verteporfin, and the antibody/medication coated nanoparticles contain at least one radioactive compound conjugated with medication and checkpoint inhibitors, Rock inhibitors, such as Fasudil, its derivatives, etc., and quenched fluorescein or another dye or indicator etc. to be released using focused ultrasonic in a thermal mode alternating with a non-thermal focused compressive or pressure ultrasound mode.

In one embodiment, the pluralities of antibody coated nanoparticles, nanospheres, liposomes, nanowires, nanorods, nanocages, nanoshells, nanotubes; magnetic, ferromagnetic, piezoelectric, pyroelectric nanotubes; zinc oxide, barium titanate, iron oxide, gold, gold silica, strontium titanate, perovskites, and polyvinylidene fluoride boron nitride nanotubes or a combination thereof are coated with a thermosensitive polymer, such as chitosan, polysaccharides, e.g., glycol chitosan, poly-L-Lysine (PLL), polyethylene imine (PEI), polylactic, polyglycolic, polyaspartic acid or copolymers, a cationic polymer such as polylysine and polyethylene imine, liposomes, or polymer micelles or nanoparticles usually poly(N-isopropyl acrylamide) carrying a dye conjugated with a rock inhibitor, antineoplastic medication, CRISPR-cas9 or Cas9/gRNA Ribonucleoprotein complexes (Cas9 RNPs) and VLP or other immune stimulators, oncolytic viruses, such as T-Vec, monoclonal antibodies, IL-2, or IL-15, or anti-CD3 (OKT3) antibody, IFN-γ or granzyme β, related apoptosis-inducing ligand (TRAIL), or the Fas ligand, bee toxins or other immune stimulators, checkpoint inhibitors, and a gene with cell penetrating agents or activatable cell penetrating agents and quenched fluorescein or another dye or indicator/medication etc. to be released using focused ultrasound in a non-thermal compressive or pressure mode along with pulses of electrical current with an adjustable signal frequency and voltage inducing a sound wave in the piezoelectric nanoparticles that can be verified by a transducer attached to the patient's body confirming the location of the tumor cells attached to the antibody coated piezoelectric nanoparticles as an electroacoustic computed tomogram to modify the defective gene using CRISPR cas9, Cas9/gRNA Ribonucleoprotein complexes (Cas9 RNP) mediated Homology-Independent Targeted Integration (HITI) or Homology Directed Repair (HDR), or to release antineoplastic medication at that place or checkpoint inhibitors, VLP, Rock inhibitors, such as Fasudil, etc., Wnt inhibitors discovering a single tumor or many metastatic cells located adjacent to the original lesion, in the lymph nodes, or in the circulation under observation by an electroacoustic computed tomogram and inducing an immune response by the release of VLP, etc. as stimulators and checkpoint inhibitors enhancing the immune response to the tumor locally and also their invisible metastatic lesions elsewhere and initiate an immune response to the tumor cells, circulating cells and their exosomes.

In one embodiment, the pluralities of antibody/medication coated nanoparticles, nanospheres, liposomes, nanowires, nanorods, nanocages, nanoshells, nanotubes; magnetic, ferromagnetic, piezoelectric, or pyroelectric nanotubes; zinc oxide, barium titanate, iron oxide, gold, gold silica, strontium titanate, perovskites, and polyvinylidene fluoride boron nitride nanotubes or a combination thereof are coated with polymer, such as chitosan, polysaccharides, e.g., glycol chitosan, poly-L-Lysine (PLL), polyethylene imine (PEI), polylactic, polyglycolic, polyaspartic acid or copolymers, a cationic polymer such as polylysine and polyethylene imine, polyethylene glycol, biotin streptavidin are injected with cell penetrating agents or activatable cell penetrating agents and quenched fluorescein, liposomes, or polymer micelles or nanoparticles usually poly(N-isopropyl acrylamide) carrying a dye or another dye or indicator, etc. in the patient's circulation, lymphatic vessels, inside a body cavity, in cerebrospinal fluid, in the eye, in the bladder, uterus in the peritoneal cavity and stimulated with pulses of electrical current with an adjustable signal frequency and voltage at predetermined intervals at different places in the body while listening to production of a sound wave which has the same frequency as the frequency of the pulses of electrical current with the adjustable signal frequency and voltage applied to the tissue indicating the presence of a suspected lesion by the production of a sound wave from the piezoelectric nanoparticle/tumor or exosomes and counting the circulating cells or the exosomes as they pass inside the electrical current to confirm non-invasively the presence of the extracellular vesicle, circulating tumor cells, etc., before or after treatment to find out soon within a week if the medication or the therapy has had an effect on the tumor and its circulating exosomes or metastatic cell numbers measured before and after the treatment or recognizing existing live cells inside a tumor after the therapy, such as in breast cancer, glioblastoma, uterus, ovarian cancer, prostate cancer, lung cancer, etc., making the tumor cells visible by an electroacoustic computed tomogram.

In one embodiment, the pluralities of antibody/medication coated nanoparticles, nanospheres, liposomes, nanowires, nanorods, nanocages, nanoshells, magnetic, non-magnetic, paramagnetic, pyroelectric, or piezoelectric nanotubes; magnetic, ferromagnetic, piezoelectric, or pyroelectric nanotubes; zinc oxide, barium titanate, iron oxide, gold, gold silica, strontium titanate, perovskites, and polyvinylidene fluoride boron nitride nanotubes and/or a combination thereof are coated with polymers, such as chitosan, polysaccharides, e.g., glycol, chitosan, poly-L-Lysine (PLL), polyethylene imine (PEI), polylactic, polyglycolic, polyaspartic acid or copolymers, a cationic polymer, such as polylysine and polyethylenimine, polyethylene glycol, biotin, and/or streptavidin and, liposomes, or polymer micelles or nanoparticles usually poly (N-isopropyl acrylamide) carrying a dye are injected with cell penetrating agents or activatable cell penetrating agents and quenched fluorescein or bubble liposomes carrying fluorescein which contain air pockets or nanoemulsions of PFC in the patient's circulation, lymphatic vessels, inside a body cavity, in cerebrospinal fluid, in the eye, in the bladder, in the uterus, or in the peritoneal cavity, and stimulated with pulses of electrical current with an adjustable signal frequency and voltage at predetermined intervals at different places in the body while listening to the production of a sound wave, which has the same frequency as the frequency of the pulses of electrical current with the adjustable signal frequency and voltage, applied to the tissue indicating the presence of a suspected lesion by the production of a sound wave by the piezoelectric nanoparticle/tumor or exosomes and counting the circulating cells or the exosomes as they pass inside the electrical current to confirm non-invasively the presence of the extracellular vesicles, circulating tumor cells, etc. before or after treatment to find out soon within a week if the medication or the therapy has had an effect on the tumor and its circulating exosomes or metastatic cell numbers measured before and after the treatment or recognizing existing live cells inside a tumor after the therapy, such as in breast cancer, glioblastoma, uterine cancer, ovarian cancer, prostate cancer, lung cancer, etc.

In one embodiment, the pluralities of antibody/medication coated nanoparticles, nanospheres, liposomes, nanowires, nanorods, nanocages, nanoshells, magnetic, non-magnetic, paramagnetic, pyroelectric piezoelectric nanotubes; magnetic, ferromagnetic, piezoelectric, pyroelectric nanotubes; zinc oxide quantum dots, quartz, barium titanate, iron oxide, gold, gold silica, strontium titanate, perovskites, and polyvinylidene fluoride boron nitride nanotubes; ferroelectric, pyroelectric or piezoelectric materials enclosed by a polymeric membrane and containing a drug, a gene, and/or a combination thereof are coated with a polymer, such as chitosan, polysaccharides, e.g., glycol, chitosan, poly-L-Lysine (PLL), polyethylene imine (PEI), polylactic, polyglycolic, polyaspartic acid or copolymers, cationic polymer, such as polylysine and polyethyleneimine, polyethylene glycol, biotin, streptavidin are injected with cell penetrating agents or activatable cell penetrating agents and quenched fluorescein or liposomes, or polymer micelles or nanoparticles usually poly(N-isopropyl acrylamide) carrying fluorescein dye or another dye or indicator/medication, etc. in the patient's circulation lymphatic vessels, inside a body cavity, in cerebrospinal fluid, in the eye, in the bladder, in the uterus, in the peritoneal cavity, and stimulated with pulses of electrical current at predetermined intervals at different places in the body while listening to the production of a sound wave which has the same frequency as the frequency of the pulses of electrical current applied to the tissue indicating the presence of a suspected lesion by the production of a sound wave by the piezoelectric nanoparticle/tumor or exosomes, recording the sound waves by an ultrasonic transducer or a microphone, and converting the sound wave to an electrical signal sending it to an imaging oscilloscope, video screen, to create 1-dimensional, 2-dimensional, or 3-dimensional images as a tomogram using a computer software indicating the presence and the shape of the lesion in the body or its absence using an electroacoustic computed tomography.

In one embodiment, the abovementioned electroacoustic computed tomography may be made as a tube, half tube, partial tube like MRI, or CT-scan or PET-scan units in which the patient can lay down and the table moves the patient in or out of the system while multiple rows or electrical connection is made like an electrocardiogram recording electrode, but in this case, they represent anode or cathodes arrays of the system so that the entire patient body may be scanned or imaged using a computer controlled recording, and electroacoustic tomography is done in a less expensive and time consuming way than is needed for an MRI with its specific room or CT-scan or PET-scan, but without exposing the patient to radiation.

In one embodiment, thermotherapy with focused low power ultrasound or alternating magnetic field is combined with the antibody/medication coated piezoelectric, pyroelectric nanotubes, nanocages, nanoshells may be combined with other antibody/medication coated nanoparticles that serve independently for drug delivery, gene delivery, etc., such as only one non-limiting example, the dendrimer poly(amidoamine) (PAMAM) can be functionalized to be biocompatible and cell penetrating. Other dendrimers are poly(amidoamine-organosilicon) (PAMAMOS), poly(propyleneimine) (PPIO), tecto, multilingual, chiral, hybrid, amphiphilic, micellar, multiple antigen peptide, and Frechet-type dendrimers. Dendrimers may be used as carriers of inhibitory genes to correct muted gene or elimination of the mutated gene(s) using CRISPR-cas9 or Cas9/gRNA Ribonucleoprotein complexes (Cas9 RNP) mediated Homology-Independent Targeted Integration (HITI) or Homology Directed Repair (HDR) under observation with electroacoustic tomography to precisely locate the lesion for gene delivery.

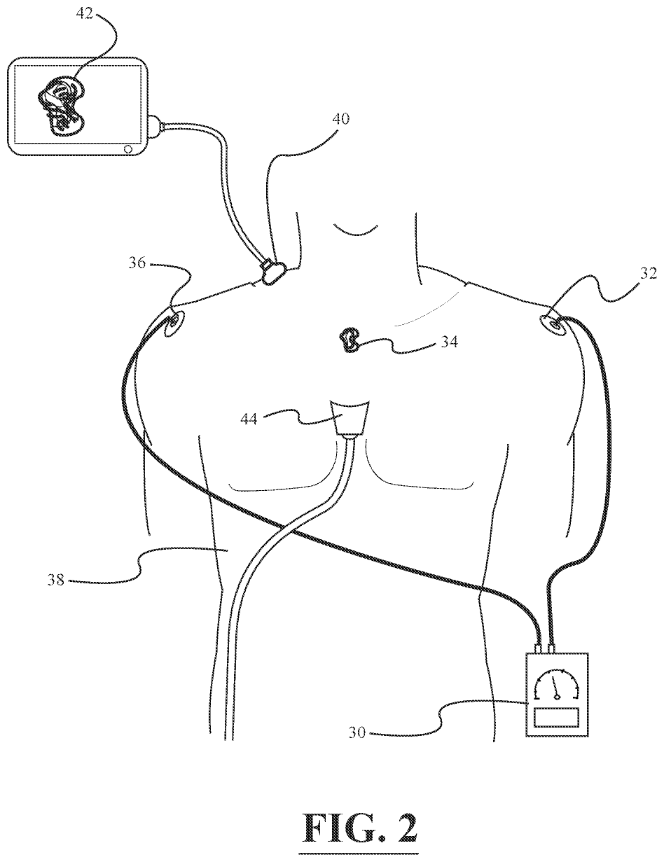

In one embodiment, thermotherapy with focused low power ultrasound or alternating magnetic field is combined with the pluralities of antibody/medication coated nanoparticles, nanospheres, liposomes, solid nanolipid, nanowires, nanorods, nanocages, nanoshells, magnetic, non-magnetic, paramagnetic, pyroelectric, piezoelectric, nanotubes; magnetic, ferromagnetic, piezoelectric, pyroelectric, nanotubes; zinc oxide, barium titanate, iron oxide, gold, gold silica, strontium titanate, perovskites, and polyvinylidene fluoride boron nitride nanotubes and/or combinations thereof are coated with polymers, such as chitosan, polysaccharides, e.g., glycol, chitosan, poly-L-Lysine (PLL), polyethylene imine (PEI), polylactic, polyglycolic, polyaspartic acid or copolymers, phospholipid microbubbles containing a harmless gas cationic polymer such as polylysine and polyethyleneimine, polyethylene glycol, biotin, streptavidin having a radioactive alpha, beta, or gamma emitter, monoclonal antibody, or checkpoint inhibitors, Wnt inhibitors or Rock inhibitors such as Fasudil, etc., or antineoplastic medication, etc. are injected with cell penetrating agents or activatable cell penetrating agents in the patient's circulation, lymphatic vessels, inside a body cavity, in cerebrospinal fluid, in the eye, in the bladder, in the uterus, in the peritoneal cavity etc. and stimulated with pulses of electrical current with an adjustable signal frequency and voltage at predetermined intervals at different places in the body while listening to the production of a sound wave at different locations, which has the same frequency as the frequency of the pulses of electrical current with the adjustable signal frequency and voltage, applied to the tissue indicating the presence of a suspected lesion by the production of a sound wave by the piezoelectric nanoparticle/tumor or exosomes complex recording the sound waves, by an ultrasonic transducer or a microphone converting the sound wave to an electrical signal, amplifying the signal, and sending it to an imaging oscilloscope, video screen, to create 1-dimensional, 2-dimensional, or 3-dimensional images of the lesion as an electroacoustic computed tomography using a computer and software indicating the presence or absence and the shape of the lesion in the entire body while the detected lesion can be treated with either a focused compressive or pressure low power ultrasound or focused high power ultrasound in a thermal mode in the same session (see FIG. 2) or later. Blood may also be obtained to confirm an increase in biomarkers in the blood and use the biomarkers to build an anti-tumor vaccine with or without VLP, IL-2, or IL-15, or anti-CD3 (OKT3) antibody, toxins, and administering them with checkpoint inhibitors such as PD-1, PD-11, CTLA-4, Jagged 1 inhibitor 15D11, etc., and Rock inhibitors, such as Fasudil, or Wnt inhibitors, such as niclosamide while antibody coated nanoparticles/checkpoint inhibitors and VLP attach to the localized or circulating tumor cells and their exosomes, which are carrying a checkpoint protein, such as PD-L1 (to disguise themselves), are recognized by the T-cells, which together with killer cells phagocytose them and enhance immune response to the tumor and its exosomes and the circulating cells, and the Rock inhibitor, or PDGF inhibitors, reduces TGF-β production after therapy and the subsequent scar formation.

In one embodiment, the antibody coated pluralities of gold, piezoelectric, iron oxide, nanocage, nanotube, nanoshell, magnetic, paramagnetic, pyroelectric, and/or piezoelectric nanoparticles, etc. are coated with thermosensitive polymers, such as chitosan, quenched with fluorescein or another dye or indicator/medication etc. and conjugated with an antineoplastic medication, gene, CRISPR-cas9 or Cas9/gRNA Ribonucleoprotein complexes (Cas9 RNPs) conjugated with cell penetrating agents or activatable cell penetrating agents injected in the patient's circulation, lymphatic vessels, inside a body cavity, cerebrospinal fluid, etc. The functionalized nanoparticles are exposed to electromagnetic radiation, microwaves, or radiofrequency radiation, or focused high power ultrasound 44 (see FIG. 2) in a thermal or non-thermal low power mode or non-focused ultrasound, or an alternating magnetic field and/or electrical current generated by a battery 30 (see FIG. 2) where low electrical current from a battery 30 passes from one side of the skin (i.e., the anode 32) through the body 38 and a lesion or tumor 34 to the cathode electrode 36 positioned on the opposite side of the skin on the body 38 to raise the temperature of the piezoelectric or pyroelectric nanoparticles that are injected inside the body 38 to be attached to the surface antigens of the normal cells or of the tumor cells and create a nanoparticle/tumor cell complex heated to 41-43 degrees C., and when exposed to pulses of electrical current generated by the battery 30 with an adjustable signal frequency and voltage, an acoustic response is produced by electrical stimulation of piezoelectric nanoparticles inside the body 38 that is called electroacoustic sounds or signals which can be captured with a transducer (e.g., ultrasound transducer 40 in FIG. 2), or microphone, converted to an electrical signal and is forwarded to a processor to be converted to a 1D, 2D, or 3D image 42 as an electroacoustic computed tomogram while the electrical pulse generated in the piezoelectric nanoparticles drives the medication, gene, in the tumor cells locally to damage the tumor cells by multiple modes of the therapy applied non-invasively under observation.

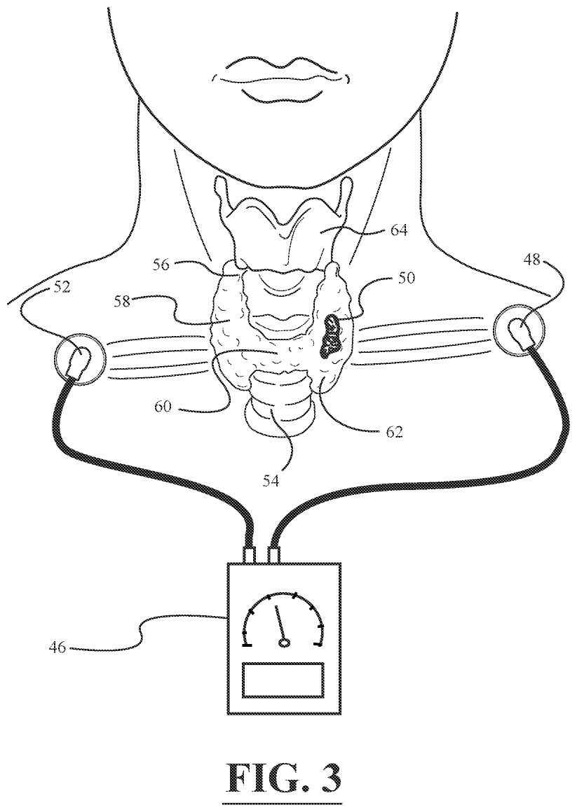

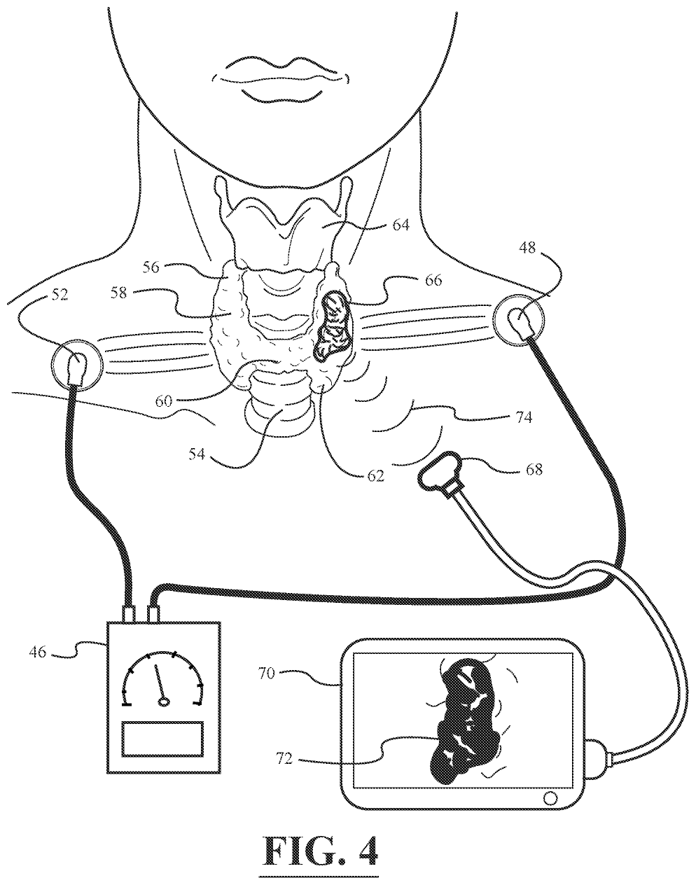

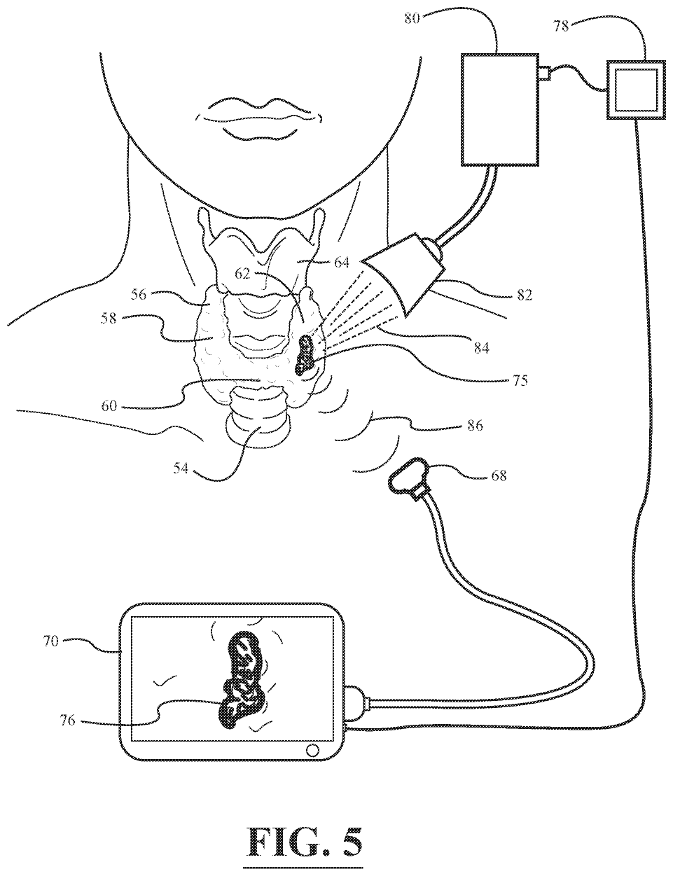

In another embodiment, in a patient with a thyroid tumor 50 (see FIG. 3), an electrical source comprises a battery device 46 with an anode 48 being positioned on a first side of the body of the patient and a cathode 52 being located on a second side of the body of the patient, the pulsed electrical current (as diagrammatically indicated by the current lines in FIG. 3) passing through the body (e.g., the neck) of the patient from the anode 48 to the cathode 52 of the battery device 46, and where the pulsed electrical current passes through the tumor 50 which has been pretreated with antibody coated piezoelectric nanoparticles conjugated with medication, and attached to the tumor cells. In FIG. 3, it can be seen that the thyroid gland 56 of the patient, which is disposed around the trachea 54, comprises the right lobe 58, the left lobe 62, and the isthmus 60 connecting the right and left lobes 58, 62. Thyroid cartilage 64 is disposed above the thyroid gland 56 in FIG. 3. In FIG. 3, the battery device 46 is operatively coupled to a controller with software for generating the pulsed electrical current passing through the body of the patient between the anode 48 and the cathode 52. Turning to FIG. 4, it can be seen that the pulsed electrical current (as diagrammatically indicated by the current lines in FIG. 4) generated by the battery device 46 creates an ultrasonic wave 74 from the piezoelectric nanoparticles that can be recorded by an ultrasonic transducer 68 located on the skin. In FIG. 4, the ultrasonic transducer 68 is connected to a processor and monitor 70, which allows an image 72 of the thyroid tumor 66 in FIG. 4 to be reconstructed from the ultrasonic wave 74 received at the transducer 68. In addition, turning to FIG. 5, it can be seen that a thyroid tumor 75 may be treated non-invasively with a focused ultrasound beam 84 generated by an ultrasound array transducer 82. The focused ultrasound beam 84 passes through the skin and simultaneously heats up the tumor tissue and attached piezoelectric nanoparticles to a temperature of 39-40 degrees C. to damage the tumor cells of the tumor 75 with the thermal energy and to depolarize the tumor cell membranes by converting the sound waves to an electric pulse to depolarize the tumor cells exposed to the ultrasound, making the cells permeable to the medication/gene used to treat the tumor 75. In FIG. 5, it can be seen that the heating of the tumor 75 by the focused ultrasound beam 84 creates harmonic backscatter ultrasonic waves 86 from the piezoelectric nanoparticles that can be recorded by an ultrasonic transducer 68 located on the skin. In FIG. 5, similar to FIG. 4, the ultrasonic transducer 68 is connected to a processor and monitor 70, which allows an image 76 of the thyroid tumor 75 to be reconstructed from the harmonic backscatter ultrasonic waves 86 received at the transducer 68. In FIG. 5, the processor and monitor 70 connected to the transducer 68 are operatively coupled to another processor 78 that executes software for controlling the energy output of the focused ultrasound delivered by the ultrasound array transducer 82. That is, the processor 78 is operatively coupled to the ultrasound power source 80 so as to enable the energy output of the focused ultrasound delivered by ultrasound array transducer 82 to be selectively varied based upon feedback from the harmonic backscatter ultrasonic waves 86 received by the ultrasonic transducer 68. In this manner, in the system of FIG. 5, the temperature at the tumor site is able to be selectively controlled by varying the energy output of the focused ultrasound delivered by ultrasound array transducer 82. Also, in FIG. 5, when the focused ultrasound is applied to the tumor 75 with piezoelectric nanoparticles attached to the tumor 75, the ultrasound activates the piezoelectric nanoparticles to produce electrons to depolarize the tumor cells, and the focused ultrasound also creates second harmonic sound waves 86 that are recorded by the transducer 68 indicating the temperature at the tumor site. The imaging system 70 in FIG. 5 is connected to the processor 78 which, in turn, is connected to the ultrasound power source 80 so as to control the temperature of the tumor site at 41-43 degrees C. or more, as needed. In this manner, the tumor cells are depolarized and heated simultaneously (i.e., combining cell thermotherapy with cell depolarization) and medication conjugated with the piezoelectric nanoparticles is released at the tumor site so as to damage the tumor cells. Because the tumor cells have already been rendered generally defenseless by virtue of their depolarization, the medication is able to easily pass through the damaged tumor cell membranes, thus entering the cytoplasms of the damaged tumor cells and destroying the tumor cells.

In one embodiment, the antibody coated pluralities of gold, piezoelectric, iron oxide, nanocage, nanotube, nanoshell, magnetic, paramagnetic, pyroelectric and piezoelectric nanoparticles, etc. are coated thermosensitive polymers, such as chitosan, liposomes, or polymer micelles or nanoparticles usually poly(N-isopropyl acrylamide) carrying a dye quenched with fluorescein or another dye or indicator/medication etc., and conjugated with an antineoplastic medication, gene, CRISPR-cas9 conjugated with cell penetrating agents or activatable cell penetrating agents injected in the patient's circulation, lymphatic vessels, inside a body cavity, cerebrospinal fluid, etc. The functionalized nanoparticles are exposed to microwaves, radiofrequency radiation, and/or electrical current generated by a battery where low electrical current from a battery passes from one side of the skin (i.e., the anode) through the body and a lesion, or tumor to the cathode electrode positioned on the opposite side of the skin on the body to raise the temperature of the piezoelectric or pyroelectric nanoparticles that are injected inside the body to be attached to the surface antigens of the normal cells or of tumor cells and create nanoparticle/tumor cell complexes to 41-43 degrees C., and when exposed to pulses of electrical current with an adjustable signal frequency and voltage, an acoustic response is produced by electrical stimulation of the piezoelectric nanoparticles inside the body that is called electroacoustic sounds or signals which can be captured with a transducer, or microphone, and converted to an electrical signal and is forwarded to a processor to be converted to a 1D, 2D, or 3D image as an electroacoustic computed tomogram while the electrical pulse generated in the piezoelectric nanoparticles drives the medication, gene, in the tumor cells locally to damage the tumor cells by multiple modes of the therapy applied non-invasively under observation.

In one embodiment, the antibody coated pluralities of gold, piezoelectric, iron oxide, nanocage, nanotube, nanoshell, magnetic, paramagnetic, pyroelectric and piezoelectric nanoparticles, etc. are coated with thermosensitive polymers, such as chitosan, quenched with fluorescein, liposomes, or polymer micelles or nanoparticles usually poly(N-isopropyl acrylamide) carrying a dye or another dye or indicator/medication etc. conjugated with an antineoplastic medication, gene, CRISPR-cas9 conjugated with cell penetrating agents or activatable cell penetrating agents injected in the patient's circulation, lymphatic vessels, inside a body cavity, cerebrospinal fluid, etc. The functionalized nanoparticles are exposed to microwaves, radiofrequency radiation, or focused ultrasound in a thermal or non-thermal mode or non-focused ultrasound, and/or electrical current generated by a battery where low electrical current from a battery passes from one side of the skin (i.e., the anode) through the body and a lesion or tumor to the cathode electrode positioned on the opposite side of the skin on the body to raise the temperature of the piezoelectric or pyroelectric nanoparticles that are injected inside the body to be attached to the surface antigens of the normal cells or of tumor cells and heat the nanoparticle/tumor cells complexes to 41-43 degrees C., and when exposed to pulses of electrical current with an adjustable signal frequency and voltage, an acoustic response is produced by electrical stimulation of the piezoelectric nanoparticles inside the body that is called electroacoustic sounds or signals which can be captured with a transducer or microphone, and converted to an electrical signal and forwarded to a processor to be converted to a 1D, 2D, or 3D image as an electroacoustic computed tomogram while the electrical pulse generated in the piezoelectric nanoparticles drives the medication, gene, in the tumor cells and the repeated low power ultrasound pulses generate an electric pulse from the piezoelectric nanoparticles that damages the tumor cell by depolarizing their cell membrane potential and to damage the tumor cells by multiple modes of the therapy applied non-invasively under observation.

In one embodiment, thermotherapy with focused high power ultrasound or alternating magnetic field, etc. is combined with the antibody coated pluralities of gold, piezoelectric, iron oxide, nanocage, nanotube, nanoshell, magnetic, paramagnetic, pyroelectric and/or piezoelectric nanoparticles, etc. are coated with thermosensitive polymers, such as chitosan, with quenched fluorescein, liposomes, or polymer micelles or nanoparticles usually poly(N-isopropyl acrylamide) carrying a dye another dye or indicator/medication, etc. conjugated with an antineoplastic medication, gene, CRISPR-cas9 conjugated with cell penetrating agents or activatable cell penetrating agents injected in the patient's circulation, lymphatic vessels, inside a body cavity, cerebrospinal fluid, etc. The functionalized nanoparticles are exposed to an alternating magnetic field and/or electrical current generated by a battery where low electrical current from a battery passes from one side of the skin (i.e., the anode) through the body and a lesion or tumor to the cathode electrode positioned on the opposite side of the skin on the body to raise the temperature of the piezoelectric or pyroelectric nanoparticles that are injected inside the body to be attached to the surface antigens of the normal cells or of tumor cells and heat the nanoparticle/tumor cell complexes to 41-43 degrees C., and when exposed to pulses of electrical current with an adjustable signal frequency and voltage, an acoustic response is produced by electrical stimulation of piezoelectric nanoparticles inside the body that we call electroacoustic sounds or signals which can be captured with a transducer, or microphone, and converted to an electrical signal and forwarded to a processor to be converted to a 1D, 2D, or 3D as an electroacoustic computed tomogram while the electrical pulse generated in the piezoelectric nanoparticles drives the medication, gene, in the tumor cells locally by depolarizing their cell membrane potential and to damage the tumor cells by multiple modes of the therapy applied non-invasively under observation.

In one embodiment, thermotherapy with focused low power ultrasound or alternating magnetic field, etc. is combined with the antibody coated pluralities of gold, piezoelectric, iron oxide, nanocage, nanotube, nanoshell, magnetic, paramagnetic, pyroelectric and/or piezoelectric nanoparticles, etc. are coated with thermosensitive polymers, such as chitosan, liposomes, or polymer micelles or nanoparticles usually poly(N-isopropyl acrylamide) carrying a dye with quenched fluorescein or another dye or indicator/medication, etc. conjugated with an antineoplastic medication, gene, CRISPR-cas9 conjugated with cell penetrating agents or activatable cell penetrating agents injected in the patient's circulation, lymphatic vessels, inside a body cavity, cerebrospinal fluid, etc. The functionalized nanoparticles are exposed to a divergent ultrasound and electrical current generated by a battery where low electrical current from a battery passes from one side of the skin (i.e., the anode) through the body and a lesion, or tumor to the cathode electrode positioned on the opposite side of the skin on the body to raise the temperature of the piezoelectric and/or pyroelectric nanoparticles that are injected inside the body to be attached to the surface antigens of the normal cells or of tumor cells and create nanoparticle/tumor cell complexes to 41-43 degrees C., and when exposed to pulses of electrical current with an adjustable signal frequency and voltage, an acoustic response is produced by electrical stimulation of the piezoelectric nanoparticles inside the body that is called electroacoustic sounds or signals which can be captured with a transducer, or microphone, and converted to an electrical signal and forwarded to a processor to be converted to a 1D, 2D, or 3D as an electroacoustic computed tomogram while the electrical pulse generated in the piezoelectric nanoparticles drives the medication, gene, in the tumor cells locally by depolarizing their cell membrane potential and to damage the tumor cells by multiple modes of the therapy applied non-invasively under observation.

In one embodiment, thermotherapy with focused low power ultrasound or alternating magnetic field, etc. is combined with the antibody coated pluralities of gold, piezoelectric, iron oxide, nanocage, nanotube, nanoshell, magnetic, paramagnetic, pyroelectric and/or piezoelectric nanoparticles, etc. are coated thermosensitive polymers, such as chitosan, liposomes, or polymer micelles or nanoparticles usually poly(N-isopropyl acrylamide) carrying a dye with quenched fluorescein or another dye or indicator/medication etc. conjugated with an antineoplastic medication, gene, CRISPR-cas9 conjugated with cell penetrating agents or activatable cell penetrating agents injected in the patient's circulation, lymphatic vessels, inside a body cavity, cerebrospinal fluid, etc. The functionalized nanoparticles are exposed to a non-focused ultrasound and electrical current generated by a battery where low electrical current from a battery passes from one side of the skin (i.e., the anode) through the body and a lesion or tumor to the cathode electrode positioned on the opposite side of the skin on the body to raise the temperature of the piezoelectric or pyroelectric nanoparticles that are injected inside the body to be attached to the surface antigens of the normal cells or of tumor cells and create nanoparticle/tumor cells complexes to 41-43 degrees, and when exposed to pulses of electrical current with an adjustable signal frequency and voltage, an acoustic response is produced by electrical stimulation of the piezoelectric nanoparticle inside the body that is called electroacoustic sounds or signals which can be captured with a transducer, or microphone, and converted to an electrical signal and forwarded to a processor to be converted to a 1D, 2D, or 3D image as an electroacoustic computed tomogram while the electrical pulse generated in the piezoelectric nanoparticles drives the medication, gene, in the tumor cells locally by depolarizing their cell membrane potential and to damage the tumor cells by multiple modes of the therapy applied non-invasively under observation.

In one embodiment, thermotherapy with focused low power ultrasound or alternating magnetic field, etc. is combined with the antibody coated pluralities of gold, piezoelectric, iron oxide, nanocage, nanotube, nanoshell, magnetic, paramagnetic, pyroelectric and/or piezoelectric nanoparticles, etc. are coated with thermosensitive polymers, such as chitosan, with quenched fluorescein or another dye or indicator/medication etc. conjugated with an antineoplastic medication, gene, CRISPR-cas9 conjugated with cell penetrating agents or activatable cell penetrating agents injected in the patient's circulation, lymphatic vessels, inside a body cavity, cerebrospinal fluid, etc. The functionalized nanoparticles are exposed to a focused low power ultrasound in a non-thermal mode (LIFU) or high power thermal mode (HIFU) generating an electrical current by the focused ultrasound from the piezoelectric nanoparticles and/or pyroelectric functionalized nanoparticles, thus creating an electrical signal from the piezoelectric nanoparticles that paralyses the cell permitting the functionalized nanoparticles coated with cell penetrating agents to enter the cell cytoplasm in the cells and release the medication, gene, CRISPR-cas9 inside the lesion or tumor by the focused ultrasound to raise the temperature of the piezoelectric or pyroelectric nanoparticles that are injected inside the body to be attached to the surface antigens of the normal cells or of tumor cells and create nanoparticle/tumor cell complexes to 41-43 degrees C. and a 1D, 2D, or 3D image as an acoustic computed tomogram while the nanoparticles drive the medication, gene, in the tumor cells locally by depolarizing their cell membrane potential and to damage the tumor cells by multiple modes of the therapy applied non-invasively under observation.

In one embodiment, thermotherapy with focused high power ultrasound or alternating magnetic field, etc. is combined with the antibody coated pluralities of gold, piezoelectric, iron oxide, nanocage, nanotube, nanoshell, magnetic, paramagnetic, pyroelectric and/or piezoelectric nanoparticles are coated with thermosensitive polymers, such as chitosan, with quenched fluorescein or another dye or indicator/medication etc. conjugated with an antineoplastic medication, gene, CRISPR-cas9 or Cas9/gRNA Ribonucleoprotein complexes (Cas9 RNPs) conjugated with cell penetrating agents or activatable cell penetrating agents injected in the patient's circulation, lymphatic vessels, inside a body cavity, cerebrospinal fluid, etc. is released that can be revealed when the nanoparticles are exposed to electromagnetic radiation, microwaves, or radiofrequency radiation, or focused ultrasound in a high power thermal or low power non-thermal mode or non-focused ultrasound, or an alternating magnetic field and/or electrical current generated by a battery where low electrical current from a battery passes from one side of the skin (i.e., the anode) through the body and a lesion or tumor to the cathode electrode positioned on the opposite side of the skin on the body to raise the temperature of the piezoelectric or pyroelectric nanoparticles that are injected inside the body to be attached to the surface antigens of the normal cells or of tumor cells and create nanoparticle/tumor cell complexes to 41-43 degrees C., and when exposed to pulses of electrical current with an adjustable signal frequency and voltage, an acoustic response is produced by electrical stimulation of piezoelectric nanoparticles inside the body that is called an electroacoustic sound or signal which can be recorded with a transducer, or microphone, and converted to an electrical signal and forwarded to a processor to be converted to a 1D, 2D, or 3D image as an electroacoustic computed tomogram while the electrical pulse generated in the piezoelectric nanoparticles drives the medication, gene, in the tumor cells locally by depolarizing their cell membrane potential and to damage the tumor cells by multiple modes of the therapy applied non-invasively.

In one embodiment, thermotherapy with focused low power ultrasound or alternating magnetic field, (<1K-300 KHz), etc. is combined with the antibody coated pluralities of gold, piezoelectric, iron oxide, nanocage, nanotube, nanoshell, magnetic, paramagnetic, pyroelectric and/or piezoelectric nanoparticles are coated with thermosensitive polymers, such as chitosan, with quenched fluorescein or another dye or indicator/medication, etc. conjugated with an antineoplastic medication, gene, CRISPR-cas9 conjugated with cell penetrating agents or activatable cell penetrating agents injected in the patient's circulation, lymphatic vessels, inside a body cavity, cerebrospinal fluid, etc. is released that can be revealed when the nanoparticles are exposed to electromagnetic radiation, combined focused high power ultrasound in a thermal or non-thermal mode or non-focused ultrasound, or electrical current generated by a battery where low electrical current from a battery passes from one side of the skin (i.e., the anode) through the body and a lesion or tumor to the cathode electrode positioned on the opposite side of the skin on the body to raise the temperature of the pluralities of the piezoelectric or pyroelectric nanoparticles that are injected inside the body to be attached to the surface antigens of the normal cells or of tumor cells and create nanoparticle/tumor cell complexes to 41-43 degrees C., and when exposed to pulses of electrical current with an adjustable signal frequency and voltage, an acoustic response is produced by electrical stimulation of piezoelectric nanoparticle inside the body that is called an electroacoustic sound or signal which can be recorded with a transducer, or microphone, converted to an electrical signal and forwarded to a processor to be converted to a 1D, 2D, or 3D image as an electroacoustic computed tomogram while the electrical pulse generated in the piezoelectric nanoparticles drives the medication, gene, in the tumor cells locally by depolarizing their cell membrane potential and to damage the tumor cells by multiple modes of the therapy applied non-invasively.

In one embodiment, where the thermal energy is an electromagnetic radiation, the pluralities of nanoparticles absorb the energy and expand, thereby creating a photoacoustic ultrasound that can be recorded by a photoacoustic or thermoacoustic imaging system indicating the degree of the temperature achieved, and the increased tumor biomarker in the circulation after the thermotherapy having an important diagnostic value (i.e., presence of a tumor by increased biomarkers) and therapeutic value for vaccine production after harvesting the circulating biomarkers and vaccine production with antibody coated nanoparticle with viral-like particles (VLP) or oncolytic viruses, such as T-Vec, monoclonal antibodies, IL-2, or IL-15, or anti-CD3 (OKT3) antibody, to increase cytotoxicity of natural killer cells, IFN-γ or granzyme β, related apoptosis-inducing ligand (TRAIL), or the Fas ligand, bee toxins or other immune stimulators along with checkpoint inhibitors and administering them with checkpoint inhibitors, such as PD-1, PD-11, CTLA-4, Jagged 1 inhibitor 15D11, etc. and Rock inhibitors, such as Fasudil, or Wnt inhibitors, such as niclosamide, for the future management of the tumor recurrences in the patient to reactivate the immune response and kill the tumor cells while inducing an immune response by the release of VLP, etc. as stimulators and checkpoint inhibitors enhance the immune response to the tumor locally and also their invisible metastatic lesions elsewhere.

In one embodiment, the photoacoustic or thermoacoustic unit is in communication with the thermal energy delivery system via a processor that can adjust thermal energy to achieve a desired temperature at the tumor site.

In one embodiment, the thermal output of the thermal energy delivery system is a focused ultrasound which heats up the tissue located at the focal point of the focused ultrasound using an antibody/medication coated ultrasound contrast agent, such as gold nanoparticles (GNPs), gold nanorods (GNRs), gold nanoshells (GNS), graphene oxides (GOs), polypyrrole (PPy) nanocapsules, or nanoshells nanocage magnetic, gold, silicone, carbon, mixture of magnetic Nps measuring the temperature non-invasively by estimation of the temperature depending on acoustic harmonic, and back scatter from 26-46 C degrees generated by the nonlinear ultrasound wave.

In one embodiment, the thermal output of the thermal energy delivery system is an electromagnetic radiation which heats up the tissue located at the focal point, or is another visible or infrared laser, or other electromagnetic radiation using an antibody/medication coated agent such as gold nanoparticles with activatable cell-penetrating peptides (ACPPs), gold nanoparticles (GNPs), gold nanorods (GNRs), gold nanoshells (GNS), graphene oxides (GOs), polypyrrole (PPy) nanocapsules, or nanoshells nanocage magnetic, gold, silicone, carbon, mixture of magnetic Nps to heat up the nanoparticles measuring the temperature non-invasively using a photoacoustic imaging system in a temperature range of 37-60 C degrees.

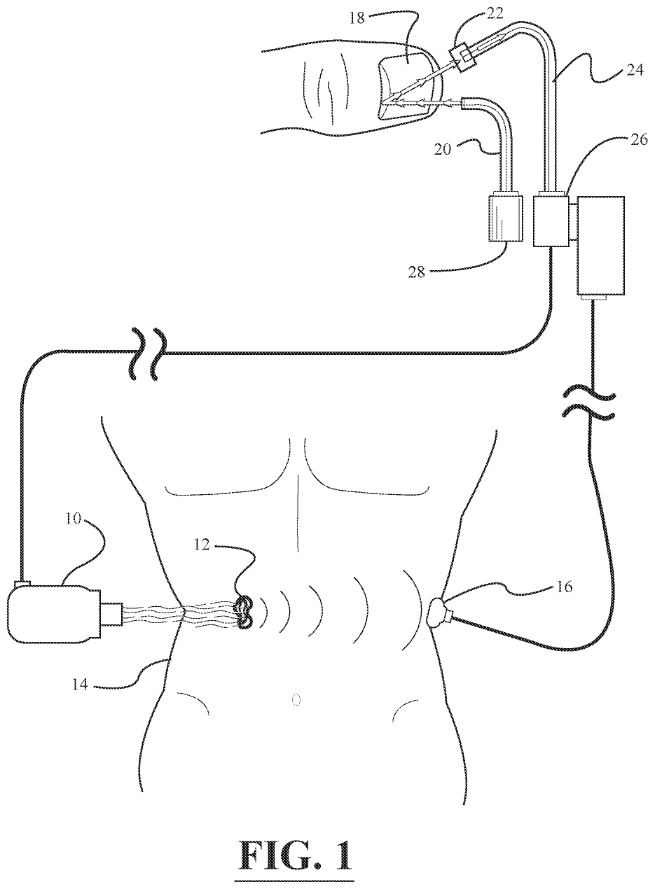

In one embodiment, with reference to FIG. 1, the thermal output of the thermal energy delivery system 10 is a focused high power ultrasound (HIFU) which heats up the tissue/tumor 12 located deep inside the body 14 at the focal point of ultrasound using an antibody/medication coated agent, such as iron oxide gold nanoparticles (GNPs) with activatable cell-penetrating peptides (ACPPs), gold graphene oxides (GOs), gold, silicone, carbon, mixture of magnetic nanoparticles (NPs), gold nanorods (GNRs), gold nanoshells (GNS) or a nanocage filled with a fluorescein dye or located inside a liposome containing quenched fluorescein or another dye or indicator, etc. that releases the dye when the nanoparticles temperature reaches a temperature of about 41-43 degrees C., thereby releasing the fluorescein in the circulation that can be observed by taking a blood sample or radiating the nail bed 18 of the patient with UV radiation delivered via a fiber optic 20 so that now non-quenched fluorescein fluoresces as green light passing through a blue filter 22 (see FIG. 1) demonstrating a hand held system with a blue wave length radiating source, such as a laser or diode 28 providing a wavelength of 380-390 nm illuminating a finger nail 18 and the capillaries in the nail bed, which in presence of unquenched fluorescein or another dye or indicator, etc. fluoresces, etc. a green light of 410 to 420 nm passing through a blue absorbing filter 22 and reached through a fiber optic 24 and a photomultiplier that converts the light to an electrical signal, then to a processor 26 that, in turn, is connected to the thermal delivery system indicating the temperature of 41-43 degrees C. is achieved, the operator can via software maintain the energy delivery for the desired time (e.g., 1-10 minutes or more, if needed) or continue the energy delivery until the next level of 43-56 degrees C. when a focused ultrasound is used and its terminal is connected to the same processor to continue delivering energy to reach the temperature of 56 degrees C. creating a popping or cavitation sound at the boiling point of the PFCL Nps when the ultrasonic receiver or the microphone 16 records a cavitation sound to be reported, via a receiver, to the processor 26 connected to either the ultrasonic unit or the alternating magnetic field (AMF) unit to stop producing the thermal energy to the tissue.

In one embodiment, one administers to a patient with a malignant tumor or benign lesion antibody coated magnetic nanoparticle conjugated with Rock inhibitors, Wnt inhibitors, or GSK inhibitors 269962, or aspirin, millimolar levels (˜5 mM), cyclo-oxygenase inhibitors, endomethacin (100-400 μM) the anti-leprotic clofazimine ˜3 μM serum concentration, and the anti-trypanosomal suramin ca. 200 metformin, Sulidac, suramin, Tigecyclin, clofazimine, pyrviniom pamoate 50-200 nM, GSK-beta inhibitors, intraperitoneal delivery at 10 mg/kg cyclodextrin conjugate ivermectin, (1-2 μM), niclosamide 0.2-0.4 inhibition of cancer cells growth (0.33-0.75 streptonigirin, salinomycin 0.3 and 10 μM serum concentration, topical Hexachlorophene, Imatinib, Gleevec/Glivec, a tyrosine kinase inhibitor, ethacrynic acid (EA) targets WNT genes such as fibronectin, cyclin D1, FDA approved riluzole conjugated with a thermosensitive polymer such as PEG, or chitosan or other polumers or nanoparticles carryng the slow release medication from porous silicon, or polylactic acid, or polyglycolic acid, along with other medications or genes with CRISPR Cas 9 that can be released under an alternating magnetic field, or 10 KHz to 300 Kz as LIFU, to shake up the polymeric material, gene, and medication without significant thermal effect or increasing the alternating magnetic field to 1 KHz to 10 KHz without producing thermal effect or more MHz to induce simultaneously thermal energy to heat up the tissue to a desired temperature as a result of the AMF frequency. In one embodiment, the thermal energy increases the surrounding tumor temperature from 37-41 C or 41 C or 50 C or more, which can be calculated depending the AMF frequency and the duration of the therapy to release the gene or medication and damage the tumor cell with increased temperature or killing them.

In one embodiment, a focused high power ultrasound is used to heat the antibody/medication coated nanoparticles conjugated with thermosensitive polymer carrying medication, dye, monoclonal antibody or check point inhibitors, coated nanoparticles to release the medication when a temperature of 43 degrees C. has been achieved and the medication is released locally without causing toxicity to the other organs of the patient and eliminating autoimmunity in the post treatment period.

In one embodiment, the external thermal energy is delivered by a combination source, such as electromagnetic radiation, alternating magnetic field, radio frequency (RF), and low power focused ultrasound either in a compressive or pressure or high power thermal mode and the nanoparticle/tumor temperature can be imaged simultaneously with the ultrasound, MRI, CT-scan, PET-scan, photoacoustic imaging unit, or combination of photoacoustic and ultrasound imaging, etc.

In one embodiment, the pluralities of functionalized nanoparticles with activatable cell-penetrating peptides (ACPPs) and Vitamin E are conjugated with metabolically active compounds, e.g., F-18FDG used to visualize tumors and metastatic lesions using positron emission tomography-computed tomography (PET-CT) scanning in addition to the external thermal energy to visualize and damage the otherwise not sensitive tumor stem cell.

In one embodiment, monoclonal antibodies (mAb) coated nanoparticles administered intravenously with checkpoint inhibitors, and antineoplastic medication, taxol derivatives or Wnt inhibitors, such as WIKI4, are conjugated with thermosensitive polymers coated pluralities of nanoparticles with activatable cell-penetrating peptides (ACPPs), nanoshells or nanocages, etc. to deliver monoclonal antibodies locally to the tumor cells while heating with a source of energy to melt the thermosensitive coating of the nanoparticle at the temperature of 41-43 degrees C. releasing the medications, and reducing the systemic side effects of intravenously or subcutaneously, intra-arterially, locally, in the body cavities, bladder, nose mouth, cerebrospinal fluid intraperitoneally delivered mAbs, checkpoint inhibitors and immune stimulators, Rock inhibitors, such as Fasudil, and its derivatives, etc., mAbs, or utomilumab antineoplastic medication, and removing them along with the released toxins (i.e., the toxic cytokines released from the tumors out of circulation) using dielectrophoresis, plasma exchange, plasmapheresis, blood dialysis at the end of the therapy, etc. and systemic medication with tocilizumab, a monoclonal antibody that targets interleukin-6 and ornithine phenylacetate an ammonia scavenger, for treatment of hepatic encephalopathy, a neuropsychiatric syndrome associated with hyperammonemia or ARF6 inhibition.

In one embodiment, the method of providing antibody-coated nanoparticles/gemtuzumab to a site, or for systemic delivery, may be combined with immunologically based B-cells or T-cells that are optionally genetically modified. Such B-cells and T-cells may attack localized and/or unlocalized cancer cells or hematological cancers (e.g., leukemias, lymphomas, etc.). In one embodiment, extra-corporal dialysis and/or plasmapheresis and systemic medication with tocilizumab, a monoclonal antibody that targets interleukin-6 and ornithine phenylacetate an ammonia scavenger, for treatment of hepatic encephalopathy, a neuropsychiatric syndrome associated with hyperammonemia in combination with the inventive method of delivering antibody-coated nanoparticles with ACPP to accomplish removal of excessive tumor protein in order to protect vital organs (e.g., kidney, liver, brain). In one embodiment, one or more of these therapies are employed at lower doses when used in combination with the method of delivering antibody/medication coated nanoparticles.

In other embodiments, nanoparticles of a material other than gold may be used. These include, without limitation, antibody/medication and thermosensitive coated diamond nanoparticles, platinum nanoparticles, combinations of gold, platinum, carbon, and/or diamond nanoparticles. Any of the above nanoparticles may contain at least one hydroxyl group. All such nanoparticles provide the various diagnostic and therapeutic applications as described above for gold nanoparticles. The sizes and shapes are the same as those described for gold nanoparticles. All such nanoparticles may be covalently attached to (poly)ethyleneglycol, (i.e., may be PEGylated).

In one embodiment, the application of thermotherapy to reach the temperature of 41-43 to 50 C to 56 degrees C. temperature releases more cytokines from the damaged tumor cells activating the immune cell response more than if the medications were administered alone without thermotherapy, eliminating the need for CAR-T cellular therapy which requires a time consuming and expensive preparation with the chance of increased toxicity and an autoimmune response.

In another embodiment, the preparation of vaccine using the biomarkers and VLP, antibody coated nanoparticles with viral-like particles (VLP) or oncolytic viruses, such as T-Vec, monoclonal antibodies, IL-2, or IL-15, or anti-CD3 (OKT3) antibody, to increase cytotoxicity of natural killer cells, IL-6, IL-1β, TNF-α, or CD40 agonist, IFN-γ or granzyme β, related apoptosis-inducing ligand (TRAIL), or the Fas ligand, bee toxins or other immune stimulators along with checkpoint inhibitors conjugated with antibody/medication coated nanoparticles and administering them with checkpoint inhibitors, such as PD-1, PD-L1, CTLA-4, Jagged 1 inhibitor 15D11, alone or preferably in combination, etc. in a formulation of polymeric slow release antibody coated nano-to microparticles of polylactic or polycaprolactone, copolymers of glycolic and lactic acid, polyanhydrides or orthoesters, etc can be injected locally, inside the tumor. In one embodiment, vaccination is done every week for a month and then every three months for the first year, then every six months and beyond to enhance immunity of the patient and prevent recurrences.

In one embodiment, the thermal output of the thermal energy delivery system is a focused high power ultrasound which heats up the tissue located at the focal point of the focused ultrasound using an antibody/medication coated ultrasound contrast agent, such as gold nanoparticles (GNPs), gold nanorods (GNRs), gold nanoshells (GNS), graphene oxides (GOs), polypyrrole (PPy) nanocapsules, or nanoshells, nanocage magnetic, gold, silicone, carbon, mixture of magnetic Nps and PFCL, quenched fluorescein, or another dye or indicator, bubble liposome fluorescein, PFC1 nanoemulsions, etc., gold nanorods (GNRs) filled with albumen microbubbles, GNR loaded albumin microbubbles for combined ultrasound and photoacoustic or thermoacoustic imaging or their combination imaging and thermotherapy therapy and the increased tumor biomarkers in the circulation after the thermotherapy has an important diagnostic value (i.e., confirming the presence of a tumor) and therapeutic value as biomarkers for the future management of the patient with recurrences by making a vaccine using the biomarkers and VLP, antibody coated nanoparticle with viral-like particles (VLP) or oncolytic viruses, such as T-Vec, monoclonal antibodies, IL-2, or IL-15, or anti-CD3 (OKT3) antibody, or polymeric antibody coated nanoparticles conjugated with IL-2 and IL-21 to increase cytotoxicity of natural killer cells, IL-6, IL-1β, IL-17, TNF-a, a CD40 agonist, IFN-γ or granzyme β, related apoptosis-inducing ligand (TRAIL), or the Fas ligand, bee toxins or other immune stimulators along with checkpoint inhibitors conjugated with antibody/medication coated nanoparticle and administering them with checkpoint inhibitors, such as PD-1, PD-11, CTLA-4, Jagged 1 inhibitor 15D11, etc. and a formulation of polymeric nano- to microparticles of polylactic or polycaprolactone, copolymers of glycolic and lactic acid, etc. having Rock inhibitors, such as Fasudil, netasudil, SAR407899, etc. and/or Wnt inhibitors, such as FH535, IWP-2, PNU-74654, IWR-Tendo, IWR-exo, niclosamide or GSK inhibitors SB-216763 etc. at non-toxic concentrations release of 1 picogram to nanogram/ml or higher concentrations daily or aspirin, millimolar levels (˜5 mM), cyclo-oxygenase inhibitors, endomethacin (100-400 μM) the anti-leprotic clofazimine ˜3 μM serum concentration, and the anti-trypanosomal suramin ca. 200 metformin, Sulidac, suramin, Tigecyclin, clofazimine, pyrviniom pamoate 50-200 nM, GSK-beta inhibitors, intraperitoneal delivery at 10 mg/kg cyclodextrin conjugate ivermectin (1-2 μM), Niclosamide 0.2-0.4 inhibition of cancer cells growth (0.33-0.75 streptonigirin, salinomycin 0.3 and 10 μM serum concentration, topical Hexachlorophene, Imatinib, Gleevec/Glivec, a tyrosine kinase inhibitor, ethacrynic acid (EA) targets Wnt genes such as fibronectin, cyclin D1, FDA approved riluzole, or integrin inhibitors such as abciximab, Eptifibatide, Tirofiban, αIIbβ3 antagonists, Natalizumab, 3 microgam/daily to be administered to the patient to attach to the potential tumor cells, exosomes, while applying thermal energy to damage tumor recurrence and induce reactivation of the immune response to eliminate them, thereby eliminating the need for repeated CAR-T cell therapy while inducing an immune response by the release of VLP, etc. as stimulators and checkpoint inhibitors that enhance the immune response to the tumor locally inside the tumor and also their invisible metastatic lesions elsewhere.

In one embodiment, monoclonal antibodies, checkpoint inhibitors or antineoplastic medications, utomilumab are conjugated with antibody coated nanoparticles, nanoshells nanocage magnetic or paramagnetic, and VLP or oncolytic viruses, such as T-Vec, monoclonal antibodies, IL-2, or IL-15, polymeric antibody coated nanoparticles conjugated with IL-2 and IL-21, or anti-CD3 (OKT3) antibody, to increase cytotoxicity of natural killer cells, IFN-γ or granzyme β, related apoptosis-inducing ligand (TRAIL), or the Fas ligand, bee toxins or other immune stimulators along with checkpoint inhibitors such as PD-1, PD-L1, CTLA-4, Jagged 1 inhibitor 15D11, etc. to deliver them to the tumor cells by systemic or local administration, releasing them from the thermosensitive polymers of the pluralities of nanoparticles with activatable cell-penetrating peptides (ACPPs) at a controlled predetermined temperature of 41-43 degrees C. via a software controlling the energy producing energy source since intravenous administration of monoclonal antibodies or check point inhibitors or antineoplastic medications or immune stimulators are administered in significantly higher dose than nanoparticle conjugated medications, VLP, and immune stimulator that are released locally at the cancer cells and have many side effects such as hyperactivation of T-cells and cytokine release causing nonspecific or specific body response (e.g., checkpoint inhibitors and vaccine therapy cause recruitment of specific T-cell to attack the tumor and potentially some specific organs that share the same checkpoint inhibitors) or cause excessive cytokine release producing Cytokine release syndrome (CRS) associated with fever, tachycardia, vascular leak, oliguria, hypotension, hyper activation of T-cells and cytokine such as those seen with checkpoint Protein Inhibitors, Rock inhibitors, such as Fasudil, and its derivatives etc., or Wnt inhibitors Ipilimumab blocks CTLA-4; pembrolizumab blocks PD-1; and nivolumab also blocks PD-1, or utomilumab inducing colitis, pancreatitis, pneumonitis, hepatitis, skin reactions or mAbs induce arthralgia, enteritis, encephalitis, Guillain-Barre syndrome, myasthenia gravis-like syndrome, and autoimmune bone marrow suppression, skin-related toxicities, hepatitis and endocrinopathies and often multiple therapy, when cytokines are used, such as Recombinant human interferon alfa (IFN), one observes fever, fatigue, headache and myalgia, thrombocytopenia and leukopenia, Vitiligo, lupus, rheumatoid arthritis, polymyalgia rheumatica, autoimmunity, neurotoxicity, and myocarditis and toxicities related to adaptive cell therapy and to enhance immune therapy.

In one embodiment, during the thermal delivery to the nanoparticle, nanoshells, or nanocages, administered intravenously one can measure two distinct temperatures inside the body regardless of their location using either an electromagnetic, focused high power ultrasound, or alternating magnetic field, while releasing the medications at temperature of 41-43 degrees C., this can achieves a theranostic or diagnostic and therapeutic effect when the nanoparticles with activatable cell-penetrating peptides (ACPPs) carry a thermosensitive polymer, such as chitosan, and a dye, such as quenched fluorescein, or another dye or indicator, etc. that can be only detected when it is released in the blood indicating the temperature of the nanoparticle and release of the medication conjugated with the chitosan by detection of unquenched fluorescein presence in the blood continuously passing through a flexible silicone tube radiated by UV radiation or other wavelength of light, further heating the nanoparticle creates a cavitation sound at temperature or either 50 degrees C. or 56 degrees C., thermotherapy with focused ultrasound or alternating magnetic field, etc. is combined with the thermal energy delivery source or combinations with low power, compressive or pressure ultrasound.

In one embodiment, thermotherapy with focused ultrasound or alternating magnetic field, etc. is combined with the thermosensitive polymeric compound chitosan and can carry DNA, siRNA, RNA, other genes or inhibitory genes and VLP, or oncolytic, intra-tumor injectables viruses, such as T-Vec, monoclonal antibodies, IL-2, or IL-15, or anti-CD3 (OKT3) antibody, to increase cytotoxicity of natural killer cells, IL-6, IL-1β, bee toxins or other immune stimulators along with checkpoint inhibitors, such as PD-1, PD-L1, CTLA-4, Jagged 1 inhibitor 15D11, etc. are released at the temperature of 41-43 degrees C. along with a quenched fluorescein dye or another dye or indicator in the circulation and can be seen in a few seconds in the circulation using a blood sample or inside a transparent butterfly tubing that fluoresces under UV radiation indicating presence of the dye indicating that the temperature 41-43 degrees C. at the site of the heated nanoparticles/tumor is achieved and the genes are released while inducing an immune response by the release of VLP, etc. as stimulators and checkpoint inhibitors enhance the immune response to the tumor locally and also their invisible metastatic lesions elsewhere, while antibody coated nanoparticles/checkpoint inhibitors and VLP attach to the localized or circulating tumor cells and their exosomes, which are carrying a checkpoint protein, such as PD-L1 (to disguise themselves), and the tumor cells are recognized by the T cells, which together with killer cells phagocytose them, and enhance the immune response to the tumor and its exosomes and the circulating cells and eliminate them.

In one embodiment, the thermosensitive polymeric compound is chitosan and can carry DNA, siRNA, RNA, other genes or inhibitory genes, that are released at the temperature of 41-43 degrees C. along with a quenched fluorescein dye, or another dye or indicator, etc. in the circulation and can be seen in a few seconds in the circulation using a blood sample or inside a transparent butterfly tubing that fluoresces under UV radiation or other wave length indicating presence of the dye indicating that the temperature 41-43 degrees C. at the site of the heated nanoparticles/tumor is achieved and the genes are released.

In one embodiment, a combination of nanoparticles with activatable cell-penetrating peptides (ACPPs) is used to have additional functionalities as well as imaging, thermoacoustic imaging for thermotherapy and imaging and tumor therapy by administering to a patient systemically or locally either antibody/medication coated copper sulfide (CuS) nanoparticles, or polymeric microbubbles of Polylactic acid or lactic acid microcapsules employing the water-in-oil-in-water double emulsion method or gold Nanorod-loaded PLA microcapsules for combined ultrasound contrast imaging and conjugated with a thermosensitive polymer, such as chitosan with DNA, siRNA, RNAi, with CRISPR enzyme or with or without macrolides, such as cyclosporine A, mycophenolic acid, tacrolimus or ascomycin or other drugs delivery at 41-43 degrees C. temperature or monoclonal antibodies, checkpoint inhibitor, anti-neoplastic medications, or antibody coated gold nanoparticles with activatable cell-penetrating peptides (ACPPs) or antibody/medication coated gold nanoshells, perfluorocarbon liquid, or antibody coated perfluorohexane (PFH)-loaded magnetic hollow iron oxide nanoparticles (HIONs) and Fe-O stretching vibration mode of Fe3O4T, or antibody coated nanoparticles, such as antibody/medication coated SPIOs or Gold Nanorod and iron oxide coating nanocapsules that absorb thermal delivery using alternating magnetic field, focused high power ultrasound and electromagnetic radiation or imaging simultaneously with MRI or CT contrast imaging, PET-scan, while having high X-ray absorption coefficient, to have multifunctional capabilities for cancer diagnosis and thermotherapy and thermoacoustic imaging for control of the temperature.

In one embodiment, the temperature of antibody/medication coated pluralities of nanoparticles administered intravenously with activatable cell-penetrating peptides (ACPPs), nanocages, nanoshells, etc. is measured and imaged using different indicators of the temperature at the tumor site during the thermotherapy indicating temperature of either 41-43 degrees C. or 44-49 degrees C. or 50 degrees C. or 56 degrees C. using electromagnetic radiation and photoacoustic or thermoacoustic temperature imaging system or a temperature using alternating magnetic field, microwave, or radiofrequency to increase the temperature from 37-43 degrees C. measured using a biocompatible dye, such as quenched fluorescein, or another dye or indicator, etc. lipid coated nanoparticles that melt at 41-43 degrees C. temperature and releases the dye in the circulation and recognized optically as in FIG. 1, or in the blood sample with UV radiation or the use of nanoshells or nanocages containing either a perfluorocarbon liquid such as perfluoro pentane or hexane that boils at the temperature of 56 degrees C., thereby creating a recordable cavitation sound recorded by a microphone of an ultrasonic receiver or, if heated with a source of energy such as alternating magnetic field (AMF) with the magnetic or paramagnetic nanoparticles with activatable cell-penetrating peptides (ACPPs) or nanoshells, etc. or focused ultrasound or at temperature of 50 degrees C. if a combination of AMF and high power FUS or compressive or pressure US and AMF is used, and the increased tumor biomarkers in the circulation after the thermotherapy has an important diagnostic value (i.e., confirming the presence of a tumor) and therapeutic value as biomarkers for the future management of the patient with recurrences by making a vaccine using the biomarkers or, tumor antigen, and antibody/medication coated nanoparticles and VLP and administering them with checkpoint inhibitors, such as PD-1, PD-11, CTLA-4, Jagged 1 inhibitor 15D11, etc., and Rock inhibitor, such as Fasudil, and its derivatives or Wnt inhibitors, such as niclosamide, to attach to the potential tumor cells while applying thermal energy to damage the recurrence of the tumor and induce reactivation of the immune response and eliminate the tumor cells and to enhance immune therapy.

In one embodiment, the administered thermosensitive polymeric/medication compound is chitosan conjugated with monoclonal antibodies (mAbs) or checkpoint inhibitors, Rock inhibitors such as Fasudil, and its derivatives, etc., VLP, or oncolytic intratumor injectable viruses, monoclonal antibodies, IL-2, or IL-15, or anti-CD3 (OKT3) antibody, to increase cytotoxicity of natural killer cells, bee toxins or other immune stimulators along with checkpoint inhibitors, such as PD-1, PD-L1, CTLA-4, Jagged 1 inhibitor 15D11 or an antineoplastic medication, daunorubicin, ara-C or cytarabine, taxol, taxane derivatives, etc. that are released at the temperature of 41-43 degrees C. along with a quenched fluorescein dye or another quenched dye or indicator/medication, PFC nanobubbles, etc. in the circulation and can be seen in a few seconds in the circulation using a blood sample or inside a transparent butterfly tubing that fluoresces under UV radiation indicating presence of the dye indicating that the temperature 41-43 degrees C. at the site of the heated nanoparticles/tumor is achieved and the increased tumor biomarkers in the circulation after the thermotherapy has an important diagnostic value (i.e., confirming the presence of a tumor) and therapeutic value as biomarkers for the future management of the patient with recurrences by making a vaccine using the biomarkers and VLP, IL-2, or IL-15, or anti-CD3 (OKT3) antibody, to increase cytotoxicity of natural killer cells, conjugated with antibody coated nanoparticles and administering them with checkpoint inhibitors, such as PD-1, PD-L1, CTLA-4, Jagged 1 inhibitor 15D11, etc. to enhance immune therapy, and Rock inhibitors, such as Fasudil or Wnt inhibitors, such as niclosamide, to attach to the potential tumor cells, while applying thermal energy to damage recurrence of the tumor and induce reactivation of the immune response to eliminate them, thereby eliminating the need to repeat CAR-T cell therapy unless the patient is immunosuppressed, vaccination is done every week or less for a month and then every three months for the first year then every six month and beyond to enhance immunity of the patient and prevent recurrences.

In one embodiment, the administered thermosensitive polymeric compound is chitosan conjugated with mAbs or checkpoint inhibitors, or antineoplastic medication, Daunorubicin, ara-C or cytarabine, taxol, taxane derivatives, etc. that are released at the temperature of 41-43 degrees C. along with a quenched fluorescein dye or another quenched dye or indicator, etc. in the circulation and can be seen in a few seconds in the circulation using a blood sample or inside a transparent butterfly tubing that fluoresces under UV radiation indicating presence of the dye indicating that the temperature 41-43 degrees C. at the site of the heated nanoparticles/tumor is achieved and the increased tumor biomarkers in the circulation after the thermotherapy has an important diagnostic value (i.e., confirming the presence of a tumor) and therapeutic value as biomarkers for the future management of the patient with recurrences by making a vaccine using the biomarkers and VLP or intratumor injectable viruses, monoclonal antibodies, IL-2, or anti-CD3 (OKT3) antibody, to increase cytotoxicity of natural killer cells, bee toxins or other immune stimulators along with checkpoint inhibitors, such as PD-1, PD-L1, CTLA-4, Jagged 1 inhibitor 15D11 conjugated with antibody/medication coated nanoparticles and administering them with Rock inhibitors, such as Fasudil, or Wnt inhibitors, such as niclosamide, to attach to the potential tumor cells, while applying thermal energy to damage recurrence of the tumor and induce reactivation of the immune response to eliminate them as enhanced immune therapy, thereby eliminating the need to repeat CAR-T cell therapy unless the patient is immunosuppressed, vaccination is done every week for a month, and then every three months for the first year, then every six months and beyond to enhance immunity of the patient and prevent recurrences along with modified killer cells in immunosuppressed patients.

In one embodiment, the administered thermosensitive polymeric compound is chitosan conjugated with mAbs or check point inhibitors, or antineoplastic medication, Artemisinins or artemisinin analogues against a variety of cancer cells, promoting apoptosis, preventing angiogenesis inhibition of Toll-like receptors, Syk, tyrosine kinase inhibitor, phospholipase Cy, PI3K/Akt, MAPK, STAT-1/3/5, NF-κB, Spl, Axitinib and Nrf2/ARE signaling pathways that are released at the temperature of 41-43 degrees C. along with a quenched fluorescein dye or another quenched dye or indicator, etc. in the circulation and can be seen in a few seconds in the circulation using a blood sample or inside a transparent butterfly tubing that fluoresces under UV radiation indicating presence of the dye indicating that the temperature 41-43 degrees C. at the site of the heated nanoparticles/tumor is achieved and the increased tumor biomarkers in the circulation after the thermotherapy has an important diagnostic value (i.e., confirming the presence of a tumor) and therapeutic value as biomarkers for the future management of the patient with recurrences by making a vaccine using the biomarkers and VLP, or intratumor injectable viruses, such as T-Vec, monoclonal antibodies, IL-2, or anti-CD3 (OKT3) antibody, to increase cytotoxicity of natural killer cells, bee toxins or other immune stimulators conjugated with antibody coated nanoparticles and administering them with checkpoint inhibitors, such as PD-1, PD-L1, CTLA-4, Jagged 1 inhibitor 15D11, to enhance immune therapy etc., and Rock inhibitors, such as Fasudil, and its derivatives, or Wnt inhibitors, such as niclosamide, to attach to the potential tumor cells while applying thermal energy to damage recurrence of the tumor and induce reactivation of the immune response to eliminate them, thereby eliminating the need to repeat CAR-T cell therapy unless the patient is immunosuppressed while antibody coated nanoparticles/checkpoint inhibitors and VLP attach to the localized or circulating tumor cells and their exosomes, which are carrying a checkpoint protein, such as PD-L1 (to disguise themselves), and the tumor cells are recognized by the T cells, which together with killer cells phagocytose them, and enhance the immune response to the tumor and its exosomes and the circulating cells.

In one embodiment, the Wnt/β-catenin pathway can be inhibited indirectly by inhibiting GSK-3 Glycogen Synthetizing Kinase, such as Lithium chloride, a simple salt, or its Phosphoaminophosphonic form at an extremely low concentration of 1-3 nano-micromolar that can only be delivered using antibody coated pluralities of nanoparticles conjugated with thermosensitive polymers, such as chitosan conjugated with ACPP, CPP, Vitamin E, and quenched fluorescein, or another quenched dye or indicator/medication, etc. and or an antineoplastic medication or monoclonal antibody to release the dye and medication by thermal energy at controlled temperature of 39-41 degrees C. to not only inhibit the Wnt pathway but also treat various Wnt/β-catenin signaling in a number of diseases such as inflammation, brain cancers, glioblastoma, glioma, prostate, breast, colon cancer, ovarian cancer, and Alzheimer's Disease.

In one embodiment, the Wnt/β-catenin pathway inhibitors can be used with sulfasalazine inhibitor of PIK3CA mutations, anti-integrins, enzymes that are involved in cellular growth control signals, that is known to inhibit xCT, and can be delivered using antibody coated pluralities of nanoparticles conjugated with thermosensitive polymers, such as chitosan, conjugated with ACPP and quenched fluorescein, or another quenched dye or indicator/medication, etc. and or an antineoplastic medication or monoclonal antibody to release the dye and medication by thermal energy at a controlled temperature of 41-42 degrees C. to not only inhibit the Wnt pathway but also treat various Wnt/β-catenin signaling in a number of diseases inhibit cCT and PIK3CA, such as in inflammation, brain cancers, glioblastoma, glioma, prostate, breast, colon cancer, and ovarian cancer.

In one embodiment, the Wnt/β-catenin pathway inhibitors or Rock inhibitors, such as Fasudil, etc., can be used with sulfasalazine inhibitor of PIK3CA mutations, enzymes that are involved in cellular growth control signals, that is known to inhibit xCT and can be delivered using antibody coated pluralities of nanoparticles conjugated with thermosensitive polymers, such as chitosan, conjugated with ACPP and quenched fluorescein or another quenched dye or indicator/medication, such as inhibition of tyrosine, monoclonal antibodies to EGFR, VEGFR, PDGFR, and c-kit Cdc42: cell division control protein 42, ERK: extracellular signal-regulated kinase, mTOR: mammalian target of rapamycin, PI3K: phosphatidylinositol 3-kinase, EGF(R): epidermal growth factor (receptor), Grb2: growth factor receptor-bound protein 2, JNK: c-Jun N-terminal kinase, MEK/MKK: mitogen-activated protein kinase kinases, PDGF(R): platelet derived growth factor (receptor), SOS: son of sevenless, TAK: TGFβ-activated kinase, TGF: transforming growth factor, and VEGF(R): vascular endothelial growth factor (receptor) to release the dye and medication by thermal energy at controlled temperature of 41-42 degrees C. to not only inhibit the Wnt pathway but also treat various Wnt/β-catenin signaling in a number of diseases inhibit cCT and PIK3CA such as in inflammation, brain cancers, glioblastoma, glioma, glioma multiforme, prostate, breast, colon cancer, ovarian cancer.

In one embodiment, the Wnt/β-catenin pathway inhibitors, such as niclosamide at 500 mg dose, ivermectin 1 gram-2 gram once a week, etc., or Rock inhibitors, such as Fasudil at 40-80 mg, etc., can be used orally at approved doses or systemically with systemic sulfasalazine inhibitor of PIK3CA mutations, enzymes that are involved in cellular growth control signals, that is known to inhibit xCT and can be delivered using antibody coated pluralities of nanoparticles conjugated with thermosensitive polymers, such as chitosan, conjugated with ACPP and quenched fluorescein or another quenched dye or indicator/medication, such as inhibition of tyrosine, monoclonal antibodies to EGFR, VEGFR, PDGFR, and c-kit Cdc42: cell division control protein 42, ERK: extracellular signal-regulated kinase, mTOR: mammalian target of rapamycin, PI3K: phosphatidylinositol 3-kinase, EGF(R): epidermal growth factor (receptor), Grb2: growth factor receptor-bound protein 2, JNK: c-Jun N-terminal kinase, MEK/MKK: mitogen-activated protein kinase kinases, PDGF(R): platelet derived growth factor (receptor), SOS: son of sevenless, TAK: TGFβ-activated kinase, TGF: transforming growth factor, and VEGF(R): vascular endothelial growth factor (receptor) to release the dye and medication by thermal energy at controlled temperature of 41-42 degrees C. to not only inhibit the Wnt pathway but also treat various Wnt/β-catenin signaling in a number of diseases inhibit cCT and PIK3CA such as in inflammation, brain cancers, glioblastoma, glioma, multiforme, prostate, breast, colon cancer, and ovarian cancer, etc.

In one embodiment, when the AMF or thermal mode of the focused ultrasound is used when the boiling point of the PFCL is reached by heating the pluralities of nanoparticles administered with activatable cell-penetrating peptides (ACPPs), cage, shells, a popping cavitation sound is produced and recorded by a microphone or an ultrasonic recorder, indicating the temperature of about 50 degrees C. when the combination of sources of energy, or a combination of high power thermal energy and a low power compressive or pressure focused ultrasound is used creating a recordable cavitation sound at the temperature of about 56 degrees C. and the medication is released from the nanoparticles with activatable cell-penetrating peptides (ACPPs) and the tumor cells to which the nanoparticles are attached are damaged by the thermal energy indicating sudden increase of tumor biomarkers in the circulation taken from blood samples for future antibody production in a potential repeated and the increased tumor biomarkers in the circulation after the thermotherapy has an important diagnostic value (i.e., confirming the presence of a tumor) and therapeutic value as biomarkers for the future management of the patient with recurrences by making a vaccine using the biomarkers and VLP or intratumor injectable intratumor injectable oncolytic viruses, monoclonal antibodies, IL-2, or anti-CD3 (OKT3) antibody, to increase cytotoxicity of natural killer cells, bee toxins or other immune stimulators conjugated with antibody coated nanoparticles and administering them with checkpoint inhibitors such as PD-1, PD-11, CTLA-4, Jagged 1 inhibitor 15D11, etc., and Rock inhibitors, such as Fasudil, or Wnt inhibitors, such as niclosamide, to attach to the potential tumor cells while applying thermal energy to damage recurrence of the tumor and induce reactivation of the immune response to eliminate them and release the immune stimulators with checkpoint inhibitors to enhance immune therapy, while antibody coated nanoparticles/checkpoint inhibitors and VLP attach to the localized or circulating tumor cells and their exosomes, which are carrying a checkpoint protein, such as PD-L1 (to disguise themselves), and the tumor cells are recognized by the T cells, which together with killer cells phagocytose them, and enhance the immune response to the tumor and its exosomes and the circulating cells.

In one embodiment, one achieves a theranostic or diagnostic and therapeutic effect when the antibody coated pluralities of nanoparticles with activatable cell-penetrating peptides (ACPPs) conjugated with a Wnt inhibitor, such as ivermectin, and a Rock inhibitor, such as Fasudil, Y-27632, netarsudil, small molecule inhibitor of ROCK1 and ROCK2 which act as an anti-inflammatory agent and inhibit Wnt activation, are encapsulated in the antibody coated liposomes or antibody coated nanoparticles conjugated with a thermosensitive polymer such as chitosan, and containing an immune stimulators, such as VLP, intratumor injectable oncolytic viruses, IL-2, or IL-15, or polymeric antibody coated nanoparticles carrying IL-2 and IL-21 or anti-CD3 (OKT3) antibody, to increase cytotoxicity of natural killer cells, IL-6, IL-1β, IL-17, interferons, TLR 7, TLR8, IFN-γ or granzyme β, related apoptosis-inducing ligand (TRAIL), or the Fas ligand, checkpoint inhibitor(s), polysaccharide K, a toxin and a photosensitizer such as Verteporfin or phthalocyanine, etc. or another dye and electromagnetic energy, such as laser, absorbing antibody coated nanoparticles with activatable cell-penetrating peptides (ACPPs), such iron oxide, gold, silicon carbon, magnetic, non-magnetic NPs or nanoshells, nanocages, nanotubes, nanorods, nanospheres, or any other shells produced (e.g., polylactic or polyglycolic acid, etc.) or piezoelectric nanoparticle administered systemically, topically, locally inside the tumor exposed to a thermal energy source such ultrasound or as laser light from 360-1000 nm or more or microwave or Radio frequency wave, to increase the temperature of the nanoparticles inducing a photoacoustic or thermoacoustic sound measuring the temperature of the heated tumor from 40-43- or more as needed and, the lipid component of the liposome and chitosan, melts, releasing medication and the dye at temperature of 41-43 C degrees C. or more to release the medication, etc. initiating a precise photodynamic effect on the tumor cells along with VLP and checkpoint inhibitors release inducing a photodynamic effect with thermotherapy and immune stimulation to kill and remove the tumor cells by the natural killer cells, activated T-cells etc., the temperature of 41-43 or more degrees C. is achieved and the medication is released from the NPs, and the achieved temperature is recognized by photoacoustic acoustic imaging for control of the temperature communicating with the thermal delivery e.g. laser unit via a processor to control the amount of energy released by the thermal energy delivery unit via a processor to stop or increase or decrease the temperature or by increasing the temperature of the nanoparticles or nanoshells, while antibody coated nanoparticles/checkpoint inhibitors and VLP attach to the localized or circulating tumor cells and their exosomes, which are carrying a checkpoint protein, such as PD-L1 etc. (to disguise themselves), and the tumor cells and exosomes are recognized by the T-cells, which together with natural killer cells phagocytose them, and enhance immune response to the tumor, and its exosomes and the circulating cells.

In one embodiment, the polymeric thermosensitive coating of the pluralities of nanoparticles administered with activatable cell-penetrating peptides (ACPPs) melts and releases the medication at the melting point of the polymer, such as chitosan or liposomes containing quenched fluorescein dye or another quenched dye or indicator, medication, etc. at a 41-43 degrees C. temperature by heating the nanoparticles with activatable cell-penetrating peptides (ACPPs) either with electromagnetic radiation and imaging the temperature continuously from 37 to 56 degrees C. or alternating magnetic field or focused ultrasound or combination of alternating magnetic field with focused or focused compressive or low power ultrasound is used for simultaneous minimal heating and/or imaging, or high power focused ultrasound and compressive or pressure ultrasound are applied alternatively every few seconds to achieve release of medication and treat the tumor cell by thermal energy simultaneously and the increased tumor biomarkers in the circulation after the thermotherapy has an important diagnostic value (i.e., confirming the presence of a tumor) and therapeutic value as biomarkers for the future management of the patient with recurrences by making a vaccine using the biomarkers and conjugated with antibody coated nanoparticle and checkpoint inhibitors, such as PD-1, Jagged 1 inhibitor 15D11, to attach to the potential tumor cells while applying thermal energy to damage recurrence of the tumor and induce reactivation of the immune response to eliminate them.

In one embodiment, these biguanides administered along with aspirin can prevent cancer formation and in cancer treatment by inhibition of mTOR signaling via AMP-activated protein kinase (AMPK)-dependent and -independent path and inhibition of COX-1/COX-2 and modulation of the NFκB or STAT3 pathway and activation AMPK, and both agents may affect Notch, Wnt/β-catenin when delivered through the thermosensitive polymeric coated functionalized pluralities of nanoparticles and ACPP intravenously, orally or intracavity or intra-arterially at one nanogram to 100 nanogram concentrations interfering with the glucose metabolism of the tumor stem cells proliferation causing them to starve.

In one embodiment, antibody/medication coated pluralities of nanoparticles administered with activatable cell-penetrating peptides (ACPPs) conjugated with Wnt inhibitor such as FH535, or IWP-2, or PNU-74654, or IWR-Tendo, or IWR-exo, or demethoxycurcumin, or CCT036477, or KY02111, or ivermectin, niclosamide, metformin, or phenformin, or in combination with a glutaminase inhibitor, such as Calithera, inhibiting glutamine uptake for the tumor to which the tumor cells are addicted, are encapsulated in nanogram concentrations within the chitosan coating of the nanoparticles, such iron oxide, gold, silicon carbon, magnetic, non-magnetic Nps or nanoshells, nanocages, nanotubes, nanorods, nanospheres, etc. inside the liposomes containing fluorescein and electromagnetic energy absorbing nanoparticles. When exposed to a thermal energy source such as, electromagnetic radiation, microwave, radiofrequency radiation, focused high power ultrasound, compressive, or pressure low power ultrasound or low frequency of 500-40,000 Hertz, AMF, the lipid component of the liposome melts at temperature of 40-41 degrees C. releasing the fluorescein in the circulation that can be observed by taking a blood sample or radiating the nail bed or any other part of the body with UV radiation so that now non-quenched fluorescein fluoresces as green light passing through a blue filter, indicating the temperature of 41-43 degrees C. is achieved and the increased tumor biomarkers in the circulation after the thermotherapy has an important diagnostic value (i.e., confirming the presence of a tumor) and therapeutic value as biomarkers for the future management of the patient with recurrences by making a vaccine using the biomarkers and VLP, IL-2, or polymeric antibody coated nanoparticles carrying IL-2 and IL-21 or anti-CD3 (OKT3) antibody, to increase cytotoxicity of natural killer cells, IL-6, IL-1β, IL-17, IFN-γ or granzyme β, conjugated with antibody coated nanoparticles and checkpoint inhibitors, such as PD-1, Jagged 1 inhibitor 15D11, to attach to the potential tumor cells while applying thermal energy to damage recurrence of the tumor and induce reactivation of the immune response to eliminate them, while antibody coated nanoparticles/checkpoint inhibitors and VLP attach to the localized or circulating tumor cells and their exosomes, which are carrying a checkpoint protein, such as PD-L1 (to disguise themselves), and the tumor cells are recognized by the T cells, which together with killer cells phagocytose them, and enhance the immune response to the tumor and its exosomes and the circulating cells and eliminate them.

In one embodiment, thermotherapy with focused ultrasound or alternating magnetic field, etc. is combined with antibody coated pluralities of nanoparticles are administered with activatable cell-penetrating peptides (ACPPs) conjugated with Wnt inhibitor such as FH535, or IWP-2, or PNU-74654, or IWR-Tendo, or IWR-exo, or demethoxycurcumin, or CCT036477, or KY02111, or ivermectin, niclosamide, metformin, or phenformin or in combination with a glutaminase inhibitor, such as Calithera, inhibiting glutamine uptake for the tumor to which the tumor cells are addicted, with and without syrosingopine are encapsulated in nanogram concentrations within the chitosan coating of the nanoparticles, such as iron oxide, gold, silicon, carbon, magnetic, non-magnetic nanoparticles or nanoshells, nanocages, nanotubes, nanorods, nanospheres, etc. inside the liposomes containing fluorescein and electromagnetic energy absorbing nanoparticles. When exposed to a thermal energy source, such as electromagnetic radiation, microwaves, radiofrequency radiation, high power focused ultrasound, compressive or pressure low power ultrasound or low frequency 1 KHz to 100 KHz AMF, the lipid component of the liposome melts at a temperature of 40-41 degrees C., thus releasing the fluorescein in the circulation that can be observed by taking a blood sample or radiating the nail bed or any other part of the body with UV radiation so that now the non-quenched fluorescein fluoresces as green light passing through a blue filter, indicating the temperature of 41-43 degrees C. is achieved and the increased tumor biomarkers in the circulation after the thermotherapy has an important diagnostic value (i.e., confirming the presence of a tumor) and therapeutic value as biomarkers for the future management of the patient with recurrences by making a vaccine using the biomarkers and VLP, IL-2, or anti-CD3 (OKT3) antibody, to increase cytotoxicity of natural killer cells, IL-6, IL-1β, IL-17, IFN-γ or granzyme β and other immune stimulators conjugated with antibody/medication coated nanoparticles and administering them with checkpoint inhibitors such as PD-1, PD-L1, CTLA-4, Jagged 1 inhibitor 15D11, etc., and Rock inhibitors, such as Fasudil, or Wnt inhibitors, such as niclosamide, to attach to the potential tumor cells while applying thermal energy to damage recurrence of the tumor and induce reactivation of the immune response to eliminate them, while antibody coated nanoparticles/checkpoint inhibitors and VLP attach to the localized or circulating tumor cells and their exosomes, which are carrying a checkpoint protein, such as PD-L1 (to disguise themselves), and the tumor cells are recognized by the T cells, which together with killer cells phagocytose them, and enhance immune response to the tumor and its exosomes and the circulating cells.

In another embodiment, the nanoshells or nanocages contain one or the perfluorocarbon liquid droplets administered along with the quenched fluorescein or another quenched dye or indicator, etc. When the thermal energy is increased, the expansion of the functionalized pluralities of nanoparticles with activatable cell-penetrating peptides (ACPPs) under the electromagnetic energy causes the nanoparticle to expand and create a photoacoustic/thermoacoustic sound wave that can be recorded by a photoacoustic/thermoacoustic unit by which the temperature at the site of the nanoparticles are recorded depending on the amplitude of the ultrasonic wave, as an additional method to release of a dye for confirmation of the tumor's temperature, and the increased tumor biomarkers in the circulation after the thermotherapy has an important diagnostic value (i.e., confirming the presence of a tumor) and therapeutic value as biomarkers and administering them with checkpoint inhibitors such as PD-1, PD-11, CTLA-4, Jagged 1 inhibitor 15D11, etc. and Rock inhibitors, such as Fasudil and its derivatives or Wnt inhibitors, such as niclosamide, for the future management of the patient with recurrences by making a vaccine using the biomarkers and VLP/checkpoint inhibitors conjugated with antibody coated nanoparticles to attach to the potential tumor cells while applying thermal energy to damage recurrence of the tumor and induce reactivation of the immune response to eliminate them.

In one embodiment, the photoacoustic or thermoacoustic unit is communicating with the thermal delivery unit via a processor to control the amount of energy released by the thermal energy delivery unit via a processor to stop or increase or decrease the temperature all below the thermal denaturation temperature of proteins which is 60 degrees C. at the site of the nanoparticles as desired to prevent excessive thermal damage to the normal surrounding cells.

In one embodiment, thermotherapy with thermal or non-thermal focused low power ultrasound or alternating magnetic field, etc. is combined with antibody/medication coated pluralities of nanoparticles are administered with activatable cell-penetrating peptides (ACPPs) CPP, conjugated with a Wnt inhibitor are encapsulated in the liposomes containing quenched fluorescein or liposomes having temperature triggered opening of a leucine zipper peptide inserted in the membrane of a dye and medication carrying liposome opens a channel through which the drug is released at the temperature or 41-43 degrees C. or opens drug-permeable pores created bubble formation from the decomposition of encapsulated ammonium bicarbonate and another dye or indicator and electromagnetic energy absorbing nanoparticles, such as iron oxide, gold, silicon carbon, magnetic, non-magnetic Nps or nanoshells, nanocages nanotubes, nanorods, nanospheres, piezoelectric nanoparticles, etc. When exposed to a thermal energy source, such as electromagnetic radiation, microwave, or radiofrequency radiation, the lipid component of the liposome melts at s temperature of 40-41 degrees releasing the fluorescein in the circulation that can be observed by taking a blood sample or radiating the nail bed or any other part of the body with UV radiation so that the now non-quenched fluorescein fluoresces as green light passing through a blue wavelength absorbing filter, indicating the temperature of 41 degrees C. is achieved, the fluorescent dye is recognized by an imaging unit communicating with the thermal delivery unit via a processor to control the amount of energy released by the thermal energy delivery unit via a processor to stop or increase or decrease the temperature using thermoacoustic imaging for control of the temperature, all below the thermal denaturation temperature of proteins, which is 60 degrees C., at the site of the nanoparticles as desired to prevent excessive thermal damage to the normal surrounding cells.

Increasing the temperature to 56 degrees C. causes another popping or cavitation sound, which is measured and recorded with a microphone or an ultrasonic receiver indicating the temperature of 56 degrees C. has been achieved at the site of the nanoparticles/tumor cells and the processor of the thermal recoding unit stops the thermal energy delivery system to prevent further thermal energy delivery and the increased tumor biomarkers in the circulation after the thermotherapy has an important diagnostic value (i.e., confirming the presence of a tumor) and therapeutic value as biomarkers for the future management of the patient with recurrences by making a vaccine using the biomarkers and VLP, or intratumor injectable oncolytic viruses, such as T-Vec, monoclonal antibodies, IL-2, or polymeric antibody coated nanoparticles carrying IL-2 and IL-21, or anti-CD3 (OKT3) antibody, to increase cytotoxicity of natural killer cells, IL-6, IL-1β, bee toxins or other immune stimulators conjugated with antibody coated nanoparticles and administering them with checkpoint inhibitors, such as PD-1, PD-11, CTLA-4, Jagged 1 inhibitor 15D11, etc., and Rock inhibitors, such as Fasudil, or Wnt inhibitors, such as niclosamide, to attach to the potential tumor cells while applying thermal energy to damage recurrence of the tumor and induce reactivation of the immune response to eliminate them while antibody coated nanoparticles/checkpoint inhibitors and VLP attach to the localized or circulating tumor cells and their exosomes, which are carrying a checkpoint protein, such as PD-L1 (to disguise themselves), and the tumor cells are recognized by the T cells, which together with killer cells phagocytose them, and enhance immune response to the tumor and its exosomes and the circulating cells.

In one embodiment, when the parameters of thermal heating are kept constant to prevent excess heating of the nanoparticles with activatable cell-penetrating peptides (ACPPs)/tumor is reached at 41-43 degrees C. when the quenched fluorescein is used with a thermosensitive polymer or lipid or liposomes having temperature triggered opening of a leucine zipper peptide inserted in the membrane of a dye and medication carrying liposome opens a channel through which the drug is released at the temperature or 41-43 degrees C. or opens drug-permeable pores created bubble formation from the decomposition of encapsulated ammonium bicarbonate and the fluorescent dye is recognized by an imaging unit communicating with the thermal delivery unit via a processor to control the amount of energy released by the thermal energy delivery unit via a processor to stop or increase or decrease the temperature or the temperature reaches about 56 degrees C. when the perfluorohexane (PFH) or perfluoropentane incorporation in the nanocage or nanoshell evaporates, thus causing an acoustic sound that can be recorded with an ultrasonic receiver that is below 60 degrees C. of protein denaturation.

In one embodiment, one achieves a theranostic or diagnostic and therapeutic effect when the antibody coated pluralities of nanoparticles with activatable cell-penetrating peptides (ACPPs) conjugated with a Wnt inhibitor, such as ivermectin, and a rock inhibitor, such as Fasudil, Y-27632, small molecule inhibitor of ROCK1 and ROCK2 which act as an anti-inflammatory agent and inhibit Wnt activation, are encapsulated in the liposomes containing quenched fluorescein or another dye or indicator and electromagnetic energy absorbing nanoparticles with activatable cell-penetrating peptides (ACPPs), such iron oxide, gold, silicon carbon, magnetic, non-magnetic Nps or nanoshells, nanocages, nanotubes, nanorods, nanospheres, or any other shells produced (e.g., polylactic or polyglycolic acid, etc.) exposed to a thermal energy source gradually or slowly with a focused low power ultrasound or an alternating magnetic field, the lipid component of the liposome melts, releasing medication and the dye at temperature of 41-43 C degrees C. to release the medication and dye and confirmed by taking a blood sample or radiating the nail bed or any other part of the body with UV radiation so that now non-quenched fluorescein fluoresces as green light passing through a blue filter 22 (FIG. 1) demonstrating a hand held system with a blue wavelength radiating source, such as a laser or diode 28 providing a wavelength of 380-390 nm illuminating a finger nail bed 18 and the capillaries in the nail bed 18, which in presence of unquenched fluorescein fluoresces a green light of 410 to 420 nm passing through a blue absorbing filter 22 and reached through a fiber optic, a photomultiplier that converts the light to an electrical signal to a processor 26 that, in turn, is connected to the thermal delivery system indicating the temperature of 41-43 degrees C. is achieved and the medication is released from the Nps, the fluorescent dye is recognized by imaging or thermoacoustic imaging for control of the temperature communicating with the thermal delivery unit via a processor to control the amount of energy released by the thermal energy delivery unit via a processor to stop or increase or decrease the temperature or by increasing the temperature of the nanoparticles or nanoshells, producing another popping or cavitation sound which is measured and recorded with a microphone or an ultrasonic receiver indicating the temperature of 56 degrees C. has been achieved at the site of the nanoparticle/nanoshells/tumor cells, the increased tumor biomarker in the circulation after the thermotherapy has an important diagnostic and therapeutic value for the future vaccine production and management of tumor recurrences in the patients.

In one embodiment, the microphone or the ultrasonic unit is in contact with the patient's body surface records or images the cavitation sound of the PFCL temperature, and communicates to the energy producing unit that the desired temperature of 56 degrees C. is achieved and stops further heating of the tissue to prevent expansion of the thermal energy via convection to the normal cells surrounding the tumor cells and the increased tumor biomarkers in the circulation after the thermotherapy has an important diagnostic value (i.e., confirming the presence of a tumor) and therapeutic value as biomarkers for the future management of the patient with recurrences by making a vaccine using the biomarkers and VLP, or intratumor injectable oncolytic viruses such as T-Vec, monoclonal antibodies, IL-2, or anti-CD3 (OKT3) antibody, to increase cytotoxicity of natural killer cells, bee toxins or other immune stimulators conjugated with antibody coated nanoparticle and administering them with checkpoint inhibitors, such as PD-1, PD-11, CTLA-4, Jagged 1 inhibitor 15D11, etc., and Rock inhibitors, such as Fasudil, or Wnt inhibitors, such as niclosamide, to attach to the potential tumor cells while applying thermal energy to damage recurrence of the tumor and induce reactivation of the immune response to eliminate them and the Rock inhibitors, or in combination with notch pathway inhibitors, anti-VEGFs, such as Bevacizumab, Ranibizumab, Aflibercept, Avastin, and anti-platelet derived growth factor (PDGF) agents, reducing TGF-β production after therapy and the subsequent scar formation.

In one embodiment, one achieves a theranostic or diagnostic and therapeutic effect when the thermal energy melts the thermosensitive polymeric coating, such as chitosan, of the antibody/medication conjugated functionalized pluralities of nanoparticles with activatable cell-penetrating peptides (ACPPs) at the temperature of 42-43 degrees C. and releases the medication and a dye, such as quenched fluorescein or another dye or indicator/medication, from the thermosensitive polymers indicating that the temperature of 43 degrees C. has been reached. For example, the fluorescence is seen in the blood sample of the patient being treated with the thermotherapy, and a processor connected to the temperature recording unit and thermal delivery unit can maintain or stop further heating of the tissue by recording and communicating the achievement of the desire temperatures via the processor's algorithm to the thermal delivery unit and the increased tumor biomarker in the circulation is obtained after the therapy has an important diagnostic and therapeutic value for the future vaccine production and management of tumor recurrences of the patient.

In one embodiment, one achieves a theranostic or diagnostic and therapeutic effect when the nanoshells or nanocage particles carry a thermosensitive polymer, such an chitosan, carrying either an antineoplastic medication, such as taxane derivatives, or a dye, such as quenched fluorescein, or another dye or indicator/medication along with a monoclonal antibody to release them at the temperature of 41-43 degrees C. that can be confirmed by taking a blood sample or radiating the nail bed 18 with UV radiation so that now non-quenched fluorescein fluoresces as green light passes through a blue filter 22 (see FIG. 1) demonstrating a hand held system with a blue wavelength radiating source, such as a laser or diode 28, providing a wavelength of 380-390 nm illuminating to the finger nail 18 and the capillaries in the nail bed 18 which in presence of unquenched fluorescein fluoresces a green light of 410 to 420 nm passing through a blue absorbing filter 22 and reached through a fiber optic 24 to a photomultiplier that converts the light to an electrical signal to a processor 26 that, in turn, is connected to the thermal delivery system indicating the temperature of 41-43 degrees C. is achieved and the fluorescent dye is recognized by an imaging unit communicating with the thermal delivery unit via a processor to control the amount of energy released by the thermal energy delivery unit via a processor to stop or increase or decrease the temperature. Further increasing the temperature by the thermal delivery unit 10 produces another a popping or cavitation sound, which is recorded with a microphone or an ultrasonic receiver 16 indicating the temperature of 56 degrees C. boiling point of the PFCL has been achieved at the site of the nanoparticle/tumor cells, and the increased tumor biomarker in the circulation after the thermotherapy confirms the presence of a tumor and has an important diagnostic and therapeutic value for vaccine formation with VLP, or intratumor injectable oncolytic viruses, monoclonal antibodies, IL-2, or anti-CD3 (OKT3) antibody, to increase cytotoxicity of natural killer cells, bee toxins or other immune stimulators and administering them with checkpoint inhibitors such as PD-1, PD-11, CTLA-4, Jagged 1 inhibitor 15D11, etc. to enhance immune therapy, and Rock inhibitors, such as Fasudil, or Wnt inhibitors, such as niclosamide, for the future management of the tumor recurrences in the patient while antibody coated nanoparticles/checkpoint inhibitors and VLP attach to the localized or circulating tumor cells and their exosomes, which are carrying a checkpoint protein, such as PD-L1 (to disguise themselves), and the tumor cells are recognized by the T cells, which together with killer cells phagocytose them, and enhance immune response to the tumor and its exosomes and the circulating cells.

In another embodiment, the thermosensitive polymers are chitosan, poly-lactic acid (PLA), poly(ethylene glycol), ethyl ether methacrylate-copoly(ethylene glycol), methyl ether methacrylate, Pluronic F127 (F127)[, poly(methylmethacrylate) (PMMA)], poly-n-isopropylacrylamide (PNIPAM), crosslinked ONIPAN hydrogels loaded with Fe3O4 nanoparticles, poly(ethyleneimine)-modified poly(ethylene oxide)-poly(propylene oxide)-poly(ethylene oxide) (PEO-PPO-PEO) block copolymer and nanoparticle-assembled capsules (NACs) using Poly(allylamine hydrochloride), and chitosan-poly(N-isopropylacrylamide).

In one embodiment, thermosensitive polymers and methods of formulating these thermosensitive polymers with or in functionalized pluralities of nanoparticles containing chemotherapy drugs and/or a radiotracer are known in the art used with external thermal delivery, such as microwave, radio frequency (RF), or high power ultrasound, etc. to release the medication under the control of a processor at temperature of 41-43 or more degrees C. as a combination of thermoradiation therapy.

In one embodiment, the thermosensitive polymers with, for example, known pH sensitive polymers, magnetically controlled release agents, magnetoliposome agents, thermosensitive liposomes entrapping iron oxide nanoparticles with activatable cell-penetrating peptides (ACPPs), etc. are used, to achieve controlled drug release with external thermal delivery such as microwave, radio frequency (RF) or ultrasound, etc. under the control of a processor at temperature of 41-43 degrees C.

In one embodiment, variable architecture thermosensitive polymers that are temperature- and pH-responsive, smart polymers, including synthetic copolymers and polymer derivatives, functionalized pluralities of nanoparticles encased in poly(N-isopropylacrylamide) (PNIPAM), poloxamers, poloxamines, polymers with thiol groups (PMA.sub.SH) are used with antineoplastic medications or monoclonal antibodies to release the medication with an external source of energy or magnetic induction under the control of a processor at temperature of 41-43 degrees C.

In one embodiment, the pluralities of functionalized pluralities of nanoparticles with activatable cell-penetrating peptides (ACPPs) and temozolomide are administered intravenously, intra-arterially, locally, etc. contain at least one radioactive isotope and coated with anti-tumor antibodies, monoclonal antibody, the nanoparticles forming an antibody labeled nanoparticle-cell complex at a target site, the radioactive nanoparticle resulting in radiation therapy at a dose sufficient to damage tumor-associated vessels at the localized target site, and the DNA of the tumor stem cells while administering thermal energy simultaneously to achieve controlled drug release with external thermal delivery such as microwave, RF, or low power ultrasound etc. under the control of a processor at temperature of 41-43 degrees C. in treatment of many brain tumors specially glioblastoma.

In one embodiment, the pluralities of radioactive functionalized nanoparticles with activatable cell-penetrating peptides (ACPPs) coated with thermosensitive polymers and antineoplastic medication, such as taxol and or temozolomide, are administered locally or systemically, intravenously, intra-arterially, inside a body cavity, and are magnetic, diamagnetic, ferromagnetic, non-ferromagnetic and/or paramagnetic nanoparticle, wherein the radioactive isotope is an alpha.-emitting isotope, a beta.-emitting isotope, or a combination thereof consisting of At.sup.211, Ac.sup.225, Bi.sup.212, Bi.sup.213, Ra.sup.223, Pb.sup.212, Tb.sup.149, I.sup.131, Cu.sup.64, I.sup.131, Bi.sup.213, and Bi.sup.212, with the dose of the isotope ranges from 10 .mu.Ci to 20,000. mu.Ci, and thermal energy is administered simultaneously to achieve controlled drug release with an external or internal thermal delivery such as laser, microwave, RF, or low power focused ultrasound, etc. under the control of a processor at a temperature of 41-43 degrees C. to damage the tumor cells and their vascular supplies, the DNA of the tumor stem cells, and release the medication, such as temozolomide in treatment of many cancers including brain tumors.

In one embodiment, the pluralities of functionalized radioactive gold or combination of gold and ferric oxide nanoparticles with activatable cell-penetrating peptides (ACPPs) coated with thermosensitive polymers and checkpoint inhibitors, such as ipilimumab nivolumab, Rock inhibitors, such as Fasudil, its derivatives, etc., anti-integrins, bevacizumab, and/or an antineoplastic medication, such as taxol and or temozolomide, are administered locally or systemically, intravenously, intra-arterially inside a body cavity, where magnetic, diamagnetic, ferromagnetic, non-ferromagnetic, and/or the paramagnetic nanoparticle is an interstitial brachytherapy nanoparticles beta radiators and thermal energy is administered simultaneously to achieve controlled drug release with an external or internal thermal delivery such as laser, microwave, RF, or high power ultrasound, etc. under the control of a processor at a temperature of 41-43 degrees C. to damage the tumor cells and their vascular supplies, the DNA of the tumor stem cells, and release the medication, such as temozolomide, in treatment of many cancers including brain tumors where thermal therapy can be repeated in the postoperative period as needed.

In one embodiment, groups of non-magnetic, paramagnetic, or magnetic functionalized pluralities of nanoparticles with activatable cell-penetrating peptides (ACPPs), CPP, and thermosensitive polymeric coating are produced where polymeric coating of these nanoparticles renders them less toxic to normal tissue, such as Iodine.sup.131 may be crosslinked with antibody-coated nanoparticles to label the tumor or bacterial cells, both for therapy and imaging. Anti-integrins, integrin-targeted therapeutics, abegrin cilengitide, abciximab, tirofiban, natalizumab eptifibatide, notch pathway inhibitors, anti-VEGFs, such as Bevacizumab, Ranibizumab, Aflibercept, Avastin, and anti-platelet derived growth factor (PDGF) agents may also be incorporated in the nanoparticles or the nanoparticles may be used simultaneously for drug delivery purposes against these tumor or bacterial cells, with the drug in PDA, PGLA, dextran, dendrimers, PEG, etc.

In one embodiment, the functionalized antibody/medication coated nanoparticles and thermosensitive polymeric coating may be quantum dots, magnetic and paramagnetic fullerene, encapsulate ferromagnetic nanoclusters with activatable cell-penetrating peptides (ACPPs), polymer based nanoparticles, colloidal gold nanoparticles, iron oxide nanoparticles, magnetic and paramagnetic fullerene, etc. The nanoparticle size may range from 2 nm to 400 nm in one embodiment, and from 2 nm to 200 nm in another embodiment.

In one embodiment, a focused ultrasound is used to heat the antibody/medication coated nanoparticle with activatable cell-penetrating peptides (ACPPs) conjugated with thermosensitive polymeric coated nanoparticles carrying a medication, dye, VLP, IL-2, IL-6, IL-1β, IL-17, IFN-γ or granzyme β, monoclonal antibody, or checkpoint inhibitors alone to release the medication when a temperature of 43 degrees C. has been achieved and the combination of monoclonal antibody, VLP and thermotherapy eliminate the need for the use of CAR-T cell that increases postoperative cytokine storm and autoimmune response, and the increased tumor biomarkers in the circulation after the therapy has an important diagnostic value (i.e., indicating presence of a tumor) and therapeutic value as biomarkers for production of vaccine and administering them with checkpoint inhibitors such as PD-1, PD-L1, CTLA-4, Jagged 1 inhibitor 15D11, etc. while antibody coated nanoparticles/checkpoint inhibitors and VLP attach to the localized or circulating tumor cells and their exosomes, which are carrying a checkpoint protein, such as PD-L1 (to disguise themselves), and the tumor cells are recognized by the T cells, which together with killer cells phagocytose them, and enhance an immune response to the tumor and its exosomes and the circulating cells, and Rock inhibitors, such as Fasudil, and its derivatives etc. or Wnt inhibitors, such as niclosamide, for the future management of the tumor recurrences in the patient, and the Rock inhibitor, in combination with anti-VEGF and PDGF inhibitors for reducing TGF-β production after therapy and the subsequent scar formation.

In one embodiment, a focused high energy ultrasound is used to heat the antibody/medication coated nanoparticle with activatable cell-penetrating peptides (ACPPs) conjugated with thermosensitive polymeric coating carrying a medication, dye, monoclonal antibody, or check point inhibitors alone to release the medication when a temperature of 43 degrees C. has been achieved and the combination of monoclonal antibody and thermotherapy eliminate the need for the use of CAR-T cell that increases postoperative cytokine storm and autoimmune response, and the increased tumor biomarkers in the circulation after the therapy has an important diagnostic value (i.e., indicating presence of a tumor) and therapeutic value as biomarkers for production of vaccine and administering them with checkpoint inhibitors such as PD-1, PD-11, CTLA-4, Jagged 1 inhibitor 15D11, etc. to enhance immune therapy and oral Rock inhibitors, such as Fasudil, (40-80 mg tablets) etc. or Wnt inhibitors, such as niclosamide (<500 mg dose), ivermectin etc. 1-2 gram dose once in a week or can be repeated as needed in another month for the future management of the tumor recurrences in the patient, while the Rock inhibitor or in combination with anti-VGEF and or PDGF inhibitors reduce TGF-β production after therapy and the subsequent scar formation.

In one embodiment, a focused low energy ultrasound is used in a compressive or pressure mode which does not heat up the functionalized nanoparticles with activatable cell-penetrating peptides (ACPPs) and thermosensitive polymeric coating (e.g., the nanocages, nanoshells, etc.), but releases the coated medication, dye, monoclonal antibody, or check point inhibitors alone by shaking up the nanoparticles with activatable cell-penetrating peptides (ACPPs) to release the medication and damage the cell by the compressive action of the focused low energy ultrasound while imaging the lesion while the delivery of medication is verified by the release of the dye/medication in the circulation, as shown in FIG. 1.

In one embodiment, a focused low power ultrasound in a compressive or pressure mode is used alternatively with a heating high energy ultrasound which releases the coated medication, dye, monoclonal antibody or check point inhibitors alone at a lower temperature of 37-38 degrees C. without damaging the tissue by shaking up the antibody/medication nanoparticles coated with cell penetrating peptides with a thermosensitive polymeric coating, while keeping the temperature at <40 degrees C. that does not damage normal cells, but makes the tumor membrane permeable to medication to release the medication and damage the tumor cell by the compressive action of the compressive mode of focused low power ultrasound while imaging the lesion and delivering the medication is verified by the release of the dye/medication in the circulation as shown in FIG. 1.

In one embodiment, a focused low energy or low power ultrasound in a compressive or pressure mode is used which does not heat up the functionalized with cell penetrating peptide and medication coated nanoparticle, but releases the coated medication, dye, monoclonal antibody or check point inhibitors by shaking up the functionalized pluralities of nanoparticles, or magnetic and paramagnetic fullerene, or encapsulate ferromagnetic nanoclusters and damages the cell membrane by the compressive action of the focused ultrasound while imaging the lesion with the compressive focused ultrasound and delivering the dye/medication is verified by the release and presence of the dye/medication in the circulation, as shown in FIG. 1.

In one embodiment, a focused low power ultrasound is used in a compressive or pressure mode alternating with the thermal mode which releases the coated medication, dye, monoclonal antibody or check point inhibitors from the functionalized nanoparticles with ACPP at a lower temperature of 41 degrees C. seen by release of the dye in the circulation, without damaging the normal tissue, by shaking up the pluralities of nanoparticles while keeping the temperature at about at or below 41 degrees C. that does not damage normal cells, but makes the tumor membrane permeable to medication, and releases the dye/medication and damages the tumor cell by the compressive action of the focused low power ultrasound while imaging the lesion and delivering the medication is verified by the release of the dye/medication in the circulation, as shown in FIG. 1.

In one embodiment, a focused low energy ultrasound in a compressive or pressure mode is alternatively used with heating focused high power ultrasound to image the lesion and release the coated medication, dye, monoclonal antibody or check point inhibitors alone at lower temperature of 41 degrees C. without damaging the normal tissue by shaking up the nanoshell or nanocage containing PFCL, while keeping the temperature at about 41 degrees C. that does not damage normal cells, but makes the tumor membrane permeable to medication to release the medication/dye, then increase the thermal energy or the duration of the thermal/compressive or pressure low power ultrasound causes the PFCL to reach boiling point at an earlier temperature of 47 to 50 degrees C. which is lower than the boiling point of the PFCL (56 degrees C.) recorded by creating a cavitation or popping sound indicating the desired temperature is achieved which is being recorded by a microphone or an ultrasonic receiver connected to the patient's body and communicates to the ultrasound delivery unit that desired temperature has been achieved, and to stop further delivery of the focused ultrasound via the processor automatically.

In one embodiment, the temperature of the heated functionalized nanoparticles/tumor cell is measured by the release of dye/fluorescein form the thermosensitive polymeric coating of the nanoparticle in the circulation that can be observed by UV radiation and a filter from the nail bed capillaries indicating a temperature of 41-43 degrees C. at the NPs/tumor cell complex (see FIG. 1) or recording a popping sound at temperature of 56 degrees C. when the nanoshell with PFCL hexane, etc. is used.

In one embodiment, the temperature of the heated functionalized nanoparticles/tumor cell is measured when a focused low power ultrasound and alternating magnetic field is used to heat up the nanocage, or nanoshells to up to the boiling point of their PFCL hexane or pentane that is about 50 to 56 degrees C. and image the temperature with thermoacoustic imaging system for control of the temperature.

In one embodiment, the temperature of the heated functionalized nanoparticles/tumor cell is measured similarly when the combination of compressive or pressure mode and thermal focused high power ultrasound is used to heat up the nanocages or nanoshells to up to the boiling point of their PFCL hexane or pentane that is about 50 to 56 degrees C.

In one embodiment, the parameters or thermal heating of the nanoparticle, nanocage or nanoshell is kept constant to heat up the antibody/medication coated pluralities of nanoparticles with activatable cell-penetrating peptides (ACPPs), the temperature reached is about 56 degrees C. that is below 60 degrees C. temperature of protein denaturation, the incorporation of perflourohexane PFH cause evaporation of PFH at a 56 degrees C. temperature causing an acoustic sound that can be recorded with an ultrasonic receiver and the simultaneous increased tumor biomarker seen in the blood samples after the thermotherapy having an important diagnostic release of the large number of tumor biomarkers and therapeutic value for making a useful vaccine and administering them with antibody coated checkpoint inhibitors, such as PD-1, PD-11, CTLA-4, Jagged 1 inhibitor 15D11, etc. and VLP to enhance immune therapy, and Rock inhibitors, such as Fasudil, in with combination anti-VEGFs, or Wnt inhibitors, such as niclosamide, to stimulate a robust immune response in the future if tumor recurrences are observed in the patient while reducing TGF-β production after therapy and the subsequent scar formation.

In one embodiment, electromagnetic radiation, microwave, radio frequency (RF) or an alternating magnetic field or focused low power ultrasound alone or with compressive or pressure mode focused ultrasound along with antibody bubble liposomes which contain air pockets or nanoemulsions of PFC carrying fluorescein, gene and/or medication coated with CPP conjugated with a thermosensitive polymer, such as chitosan, nanocages or nanoshells filled and perfluorocarbon liquid with magnetic nanoparticles, nanocages or nanoshells can be heated by different thermal energy sources to create three distinct temperatures at the tumor site, image the temperature with thermoacoustic imaging system for control of the temperature 41-43 degrees C. measured by dye/medication release in the circulation or 50-56 degrees C. by recorded cavitation sound when combination of compressive or pressure mode and heating of focused high energy ultrasound or alternating magnetic field are used to create a cavitation sound or the temperature of 56 degrees C. if focused high power ultrasound or AMF or laser is used alone along with the PFCL Hexane nanoshells or nanocages.

In one embodiment, either of the temperature indicators communicate via a processor that the desired temperature is achieved by a given thermal energy, to control the temperature and the duration of the desired temperature in the tissue, thereby separating the thermal drug delivery process from the cells destructive thermal delivery, as needed, via a processor that connects the temperature imaging system to the thermal delivery unit.

In one embodiment, the control of the functionalized nanoparticles/tumor cell complex temperature is controlled at the desired level without overcooking the normal tissue and the increased tumor biomarkers in the circulation after the thermotherapy has an important diagnostic value by measuring and confirming the presence of a tumor increased serum biomarkers, and dye release and therapeutic value for collecting biomarkers shortly after thermotherapy from the circulation for the future vaccine production for management of the patient with recurrences by making a vaccine using VLP, or intratumor injectable oncolytic viruses, monoclonal antibodies, IL-2, bee toxins or other immune stimulators along with antibody coated nanoparticles and administering them with checkpoint inhibitors, such as PD-1, PD-L1, CTLA-4, Jagged 1 inhibitor 15D11, etc., and Rock inhibitors, such as Fasudil, or Wnt inhibitors, such as niclosamide, to attach to the recurrent tumor cells while applying thermal energy to damage tumor recurrences and induce reactivation of the immune response to eliminate the tumor and while antibody coated nanoparticles/checkpoint inhibitors and VLP attach to the localized or circulating tumor cells and their exosomes, which are carrying a checkpoint protein, such as PD-L1 (to disguise themselves), and the tumor cells are recognized by the T cells, which together with killer cells phagocytose them, and enhance the immune response to the tumor and its exosomes and the circulating cells.

In one embodiment, regardless of the thermal energy used, the increased tumor biomarkers in the circulation after the thermotherapy has an important diagnostic value confirming the presence of a tumor and therapeutic value as biomarkers for the future vaccine production with management of the patient with recurrences by making a vaccine using VLP, or intratumor injectable oncolytic viruses, monoclonal antibodies, IL-2, bee toxins or other immune stimulators conjugated with antibody/medication coated nanoparticles and checkpoint inhibitors, such as PD-1, Jagged 1 inhibitor 15D11, etc., and Rock inhibitors or Wnt inhibitors, such as niclosamide, to attach to the tumor cells while applying thermal energy to damage recurrence of the tumor and induce reactivation of the immune response to eliminate the tumor.

In one embodiment, the antibody/medication coated nanoparticle or nanoshell solution containing 2.67 nM gold nanorods, 2% human serum albumin, 0.04% (w/v) avidin and filling with PFC (C 3F 8) gas, then modified via biotin-avidin technique to result in anti VEGF (Avastin), aflibercept to target by ultrasonography, the area of angiogenesis, such as a tumor or wet form of age related macular degeneration The GNRs could induce photoacoustic or thermoacoustic imaging and thermal therapy under electromagnetic radiation or focused low power ultrasound showing the theranostic value of this modality on keeping the temperature at the desired temperature of 41-43 degrees C. not to damage the surrounding normal tissue while treating a tumor (e.g., glioblastoma) or ARMD at low temperature and release medication and Wnt inhibitor of Rock inhibitors (Fasudil, Y-27632, small molecule inhibitor of ROCK1 and ROCK2 which act as an anti-inflammatory agent and inhibit Wnt activation, from the thermosensitive nanoparticle under the control of the temperature and the increased glioblastoma biomarkers, such as co-receptor, glypican 2 (GLP2) or cell surface GPC2 in the circulation after the therapy has an important diagnostic value (i.e., indicating the presence of a tumor) and therapeutic value as biomarkers for vaccine production and administering them with antibody coated VLP, checkpoint inhibitors, such as PD-1, PD-11, CTLA-4, Jagged 1 inhibitor 15D11, etc. to enhance immune therapy and Rock inhibitors, such as Fasudil, netarsudil, its derivatives, etc., or Wnt inhibitors, such as niclosamide, for the future management of the patient in case of the recurrences.

In one embodiment, the antibody/medication coated nanoparticle or nanoshell solution containing 2.67 nM gold nanorods, 2% human serum albumin, 0.04% (w/v) avidin and filling with PFC (C 3F 8) gas, then modified via biotin-avidin technique to result in anti VEGF (Avastin), aflibercept or Axitinib and quenched fluorescein, bubble liposomes carrying fluorescein which contain air pockets or nanoemulsions of PFC, gen or another dye or indicator, in a thermosensitive coating of the functionalized pluralities of nanoparticles with activatable cell-penetrating peptides (ACPPs) to target by ultrasonography the area of angiogenesis, such as tumor or suspicious breast cancer or ovarian cancer, while GNRs could induce photoacoustic or thermoacoustic imaging and thermal therapy under AMF or focused low power ultrasound showing the theranostic value of this modality on keeping the temperature at the desired temperature of 41-43 degrees C. and 56 degrees C. so as not to damage the surrounding normal tissue, while treating a tumor (e.g., small breast cancer lesions or ovarian tumors) at low temperature and release medication and Wnt inhibitor or anti-integrin or Rock inhibitors, such as Fasudil (HA-1077 a selective RhoA/Rho kinase (ROCK) inhibitor), Y-27632, small molecule inhibitor of ROCK1 and ROCK2 which act as an anti-inflammatory agent and inhibit Wnt activation, from the thermosensitive nanoparticle under the control of the temperature to prevent excessive inflammatory response in the treated organ and the increased tumor biomarkers in the circulation after the thermotherapy has an important diagnostic (i.e., indicating presence of a tumor) and therapeutic value as biomarkers for the future management of the patient.

In one embodiment, the thermal energy is applied to the body to find out if the antibody/medication coated pluralities of nanoparticles with activatable cell-penetrating peptides (ACPPs) nanoshells with PFCL or nanocages, etc. are accumulated at a potential place, such as brain, breast, prostate, ovaries, etc., the nanoparticles when heated with thermal energy, such as AMF or focused low power ultrasound that can be recorded by a receiver and image the temperature, while recognizing the location of the potential tumor also in conjunction with MRI, or CT-scan or PET-scan, etc.

In one embodiment, the tumor is verified with functionalized pluralities of nanoparticles with activatable cell-penetrating peptides (ACPPs) coated with multiple antibodies to biomarkers found in the serum, administered intravenously, intra-arterially, in a body cavity, subcutaneously to seek lymphatic vessels and attach to the tumor receptors located in various organs to be heated with either laser, focused high energy ultrasound or alternating magnetic field or microwave to either produce a photoacoustic or thermoacoustic sound or other sounds indicating expansion or nanoparticle or its content such as a gas, perfluorocarbon liquid expansion indicating the transition temperature from fluid to gas of 56 degrees C., which is recorded by a microphone or an ultrasound receiver and the temperature is imaged with a thermoacoustic imaging system for control of the temperature and the increased tumor biomarkers in the circulation after the thermotherapy has an important diagnostic (i.e., indicating presence of a tumor) and therapeutic value as biomarkers for the future management of the patient.

In one embodiment, the recoding or temperature imaging system is connected to the thermal delivery unit via a processor to prevent increase of the temperature above predetermined level or maintaining or reducing the thermal energy or stopping it.

In one embodiment, two indicators are used to indicate the temperature at the level of the tumor. One indicator is quenched fluorescein, bubble liposomes carrying fluorescein which contain air pockets or nanoemulsions of PFC, or a non-toxic dye/medication encapsulated in small functionalized liposomes that are administered simultaneously with other functionalized pluralities of nanoparticles that can act as a contrast agent and heat up when exposed to the thermal energy, where the liposome breaks up at temperature of about 42 degrees C. and release its content fluorescein that can be easily recognized in the circulating blood indicating the temperature of 41 degrees C. is achieved increasing the thermal energy heats up nanoparticles or nanocages to reach the transition or boiling point of perfluorocarbon liquid at 56 degrees C. with a popping cavitation sound recorded by a transducer indicating the second desired temperature is achieved and via the processor to stop further heating of the tissue and the increased tumor biomarkers in the circulation after the thermotherapy has an important diagnostic (i.e., indicating presence of a tumor) and therapeutic value as biomarkers for the future management of the patient.

In one embodiment, an alternating magnetic field can heat up the antibody/medication coated nanoparticle or nanoshells in a patient having an ovarian tumor incorporated with a liquid perfluorocarbon, such as perfluoropentane, hexane, octane shells coated with Wnt inhibitor, anti-neoplastic medication and a rock inhibitor such as fasudil Rock inhibitors (HA-1077 a selective RhoA/Rho kinase (ROCK) inhibitor), Y-27632, small molecule inhibitor of ROCK1 and ROCK2 which act as an anti-inflammatory agent and inhibits Wnt activation, or anti-integrin administered in a patient having an ovarian tumor that expands rapidly when the tissue is heated by a source of energy such as AMF or focused high power and high intensity ultrasound to reach a temperature 43 degrees C. or beyond 43 degrees C. to 56 degrees C. which is the boiling point of these PFCLs, thereby creating a sudden gas expansion and a popping or cavitation sound that can be recorded by a microphone indicating that the 56 degrees C. temperature is achieved.

In one embodiment, the functionalized pluralities of nanoparticles with activatable cell-penetrating peptides (ACPPs) are micelles of perfluorocarbon liquids having a hydrophilic head having thermosensitive polymer such as chitosan that release the medication/dye at 41-42 degrees C. when the polymer melts and an increased temperature to 56 degrees C. creates a cavitation ultrasonic sound that is recorded indicating damage to the tumor cell membrane by thermal energy.

In one embodiment, the thermosensitive polymer of the nanoparticles or micelles carries a dye, such as fluorescein, which is released at the temperature of 41-43 degrees C. in the circulation and its presence in the serum can be verified rapidly using a UV radiation indicating that the thermal energy created a temperature of 41-43 degrees C. at the site of the nanoparticles.

In one embodiment, the thermosensitive polymer carries dye, medication, or a gene to be released at temperature of 42 degrees C. to affect the tumor or kill the tumor cells at 56 degrees C. thereby increasing the amount of the antigen released from the tumor cells which attracts more immune response to the biomarkers that are not normally available in the circulation and collecting these biomarkers can build the basis of a better cancer vaccine and administering them with antibody coated VLP or other immune stimulators and checkpoint inhibitors, such as PD-1, PD-11, CTLA-4, Jagged 1 inhibitor 15D11, etc. to enhance immune therapy, and Rock inhibitors, such as Fasudil, in combination with anti-VEGFs, or integrin inhibitors, or Wnt inhibitors, such as niclosamide reducing TGF-β production after therapy and the subsequent scar formation and inflammation.

In one embodiment, the functionalized pluralities of nanoparticles with activatable cell-penetrating peptides (ACPPs) carry tumor monoclonal antibodies along with a Wnt inhibitor or integrin inhibitors as the tumor is heated to 42 degrees C. and its membrane becomes more permeable to the medications or gene released.

In one embodiment, an alternating magnetic field can heat up the antibody coated nanoparticles or nanoshells in a patient having an ovarian tumor incorporated with a liquid perfluorocarbon, such as perfluoropentane, hexane, octane shells coated with Wnt inhibitor, anti-neoplastic medication and a rock inhibitor such as fasudil which acts as an anti-inflammatory agent and inhibits Wnt activation, administered in a patient having an ovarian tumor that expands rapidly when the tissue is heated by a source of energy such as AMF or focused high power ultrasound to reach a temperature 43 degrees C. or beyond 43 degrees C. to 56 degrees C., and the temperature is measured with thermoacoustic imaging system for control of the temperature, which is the boiling point of these PFCLs, thereby creating a sudden gas expansion and a popping cavitation sound that can be recorded by a microphone indicating that the 56 degrees C. temperature is achieved.

In one embodiment, the functionalized pluralities of nanoparticles with activatable cell-penetrating peptides (ACPPs) are micelles of perfluorocarbon liquids having a hydrophilic head with a thermosensitive polymer coating, such as chitosan, or liposomes having temperature triggered opening of a leucine zipper peptide inserted in the membrane of a dye and medication carrying liposome opens a channel through which the drug is released or the temperature or 41-43 C degree or opens drug-permeable pores created bubble formation from the decomposition of encapsulated ammonium bicarbonate that release the medication/dye at 41-42 degrees C. when the polymer melts and an increased temperature to 56 degrees C. creates a cavitation ultrasonic sound that is recorded, indicating damage to the tumor cell membrane by thermal energy.

In one embodiment, the functionalized pluralities of nanoparticles with activatable cell-penetrating peptides (ACPPs) are coated with thermosensitive polymers conjugated with medication, monoclonal antibodies and check point inhibitors, and are injected intravenously, intra-arterially, locally, systemically, subcutaneously, subconjunctival, orally, or by inhalation. After administration, the nanoparticles find the tumor cells expressing the tumor surface antigen, attach to the cell membrane receptors of the tumor cells, during the thermotherapy at temperature of 41-43 degrees C. when the thermosensitive polymer melts, the medication, monoclonal antibodies and checkpoint inhibitors and VLP, etc. from the thermosensitive polymers are released precisely at the tumor site, eliminating the present practice of administrating the unbound medication, or unbound checkpoint inhibitors in the circulation, which can kill the exposed normal cells by the medication or attack the cells of the body which also express normally checkpoint inhibitors, Rock inhibitors, such as Fasudil, netarsudil, its derivatives in combination with anti-VEGFs, etc. to reduce the inflammatory process and production of TGF-β. The latter induces various side effects as autoimmune response to various organs of the patient, such as the lung, digestive tract, skin, heart or kidney etc. that are difficult to manage while eliminating the tumor with a precise and localized immune response.

In one embodiment, the functionalized or liposomes or nanoparticles are coated with thermosensitive polymers, conjugated with a dye and with Wnt inhibitors that reduces the post-treatment severe inflammatory response and reduces the impulse for tumor cell multiplication by inactivation of the Wnt pathway, and the increased tumor biomarkers in the circulation after the thermotherapy has an important diagnostic (i.e., indicating presence of a tumor) and therapeutic value as biomarkers for the future management of the patient.

In one embodiment, the pluralities of antibody/medication coated functionalized pluralities of nanoparticles or liposomes having temperature triggered opening of a leucine zipper peptide inserted in the membrane of a dye and medication carrying liposome opens a channel through which the drug is released or the temperature or 41-43 C degree or opens drug-permeable pores created bubble formation from the decomposition of encapsulated ammonium bicarbonate conjugated with Wnt inhibitors and Rock inhibitors or integrin inhibitors which act as anti-inflammatory agents and inhibit Wnt activation, and thermosensitive coated polymers or a combination of small liposomes or micelles containing nanoparticles that act as thermal energy contrast agents are administered intravenously, intra-arterially, locally, inside the tumor, etc. to image the temperature using contrast agents, such as liposomes containing nanoshells, or nanocages containing in addition either a medication or a dye or an high power high intensity ultrasound contrast agent, such as PFCL, that creates a sound wave when heated under the control of the thermal energy delivery unit to reach the boiling point of the PFCL at temperature at 56 degrees C. temperature creating a popping or cavitation sound, then recording it by a microphone indicating the 56 degrees C. has been achieved and to damage to the tumor cell and do not heat up the tissue further to limit thermal damage by heat spillover to the normal cells surrounding the tumor and the increased tumor biomarkers in the circulation after the thermotherapy has an important diagnostic (i.e., indicating presence of a tumor) and therapeutic value as biomarkers for the future management of the patient and enhancing the effect of immunotherapy or medication by thermotherapy and preventing production of medication resistance in these diseases.

In one embodiment, one treats excessive an immune response to the immune therapy by stopping the immune therapy shortly after it appears that the therapeutic goal has been achieved by administration of medications with antibody coated nanoparticles with activatable cell-penetrating peptides (ACPPs) conjugated with a thermosensitive polymer that releases them at the desired site at the temperature of 41-43 degrees C. at lower concentrations than was previously possible with standard intravenous administration and at the site of a tumor, not the entire body, thereby preventing the tissue over-reaction to the immune therapy and enhancing the effect of immunotherapy or medication by thermotherapy and preventing production of medication resistance in these diseases.

In one embodiment, the functionalized pluralities of nanoparticles with activatable cell-penetrating peptides (ACPPs) are conjugated with the rock inhibitor Fasudil (HA-1077), a selective RhoA/Rho kinase (ROCK) inhibitor, or Y-27632, small molecule inhibitor of ROCK1 and ROCK2, or integrin inhibitors, etc. to dampen postoperative cytokine inflammatory response and also block the Wnt pathway inside the proliferating cancer cells or inflammatory cells seen after monoclonal antibodies or chemotherapy.

In one embodiment, the functionalized pluralities of nanoparticles with activatable cell-penetrating peptides (ACPPs) contain medications with the rock inhibitor Fasudil (HA-1077), a selective RhoA/Rho kinase (ROCK) inhibitor, or Y-27632, small molecule inhibitor of ROCK1 and ROCK2 or integrin inhibitors such as NEDD9 scaffold protein, antibody LM609 and Nitroproline derivatives, etc. along with slow release polymers, such as polylactic or polyglycolic acid or combinations of them carrying immunosuppressive agents to reduce the cytokine response in CAR-T cell immunotherapy.

In one embodiment, where there are tumor recurrences after initial CAR-T cells therapy in hematological malignancies such as lymphomas or leukemias or in solid tumors, such as melanoma, glioblastoma, ovarian, breast cancer, intestinal and colon cancer, prostate cancer, sarcoma, bone cancer, one administers intravenously, intraperitoneally, or subcutaneously, a combination of antibody coated nanoparticles conjugated with an antineoplastic medication along with Wnt inhibitors or a Rock inhibitors, ipilimumab, nivolumab, avastin, or aflibercept, and applies external thermal energy to heat up the functionalized pluralities of nanoparticles with activatable cell-penetrating peptides (ACPPs) to release the medications at the temperature of 41-43 degrees C. or higher to inhibit Wnt activation by the metastatic cell and kill the tumor cells by increasing the nanoparticles temperature to 56 degrees C. to kill the tumor cells and obtain the increased tumor biomarkers from the circulation after the thermotherapy, to confirm diagnosis the recurrent cancer and cold storage of the biomarkers for future management of the patient with recurrences or making a vaccine combining the biomarkers, VLP, or intratumor injectable oncolytic viruses, monoclonal antibodies, IL-2, IL-6, IL-1β, IL-17, bee toxins or other immune stimulators conjugated with antibody/medication polymeric coated nanoparticles and checkpoint inhibitors such as PD-1, Jagged 1 inhibitor 15D11, for a lasting immune stimulation, etc., and Rock inhibitors or Wnt inhibitors, such as niclosamide, to be administered as needed, subcutaneously or systemically to attach the nanoparticles with activatable cell-penetrating peptides (ACPPs) to the potential tumor cells, applying thermal energy to release the medication from the thermosensitive polymer coated nanoparticles and damage the tumor recurrence and to reactivate the immune response for a longer time up to months and eliminate the tumor cells and its potential metastatic lesions.

In one embodiment, where there are tumor recurrences after initial CAR-T cells therapy in hematological malignancies such as lymphomas or leukemias or in solid tumors, such as ocular or skin melanoma, glioblastoma, ovarian, breast cancer, intestinal and colon cancer, prostate cancer, sarcoma, bone cancer, one administers intravenously, intraperitoneally, or subcutaneously, a combination of antibody coated pluralities of nanoparticles conjugated with thermosensitive polymers and an antineoplastic medication along with Wnt inhibitors or a Rock inhibitors, and ipilimumab, nivolumab, avastin, or aflibercept, or diphenylpyrazole and application of external thermal energy to heat up the functionalized pluralities of nanoparticles with activatable cell-penetrating peptides (ACPPs) to release the medications at the temperature of 41-43 degrees C. or higher to inhibit Wnt activation by the metastatic cell and kill the tumor cells by increasing the nanoparticles temperature to 56 degrees C. to kill the tumor cells and obtain the increased tumor biomarkers from the circulation after the thermotherapy, to confirm diagnosis the recurrent cancer and cold storage of the biomarkers for future management of the patient with recurrences or making a vaccine combining the biomarkers, VLP or intratumor injectable oncolytic viruses, monoclonal antibodies, IL-2, bee toxins or other immune stimulators and checkpoint inhibitors conjugated with antibody/medication, an α-synuclein, diphenyl-pyrazole or compound anle138b that blocks AP channels leading to dysregulation of autophagy, in advanced ocular or skin melanoma, coated nanoparticles to be administered as needed, subcutaneously or systemically to attach the nanoparticles applying thermal energy to release the medication from the thermosensitive polymer coated pluralities of nanoparticles and damage the tumor/melanoma recurrence and to reactivate the immune response and eliminate the tumor while antibody coated nanoparticles/checkpoint inhibitors and VLP attach to the localized or circulating tumor cells and their exosomes, which are carrying a checkpoint protein, such as PD-L1 (to disguise themselves), and the tumor cells are recognized by the T cells, which together with killer cells phagocytose them, and enhance immune response to the tumor and its exosomes and the circulating cells.

In one embodiment, Ivermectin or Niclosamide can be given orally at a low dose in conjunction with the standard immunothermotherapy procedure.

In one embodiment, all medications are coated with a plurality of functionalized nanoparticles for local delivery from the thermosensitive nanoparticle to release the medication when the temperature reaches 41-43 degrees C. under the control of the thermal energy delivery system processor connected to the thermal imaging recorder system and its software algorithm to control the temperature or prevent overheating the tissue.

The Ras oncogenes and the proteins (Ras) encoded genes have had an important roles in the pathogenesis of the human cancer affecting >50% of colon and >90% of pancreatic cancers. Ras proteins, are membrane-bound GTP-binding proteins that serve as molecular switches in mutagenic signal transduction.

In one embodiment, inhibitors, such as Farnesyl or transferase, are used to block Ras activation and other relevant pathway inhibitors of PK1 and PK8 and P13K, RAF/MEK/ERK, PI3K/AKT/mTOR and Ra1A/B pathways that can be inhibited by administering gemcitabine, wortmannin, LY294002 and tipifarnib alone or in combination with Gemcitabine or rapamycin and AZD8055(aTORKi), everolimus, and avastin, aflibercept and quenched dye or another indicator conjugated with antibody/medication coated thermosensitive polymers and/or pluralities of nanoparticles with ACPP intravenously, intra-arterially, in the cerebrospinal fluid, locally, intraperitoneally, and release the dye and the medication by applying external, local, or internal thermal energy and damage the tumor cells that are damaged by the temperature of 41-43 degrees C. to 56 degrees C., thereby enhancing the effect of immunotherapy or medication and prevent production of medication resistance in these diseases, such as pancreatic cancer, breast cancer and brain cancer, including glioblastoma, glioma, prostate cancer, ovarian cancer, and release the antigenic cell content in the circulation to incite an enhanced immune response and obtaining new biomarkers from the blood to produce vaccine and administering them with checkpoint inhibitors, such as PD-1, PD-11, CTLA-4, Jagged 1 inhibitor 15D11, etc., and Rock inhibitors, such as Fasudil, or Wnt inhibitors, such as niclosamide.

In one embodiment with tumor mutation inhibition with thermotherapy, the Wnt inhibitor Ivermectin is PEGylated with a lipid and conjugated with antibody coated nanoparticles to release the medication precisely at the site of the tumor and prevent potential toxicity of ivermectin if given in a high dose systemically that activates P2X4/P2X7 and Pannexin-1 channels releasing ATP that enhances rapidly the toxicity of P2X7 receptor inducing sever tissue necrosis, whereas the controlled local drug delivery with thermally heating the tumor cells melts the thermosensitive polymer releasing the medication from the thermosensitive coated NPs slowly at 41-43 degrees C. locally without the overreaction side effects seen after systemic, or oral ivermectin administration, thereby targeting multiple cancer cells, such as leukemia, glioblastoma, or glioma, or ovarian cancer, or breast cancer, Triple negative breast cancer cells, gastric cancer, colon cancer, or melanomas, etc. to damage the tumor cell with the combination with thermotherapy and enhancing the effect of immunotherapy or medication by thermotherapy and preventing production of medication resistance in these cancers.

In one embodiment, Ivermectin a Wnt/β-catenin pathway inhibitor prepared as PEGylated lipid nanoemulsion conjugated with monoclonal antibody coated functionalized pluralities of nanoparticles with activatable cell-penetrating peptides (ACPPs), nanoshell or nanocage administered systemically intravenously, intra-arterially locally, intraperitoneally, or in the cerebrospinal fluid to attach to the cancer cells. Also, the increased tumor biomarkers in the circulation after the thermotherapy has an important diagnostic implication for a presence of a tumor in the body and obtaining increased tumor biomarkers from the circulation after the thermotherapy, to confirm diagnosis of the specific cancer and cold storage of the new biomarkers for future management of the patient with potential recurrences by making a vaccine by combining the biomarkers conjugated with VLP, or intratumor injectable oncolytic viruses, monoclonal antibodies, IL-2, bee toxins or other immune stimulators and/or conjugating them with antibody/medication polymeric coated nanoparticles and checkpoint inhibitors, such as PD-1, Jagged 1 inhibitor 15D11, etc., and Rock inhibitors or Wnt inhibitors, such as niclosamide, administering them, subcutaneously or systemically, intravenously at very low dose to a patient, thereby the nanoparticles with the activatable cell-penetrating peptides (ACPPs) attach to the tumor cells, applying thermal energy to release the medication from the thermosensitive polymer coated nanoparticles and damage the tumor recurrence and to reactivate the immune response and eliminate the tumor cells.

In one embodiment, the pluralities of antibody/medication coated nanoparticles conjugated with either thermosensitive coated polymers or a combination of small liposomes containing functionalized pluralities of nanoparticles with activatable cell-penetrating peptides (ACPPs) that act as thermal energy contrast agents are administered intravenously, intra-arterially, locally, intra-arterially, targeting multiple cancers such as leukemia, glioblastoma with cell surface GPC2 or glioma, or retinoblastoma or ovarian cancer or breast cancer, triple-negative breast cancer cells, gastric cancer colon cancer, or melanomas, acute lymphocytic leukemia (ALL), etc. to image the lesion and the temperature using contrast agents, such as a liposome containing nanoshells, or nanocages containing, in addition, either a medication Wnt inhibitor, or integrin inhibitors such as NEDD9 scaffold protein, antibody LM609 and Nitroproline derivatives, etc., Avastin, aflibercept, ipilimumab nivolumab, or rock inhibitor in liposomal formulations, or buformin or phenformin, gemtuzumab or a dye or an ultrasound contrast agent such as PFCL to create a popping sound when heated under the control of the thermal energy delivery unit to reach the boiling point of the PFCL at temperature of 56 degrees C. temperature recording it by a microphone indicating the 56 degrees C. temperature has been achieved and to damage to the tumor cell, and not heat up the tissue further to limit thermal damage by heat spillover to the normal cells surrounding the tumor, and the increased tumor biomarkers in the circulation after the thermotherapy has an important diagnostic value by proving the presence of a tumor and/or use the biomarkers to make a vaccine for the future management of the patient recurrences.

In one embodiment, the antibody/medication and α-synuclein coated nanoparticle is conjugated with ACPP and a member of specific binding pair, a cellular receptor-agonist or antagonist pair, an enzyme-substrate pair, and/or an antibody-antigen pair, a virus, or virus like particles (VLP) or intratumor injectable oncolytic viruses, monoclonal antibodies, IL-2, or polymeric antibody coated nanoparticles carrying IL-2 and IL-21 or anti-CD3 (OKT3) antibody, to increase cytotoxicity of natural killer cells, bee toxins or other immune stimulators and Wnt inhibitors or Rock inhibitors with or without macrolides, such as cyclosporine A, mycophenolic acid, tactolimus, or ascomycin, is injected with monoclonal antibodies against the desired cancer along with a thermosensitive polymer, such as chitosan, carrying an antineoplastic medication, etc. and unquenched fluorescein or bubble liposomes carrying fluorescein which contain air pockets or nanoemulsions of PFC to be exposed to thermal energy releasing the medications at the temperature of 41-43 degrees C. as evidenced by the release of the dye in the circulation to enhance the patient's immune response to the tumor such as glioblastoma, melanoma, ocular melanoma, metastatic melanoma, breast cancer, ovarian cancer, etc. and induce an enhanced immune therapy.

In one embodiment, the antibody coated nanoparticle is conjugated with ACPP and a virus, or virus like particles (VLP), or intratumor injectable oncolytic viruses, monoclonal antibodies, IL-2, IL-6, IL-1β, IL-17, bee toxins or other immune stimulators along with checkpoint inhibitors, such as PD-1, PD-L1, CTLA-4, Jagged 1 inhibitor 15D11, and Wnt inhibitors or integrin inhibitors such as NEDD9 scaffold protein, antibody LM609 and Nitroproline derivatives, etc. or Rock inhibitors, and is injected with a monoclonal antibody against the desired cancer along with a thermosensitive polymer, such as chitosan, carrying an antineoplastic medication and unquenched fluorescein, or bubble liposomes carrying fluorescein which contain air pockets or nanoemulsions of PFC to be exposed to thermal energy releasing the medications, etc. at the temperature of 41-43 degrees C. as evidenced by the release of the dye in the circulation to enhance the patient's immune response to the tumor such as glioblastoma, melanoma, ocular melanoma, metastatic melanoma, breast cancer, ovarian cancer, prostate, lung cancer, etc. along with enhanced immune therapy.

In one embodiment, the antibody coated nanoparticle is conjugated with ACPP and with a member of a specific binding pair, such as a streptavidin-biotin pair, a cellular receptor-agonist or antagonist pair, an enzyme-substrate pair, and/or an antibody-antigen pair. The antibody coated nanoparticle may be associated with a virus, e.g., a modified virus, Imlygic, Telomelysin, VCP inhibitor, or virus-like particles (VLP) made from modified plant viruses or intratumor injectable oncolytic viruses, monoclonal antibodies, IL-2, bee toxins or other immune stimulators along with checkpoint inhibitors, such as PD-1, PD-11, CTLA-4, Jagged 1 inhibitor 15D11. VLP, plant viral capsids or membranes lacking the genetic components of the virus and a Wnt inhibitor or Rock inhibitor is injected with monoclonal antibody/medication against the desired cancer along with a thermosensitive polymer, such as chitosan, carrying an antineoplastic medication and unquenched fluorescein, or bubble liposomes carrying fluorescein which contain air pockets or nanoemulsions of PFC to be exposed to thermal energy releasing the medications, etc. at the temperature of 41-43 degrees C. as evidenced by the release of the dye in the circulation to enhance the patient's immune response to the tumor, such as glioblastoma, melanoma, ocular melanoma, metastatic melanoma, breast cancer, ovarian cancer, prostate, lung cancer, etc.

In one embodiment, the antibody coated nanoparticle is conjugated with ACPP and with a virus, e.g., a modified virus, a tumoricidal virus, and/or an adeno-associated virus (AAV) or virus like particles (VLP) made from modified plant viruses or intratumor injectable oncolytic viruses, monoclonal antibodies, IL-2, or polymeric antibody coated nanoparticles carrying IL-2 and IL-21, bee toxins or other immune stimulators along with checkpoint inhibitors, such as PD-1, PD-L1, CTLA-4, Jagged 1 inhibitor 15D11. VLP, plant viral capsids or membranes lacking the genetic components of the virus, and thus unable to multiply in the organism, and a Wnt inhibitor or Rock inhibitor is injected with monoclonal antibodies against the desired cancer along with a thermosensitive polymer, such as chitosan, carrying an antineoplastic medication and unquenched fluorescein or bubble liposomes carrying fluorescein which contain air pockets or nanoemulsions of PFC to be exposed to thermal energy releasing the medications etc. at the temperature of 41-43 degrees as evidenced by the release of the dye in the circulation to enhance the patient's immune response to the tumor, such as glioblastoma, melanoma, ocular melanoma, metastatic melanoma, breast cancer, ovarian cancer, prostate, lung cancer, etc.

In one embodiment, ivermectin causes an in increase cell permeability makes the cell more susceptible to up take of chemotherapy, while addition of Rock inhibitors and diphenylpyrazole reduce the side effects of inflammation in the immunotherapy and thermotherapy to prevent cytokine storm.

In one embodiment, ivermectin given as a non-toxic dose orally causes an increase cell permeability of the tumor makes the tumor cell more susceptible to up take of chemotherapy, while addition of Rock inhibitors, such as Fasudil, orally and diphenylpyrazole reduce the side effects of inflammation in the immunotherapy and thermotherapy to prevent cytokine storm.

In one embodiment, rock inhibitors Fasudil (HA-1077 a selective RhoA/Rho kinase (ROCK) inhibitor, or Y-27632, small molecule inhibitor of ROCK1 and ROCK2, or netarsudil or integrin inhibitors, such as NEDD9 scaffold protein, antibody LM609 and Nitroproline derivatives, etc. in liposomal preparation are administered systemically intravenously, intra-arterially locally, intra peritoneal, or in the cerebrospinal fluid with Biologic Response Modifiers using functionalized pluralities of nanoparticles with activatable cell-penetrating peptides (ACPPs) coated with rock inhibitors, Wnt inhibitors, Temozolomide, Cetuximab in thermosensitive polymers to release the medication at the desired place at a desired time or combine them with standard anti-inflammatory agents, etc., such as Steroids, Dexamethasone NASIDs, etc. and deliver pluralities of nanoparticles (i.e., biodegradable or non-biodegradable nanoparticles) administered systemically intravenously, intra-arterially locally, intra peritoneal or in the cerebrospinal fluid as thermal contrast agents to prevent a cytokine storm in cancer immune therapy and remove cytokines after the therapy by electrophoresis, plasmapheresis or plasma exchange, or ARF6 inhibition, etc. to prevent the cytokine storm.

In another embodiment, monoclonal antibody therapies and other biologics all are conjugated with the antibody/medication coated pluralities of nanoparticles with activatable cell-penetrating peptides (ACPPs), other biologics include soluble receptors, cytokines, and natural cytokine antagonist administered in the thermosensitive polymeric coating of the functionalized nanoparticles systemically, intravenously, locally, intra-arterially, or in a body cavity to target the tumor cells precisely to prevent the cytokine storm.

In another embodiment, humanized and chimeric antibodies are administered via thermosensitive polymer coated antibody conjugated pluralities of nanoparticles with activatable cell-penetrating peptides (ACPPs) intravenously or subcutaneously, and include TNF-alpha inhibitors, anti-B lymphocyte therapy, IL-6 inhibitors, cytotoxic T-lymphocyte-associated protein 4 (CTLA-4) fusion inhibitors, and IL-2 receptor antagonists and target lanthionine synthetase C-like 2 (LANCL2) for treating autoimmune disease administered in the thermosensitive polymeric coating of the functionalized nanoparticle systemically, intravenously, intra-arterially, locally, intraperitoneally, or in the cerebrospinal fluid, and release them at a desired place with thermotherapy to prevent a cytokine storm.

In one embodiment, rock inhibitors Fasudil (HA-1077 a selective RhoA/Rho kinase (ROCK) inhibitor, or Y-27632, small molecule inhibitor of ROCK1 and ROCK2, etc. with Infliximab administered in a thermosensitive polymeric coating of the functionalized nanoparticle with activatable cell-penetrating peptides (ACPPs) systemically, intravenously, intra-arterially, locally, orally, intraperitoneally, or in the cerebrospinal fluid and release them at a desired place with thermotherapy to prevent a cytokine storm.

In one embodiment, rock inhibitors Fasudil (HA-1077) a selective RhoA/Rho kinase (ROCK) inhibitor, or Y-27632, small molecule inhibitor of ROCK1 and ROCK2, or integrin inhibitors, such as NEDD9 scaffold protein, antibody LM609 and Nitroproline derivatives, etc. in liposomal preparation are administered systemically, intravenously, intra-arterially, locally, intraperitoneally, orally or in the cerebrospinal fluid, with an antibody coated plurality of nanoparticles with activatable cell-penetrating peptides (ACPPs), conjugated with thermosensitive nanoparticles and Adalimumab which is a humanized antibody administered subcutaneously, and release them at a desired place with thermo-immunotherapy to prevent a cytokine storm and to enhance the effect of immunotherapy or medication by thermotherapy and prevent production of medication resistance in these cancers.

In one embodiment, rock inhibitors Fasudil (HA-1077), a selective RhoA/Rho kinase (ROCK) inhibitor, or Y-27632, a small molecule inhibitor of ROCK1 and ROCK2, etc. in a functionalized liposomal preparation are administered systemically intravenously, intra-arterially, locally, intraperitoneally, or in the cerebrospinal fluid, and they are released at a desired place with thermotherapy to prevent a cytokine storm and to enhance the effect of immunotherapy or medication by thermotherapy while preventing production of medication resistance in these cancers.

In one embodiment, rock inhibitors Fasudil (HA-1077), a selective RhoA/Rho kinase (ROCK) inhibitor, or Y-27632, small molecule inhibitor of ROCK1 and ROCK2 with antibody/medication coated pluralities of nanoparticles conjugated with thermosensitive nanoparticles with activatable cell-penetrating peptides (ACPPs) and Golimumab, are delivered as a subcutaneous injection in cases that are refractory to treatment with calcineurin inhibitors, corticosteroid combination therapy and release them at a desired place with thermo-immunotherapy to prevent a cytokine storm.

In one embodiment, rock inhibitors Fasudil (HA-1077), a selective RhoA/Rho kinase (ROCK) inhibitor, or Y-27632, small molecule inhibitor of ROCK1 and ROCK2, or integrin inhibitors such as NEDD9 scaffold protein, antibody LM609 and Nitroproline derivatives, etc. are administered in a liposomal preparation containing quenched fluorescein or bubble liposomes carrying fluorescein which contain air pockets or nanoemulsions of PFC with antibody coated pluralities of nanoparticles with activatable cell-penetrating peptides (ACPPs), conjugated with thermosensitive nanoparticles and Certolizumab pegol, dosing depends on the condition for which it is given, although in most cases it is given as a 400-mg weekly subcutaneous injection as needed and released at a desired place with thermo-immunotherapy to prevent a cytokine storm.

In one embodiment, rock inhibitors Fasudil (HA-1077), a selective RhoA/Rho kinase (ROCK) inhibitor, or Y-27632, small molecule inhibitor of ROCK1 and ROCK2, etc., Avastin, aflibercept, ipilimumab, and/or nivolumab in liposomal preparation containing quenched fluorescein or another dye or indicator/medication or bubble liposomes carrying fluorescein/gene which contain air pockets or nanoemulsions of PFC are administered with antibody coated nanoparticles with activatable cell-penetrating peptides (ACPPs), conjugated with thermosensitive nanoparticles with cell-penetrating peptides (CPPs) and Rituximab, an anti-CD20 molecule given via intravenous infusion, to kill B lymphocytes, and thus eliminate them from the inflammatory process and release them at a desired place with thermo-immunotherapy to prevent a cytokine storm.

In one embodiment, rock inhibitors in liposomal preparation containing fluorescein are administered systemically, intravenously, intra-arterially, locally, intraperitoneally, or in the cerebrospinal fluid, with antibody coated pluralities of nanoparticles with cell-penetrating peptides (CPPs), conjugated with thermosensitive nanoparticles and the IL-6 inhibitor tocilizumab and released at a desired place with thermo-immunotherapy to prevent a cytokine storm.

In one embodiment, rock inhibitors, such as Botox or botulinum toxin at picogram to one nanogram concentrations is given subcutaneously, locally, or less than one picogram concentration with antibody coated pluralities of nanoparticles with cell-penetrating peptides (CPPs), conjugated with thermosensitive nanoparticles and the IL-6 inhibitor, tocilizumab, and released at a desired place with thermo-immunotherapy to prevent a cytokine storm.

In one embodiment, rock inhibitors in a liposomal preparation containing quenched fluorescein or another indicator or bubble liposomes carrying fluorescein which contain air pockets or nanoemulsions of PFC are administered systemically, intravenously, intra-arterially, locally, intraperitoneally, or in the cerebrospinal fluid, with antibody coated pluralities of nanoparticles with cell-penetrating peptides (CPPs), conjugated with thermosensitive nanoparticles with cell-penetrating peptides (CPPs) and the CTLA-4 fusion inhibitor abatacept, ipilimumab, nivolumab, and they are released at a desired place with thermo-immunotherapy to prevent a cytokine storm, while preventing production of medication resistance in these cancers.

In one embodiment, rock inhibitors Fasudil (HA-1077), a selective RhoA/Rho kinase (ROCK) inhibitor, or Y-27632, a small molecule inhibitor of ROCK1 and ROCK2, etc. are administered systemically intravenously, intra-arterially, locally, intraperitoneally, or in the cerebrospinal fluid, in a liposomal preparation containing fluorescein with antibody coated pluralities of nanoparticles with cell-penetrating peptides (CPPs), conjugated with a thermosensitive nanoparticles and with the IL-2 receptor antagonist daclizumab and they are released at a desired place with thermotherapy and temperature of 41-43 degrees C. to prevent a cytokine storm, and prevent production of medication resistance in these cancers.

In one embodiment, rock inhibitors, such as Botox, at picogram concentrations are administered with antibody coated pluralities of nanoparticles with cell-penetrating peptides (CPPs), conjugated with thermosensitive nanoparticles and Alkylating agents and ipilimumab, nivolumab, administered systemically intravenously, intra-arterially locally, intraperitoneally, or in the cerebrospinal fluid, and they are released at a desired place with thermotherapy at the temperature of 41-43 degrees C. to prevent a cytokine storm.

In one embodiment, rock inhibitors Fasudil (HA-1077), a selective RhoA/Rho kinase (ROCK) inhibitor, or Y-27632, a small molecule inhibitor of ROCK1 and ROCK2, etc. or integrin inhibitors such as NEDD9 scaffold protein, antibody LM609 and Nitroproline derivatives, etc. in a liposomal preparation containing quenched fluorescein, or another indicator/medication are administered with antibody coated pluralities of nanoparticles with cell-penetrating peptides (CPPs), conjugated with thermosensitive nanoparticles with cell-penetrating peptides (CPPs), and small molecule Wnt inhibitors and a biologic response modifier or calcineurin inhibitor and paclitaxel systemically intravenously, intra-arterially locally, intraperitoneally, or in the cerebrospinal fluid, and they are released at a desired place with thermotherapy at the temperature of 41-43 degrees C. to reduce inflammatory processes.

In one embodiment, rock inhibitors Fasudil (HA-1077), a selective RhoA/Rho kinase (ROCK) inhibitor, or Y-27632, small molecule inhibitor of ROCK1 and ROCK2, Ripasudil, netarsudil, etc. in a liposomal preparation containing fluorescein are administered with antibody coated pluralities of nanoparticles with cell-penetrating peptides (CPPs), conjugated with thermosensitive nanoparticles and/or small molecules or Canakinumab is approved for use in juvenile idiopathic arthritis administered systemically, intravenously, intra-arterially, locally, intraperitoneally, or in the cerebrospinal fluid, and they are released at a desired place with thermotherapy at the temperature of 41-43 degrees C. to reduce inflammatory processes while prevent production of medication resistance in these cancers.

In one embodiment, Rock inhibitors are administered systemically, intravenously, intra-arterially, locally, intraperitoneally, or in the cerebrospinal fluid, with antibody coated pluralities of nanoparticles with cell-penetrating peptides (CPPs), conjugated with thermosensitive nanoparticles and Gevokizumab with or without small molecule Wnt inhibitor and they are released at a desired place with thermotherapy and a temperature of 41-43 degrees C.

In one embodiment, Rock inhibitors are administered systemically, intravenously, intra-arterially, locally, intraperitoneally, or in the cerebrospinal fluid, with antibody coated pluralities of nanoparticles with cell-penetrating peptides (CPPs), conjugated with thermosensitive nanoparticles and Gevokizumab with or without small molecule Wnt inhibitor, and they are released at a desired place with thermotherapy and a temperature of 41-43 degrees C. or the Rock inhibitors or Wnt inhibitors are given orally at a non-toxic dose once or can be repeated monthly if needed.

In one embodiment, rock inhibitors Fasudil (HA-1077), a selective RhoA/Rho kinase (ROCK) inhibitor, or Y-27632, a small molecule inhibitor of ROCK1 and ROCK2, etc. or integrin inhibitors such as NEDD9 scaffold protein, antibody LM609 and Nitroproline derivatives, etc. in a liposomal preparation containing quenched fluorescein are administered with antibody coated nanoparticles with cell-penetrating peptides (CPPs), conjugated with thermosensitive nanoparticles and Alemtuzumab, which has been shown to be beneficial for use in B-cell chronic lymphocytic leukemia, and they are released at a desired place with thermo-immunotherapy at the temperature of 41-43 degrees C. to reduce inflammatory processes while preventing production of medication resistance in these cancers because the cancer cells do not survive the hyperthermia.

In one embodiment, Rock inhibitors are administered systemically, intravenously, intra-arterially, locally, inside the tumor, intraperitoneally, or in the cerebrospinal fluid with antibody coated nanoparticles, conjugated with thermosensitive nanoparticles with cell-penetrating peptides (CPPs) and with Sarilumab a monoclonal antibody that targets IL-6, preventing T- and B-cell activation and differentiation and releases them at a desired place with thermotherapy at the temperature of 41-43 degrees C. to reduce inflammatory processes.

In one embodiment, rock inhibitors are administered systemically intravenously, intra-arterially locally, intraperitoneally, or in the cerebrospinal fluid with antibody/medication coated pluralities of nanoparticles, conjugated with thermosensitive nanoparticles with cell-penetrating peptides (CPPs) and Sirolimus and release them at a desired place with thermotherapy at the temperature of 41-43 degrees C. to reduce inflammatory processes while preventing production of medication resistance in these cancers because the cancer cells do not survive the hyperthermia.

In one embodiment, Rock inhibitors in liposomal preparation containing quenched fluorescein are administered systemically intravenously, intra-arterially locally, intraperitoneally, or in the cerebrospinal fluid with antibody coated pluralities of nanoparticles conjugated with thermosensitive nanoparticles with cell-penetrating peptides (CPPs) and Interferon-α2a and release them at a desired place with thermotherapy at the temperature of 41-43 degrees C. while preventing production of medication resistance in these cancers because the cancer cells do not survive the hyperthermia.

In one embodiment, radiolabeled antibodies, such as Ibritumomab tiuxetan, is conjugated with a thermosensitive polymer, such as chitosan, coating antibody coated nanoparticle to deliver the medication to the tumor cell systemically intravenously, intra-arterially locally, intraperitoneally, or in the cerebrospinal fluid and release them at a desired place with thermotherapy at the temperature of 41-43 degrees C. to reduce side effect of tiuxetan and Ibritumomab.

In one embodiment, the thermosensitive polymer of the antibody/medication coated nanoparticles with ACPP, containing a radiolabeled medication is administered systemically intravenously, intra-arterially locally, intraperitoneally, or in the cerebrospinal fluid to damage the tumor cells as a combination thermoradiotherapy and reduce their side effect while preventing production of medication resistance in these cancers because the cancer cells do not survive the hyperthermia.

In one embodiment, the thermosensitive polymer of the antibody/medication coated pluralities of nanoparticles, such as gold nanoparticles that enhance the effect of external x-ray or particle beam radiation contains mebendazole, taxol derivatives, an anti-CCR2 antibody results in blockade of radiation-induced monocytic myeloid-derived suppressor cells infiltration that create radiation resistance, and fibrous tissue scars, in addition Rock inhibitors, such as Fasudil etc., or integrin inhibitors such as NEDD9 scaffold protein, antibody LM609 and Nitroproline derivatives, etc. intra-arterially locally, in the body cavity given to reduce inflammatory process and scar tissue, and quenched fluorescein or another indicator with Wnt inhibitors administered systemically intravenously, intra-arterially locally, intraperitoneally, or in the cerebrospinal fluid released with controlled thermotherapy when administered medication to damage the tumor cells as a combination thermoradiotherapy in glioblastoma, medulloblastoma, retinoblastoma, melanoma or other brain tumors, lung cancer acting on the microtubes of the cancer cells while preventing production of medication resistance in these cancers because the cancer cells do not survive the hyperthermia, etc.

In one embodiment, the thermosensitive polymer of the antibody/medication coated nanoparticles contains mebendazole, taxol derivatives, and quenched fluorescein or another indicator with Rock inhibitors, checkpoint inhibitors administered systemically, intravenously, intra-arterially locally, intraperitoneally, or in the cerebrospinal fluid released with controlled thermotherapy when administered medication to damage the tumor cells as a combination thermoradiotherapy in glioblastoma, medulloblastoma, retinoblastoma, melanoma or other brain tumors, lung cancer acting on the microtubes of the cancer cells while preventing production of medication resistance in these cancers because the cancer cells do not survive the hyperthermia, etc.

In one embodiment, one or multiple agents are used to treat a number of cancers alone with a monoclonal antibody conjugated with thermosensitive antibody coated pluralities of nanoparticles with cell-penetrating peptides (CPPs), nanoshell or nanocage treatment with temperature sensitive polymers conjugated with the nanoparticles having ipilimumab, nivolumab, and/or temozolomide combined a small molecule Wnt inhibitor, ivermectin, or niclosamide along with paclitaxel, or mebendazole, VLP or intratumor injectable oncolytic viruses, monoclonal antibodies, IL-2, or polymeric antibody coated nanoparticles carrying IL-2 and IL-21, bee toxins or other immune stimulators along with checkpoint inhibitors, such as PD-1, PD-11, CTLA-4, Jagged 1 inhibitor 15D11 administered intravenously or intra-arterially by injection, systemically intravenously, locally, intraocularly, in the tumor, intraperitoneally or in the cerebrospinal fluid at nanogram doses to be released from the antibody/medication coated nanoparticle at the tumor site using thermotherapy with either alternating magnetic field or focused low power ultrasound, a laser and simultaneous release of a dye such as quenched fluorescein or indocyanine green from the nanoparticle's thermosensitive polymer or magnetic nanoshells containing fluorescein and PFH under observation with MRI or focused ultrasonic units, a CT-scan, or PET-scan, and release the medication at a desired place with thermotherapy at the temperature of 41-43 degrees C. or higher up to 60 or more degrees C. as recorded by a microphone or an ultrasonic receiver, and imaged with a thermoacoustic imaging system for control of the temperature and to induce an enhanced immunotherapy to damage the tumor cells while verifying the medication/dye release from the blood samples and obtain newly released biomarkers from the tumor cells in the circulation to make a vaccine with VLP and/or IL-2 combined with antibody coated nanoparticles.

In one embodiment, the focused low power ultrasound is combined with the alternating magnetic field to simultaneously heat and image the tissue while heating and killing the tumor at a lower time duration, the pluralities of nanoparticles with cell-penetrating peptides (CPPs) are coated with thermosensitive polymers for drug release at temp. of 41-43 degrees C., imaged with a thermoacoustic imaging system or dual imaging system with photoacoustic and ultrasound imaging for control of the temperature and the popping sound of the cavitation sound is heard at the temperature of 56 degrees C. killing the tumor cells releasing their antigenic content to stimulate immune response to remove all the tumor cells, thereby eliminating the need for cellular immune therapy, and the increased tumor biomarkers in the circulation after the thermotherapy has an important diagnostic (i.e., indicating presence of a tumor) and therapeutic value as biomarkers for the future management of the patient.

In one embodiment, the focused ultrasound can be directed by a computer controlled head to the exact location and borders of the tumor mass, which may be marked by focused high power ultrasound to heat up and create bubbles after heating the nanoshell or nanocage filled with PFCL hexane to its transition temperature of 56 degrees C. that can be recorded by an additional ultrasound receiver or a microphone as a cavitation sound creating a precise ablation of a tumor under ultrasonic observation and drug Wnt inhibitors and a Rho inhibitor or mebendazole, diphenylpyrazole, release to kill the tumor while dampening the post-therapy inflammation, thus enhancing the person's immune response cells eliminating the need for cellular immune therapy, and the increased tumor biomarkers in the circulation after the thermotherapy has an important diagnostic (i.e., indicating presence of a tumor) and therapeutic value as biomarkers for the future management of the patient having glioblastoma, medulloblastoma, ovarian tumor, or breast cancer, retinoblastoma, glioma, etc.

In one embodiment, a known Wnt inhibitor, such as Ivermectin or mebendazole, conjugated with ipilimumab, nivolumab, and thermosensitive polymer antibody coated nanoparticles, nanocages, nanoshells having the temperature markers fluorescein and PFCL intravenously, intra-arterially locally, or in the cerebrospinal fluid systemically intravenously, locally, intraperitoneally to attach to the tumor receptors and heated with either an alternating magnetic field, or ultrasound or microwave to heat up the tumors and release the medication under control of the temperature or 41-43 degrees C., then 56 degrees C., imaged with a thermoacoustic imaging system for control of the temperature and a processor to release the medication and kill the tumor simultaneously by thermal energy releasing more biomarkers from the tumor to enhance immune response cells by releasing increased tumor biomarkers in the circulation and eliminating the need for cellular immune therapy, and the increased tumor biomarkers in the circulation after the thermotherapy has an important diagnostic (i.e., indicating presence of a tumor) and therapeutic value as biomarkers for vaccine production for the future management of the patient having glioblastoma, medulloblastoma, ovarian tumor, or breast cancer, retinoblastoma, gliomas, and administering them along with checkpoint inhibitors, such as PD-1, Jagged 1 inhibitor 15D11, etc., and Rock inhibitors or Wnt inhibitors, such as niclosamide etc.

In one embodiment, a known Wnt inhibitor, such as Ivermectin and taxol or other medication, conjugated with thermosensitive polymer antibody/medication coated nanoparticles, nanocages, nanoshells having the temperature markers quenched fluorescein or another indicator and PFCL intravenously, intra-arterially locally, or in the cerebrospinal fluid to attach to the tumor receptors and heated with either an alternating magnetic field, or focused low power ultrasound or microwave to heat up the tumors and release the medication under control of the temperature and a processor connected to thermal energy delivery system and thermal imaging unit to release the medication and kill the tumor simultaneously at a defined thermal energy temperature, and the increased tumor biomarkers in the circulation after the thermotherapy has an important diagnostic (i.e., indicating presence of a tumor) and therapeutic value as biomarkers for the future management of the patient and production of vaccine for the patient's tumor and administering it with checkpoint inhibitors, such as PD-1, Jagged 1 inhibitor 15D11, etc., and Rock inhibitors, such as Fasudil, or Wnt inhibitors, such as niclosamide.

In one embodiment, a small molecule Wnt inhibitor and taxane are conjugated with a thermosensitive polymer having ipilimumab and/or nivolumab, antibody coated nanoparticles, nanocages, or nanoshells having the temperature markers quenched fluorescein and PFCL intravenously, intra-arterially locally, inside the tumor, in the bladder, or in the cerebrospinal fluid to attach to the tumor receptors and heated with either an alternating magnetic field, low power ultrasound, or microwaves to heat up the tumors and release the medication under control of the temperature and a processor to release the medication and kill the tumor simultaneously by thermal energy and the increased tumor biomarkers in the circulation after the thermotherapy has an important diagnostic (i.e., indicating presence of a tumor) and therapeutic value as biomarkers for the future of the patient and production of vaccine for the patient's tumor and administering it with checkpoint inhibitors such as PD-1, Jagged 1 inhibitor 15D11, etc., and Rock inhibitors, such as Fasudil, or Wnt inhibitors, such as niclosamide.

In one embodiment, a known Wnt inhibitor, Ivermectin PEGylated lipid nanoemulsion conjugated along with the Rock inhibitor Fasudil (HA-1077), a selective RhoA/Rho kinase (ROCK) inhibitor, or Y-27632, a small molecule inhibitor of ROCK1 and ROCK2, etc. and decitabine with thermosensitive polymer antibody coated nanoparticles, nanocages, or nanoshells having the temperature markers fluorescein and PFCL intravenously, intra-arterially locally, or in the cerebrospinal fluid to attach to the tumor receptors and heated with either an alternating magnetic field, or low power ultrasound or microwave or radiofrequency to heat up the tumors and release the medication under control of the temperature and a processor to release the medication and kill the tumor simultaneously by thermal energy 43 degrees C., imaged with a thermoacoustic imaging system for control of the temperature to reduce the production of inflammatory cytokines by macrophages and the increased tumor biomarkers in the circulation after the thermotherapy has an important diagnostic (i.e., indicating presence of a tumor) and therapeutic value as biomarkers for the future management of the patient and production of vaccine for the patient's tumor and administering it with VLP and checkpoint inhibitors, such as PD-1, Jagged 1 inhibitor 15D11, etc., and Rock inhibitors, such as Fasudil, or Wnt inhibitors, such as niclosamide to enhance immunotherapy, while Rock inhibitors and Wnt inhibtors, in combination with anti-VEGFs, to reduce TGF-β production after therapy and the subsequent scar inflammation and formation.

In one embodiment, Ivermectin the Wnt/β-catenin pathway prepared as PEGylated lipid nanoemulsion conjugated with a monoclonal antibody and ipilimumab or nivolumab and coated pluralities of nanoparticles with cell-penetrating peptides (CPPs), nanoshells or nanocages administered systemically intravenously, intra-arterially, locally, or in the cerebrospinal fluid and other body cavities or tissues through lymphatic vessels to reach the lymph nodes to attach to the tumor receptors and heated with either an alternating magnetic field, low power ultrasound, or microwaves to heat up the tumors and release the medication under control of the temperature, imaged with a thermoacoustic imaging system for control of the temperature, a processor connected to the thermal delivery unit to release the medication at about 41-43 degrees C. when the lipid melts targeting multiple cancers, such as leukemia, glioblastoma, glioma, or ovarian cancer or breast cancer, triple negative breast cancer cells, gastric cancer, colon cancer, ovarian cancer, or melanomas, etc. and the increased tumor biomarkers in the circulation after the thermotherapy has an important diagnostic (i.e., indicating presence of a tumor) and therapeutic value as biomarkers for the future management of the tumor recurrences in the patient and production of vaccine for the patient's tumor and administering it with antibody coated VLP, checkpoint inhibitors such as PD-1, Jagged 1 inhibitor 15D11, etc., and Rock inhibitors, such as Fasudil, or Wnt inhibitors, such as niclosamide, etc. to enhance immune therapy while reducing inflammation.

In one embodiment, a known Wnt inhibitor and Wnt-TCF pathway Ivermectin the Wnt/β-catenin pathway prepared as PEGylated lipid nanoemulsion conjugated and a rock inhibitor Fasudil (HA-1077), a selective RhoA/Rho kinase (ROCK) inhibitor, or Y-27632, small molecule inhibitor of ROCK1 and ROCK2, etc. are conjugated with a thermosensitive polymer antibody coated nanoparticles, nanocages, or nanoshells having the temperature markers fluorescein and PFCL intravenously, intra-arterially, locally, or is the cerebrospinal fluid, etc. to attach to the tumor receptors and heated with either an alternating magnetic field, focused high power ultrasound, or microwave to heat up the tumors and release the medication under control of the temperature and a processor to release the medication and kill the tumor simultaneously by thermal energy while preventing production of medication resistance in these cancers because the cancer cells do not survive the hyperthermia.

In one embodiment, the oral dose for ivermectin is about 40-150 microgram/kg, once in 7 days and the systemic dose is (10-100 ng/ml) in 1-5 (ml) in physiologic solution, or more doses as needed, and the oral dose of Fasudil is 40 mg-80 mg and the systemic dose is 50 nanograms to a few micrograms.

In one embodiment, a small molecule Wnt inhibitor PKF118-310 the Wnt/β-catenin pathway inhibitor and fasudil, a rock inhibitor Fasudil (HA-1077), a selective RhoA/Rho kinase (ROCK) inhibitor, or Y-27632, small molecule inhibitor of ROCK1 and ROCK2, etc. prepared as PEGylated lipid nanoemulsion conjugated and a rock inhibitor conjugated with a thermosensitive polymer antibody coated nanoparticles, nanocages, or nanoshells having the temperature markers quenched fluorescein or another indicator and PFCL administered intravenously, intra-arterially locally, or in the cerebrospinal fluid to attach to the tumor receptors and heated with either an alternating magnetic field, focused ultrasound, or microwaves to heat up the tumors and release the medication and/or gemtuzumab under control of the temperature, imaged with a thermoacoustic imaging system for control of the temperature and a processor to release the medication and kill the leukemic cells, inducing leukemic cell death at a low micro molar concentration of Ivermectin or other hematological malignancies simultaneously by thermal energy and the increased tumor biomarkers in the circulation after the thermotherapy has an important diagnostic (i.e., indicating presence of a tumor) and therapeutic value as biomarkers vaccine for the future management of the tumor recurrences in the patient and production of vaccine for the patient's tumor and administering it with antibody coated nanoparticles with VLP etc. and checkpoint inhibitors, such as PD-1, Jagged 1 inhibitor 15D11, etc., and Rock inhibitors, such as Fasudil, or Wnt inhibitors, such as niclosamide for enhanced immunotherapy.

In one embodiment, a small molecule Wnt inhibitor PKF118-310 the Wnt/β-catenin pathway inhibitor and fasudil, a rock inhibitor Fasudil (HA-1077), a selective RhoA/Rho kinase (ROCK) inhibitor, or Y-27632, a small molecule inhibitor of ROCK1 and ROCK2, etc. is prepared as PEGylated lipid nanoemulsion conjugated and a rock inhibitor and metformin, buformin, or phenformin to inhibit glucose metabolism in the tumor cells and cinnamaldehyde that indices apoptosis and conjugated with a thermosensitive polymer antibody coated pluralities of nanoparticles, nanocages, or nanoshells having the temperature markers quenched fluorescein or another indicator and PFCL administered intravenously, intra-arterially locally, or in the cerebrospinal fluid to attach to the tumor receptors and heated with either an alternating magnetic field, focused ultrasound, or microwaves to heat up the tumors and release the medication under control of the temperature, imaged with a thermoacoustic imaging system for control of the temperature and a processor to release the medication and kill the tumor cells and starve those that might escape treatment preventing their important glucose metabolism.

In one embodiment, a small molecule Wnt inhibitor PKF118-310, the Wnt/β-catenin pathway inhibitor and fasudil, a rock inhibitor Fasudil (HA-1077), a selective RhoA/Rho kinase (ROCK) inhibitor, or Y-27632, a small molecule inhibitor of ROCK1 and ROCK2, etc. or integrin inhibitors such as NEDD9 scaffold protein, antibody LM609 and Nitroproline derivatives, etc. is prepared as PEGylated lipid nanoemulsion conjugated and a rock inhibitor and metformin, buformin, or phenformin and syrosingopine to inhibit glucose metabolism in the tumor cells, or in combination with a glutaminase inhibitor, such as Calithera inhibiting glutamine uptake for the tumor to which the tumor cells are addicted, and conjugated with a thermosensitive polymer antibody/medication coated nanoparticles, nanocages, or nanoshells having the temperature markers quenched fluorescein or another indicator and PFCL administered intravenously, intra-arterially locally, or in the cerebrospinal fluid to attach to the tumor receptors and heated with either an alternating magnetic field, low power ultrasound, or microwaves to heat up the tumors and release the medication under control of the temperature, imaged with a thermoacoustic imaging or dual system imaging with photoacoustic and ultrasound for control of the temperature and a processor to release the medication and kill the tumor cells and starve those that might escape treatment preventing their important glucose metabolism.

In one embodiment, a known Wnt inhibitor Ivermectin and doxorubicin (Adriamycin) or taxol derivatives are conjugated with thermosensitive polymer antibody coated nanoparticles, nanocages, or nanoshells having the temperature markers quenched fluorescein or another indicator and PFCL conjugated with Niclosamide, Rock inhibitor Fasudil (HA-1077), a selective RhoA/Rho kinase (ROCK) inhibitor, or Y-27632, a small molecule inhibitor of ROCK1 and ROCK2 and femformin, etc. in liposomal preparation containing fluorescein, metformin, or buformin, cysteine protease inhibitors, such as stefins, and antineoplastic medication or monoclonal antibody rituximab intravenously, intra-arterially locally, or in the cerebrospinal fluid to attach to the tumor receptors and heated with either an alternating magnetic field, low power ultrasound, or microwaves to heat up the tumors and release the medication under control of the temperature with a thermoacoustic imaging system for control of the temperature initially and a processor to control the temperature, imaged at 41-43 degrees C. with the release of quenched fluorescein to release the medication and kill the tumor simultaneously by thermal energy subsequently at 50-56 degrees C. with the low power focused ultrasound receiver showing the ultrasound cavitation at a boiling point of PFCL in triple negative breast cancer or in ovarian cancer, glioblastoma, melanoma, retinoblastoma, gastric cancer, etc. and the increased tumor biomarkers in the circulation after the thermotherapy has an important diagnostic value (i.e., confirming the presence of a tumor) and therapeutic value as biomarkers for the future management of the patient with recurrences by making a vaccine using the biomarkers and VLP or intratumor injectable oncolytic viruses, monoclonal antibodies, IL-2, or the anti-CD3 (OKT3) antibody, to increase cytotoxicity of natural killer cells, bee toxins or other immune stimulators conjugated with antibody coated nanoparticles to attach to the potential tumor cells and administering it with checkpoint inhibitors, such as PD-1, Jagged 1 inhibitor 15D11, etc., and Rock inhibitors, such as Fasudil, or Wnt inhibitors, such as niclosamide, while applying thermal energy to damage recurrence of the tumor and induce reactivation of the immune response to eliminate them and prevent the tumor's important glucose metabolism with metformin, buformin, or in combination with a glutaminase inhibitor, such as Calithera inhibiting glutamine uptake for the tumor to which the tumor cells are addicted, while antibody coated nanoparticles/checkpoint inhibitors and VLP attach to the localized or circulating tumor cells and their exosomes, which are carrying a checkpoint protein, such as PD-L1 (to disguise themselves), and the tumor cells are recognized by the T cells, which together with killer cells phagocytose them, and enhance the immune response to the tumor and its exosomes and the circulating cells.

In one embodiment, a known Wnt inhibitor, Ivermectin and doxorubicin (Adriamycin) or taxol derivatives are conjugated with thermosensitive polymer antibody coated nanoparticles, nanocages, or nanoshells having the temperature markers quenched fluorescein or another dye or indicator and PFCL conjugated with Niclosamide, Rock inhibitor Fasudil (HA-1077), a selective RhoA/Rho kinase (ROCK) inhibitor, or Y-27632, a small molecule inhibitor of ROCK1 and ROCK2 and femformin, etc. in liposomal preparation containing fluorescein, metformin, or buformin, or metformin cysteine protease inhibitors, such as stefins, and with or without syrosingopine, cinnamaldehyde and an antineoplastic medication or monoclonal antibody rituximab intravenously, intra-arterially locally, or in the cerebrospinal fluid to attach to the tumor receptors and heated with either an alternating magnetic field, ultrasound, or microwaves to heat up the tumors and release the medication under control of the temperature, imaged with a thermoacoustic imaging system for control of the temperature and a processor to control the temperature initially at 41-43 degrees C. with the release of quenched fluorescein to release the medication and kill the tumor simultaneously by thermal energy subsequently at 50-56 degrees C. with the high energy ultrasound receiver showing the ultrasound cavitation at a boiling point of the PFCL in triple negative breast cancer or in ovarian cancer, glioblastoma, melanoma, retinoblastoma, gastric cancer, etc. and the increased tumor biomarkers in the circulation after the thermotherapy has an important diagnostic value (i.e., confirming the presence of a tumor) and therapeutic value as biomarkers for the future management of the patient with recurrences by making a vaccine using the biomarkers and VLP or intratumor injectable oncolytic viruses conjugated with antibody coated nanoparticles and administering it with checkpoint inhibitors, such as PD-1, Jagged 1 inhibitor 15D11, etc., and Rock inhibitors, such Fasudil, or Wnt inhibitors, such as niclosamide, to attach to the potential tumor cells while applying thermal energy to damage recurrence of the tumor and induce reactivation of the immune response to eliminate them and prevent the tumor's important glucose metabolism with metformin, buformin and syrosingopine or cinnamaldehyde.

In one embodiment, a known Wnt inhibitor, ivermectin, niclosamide, metformin, or buformin, or in combination with a glutaminase inhibitor, such as Calithera inhibiting glutamine uptake for the tumor to which the tumor cells are addicted, are conjugated with thermosensitive polymer antibody/medication coated nanoparticles, nanocages, nanoshells having the temperature markers quenched fluorescein or another indicator and PFCL intravenously, intra-arterially locally, inside the tumor, or in the cerebrospinal fluid to attach to the tumor receptors and heated with either an alternating magnetic field, LIFU or high power ultrasound, or microwaves to heat up the tumors and release the medication under control of the temperature of 41-43 degrees C. or 50-56 degrees C., imaged with a thermoacoustic imaging system for control of the temperature and a processor to release the medication and kill the tumor simultaneously by thermal energy in treatment of glioblastoma or glioma, or ovarian cancer or breast cancer and the increased tumor biomarkers in the circulation after the thermotherapy has an important diagnostic (i.e. indicating presence of a tumor) and therapeutic value as biomarkers for the future management of the patient and eliminating the need for CAR-T administration to the patient.

In one embodiment, a known Wnt inhibitor, Niclosamide and taxol, or daunorubicin, ara-C or cytarabine or metformin, buformin or phenformin with or without syrosingopine are conjugated with a thermosensitive polymer antibody coated nanoparticles and ACPP, nanocages or nanoshells having the temperature markers quenched fluorescein and PFCL intravenously, intra-arterially locally, or in the cerebrospinal fluid to attach to the tumor receptors and heated with either an alternating magnetic field, ultrasound, or microwaves to heat up the tumors and release the medication under control of the temperature and a processor to release the medication and kill the tumor simultaneously by thermal energy in treatment of glioblastoma or glioma, or ovarian cancer or breast cancer, gastric cancer, colon cancer, or melanomas, Mantle cell lymphoma preferentially killing the cancer stem cells, etc. and the increased tumor biomarkers in the circulation after the thermotherapy has an important diagnostic (i.e., indicating presence of a tumor) and therapeutic value as biomarkers for vaccine production with or without VLP attached to antibody coated nanoparticles and administering it with checkpoint inhibitors such as PD-1, Jagged 1 inhibitor 15D11, etc., and Rock inhibitors, such as Fasudil, or Wnt inhibitors, such as niclosamide, with cell-penetrating peptides (CPPs) for the management of the future recurrences in the patient and eliminating the need for CAR-T administration to the patient, and preventing their important glucose metabolism with metformin, buformin or phenformin while antibody coated nanoparticles/checkpoint inhibitors and VLP attach to the localized or circulating tumor cells and their exosomes, which are carrying a checkpoint protein, such as PD-L1 (to disguise themselves), and the tumor cells are recognized by the T cells, which together with killer cells phagocytose them, and enhance the immune response to the tumor and its exosomes and the circulating cells.

In one embodiment, one eliminates the defense mechanism of the tumor cells, circulating tumor cells, and their exosomes by systemic, intravenous, interarterial, or local inside the tumor injection of antibody coated polymeric nanoparticles, preferably coated with thermosensitive polymers made of biodegradable polymeric compositions, such as orthoesters, anhydrides, amides, calcium alginate polysaccharides functionalized celluloses, carboxymethylcellulose polycaprolactone copolymers of glycolic and lactic acid, polymers of D-lactic acid, L-lactic acid, racemic lactic acid, glycolic acid, porous silicon, liposomes, micelles of 2-500 nm but preferably 10-50 nm combined with immune stimulators, such as IL-2, IL-15, IL-17 or polymeric antibody coated nanoparticles carrying IL-2 and IL-21 or the anti-CD3 (OKT3) antibody, to increase cytotoxicity of natural killer cells, IL-6, IL-1β, or TNF, TNF-α, or toll-like receptors TLR 7, TLR 8 and IFN-γ or granzyme (3 related apoptosis-inducing ligand (TRAIL), or the Fas ligand to attract immune cells to the immune stimulators and simultaneously phagoocytize the circulating exosomes, circulating tumor cells, or the original tumor, to prevent a cytokine storm, the blood runs through a tube that is heated with an external source of energy such as a laser, microwave, radiofrequency radiation, alternating magnetic field (AMF) or HIFU or non-thermal LIFU, etc. to heat up the blood to a temperature of 40-41 degrees C. for a very short time to melt the polymeric nanoparticles which are made from the liposomes and clear the blood by dielectophoresis before its re-administration in the circulation along with a macrolide or Rock inhibitors with the concentration of 1-100 or more nanograms to prevent a systemic inflammatory response.

In one embodiment, one eliminates the defense mechanism of the tumor cells, circulating tumor cells, and their exosomes by systemic, intravenous, interarterial, or local inside the tumor injection of antibody coated polymeric nanoparticles, preferably thermosensitive polymers made of biodegradable polymeric compositions of orthoesters, anhydrides, amides, calcium alginate polysaccharides, functionalized celluloses, carboxymethylcellulose polycaprolactone, copolymers of glycolic and lactic acid, polymers of D-lactic acid, L-lactic acid, racemic lactic acid, glycolic acid, liposomes, micelles of 2-500 nm, but preferably 10-50 nm combined with immune stimulators, such as IL-2 or IL-15, IL-17 or polymeric antibody coated nanoparticles carrying IL-2 and IL-21 or the anti-CD3 (OKT3) antibody, to increase cytotoxicity of natural killer cells, IL-6, IL-7, IFN-γ or granzyme β, related apoptosis-inducing ligand (TRAIL), or the Fas ligand, combined with a checkpoint inhibitor such as PD-1 or PD-L1, CTLA-4, Jagged 1 inhibitor 15D11, or in combination with one more other checkpoint inhibitors to not only attach to the tumor cells, but also to the circulating tumor cells, and to block the action of the tumor cells in disguising themselves, followed by plasmapheresis, thermotherapy of the blood and dielectropheresis to eliminate the dead cells, exosomes, etc. The process can be repeated in another session as needed to eliminate metastatic disease.

In one embodiment, one eliminates the defense mechanism of the tumor cells, circulating tumor cells, and their exosomes by systemic, intravenous, interarterial or local inside the tumor injection of antibody coated polymeric nanoparticles, preferably thermosensitive polymers made of biodegradable polymeric compositions, such as orthoesters, anhydrides, amides, calcium alginate polysaccharides functionalized celluloses, carboxymethylcellulose polycaprolactone, copolymers of glycolic and lactic acid, polymers of D-lactic acid, L-lactic acid, racemic lactic acid, glycolic acid, liposomes, micelles of 2-500 nm but preferably 10-50 nm combined with immune stimulators, such as IL-2, IL-15, or polymeric antibody coated nanoparticles carrying IL-2 and IL-21, IL-6, IL-7, TLR 6, or TLR 7, IFN-γ or granzyme β, related apoptosis-inducing ligand (TRAIL), or the Fas ligand, and antibody coated VLP, combined or separate polymeric antibody coated nanoparticles with a checkpoint inhibitor, such as PD-1 or PD-L1, CTLA-4, Jagged 1 inhibitor 15D11, or in combination with one or more other antibody coated polymeric nanoparticles conjugated with two or more checkpoint inhibitors and injected inside the tumor, locally anywhere the tumor is located including inside the skin or mucosa, in combination with or without thermotherapy to release the immunostimulators immediately or over a period of time to incite an immune response eliminating the tumor and its metastatic cells, exosomes, followed with plasmapheresis, etc. to clean up the blood from the toxins, the polymeric nanoparticles provide a long lasting immune response inside the tumor or its metastatic lesions. The procedure can be repeated in the future as needed.

In one embodiment, one eliminates the defense mechanism of the tumor cells, circulating tumor cells, and their exosomes by systemic, intravenous, interarterial, or local inside the tumor injection of antibody coated polymeric nanoparticles, preferably coated with thermosensitive polymers made of biodegradable polymeric compositions, such as orthoesters, anhydrides, amides, calcium alginate polysaccharides, functionalized celluloses, carboxymethylcellulose polycaprolactone, copolymers of glycolic and lactic acid, polymers of D-lactic acid, L-lactic acid, racemic lactic acid, glycolic acid, liposomes, micelles of 2-500 nm but preferably 10-50 nm combined with immune stimulators, such as IL-2 or polymeric antibody coated nanoparticles carrying IL-2 and IL-21, or the anti-CD3 (OKT3) antibody, to increase cytotoxicity of natural killer cells, or IL-15, IL-6, IL-7, IFN-γ or granzyme β, related apoptosis-inducing ligand (TRAIL), or the Fas ligand, combined with a checkpoint inhibitor such as PD-1 or PD-L1, CTLA-4, Jagged 1 inhibitor 15D11, or in combination with one more other checkpoint inhibitors and antibody coated nanoparticles carrying one or more chemotherapeutic agents, and with the application of external thermal energy, such as HIFU or LIFU, or low frequency <40 kHz, alternating magnetic field (AMF) or high frequency Megahertz, AMF, if the nanoparticles are magnetic, or laser beam, to not only attach to the tumor cells but also to the circulating tumor cells and block the action of the tumor cells in disguising themselves to incite an immune response with or without thermotherapy for release of the polymeric medications, eliminating the tumor, its exosomes, and its metastatic cells, damage their metabolism by the chemotherapeutic agents followed with plasmapheresis, etc. to clean up the blood from the toxins, the polymeric nanoparticles provide a long lasting immune response inside the tumor or its metastatic lesions, the procedure can be repeated in the future as needed along with plasmaphoresis.

In one embodiment, an immune response is initiated to eliminate not only the cancer cells, but also their circulating cells, and their exosomes that have the tumor biomarkers, and contribute to the cancer immune resistance by administering locally, inside the tumor and/or systemically antibody coated polymeric nanoparticles conjugated with immune stimulators such as IL-1, TNF, TNF-α, or toll-like receptors TLR 7 or administering antibody coated polymeric nanoparticles conjugated with immune stimulators such as VLP, IL-2, or polymeric antibody coated nanoparticles carrying IL-2 and IL-21, or the anti-CD3 (OKT3) antibody, to be attached to the circulating tumor cells and exosomes to be attacked by the body's natural killer cells or by administering natural killer cells or other immune cells and eliminating the circulating tumor cells and their exosomes, and thereby preventing tumor resistance to the cellular immune response.

In one embodiment, an immune response is initiated to eliminate not only the cancer cells, but also their circulating cells, and their exosomes that have the tumor biomarkers, and contribute to the cancer immune resistance by administering locally or systemically antibody coated polymeric nanoparticles conjugated with immune stimulators such as IL-1 or TNF, TNF-α, or toll-like receptors TLR 7 or administering antibody coated polymeric nanoparticles conjugated with immune stimulators such as VLP, IL-2, or polymeric antibody coated nanoparticles carrying IL-2 and IL-21, or the anti-CD3 (OKT3) antibody, to be attached to the circulating tumor cells and exosomes, and to be attacked by the body's natural killer cells or by administered natural killer cells or other immune cells, and combined or sequential administration of two or more checkpoint inhibitors with or without chemotherapy and eliminating the circulating tumor cells and exosomes, thereby preventing tumor resistance to the cellular immune response, followed by plasmaphoresis to clean up the blood from the released toxins and prevent a cytokine storm in the body.

In one embodiment, one injects polymeric antibody coated nanoparticles carrying IL-2 or IL-21, IL-15, or TLR 7 inside the tumor, or in the circulation prior to administration of cellular immune therapy with natural killer cells, to increase the natural cytotoxicity and survival of the natural killer cells, while the antibody coated nanoparticles attach to the circulating exosomes or circulating tumor cells to be attacked by natural killer cells of the body or administered intravenously, simultaneously, or sequentially with the nanoparticles to stimulate natural killer cells and other immune cells to eliminate the tumor exosomes carrying PD-L1 which prevent them from blocking the cellular immune response. Thus, the circulating exosomes, circulating tumor cells, or the original cancer cells can be indirectly recognized either by their antigenic membrane receptors, or by the antibody coated polymeric nanoparticles conjugated with IL-2, IL-15, or VLP attached to the cell membranes or these cells or exosomes and increase the cytotoxicity of the exosomes and the circulating tumor cells and eliminate the circulating tumor exosomes and stimulate a further immune cellular response, followed by plasmaphoresis to clean up the blood from the released toxins and prevent a cytokine storm in the body.

In one embodiment, the intralesional injection of polymeric antibody coated nanoparticles carrying IL-2, IL-21, IL-15, or TLR 7 and VLPs is applied inside the tumor, along with administration of cellular immune therapy with natural killer cells, to increase the natural cytotoxicity and survival of the natural killer cells, while antibody coated nanoparticles attach to the circulating exosomes or circulating cells to be attacked by natural killer cells of the body, or the treatment is administered intravenously, simultaneously, or sequentially with the nanoparticles to stimulate natural killer cells and other immune cells to eliminate the tumor exosomes carrying PD-L1 which prevent them from blocking the cellular immune response.

In one embodiment, the intralesional injection of polymeric antibody coated nanoparticles carrying IL-2, IL-21, IL-15, or TLR 7 and VLPs is applied inside the tumor, along with intralesional administration of cellular immune therapy with natural killer cells, along with thermotherapy to the temperature of 40-41 degrees C. to sensitize the tumor cells and make them loose their defense mechanism while being attacked by the killer cells, along with checkpoint inhibitors to increases natural cytotoxicity and survival of the natural killer cells, while antibody coated nanoparticles attach to the circulating exosomes or circulating tumor cells to be attacked by natural killer cells of the body or administered intravenously, simultaneously, or sequentially with the nanoparticles to stimulate natural killers and to enhance their immune response, and other immune cells carrying PD-L1 eliminate the tumor exosomes, which prevent the tumor cells and exosomes from blocking the cellular immune response.

In one embodiment, the above described procedure is combined with local or external radiotherapy.

In another embodiment, one administers systemically or locally inside the tumor both antibody coated polymeric nanoparticles conjugated with thiol/CRISPR and a guide RNA with silicate and suspended in silicate and a cationic amphiphilic polymer such as ACPP for cell penetration and simultaneous administration of intratumoral injection of the polymeric antibody coated nanoparticle/VLP inside the tumor and upon release from polymeric carrier, the gene-modifying nanoparticles/immune stimulators enter the tumor cells and modify their genetic components that are responsible for building the exosomes and converting the tumor cells to an antigenic source that stimulates the immune cell response and eliminate the tumor cells, their circulating cells, their metastatic lesions, and their exosomes followed by plasmapheresis, and kidney dialysis to clean up the blood from the released toxins and prevent a cytokine storm in the body.

In one embodiment, in general the CD8 lymphocytes or the killer cells can become exhausted in the presence of glucose that leads to a low pH in the tissue tumor environment. However, by administration of viral-like particles or virocidals or oncolytic viruses, the killer cells assume also an antiviral activity releasing interleukin 15 that renders them stimulated to not only attack the viral particles but simultaneously the tumor cells, their exosomes, and/or the extra-cellular vesicles of the tumors and their metastatic lesions.

In one embodiment, the administration of culture grown modified killer cells is associated with simultaneous antibody coated nanoparticles conjugated with VLP or virocidals and interleukin-15 to further stimulate anti-tumor action of the natural killer cells against the exosomes, extracellular vesicles, and circulating tumor cells and the tumor cells everywhere.

In one embodiment, a known Wnt inhibitor, niclosamide and taxol, or daunorubicin, ara-C or cytarabine or metformin, buformin or phenformin with or without syrosingopine are conjugated with a thermosensitive polymer antibody/medication coated nanoparticles and ACPP, nanocages or nanoshells having the temperature markers quenched fluorescein and PFCL intravenously, intra-arterially locally, or in the cerebrospinal fluid to attach to the tumor receptors and heated with either an alternating magnetic field, low power ultrasound, or microwaves to heat up the tumors and release the medication under control of the temperature and a processor to release the medication and kill the tumor simultaneously by thermal energy, imaged with a thermoacoustic imaging system for control of the temperature in treatment of glioblastoma or glioma, or ovarian cancer or breast cancer, gastric cancer, colon cancer, or melanomas, Mantle cell lymphoma preferentially killing the cancer stem cells, etc. and the increased tumor biomarkers in the circulation after the thermotherapy has an important diagnostic (i.e., indicating presence of a tumor) and therapeutic value as biomarkers for vaccine production with or without VLP or intratumoral injectable oncolytic viruses attached to antibody coated nanoparticles and administering them with checkpoint inhibitors, such as PD-1, Jagged 1 inhibitor 15D11, etc., and Rock inhibitors, such as Fasudil, netarsudil or integrin inhibitors, such as NEDD9 scaffold protein, antibody LM609 and Nitroproline derivatives, etc. or Wnt inhibitors, such as niclosamide, with cell-penetrating peptides (CPPs) for the management of the future recurrences in the patient and eliminating the need for CAR-T administration to the patient, and preventing their important glucose metabolism with metformin, buformin or phenformin and syrosingopine.

In one embodiment, a known Wnt inhibitor, niclosamide and VLPs, and checkpoint inhibitors and metformin, or buformin or in combination with a glutaminase inhibitor, such as Calithera, inhibiting glutamine uptake for the tumor to which the tumor cells are addicted, are conjugated with a thermosensitive polymer antibody coated nanoparticles with ACPP, nanocages, or nanoshells having the temperature markers quenched fluorescein or another indicator and PFCL intravenously, intra-arterially, locally in the tumor, or in the cerebrospinal fluid to attach to the tumor receptors and heated with either an alternating magnetic field, high power ultrasound, or microwaves to heat up the tumors and release the medication under control of the temperature of 42 to 43 degrees C. and, imaged with a thermoacoustic imaging system or dual imaging photoacoustic and ultrasound for control of the temperature and a processor to release the medication and kill the tumor simultaneously by thermal energy in treatment of glioblastoma or glioma, or ovarian cancer or breast cancer and the increased tumor biomarkers in the circulation after the thermotherapy has an important diagnostic (i.e. indicating presence of a tumor) and therapeutic value as biomarkers for vaccine production with or without VLP or intratumor injectable oncolytic viruses attached to antibody coated nanoparticles, and administering them with checkpoint inhibitors, such as PD-1, Jagged 1 inhibitor 15D11, etc. or a combination of them, with or without chemotherapeutic agents and Rock inhibitors, such as Fasudil, or Wnt inhibitors, such as niclosamide, with cell-penetrating peptides (CPPs) for the management of the future recurrences in the patient and eliminating the need for CAR-T administration to the patient and preventing their important glucose metabolism at the tumor site and enhance immunotherapy.

In one embodiment, the Wnt inhibitor, ivermectin, may be given separately orally or systemically at low concentration of 1-90 nanograms/ml with thermotherapy of the glioblastoma, breast, ovarian, prostate cancer, lymphoma, leukemia along with thermotherapy using antibody/medication coated nanoparticles, nanocages, or nanoshells having the temperature markers quenched fluorescein and PFCL intravenously, intra-arterially locally, intra-arterially locally, inside the tumor, in the body cavity such as bladder, mouth, nasal cavity, or in the cerebrospinal fluid to attach to the tumor receptors and heated with either an alternating magnetic field, low power ultrasound, or microwaves to heat up the tumors and release the medication under control of the temperature, imaged with a thermoacoustic imaging system or dual imaging system for control of the temperature, and a processor to release the medication and kill the tumor simultaneously by thermal energy in treatment of glioblastoma or glioma, or ovarian cancer or breast cancer and the increased tumor biomarkers in the circulation after the thermotherapy has an important diagnostic (i.e., indicating presence of a tumor) and therapeutic value as biomarkers for vaccine production with or without VLP attached to antibody coated nanoparticles, and administering them with VLP, checkpoint inhibitors, such as PD-1, Jagged 1 inhibitor 15D11, etc., and Rock inhibitors, such as Fasudil, or Wnt inhibitors, such as niclosamide, with cell-penetrating peptides (CPPs) for the management of the future recurrences in the patient and eliminating the need for CAR-T administration to the patient.

In one embodiment, the treatment can be repeated in weekly or monthly intervals until the cancer disappears.

In one embodiment, a known Wnt inhibitor, niclosamide, and rituximab are conjugated with thermosensitive polymer antibody/medication coated nanoparticles, nanocages, or nanoshells having the temperature markers quenched fluorescein and PFCL intravenously, and checkpoint inhibitors, Rock inhibitors, such as Fasudil, etc., intra-arterially locally, inside the tumor, in the body cavity, such as in the bladder, or in the cerebrospinal fluid to attach to the tumor receptors and heated with either an alternating magnetic field, focused ultrasound, or microwaves to heat up the tumors and release the medication under control of the temperature, imaged with a thermoacoustic imaging system for control of the temperature and a processor to release the medication and kill the tumor simultaneously by thermal energy in treatment of glioblastoma or glioma, or ovarian cancer or breast cancer, gastric cancer colon cancer, or melanomas, bladder or prostate cancer, etc.

In one embodiment, a known Wnt inhibitor Niclosamide and rituximab are conjugated with thermosensitive polymer antibody/medication coated nanoparticles, nanocages, or nanoshells having the temperature markers quenched fluorescein and PFCL intravenously, and checkpoint inhibitors, but Rock inhibitors, such as Fasudil, etc., given orally or locally, intra-arterially locally, inside the tumor, in the body cavity, such as in the bladder, or in the cerebrospinal fluid to attach to the tumor receptors and heated with either an alternating magnetic field, high power focused ultrasound, or microwaves to heat up the tumors and release the medication under control of the temperature and a processor to release the medication and kill the tumor simultaneously by thermal energy in treatment of glioblastoma or glioma, or ovarian cancer or breast cancer, gastric cancer colon cancer, or melanomas, bladder or prostate cancer, etc.

In one embodiment, a known Wnt inhibitor is conjugated with rock inhibitor Fasudil (HA-1077), a selective RhoA/Rho kinase (ROCK) inhibitor, or Y-27632, a small molecule inhibitor of ROCK1 and ROCK2, etc. or integrin inhibitors, such as NEDD9 scaffold protein, antibody LM609 and Nitroproline derivatives, etc. in a liposomal preparation containing fluorescein or metformin, buformin, or phenformin and thermosensitive polymer antibody coated nanoparticles, nanocages, or nanoshells having the temperature markers quenched fluorescein or other dyes or indicators and PFCL intravenously, intra-arterially locally, inside the tumor, or in the cerebrospinal fluid to attach to the tumor receptors and heated with either an alternating magnetic field, low power ultrasound, or microwaves to heat up the tumors and release the medication under control of the temperature, imaged with a thermoacoustic imaging system for control of the temperature and a processor to release the medication targeting multiple signaling pathways (NF-κB, Notch, ROS, mTORC1, and Stat3 and kill the tumors simultaneously by thermal energy in the treatment of glioblastoma or glioma, or ovarian cancer or breast cancer, gastric cancer, colon cancer, or melanomas etc. and the increased tumor biomarkers in the circulation after the thermotherapy has an important diagnostic (i.e. indicating presence of a tumor) and therapeutic value as biomarkers in vaccine production along with antibody coated nanoparticles conjugated with VLP, and administering them with checkpoint inhibitors, such as PD-1, Jagged 1 inhibitor 15D11, etc. for enhanced localized immunotherapy and Rock inhibitors, such as Fasudil or Wnt inhibitors, such as niclosamide, for the future management of the recurrences in patients and reduce the cytokine storm.

In one embodiment, ivermectin or a small molecule Wnt inhibitor PKF118-310 or Curcumin, and doxycycline derivatives, or vitamin D are conjugated with thermosensitive polymer antibody/medication coated nanoparticles with CPP, nanocages, or nanoshells having the temperature markers quenched fluorescein, methylene blue, or other dyes or indicators and PFCL intravenously, intra-arterially, locally, inside the tumor, or in the cerebrospinal fluid to attach to the tumor receptors and heated with either an alternating magnetic field, high power ultrasound, or microwaves to heat up the tumors and release the medication under control of the temperature 43 degrees C. to 50-56 degrees C. temperature, imaged with a thermoacoustic imaging system for control of the temperature and a processor to release the medication and kill the tumor simultaneously by thermal energy in treatment of glioblastoma or glioma, or ovarian cancer, colon cancer, prostate cancer, or breast cancer, mammary stem cells cancers and the increased tumor biomarkers in the circulation after the thermotherapy has an important diagnostic (i.e. indicating presence of a tumor) and therapeutic value as biomarkers for the future management of the patient removing the cytokine from the plasma to avoid autoantibody and reduce the cytokine storm by the use of electrophoresis, plasmapheresis, or plasma exchange or ARF6 inhibition.

In one embodiment, in cytokine storm, with elevated levels of tumor necrosis factor (TNF) a, interleukin-6, granulocyte colony-stimulating factor, interleukin-1β, and interleukin-17 one administers systemic tocilizumab at a dose of 8 mg per kilogram and or Intravenous immune globulin (IVIG)2 g per kilogram, or nanoparticles coated with thermosensitive polymers carrying, Rock inhibitors, mycophenolic acid, cyclosporine A, or other macrolides, infliximab, an anti-TNF-α antibody.

In one embodiment, Niclosamide, a salicylamide targets the Wnt/β-catenin pathway prepared as a PEGylated lipid nanoemulsion conjugated with carboplatin, monoclonal antibody, metformin, buformin or phenformin coated pluralities of nanoparticles with cell-penetrating peptides (CPPs), nanoshells, or nanocages administered systemically intravenously, intra-arterially locally, inside the tumor, intra-arterially, in the body cavity, such as in the bladder, or in the cerebrospinal fluid to attach to the tumor receptors and heated with either an alternating magnetic field, focused, or microwaves to heat up the tumors and release the medication under control of the temperature, imaged with a thermoacoustic imaging system or dual imaging system for control of the temperature, a processor connected to the thermal delivery and thermal imaging unit to release the medication at about 41-43 degrees C. when the lipid melts targeting multiple cancers, such as leukemia, glioblastoma or glioma, or ovarian cancer, drug-resistant ovarian cancer, ovarian tumor-initiating cells (OTIC) or breast cancer, gastric cancer, colon cancer, or melanomas, etc. and the increased tumor biomarkers in the circulation after the thermotherapy has an important diagnostic (i.e., indicating presence of a tumor) and therapeutic value as biomarkers for vaccine production with or without VLP attached to antibody coated nanoparticles and administering them with, VLP, checkpoint inhibitors, such as PD-1, Jagged 1 inhibitor 15D11, etc. and Rock inhibitors, such as Fasudil, or Wnt inhibitors, such as niclosamide, with cell-penetrating peptides (CPPs) for the management the future recurrences in the patient and eliminating the need for CAR-T administration to the patient.

In one embodiment, Niclosamide, a salicylamide targets the Wnt/β-catenin pathway prepared as PEGylated lipid nanoemulsion conjugated with carboplatin, or other antineoplastic medications, or temozolomide, monoclonal antibody coated pluralities of nanoparticles with cell-penetrating peptides (CPPs), nanoshells, or nanocages administered systemically intravenously, intra-arterially locally, inside the tumor, in the body cavity, such as in the bladder, mouth, nasal cavity, intraperitoneally, or in the cerebrospinal fluid to attach to the tumor receptors and heated with either an alternating magnetic field, focused ultrasound, or microwaves to heat up the tumors and release the medication under control of the temperature, imaged with a thermoacoustic imaging system for control of the temperature with a processor connected to the thermal delivery and thermal imaging unit to release the medication at about 41-43 degrees C. when the lipid melts targeting multiple cancers such as leukemia, glioblastoma or glioma, or ovarian cancer, drug-resistant ovarian cancer, ovarian tumor-initiating cells (OTIC) or breast cancer, gastric cancer, colon cancer, or melanomas, etc. or inhibit intracellular WNT/CTNNB1-, NOTCH-, mTOR-, and NF-κB signaling cascades and the increased tumor biomarkers in the circulation after the thermotherapy has an diagnostic (i.e., indicating presence of a tumor) and therapeutic value as biomarkers for vaccine production with or without VLP attached to antibody coated nanoparticles, and administering them with checkpoint inhibitors, such as PD-1, Jagged 1 inhibitor 15D11, etc., and Rock inhibitors, such as Fasudil, or Wnt inhibitors, such as niclosamide, with cell-penetrating peptides (CPPs) for the management the future recurrences in the patient and eliminating the need for CAR-T administration to the patient.

In one embodiment, niclosamide is given orally instead of intravenously at the dose of 1-2 grams or less for one day and repeated monthly as needed simultaneously with thermoimmunotherapy to achieve the killing of the tumors such as glioblastoma, ovarian cancer, breast cancer, or medulloblastoma, and the therapy is repeated as needed.

In one embodiment, antibody coated nanoparticles conjugated with niclosamide is given intraperitoneally along with the thermoimmunotherapy as a cyclodextrin conjugate at 0.5 mg/Kg or not exceeding 1 mg/Kg along with the described thermoimmunotherapy to achieve killing of the tumors, such as glioblastoma, ovarian cancer, breast cancer, medulloblastoma, and the therapy is repeated as needed.

In one embodiment, in the post-operative period when tumor recurrences might occur, one can still activate the CAR-T cells present in the bone marrow of the patient who have been treated previously with CAR-T cells or natural killer cells or modified natural killer cells by stimulating their proliferate to attack the tumor recurrences by using the vaccine prepared from cold stored biomarkers with or without VLP, IL-2 and IL 21 of the patient's blood obtained after the initial thermotherapy, and administering them with checkpoint inhibitors, such as PD-1, Jagged 1 inhibitor 15D11, etc., and Rock inhibitors, such as Fasudil, or Wnt inhibitors, such as niclosamide, conjugated with thermosensitive polymer/quenched fluorescein, methylene blue, or other indicators/medication and the antibody coated pluralities of nanoparticles, and administering them with checkpoint inhibitors, such as PD-1, Jagged 1 inhibitor 15D11, etc., and Rock inhibitors, such as Fasudil, or Wnt inhibitors, such as niclosamide, with cell-penetrating peptides (CPPs) to achieve release of the vaccine/dye by controlled thermotherapy at the temperature 41-43 degrees C. to damage the tumor cells and stimulating the still existing CAR-T cells in the bone marrow or administering simultaneously natural killer cells or modified natural killer cells with the vaccine. The vaccination may be repeated in weekly or monthly intervals, every three months or six months, even if no sign of the tumor metastasis is found demonstrated by disappearance of the blood biomarkers to enhance the patients immunity for a life long.

In one embodiment, the vaccination is done every week for a month and then every three months for the first year, then every six months and beyond to enhance immunity of the patient for the life of the patient to prevent recurrences of the same tumor or occurrence of other tumors. In one embodiment, the vaccination is combined with administration of genetically natural killer cells intravenously or locally or elsewhere as needed.

In one embodiment, a known enzyme that dissolves the tumor cells' membrane and mitochondrial cell membrane (i.e., granzyme) is conjugated with a thermosensitive polymer antibody/medication coated nanoparticles and ACPP, gold, silica, magnetic, paramagnetic, and nonmagnetic nanoparticles, nanocages or nanoshells, with or without macrolides, such as cyclosporine A, mycophenolic acid, tacrolimus or ascomycin, having the temperature markers quenched fluorescein, methylene blue, or other indicators and PFCL, bubble liposomes containing air pockets or nanoemulsions of PFC carrying fluorescein gene intravenously, intra-arterially locally, inside the tumor, or in the cerebrospinal fluid to attach to the tumor receptors and heated with either an alternating magnetic field, focused ultrasound, or microwaves, radiofrequency to heat up the nanoparticle/tumors complex and release the medication under control of the temperature, and imaged with a thermoacoustic imaging system for control of the temperature and a processor at 41-43 degrees C. to release the medication/dye and kill the tumor simultaneously by thermal energy in treatment of glioblastoma or glioma, or ovarian cancer or breast cancer, gastric cancer, colon cancer, or melanomas, Mantle cell lymphoma preferentially killing the cancer stem cells, etc. and the increased tumor biomarkers in the circulation after the thermotherapy has an important diagnostic (i.e., indicating presence of a tumor) and therapeutic value as biomarkers for vaccine production with or without VLP attached to antibody/medication coated nanoparticles and administering them with checkpoint inhibitors such as PD-1, Jagged 1 inhibitor 15D11, etc. and Rock inhibitor, Fasudil or Wnt inhibitors, such as niclosamide, with cell-penetrating peptides (CPPs) for the management of the future recurrences in the patient and eliminating the need for CAR-T administration to the patient.

In one embodiment, a known enzyme that dissolves the tumor cells' membrane and mitochondrial cell membrane (e.g., granzyme, metalloproteinase (MMP), chymotrypsin, as pepsin, alpha chymotrypsin, or trypsin) with or without macrolides, such as cyclosporine A, mycophenolic acid, tacrolimus or ascomycin, is conjugated with a thermosensitive polymer antibody/medication coated nanoparticles and ACPP, gold, silica, magnetic, paramagnetic, and nonmagnetic nanoparticles, nanocages or nanoshells having the temperature markers quenched fluorescein, methylene blue, or other indicators and PFCL, or bubble liposomes containing air pockets or nanoemulsions of PFC carrying fluorescein, gene and intravenously, intra-arterially locally, inside the tumor, or in the cerebrospinal fluid to attach to the tumor receptors and heated with either an alternating magnetic field, ultrasound, microwaves, or radiofrequency to heat up the nanoparticle/tumors complex and release the medication under control of the temperature, and imaged with a thermoacoustic imaging system for control of the temperature and a processor to release the medication/dye at 41-43 degrees C. and kill the tumor simultaneously by thermal energy in treatment of glioblastoma or glioma, or ovarian cancer or breast cancer, gastric cancer, colon cancer, or melanomas, Mantle cell lymphoma preferentially killing the cancer stem cells, etc. and the increased tumor biomarkers in the circulation after the thermotherapy has an important diagnostic (i.e., indicating presence of a tumor) and therapeutic value as biomarkers for vaccine production with or without VLP attached to antibody/medication coated nanoparticles and administering them with checkpoint inhibitors, such as PD-1, Jagged 1 inhibitor 15D11, etc. and Rock inhibitors, such as Fasudil, or Wnt inhibitors, such as niclosamide, with cell-penetrating peptides (CPPs) for the management of the future recurrences in the patient and eliminating the need for CAR-T administration to the patient.

In one embodiment, a known enzyme that dissolves the tumor cells' membrane and mitochondrial cell membrane (e.g., granzyme, metalloproteinase (MMP), chymotrypsin, as pepsin, alpha chymotrypsin, or trypsin) is conjugated with a thermosensitive polymer antibody/medication coated nanoparticles or liposomes and ACPP, VLP, and checkpoint inhibitors, but Rock inhibitors, such as Fasudil, etc. given orally or locally inside the tumor or integrin inhibitors, such as NEDD9 scaffold protein, antibody LM609 and Nitroproline derivatives, etc. with gold, silica, magnetic, paramagnetic, and nonmagnetic nanoparticles, nanocages or nanoshells having the temperature markers quenched fluorescein or other indicators, such as bubble liposomes containing air pockets or nanoemulsions of PFC carrying fluorescein gene and drug, with or without macrolides, such as cyclosporine A, mycophenolic acid, tacrolimus or ascomycin, intravenously, intra-arterially locally, inside the tumor, or in the cerebrospinal fluid to attach to the tumor receptors and heated with either an alternating magnetic field, ultrasound, microwaves, or radiofrequency to heat up the nanoparticle/tumors complex and release the medication under control of the temperature, and imaged with a thermoacoustic imaging system for control of the temperature and a processor to release the medication/dye at 41-43 degrees C. and kill the tumor simultaneously by thermal energy in treatment of glioblastoma or glioma, or ovarian cancer or breast cancer, gastric cancer, colon cancer, or melanomas, Mantle cell lymphoma preferentially killing the cancer stem cells, etc. and the increased tumor biomarkers in the circulation after the thermotherapy has an important diagnostic (i.e., indicating presence of a tumor) and therapeutic value as biomarkers for vaccine production with or without VLP attached to antibody coated nanoparticles and administering them with checkpoint inhibitors such as PD-1, Jagged 1 inhibitor 15D11, etc. and Rock inhibitors, such as Fasudil or Wnt inhibitors, such as niclosamide, with cell-penetrating peptides (CPPs) for the management of the future recurrences in the patient and eliminating the need for CAR-T administration to the patient.

In one embodiment, the pluralities of antibody/medication coated nanoparticles including piezoelectric nanoparticles carry with their thermosensitive polymeric coating one or multiple antineoplastic medications, checkpoint inhibitors, Perforin, cytolysins, complement component 9(C9) granzyme, related apoptosis-inducing ligand (TRAIL), or the Fas ligand, VLPs, etc. are used for treatment of cancer using a combination of focused ultrasound, alternating magnetic field, or electromagnetic radiation, non-invasively at a temperature 41-43 degrees C. under the control of a processor controlling the thermal energy, imaged with a thermoacoustic imaging system for control of the temperature, along with low-intensity (1-3 V/cm), intermediate frequency (100-300 kHz), electric fields achieving thermotherapy, electroacoustic imaging and dielectric effect on the cellular components of the tumors, such as brain tumors, lung cancer, ovarian cancer, breast cancer, prostate cancer, and other cancers, which can be repeated numerous times post therapy to eliminate the cancer with simultaneous aspiration of a lysed tumor mass through a 20-30 gauge needle to reduce the toxic burden on the body while the increased tumor biomarkers in the circulation after the thermotherapy has an important diagnostic (i.e., indicating presence of a tumor) and therapeutic value as biomarkers for vaccine production with or without VLP attached to antibody coated nanoparticles, and administering them with checkpoint inhibitors such as PD-1, Jagged 1 inhibitor 15D11, etc. and Rock inhibitor, such as Fasudil, or Wnt inhibitors, such as niclosamide, with cell-penetrating peptides (CPPs) for the management of the future recurrences in the patient and eliminating the need for CAR-T administration to the patient, while doing simultaneously plasmapheresis, kidney dialysis, dielectrophoresis of the blood clears tumor toxins, dead killer cells, etc. from the blood and prevents a cytotoxic toxic storm and systemic medication with tocilizumab, a monoclonal antibody that targets interleukin-6 and ornithine phenylacetate an ammonia scavenger, or ARF6 inhibition for treatment of hepatic encephalopathy, a neuropsychiatric syndrome associated with hyperammonemia.

In one embodiment, a known enzyme that dissolves the wall of the bacteria, viruses, fungi, parasites, such as MRSA, granzyme, metalloproteinase (MMP), chymotrypsin, as pepsin, alpha chymotrypsin, trypsin conjugated with a thermosensitive polymer antibody/medication coated nanoparticles of iron, iron oxide, gold, silica, magnetic, paramagnetic, and nonmagnetic nanoparticles, nanocages or nanoshells having the temperature markers quenched fluorescein, methylene blue, or other indicators or with Liposomes having temperature triggered opening of a leucine zipper peptide inserted in the membrane of a dye and medication carrying a liposome opens a channel through which the drug is released at the temperature or 41-43 degrees C. or opens drug-permeable pores created bubble formation from the decomposition of encapsulated ammonium bicarbonate injected intravenously, intra-arterially, locally, inside the tumor, or in the cerebrospinal fluid to attach to the bacterial surface antigens and heated with either an alternating magnetic field, ultrasound, microwaves, or radiofrequency to heat up the nanoparticle/bacteria, viruses complex and release the medication under control of the temperature, and imaged with a thermoacoustic imaging system for control of the temperature and a processor to release the medication/dye at 41-43 degrees C. to damage the wall of bacteria, viruses, etc. kill them simultaneously by thermal energy treatment in the patient, while doing simultaneously plasmapheresis, kidney dialysis, dielectrophoresis of the blood clears tumor toxins, dead killer cells, etc. from the blood and prevents a toxic storm and prevents damage to the kidney, liver, heart, etc. and systemic medication with tocilizumab, a monoclonal antibody that targets interleukin-6 and ornithine phenylacetate an ammonia scavenger, for treatment of hepatic encephalopathy, a neuropsychiatric syndrome associated with hyperammonemia or ARF6 inhibition.

In one embodiment, thermal and electrical energy is used to influence the permeability of the tumor cell membrane so as to utilize the effect or thermotherapy with pluralities of nanoparticles or liposomes having temperature triggered opening of a leucine zipper peptide inserted in the membrane of a dye and medication carrying liposome opens a channel through which the drug is released at the temperature or 41-43 degrees C. or opens drug-permeable pores created bubble formation from the decomposition of encapsulated ammonium bicarbonate injected in the body to enhance the effect of chemotherapy or immune therapy, gene therapy, gene modification using antibody coated nanoparticles conjugated with CRISPR-cas9 in malignant cells, such as brain and spinal cord tumors, breast cancer, lung cancer, prostate and ovarian cancer and melanoma, glioblastoma, retinoblastoma, meduloblastoma, gastrointestinal and genitourinary tumor of sarcoma, or benign expanding tumor, such as memningioma, uterus fibroma, etc., based on the antibody coated conjugated nanoparticles with CRISPR/Cas9 system, or Cas9/gRNA Ribonucleoprotein complexes (Cas9 RNPs), in which a DNA-cutting Cas9 enzyme targets the checkpoint inhibitor regions removing checkpoint inhibitors, but Rock inhibitors, such as Fasudil, etc. given orally or locally, inside the tumor, or systemically, genes of the tumor cells and replace them with suicide genes defined as genes whose products cause cell death, e.g., thymidine kinase (HS-TK), cytosine deaminase, etc., pro-apoptatic genes, etc. through antibody coated nanoparticles conjugated with CRISPR cas9 or Cas9/gRNA Ribonucleoprotein complexes (Cas9 RNPs), mediated Homology-Independent Targeted Integration (HITI) or Homology Directed Repair (HDR) further enhancing an immune response which is not being blocked by the checkpoint inhibitors of the tumor cells. Antibody coated nanoparticles are used having pyroelectric or piezoelectric characteristics, and the antibody coated nanoparticle tube complex is imaged with an electroacoustic computed tomography imaging system.

In one embodiment, the antibody/medication coated pluralities of gold, piezoelectric, iron oxide, nanocage, nanotube, nanoshell, magnetic, paramagnetic, pyroelectric and/or piezoelectric nanoparticles are coated with thermosensitive polymers, or with liposomes having temperature triggered opening of a leucine zipper peptide inserted in the membrane of a dye and medication carrying liposome opens a channel through which the drug is released at the temperature or 41-43 degrees C. or opens drug-permeable pores created bubble formation from the decomposition of encapsulated ammonium bicarbonate or polymers, such as chitosan, quenched with fluorescein, methylene blue, or other indicators conjugated with antineoplastic medication, gene, CRISPR-cas9 or Cas9/gRNA Ribonucleoprotein complexes (Cas9 RNPs) conjugated with cell penetrating agents or activatable cell penetrating agents injected in the patient's circulation, lymphatic vessels, inside a body cavity, cerebrospinal fluid, etc. is released that can be revealed when the nanoparticles are exposed to electromagnetic radiation, combined focused ultrasound in a thermal or non-thermal mode LIFU or non-focused ultrasound, or electrical current generated by a battery where low electrical current from a battery passes from one side of the skin (i.e., the anode) through the body and a lesion or tumor to the cathode electrode positioned on the opposite side of the skin on the body to raise the temperature of the pluralities of the piezoelectric or pyroelectric nanoparticles that are injected inside the body to be attached to the surface antigens of the normal cells or of tumor cells and create nanoparticle/tumor cell complexes to 41-43 degrees C., and when exposed to pulses of electrical current with an adjustable signal frequency and voltage, an acoustic response is produced by electrical stimulation of piezoelectric nanoparticle inside the body that is called an electroacoustic sound or signal which can be recorded with a transducer, or microphone, converted to an electrical signal and forwarded to a processor to be converted to a 1D, 2D, or 3D image as an electroacoustic computed tomogram while the electrical pulse generated in the piezoelectric nanoparticles drives the medication or gene in the tumor cells locally to damage the tumor cells by multiple modes of the therapy applied non-invasively based on the antibody coated nanoparticles conjugated with the CRISPR/Cas9 system, in which a DNA-cutting Cas9 enzyme targets the checkpoint inhibitor regions of the tumor cells removing checkpoint inhibitors genes and replacing them with suicide genes, defined as genes whose products cause cell death, e.g., thymidine kinase (HS-TK), cytosine deaminase, etc., pro-apoptic genes, etc. through the antibody coated nanoparticles conjugated with CRISPR cas9 or Cas9/gRNA Ribonucleoprotein complexes (Cas9 RNPs) mediated Homology-Independent Targeted Integration (HITI) or Homology Directed Repair (HDR) further enhancing an immune response which is not being blocked by the checkpoint inhibitors of the tumor cells in combination with Rock inhibitors, such as Fasudil, etc. given orally or locally, inside the tumor, or systemically to reduce inflammatory cells and inhibit production of TGF-β and fibrous tissue.

In one embodiment, during the thermal delivery to the antibody/medication coated nanoparticles, nanoshells, or nanocages, magnetic, non-magnetic, paramagnetic, organic piezoelectric, pyroelectric, fluorine, dendrimers, quartz or combinations thereof, or with liposomes having temperature triggered opening of a leucine zipper peptide inserted in the membrane of a dye and medication carrying liposome opens a channel through which the drug is released at the temperature or 41-43 degrees C. or opens drug-permeable pores created bubble formation from the decomposition of encapsulated ammonium bicarbonate, conjugated with a thermosensitive polymer, such as chitosan, or a medication, gene, etc., or bubble liposomes containing air pockets or nanoemulsions of PFC carrying fluorescein gene, and after their injection in the body, one can measure two distinct temperatures inside the body regardless of their location using either an electromagnetic, focused ultrasound, or alternating magnetic field, while releasing the medications, imaged with a thermoacoustic imaging system for control of the temperature at a temperature of 41-43 degrees C., this can achieve a theranostic or diagnostic and therapeutic effect when the nanoparticles with activatable cell-penetrating peptides (ACPPs) carry a thermosensitive polymer, such as chitosan, and a dye or another dye or indicator etc., such as quenched fluorescein, methylene blue, or other indicators that can be only detected when it is released in the blood indicating the melting temperature of the thermosensitive coating or that 41-43 degrees C. is reached and the nanoparticle release of the medication conjugated with the chitosan by detection of unquenched fluorescein or another dye or indicator present in the blood continuously passing through a flexible silicone tube, nail bed, or any other part of the body, radiated by UV radiation or light of another wavelength to fluoresce, or other dyes, such as indocyanin green at infrared wavelengths using the nail bed or any other visible vessels such as conjunctival vessels, etc. for illumination and recording the fluorescence wavelength through a filter that eliminates the radiating from the fluorescent wave length indicating release of dye, medication, gene etc. further heating the nanoparticle creates a cavitation sound at a temperature of either 50 degrees C. or 56 degrees C., achieved at the areas of the body depending on the thermal energy delivery source or combinations with compressive or pressure mode ultrasound, laser, microwave, radiofrequency (RF), alternating magnetic field, or any other source of the energy from outside the body or inside the body, and imaged with a thermoacoustic or photoacoustic imaging system for control of the temperature.

In one embodiment, thermal and electrical energy is used to influence the permeability of the tumor cell membrane so as to utilize the effect or thermotherapy to enhance the effect of chemotherapy or immune therapy, gene therapy, gene modification using CRISPR-cas9 in malignant cells, such as brain and spinal cord tumors, breast cancer, lung cancer, prostate and ovarian cancer and melanoma, glioblastoma, retinoblastoma, meduloblastoma, gastrointestinal and genitourinary tumor of sarcoma, or benign expanding tumors, such as meningioma, uterus fibroma, etc., based on the antibody coated nanoparticles conjugated with CRISPR/Cas9 system, in which a DNA-cutting Cas9 enzyme or combination with antibody/medication coated nanoparticles or with liposomes injected in the body having temperature triggered opening of a leucine zipper peptide inserted in the membrane of a dye and medication carrying liposome opens a channel through which the drug is released at the temperature or 41-43 degrees C. or opens drug-permeable pores created bubble formation from the decomposition of encapsulated ammonium bicarbonate, targets the checkpoint inhibitor regions removing checkpoint inhibitors genes of the T-lymphocytes and killer cells or CAR-cells to make them non-responsive to the tumor checkpoint inhibitors of the tumor cells through CRISPR cas9 mediated Homology-Independent Targeted Integration (HITI) or Homology Directed Repair (HDR) further enhancing an immune response which is not being blocked by the checkpoint inhibitors of the tumor cells. Antibody coated nanoparticles are used having pyroelectric or piezoelectric characteristics, and the nanoparticle tube complex is imaged with an electroacoustic computed tomography imaging system.

In one embodiment, biomolecules including genes are transferred to the nucleus of the tumor cells using inorganic and/or organic solar cell nanoparticles, including hybrids, using gene editing by programmable directed nucleases. In this embodiment, these nucleases bind to and cut DNA that matches guide RNAs in regions containing clustered regularly interspersed short palindromic repeats (CRISPR). Cas9 or Cas9/gRNA Ribonucleoprotein complexes (Cas9 RNPs), the CRISPR associated protein 9, the endonuclease and/or the CRISPR/Cas9 complex, or Cas9/gRNA Ribonucleoprotein complexes (Cas9 RNPs), upon cell entry, seek DNA containing a target sequence, e.g., a specific mutation in the mitochondria or the nucleus, the cleavage functions of the endonuclease and/or CRISRP/Cas9 complex or Cas9/gRNA Ribonucleoprotein complexes (Cas9 RNPs) destroy the disease-producing DNA by excising it or replacing it with an un-mutated, i.e., normal DNA segment, as known in the art to treat the patient, the nuclease cleaves host DNA at the target site, with one cut, CRISPR can insert a new customized gene sequence; and with two cuts, two CRISPRs can excise the DNA, providing the host with a new DNA.

In one embodiment, CdSe/Au particles or Gold, quartz, perovskites are used along with light and ultrasound or AMF, to stimulate gold nanoparticles, thiol, DNA-Gold-guide RNA-CRISPR-P[Asp(DET)] complex (poly[N-[N-(2-aminoethyl)-2-aminoethyl]aspartamide] as cationic nanoparticles or suspended in N,N-dimethylformamide containing a detergent (e.g., 1% Triton X-100) or hexadimethrine bromide and exposed to polyethylene glycol-(CH₂)₁₀-SH to coordinate the thiol to the Au end and the nanoparticles may be coated with biocompatible mono- or bilayers of a phospholipid, a protein, or a peptide polyethylene glycol (PEG) can be used as a scaffold to aid in biocompatibility of the particle and the donor DNA can be conjugated together to generate a single molecule (gDonor)-Cas9 complexed to gDonor.

In one embodiment, one uses antibody conjugated nanoparticles as vectors to transfer the gene and modify the gene of the natural killer cells or CAR-T cells along with CRISPR-cas9 or Cpf1 or Cas9/gRNA Ribonucleoprotein complexes (Cas9 RNPs) using the nanoparticles as a vector to deliver the gene(s) inside the cells, or to cut out the mutated gene, eliminating the side effects of viral vector such as immune activation, as observed often with the viral vector gene delivery. The technology is described in U.S. Pat. No. 10,022,457, which is incorporated by reference in its entirety herein.

In one embodiment, the natural killer cells or genetically modified natural killer (NK) cells are used in place of CAR-T cells and/or are developed in the lab to express the CARs receptors as CAR NK linked to activating receptor NKD2D and cytotoxic-chain with minimum or no off-target toxicity, while being regulated by HLA genes, but as a part of innate immune system it is activated by bacteria and viral exposure IL-2, IL-15, or polymeric antibody coated nanoparticles carrying IL-2 and IL-21 or TLR 7, or the anti-CD3 (OKT3) antibody, to increase cytotoxicity of natural killer cells, conjugated with nanoparticles and similarly with VLPs alone conjugated with polymeric or non-polymeric nanoparticles or they can be stimulated in a tissue culture by introducing an opsin gene in them and stimulating them with light pulses to induce polarization and depolarization of the cell membrane enhancing the replication of the NK cells, alternatively the NK cells or γδ T-cells are fed with QD nanoparticles and to stimulate them with the light pulse in culture media to polarize and depolarize their cell membranes and stimulate their multiplication in tissue culture and administered to the patient as needed by intravenous or intratumoral administration along with nanoparticles conjugated with immune stimulators such as IL-2, IL-15, or the anti-CD3 (OKT3) antibody, to increase cytotoxicity of natural killer cells or VLPs.

In one embodiment, the genetic components of the NK cells are modified not using a viral vector, but instead using the stem cells of killer cells or natural killer cells of the patient and using nanoparticles for gene delivery and CRISPR to modify the genetic components of the NK cells.

In one embodiment, for gene transfer, one uses CRISPR-conjugated to the desired nanoparticles such as gold, etc. via thiol to create a strong electrostatic bound, CRISPR-NP are then conjugated with Cas9 and gRNA to be used in non-homologous end joining (NHEJ) or nanoparticle-DNA conjugate is hybridized with the donor DNA, creating NP-donor DNA suspended in sodium silicate, generating NP-Donor-Cas9 RNP-silicate, which is re-suspended in a cationic polymer such as cyclodextrin or calixarene-based polycationic amphiphiles polymer as gene delivery systems or PAsp(DET) to enhance cell penetration and escape from the endosomes, before it is transferred to the nucleus to be used in Homology Directed repair (HDR) after administration to the killer cells or their stem cells with appropriate gene(s) in the tissue culture along with IL-2, or polymeric antibody coated nanoparticles carrying IL-2 and IL-21 or TLR 7, for NK cell growth and the anti-CD3 (OKT3) antibody, to increase cytotoxicity of natural killer cells.

In one embodiment, the nanoparticles (NPs) can be metallic, such as gold or ferric oxide, combination of silica/gold, quantum dots (QDs), polymeric organic, cationic polymeric NPs, PAsp(DET), piezoelectric, such as perovskites, quartz or other vectors such as Naked DNA, etc. with the size of 5-50 nm.

In one embodiment, the nanoparticle is gold or ferromagnetic covered with gold before conjugated with thiol and with the CRISPR gRNA-cationic polymer and/or gold NP-Donor DNA and suspended in silicate and a cationic polymer.

In one embodiment, one can attach multiple genes to the nanoparticles via thiol or amine or amide, before suspending in silicate and cationic polymer to encourage cell penetration and escape from the endosome after their administration to the tissue culture. In one embodiment, the nanoparticle is PAsp(DET) or gold to which CRISPR or donor DNA is attached via thiol, before suspending the complex in sodium silicate to be followed by another cationic polymer to enhance cell penetration and endosomal escape and gene(s) delivery to the nucleus after their administration to the stem cells of the natural killer cells in tissue culture with IL-2, or polymeric antibody coated nanoparticles carrying IL-2 and IL-21 or IL-15 for their growth and or the anti-CD3 (OKT3) antibody, to increase cytotoxicity of natural killer cells.

In one embodiment, the natural killer and T-cells carry the genetic defect that they have inherited, and are modified in cell culture prior to the administration to the eye, these stem cells may be cultured in a tissue culture to modify their genetic composition, these stem cells, embryonic stem cells, mesenchymal stem cells, neuronal stem cells, pluripotent stem cells, glial stem cells, etc. using viral particles in addition to nucleases, zinc finger nucleases, or CRISPR-cas9 using the technology known as non-homologous end joining (NHEJ) or homologous directed repair (HDR).

In one embodiment, one administers systemically or locally intralesion antibody coated nanoparticles conjugated with thiol with the CRISPR gRNA-cationic polymer and/or gold NP-donor DNA and suspended in silicate and a cationic polymer to eliminate the tumor's checkpoint inhibitors.

In one embodiment, thermal and electrical energy is used to influence the permeability of the tumor cell membrane so as to utilize the effect or thermotherapy to enhance the effect of chemotherapy or immune therapy, gene therapy, gene modification using antibody coated nanoparticles having pyroelectric or piezoelectric characteristics, and the nanoparticle tube complex is imaged with an electroacoustic computed tomography imaging system and conjugated with CRISPR-cas9 mediated Homology-Independent Targeted Integration (HITI) or Homology Directed Repair (HDR) further enhancing an immune response which is not being blocked by the checkpoint inhibitors in malignant cells, such as brain tumors and spinal cord tumors, breast cancer, lung cancer, prostate and ovarian cancer and melanoma, glioblastoma, retinoblastoma, meduloblastoma, gastrointestinal and genitourinary tumor of sarcoma, or benign expanding tumors, such as meningioma, uterus fibroma, etc., based on the antibody coated nanoparticles and liposome with temperature triggered opening of a leucine zipper peptide inserted in the membrane of a dye and medication carrying liposome opens a channel through which the drug is released at the temperature of 41-43 degrees C. or opens drug-permeable pores created bubble formation from the decomposition of encapsulated ammonium bicarbonate, after heating with an energy source targets, or in vitro tissue culture.

In one embodiment, in vitro culture sensitization to tumor antigen and VLP coated nanoparticles and simultaneous modification of T-cells with CRISPR Cas9 with antibody/medication coated nanoparticles or with liposomes injected in the tissue culture having temperature triggered opening of a leucine zipper peptide inserted in the membrane of a dye and medication carrying liposome after heating with an energy source opens a channel through which the drug is released at the temperature of 41-43 degrees C. or opens drug-permeable pores created bubble formation from the decomposition of encapsulated ammonium bicarbonate, eliminating checkpoint inhibitors of the T-lymphocytes, killer cells, Car-cells resulting in losing their response to checkpoint inhibitors of a tumor while maintaining T-Cell action against the tumor, preventing autoimmunity formation and the attacking normal cells in the body, and using the same concept for tumor vaccination with or without presence of a new metastatic disease.

In one embodiment, in metastatic diseases with a potential cytokine storm, one can induce in addition to vaccine therapy an oral immunity by growing the patient's tumor cell in sufficient quantity in vitro and radiating them by cobalt beta or exposing them to another source of thermal or non-thermal radiation to damage the genetic component of the tumor or kill them, while maintaining the antigenicity of the tumor cell proteins, then administer them orally after encapsulating them with Rock inhibitors such as Fasudil, netarsudil or other small molecule Rho kinase inhibitors or integrin inhibitors, such as NEDD9 scaffold protein, antibody LM609 and Nitroproline derivatives, etc. or GSK 269962 inhibitor, in addition to oral Wnt inhibitors, such as ivermectin, niclosamide, or aspirin, millimolar levels (˜5 mM), cyclo-oxygenase inhibitors, endomethacin (100-400 μM) the anti-leprotic clofazimine ˜3 μM serum concentration, and the anti-trypanosomal suramin ca. 200 metformin, Sulidac, suramin, Tigecyclin, clofazimine, pyrviniom pamoate 50-200 nM, GSK-beta inhibitors, intraperitoneal delivery at 10 mg/kg cyclodextrin conjugate ivermectin (1-2 μM), Niclosamide 0.2-0.4 inhibition of cancer cells growth (0.33-0.75 streptonigirin, salinomycin 0.3 and 10 μM serum concentration, topical Hexachlorophene, Imatinib, Gleevec/Glivec, is a tyrosine kinase inhibitor, ethacrynic acid (EA) targets Wnt genes such as fibronectin, cyclin D1, FDA approved riluzole, apicularen, bafilomycin, and integrin inhibitors to reduce inflammatory and TGF-β production after, subcutaneous, intramuscular or intravenous inside the tumor or intraperitoneal vaccination in the patient who have developed an autoimmune response after immune therapy.

In one embodiment, in a patient with metastatic disease where a cytokine storm can be expected after standard immunotherapy, antibody coated nanoparticles are conjugated with non-toxic doses of Rock inhibitors or Wnt inhibitors, or integrin inhibitors, such as NEDD9 scaffold protein, antibody LM609 and Nitroproline derivatives, etc. combined with macrolide immunosuppressants such as cyclosporine A, mycophenolic acid, tacrolimus, ascomycin, etc. to suppress excessive immune response and inflammation in the normal tissue by blocking the IL-17, etc. from the memory of Th17 cells with the existing severe immune response in the skin, gut, joint, etc.

In one embodiment, in a patient with metastatic disease, the use of antibody coated pluralities of nanoparticles can be conjugated with Rock inhibitors, Wnt inhibitors, and GSK 269962 inhibitors along with antibody coated nanoparticles with cyclosporine A, or mycophenolic acid or tacrolimus etc. to significantly damage the tumor cells and their metastatic lesion with or without thermotherapy.

In one embodiment, the treatment of a tumor with a metastatic lesion is done with simultaneous administration of pluralities of antibody coated nanoparticles conjugated with various medications, such as Wnt inhibitors, GSK inhibitors, or Rock inhibitors, or integrin inhibitors such as NEDD9 scaffold protein, antibody LM609 and Nitroproline derivatives, etc. with macrolide antibodies such as cyclosporine A, Ascomycin, mycophenolic acid, or tacrolimus, anti VEGFs, such as Avastin, etc. and simultaneous administration of checkpoint inhibitors, such as PD-1, CTLA-4, Jagged 1 inhibitor 15D11, etc. and immune stimulators, such as VLPs, IL-2, or polymeric antibody coated nanoparticles carrying IL-2 and IL-21 or the anti-CD3 (OKT3) antibody, to increase cytotoxicity of natural killer cells or IL-15, IL-6, IL-4, TLR 7, TLR 8, IFN-γ or granzyme β, related apoptosis-inducing ligand (TRAIL), or the Fas ligand, etc. and simultaneous subcutaneous or intratumoral or intravenous vaccination with antibody coated nanoparticles conjugated with VLPs or other modified Reoviruses, or granzyme β, related apoptosis-inducing ligand (TRAIL), or the Fas ligand, and IL2, IL-6, IFN-γ, TLR 4, 7, 8 where after the initial therapy, the vaccination will continue at weekly intervals, then monthly, then every 3 month intervals, and evaluated by presence of the circulating tumor exosomes, or circulating tumor cells, or presence of tumor cells that have devoured the macrophages or other normal cells thereby becoming hybrid tumor cells diagnosed by obtaining blood from the patients in the postoperative period, the presence of the patient's tumor genetic material in these cells, incubating the tumors in the culture, and administering thermosensitive nanoparticles, administering laser radiation to kill these cells and obtain new lysates to produce a new vaccine.

In one embodiment, one administers locally inside the tumor followed by vaccine therapy in combination two or more checkpoint inhibitors, and antibody coated nanoparticles with PI3K inhibitors, such as Idalisib, copanlisib, Duvelisib, wortmanin with antibody coated nanoparticles, a vincristine or PI3 kinase inhibitor GDC0941 (pictilisib), and the tubulin inhibitor vincristine and/or antibody coated nanoparticles RANi or RNAs, etc. along with antibody coated nanoparticles, VLPs, or intratumor injectable oncolytic viruses, etc. alone or in combination.

In one embodiment, as a routine plasmapheresis, dielectrophoresis, and a kidney dialysis is done to remove the dead normal cells and hybrid cells and exosomes, and toxins, etc. from the blood that can still be cleaned and repurposed to the patient.

In one embodiment, the antibodies obtained from the patient's body or in tissue culture from the hybrid cells are used to coat the nanoparticles conjugated with CRSPR-cas9 and with cytotoxic siRNA to damage the hybrid tumor cells. In one embodiment, the nanoparticles carry both the tumor antibody and the hybrid cell antibody to deliver cytotoxic siRNA to both kinds of the cells and protect the normal cells. In one embodiment, this procedure is repeated with simultaneous thermotherapy with an external source of energy or radiotherapy with radioactive nanoparticles, or external radiation to damage these cells, and the treatment is repeated weekly or monthly along with either Rock inhibitors, Wnt inhibitors, or the GSK 269962 inhibitor lithium along with antibody coated nanoparticles to inhibit GSK, and repeated until the hybrid or tumor cells are not found from the patient's blood samples.

In one embodiment, the combination therapy for the late metastatic disease may produce an autoimmune response, here the gene therapy is combined with Rock inhibitors, etc. and macrolides to reduce the post-treatment immune and inflammatory response.

In one embodiment, the pluralities of antibody coated nanoparticles are conjugated with Rock inhibitors, Wnt inhibitors, GSK inhibitors, antiVEGFs, such as Avastin, NSAIDS, such as Diclofenac, acethylsalisulic acid, aspirin, Lovastatins to block phosphoinositol activation as a results of hypoxia induced factor-1 which induces VEGF production.

In one embodiment, the pluralities of antibody coated nanoparticles, liposomes, or micelles are conjugated with Rock inhibitors (e.g., botulinum toxin), integrin inhibitors, Wnt inhibitors, GSK inhibitors, anti-VEGFs, such as Avastin, or NSAIDS, such as Diclofenac, acethylsalisulic acid, aspirin, opioids, morphine, tramadol, etc., or cannabis derivatives to combat a severe pain sensation associated with cancer involving bone, pancreas, or in patients with severe inflammation and pain in the joints, spine, neck, in rheumatic diseases, prostate pain after surgery, pelvic pain or after traumatic head and neck injuries or neuromuscular pain where the thermotherapy at a temperature of 39-43 degrees C. is applied with electromagnetic radiation, microwave, ultrasound, LIFU or HIFU, alternating magnetic field or RF that releases the medication locally at the site to relieve pain sensation.

In one embodiment, the pluralities of antibody coated nanoparticles, liposomes, or micelles are conjugated with antibiotics, antifungal, antivirals, antiparasitics, etc., Rock inhibitors (e.g., botulinum toxin), integrin inhibitors, Wnt inhibitors, GSK inhibitors, or NSAIDS, such as Diclofenac, acethylsalisulic acid, aspirin, opioids, morphine, tramadol, etc., cannabis derivatives to combat severe pain sensation associated with infectious diseases where the medication can be applied topically to the skin or mucosa, or injected inside a body cavity, such as the bladder, etc., locally or systemically administered or inhaled and released by controlled thermotherapy, applied electromagnetic radiation, microwave, ultrasound (i.e., LIFU or HIFU), alternating magnetic field or RF that releases the medication locally at the site at a controlled temperature of 37-41 degrees C. to treated any inflammatory disease.

In one embodiment, in a localized inflammatory disease, e.g., a rheumatic disease in the joint, induces a local increase in the tissue temperature that can be measured using photoacoustic temperature imaging or in combination with ultrasound as a dual imaging system, the effect of a local or systemic therapy in these diseases can be measured after treatment using the aforementioned methods as non-invasive local thermometry which will indicate reduced temperature or normalization of the tissue temperature in the subsequent evaluation of the patient, or a similar reduced temperature after cancer therapy can be also measured at the tumor site indicating a positive response to the therapy.

In one embodiment, HIF1A induces Myelodysplastic Syndromes that can be treated with antibody coated nanoparticles conjugated with Wnt inhibitors, GSK269962 inhibitor and anti-VEGFs, such as Bevacizumab, Ranibizumab, Aflibercept, Avastin, and anti-platelet derived growth factor (PDGF) agents.

In some cancer patients, there is a deficiency of cyclin-dependent kinase 12 (CDK12) which is involved in repair of DNA that can be diagnosed by genomic analysis, the deficiency or mismatch of CDK12 has been found in numerous tumors, such as cancer of the prostate, breast, gastrointestinal tract, bladder, uterine, and ovarian cancers that respond positively to the administration of poly(adenosine diphosphate [ADP] ribose) polymerase (PARP) inhibitors or platinum chemotherapies, such as cisplatin or carboplatin. In one embodiment, pluralities of antibody coated nanoparticles are conjugated with polymeric nanoparticles carrying Rock or Wnt inhibitors, GSK inhibitors, integrin inhibitors and PARP and/or platinum chemotherapy and VLP or intratumor injectable oncolytic viruses, anti-VEGF, such as Bevacizumab, Ranibizumab, Aflibercept, Avastin, and anti-platelet derived growth factor (PDGF) agents, and one or more checkpoint inhibitors are injected locally inside the tumor, or systemically, intravenously, or intra-arterially into the feeding artery, applying the nanoparticles to the tumor cells prior to their systemic uptake by the Reticuloendothelial cells (REC), and optionally applying external thermotherapy, laser, LIFU, HIFU, RF, alternating magnetic field (AMF) in non-thermal or thermal frequencies while controlling the temperature with a thermal imaging unit and a software from 37 degrees C. to 41 degrees C. or more as needed, releasing the medication to kill damaged tumor cells, their exosomes, or circulating tumor cells (CTC) or their exosomes.

In one embodiment, multiple checkpoint inhibitors are administered locally inside the tumor with a combination of VLPs or intratumor injectable oncolytic viruses along with injection of natural killer (NK) cells and antibody coated nanoparticles carrying chemotherapeutic agents, TNF, IL-2, or polymeric antibody coated nanoparticles carrying IL-2 and IL-21, or IL-15, IL-6, TLR 7, CD 40 agonist, IFN-γ or granzyme β, related apoptosis-inducing ligand (TRAIL), or the Fas ligand, and local radioactive antibody coated nanoparticles, such as radioactive iodine, etc. or external radiation, such as stereotactic radiotherapy, localized radioactive plaques, cobalt, ruthinum, etc. or proton beam radiation or helium ion radiation or palladium-103 plaque radiation and thermotherapy with an external energy source, laser, focused ultrasound, microwave, or radiofrequency or local or external radiation, etc., such as in localized malignant tumors of the eye, skin, or mucosal tumors or malignant tumors of the nose, throat, or prostate, along with intralesional Rock inhibitors, integrin inhibitors, such as abegrin cilengitide, abciximab, tirofiban, natalizumab, eptifibatide or a low concentration of 1 microgram to 10 micrograms as a solution so as to reduce excessive TGF-beta and scarring, and excessive inflammation, etc.

In one embodiment, multiple checkpoint inhibitors are administered locally inside the tumor with a combination of antibody coated polymeric or metallic nanoparticles, such as gold, or ferric oxide, etc. conjugated with VLPs or intratumor injectable oncolytic viruses along with antibody coated nanoparticles carring chemotherapeutic agents, TNF, IL-2, or polymeric antibody coated nanoparticles carrying IL-2 and IL-21, or IL-15, or the anti-CD3 (OKT3) antibody, to increase the cytoxicity of natural killer cells, or IL-15, IL-6, IL-1β, IL-17, TLR 7, CD 40 agonist, IFN-γ or granzyme β, related apoptosis-inducing ligand (TRAIL), or the Fas ligand, and local radioactive antibody coated nanoparticles, such as radioactive iodine, etc. or external radiation such as stereotactic radiotherapy, localized radioactive plaques, cobalt, ruthinum, etc. or proton beam radiation or helium ion radiation or palladium-103 plaque radiation where the metallic nanoparticles can enhance the effect of radiation in localized malignant tumors of the eye, skin or mucosal tumors or malignant tumors of the nose, throat, or prostate, etc. while intralesional Rock inhibitors, integrin inhibitors, such as abegrin cilengitide, abciximab, tirofiban, natalizumab eptifibatide or a low concentration of 1 microgram to 10 micrograms as a solution reduces excessive TGF-beta and scarring.

In one embodiment, thermotherapy is applied with an external energy source, laser, focused ultrasound, microwaves, or radiofrequency radiation, AMF, etc.

In one embodiment, the external radiotherapy or radioactive plaque is combined with external or internal thermotherapy along with intralesional administration of multiple checkpoint inhibitors that are administered locally inside the tumor with a combination of antibody coated polymeric or metallic nanoparticles such as gold, or ferric oxide, etc. conjugated with VLPs or intratumor injectable oncolytic viruses along with antibody coated nanoparticles carrying chemotherapeutic agents, TNF, IL-2, or polymeric antibody coated nanoparticles carrying IL-2 and IL-21, or the anti-CD3 (OKT3) antibody, to increase the cytoxicity of natural killer cells or IL-15, IL-6, IL-1β, IL-17, TLR 7, CD 40 agonist, IFN-γ or granzyme (3 to enhance cellular immune therapy or along with simultaneous injection of natural killer cells or modified natural killer cells or CAR-T cells and Rock inhibitors.

In one embodiment, excessive production of cytokines from the tumor, or as a result of therapy, change the tumor microenvironment and as a result, the cytotoxic T-lymphocytes become exhausted and lose the normally present anti-tumor action and in contrast produce on their surface membrane inhibitory markers and angiogenetic factors, and cannot produce other anti-tumor chemokines, such as IFN-γ or granzyme β, thus, encouraging tumor resistance and development of metastatic disease or chemotherapy resistant tumor cells, release of IL-1 activates NF-κB encouraging metastatic disease and limits the use of immunotherapy with T-Cell Receptor (TCR) or chimeric antigen receptor (CAR) in these patients. In one embodiment, with exhausted cytotoxic lymphocytes, the patient is treated with antibody coated nanoparticles conjugated with a Rock inhibitor, such as Fasudil, etc. or Wnt inhibitors or GSK269962 inhibitors or integrin inhibitors in combination with anti-VEGFs, such as Bevacizumab, Ranibizumab, Aflibercept, Avastin, and anti-platelet derived growth factor (PDGF) agents, with or without macrolides and subsequent plasmapheresis or dielectrophoresis to remove toxins and dead cells from the patient's body and then the clean blood can be re-infused to the patient. In one embodiment, the treatment is done systemically. In another embodiment, the treatment is done locally inside the tumor or systemically combined with anti-VEGF coated nanoparticles.

In another embodiment, the pluralities of antibody coated nanoparticles are conjugated with Wnt inhibitors, Rock inhibitors, or GSK inhibitors in combination with a MAP/ERK inhibitor, such as sorafenib, an RAF kinase inhibitor, or Raf inhibitor sB 590885, PLX4720, RAF 265, XL281, ecorafenib, darafenib, vemurafenib, or MEK inhibitors, such as coimetinib, Bimimetinib, selumetinib or Trametinib or GSK 112020212 along with anti-VEGF such as Avastin, and checkpoint inhibitors, and IL-2, or IL-15, or polymeric antibody coated nanoparticles carrying IL-2 and IL-21, IL-6, IFN-γ or granzyme β, VLPs, or intratumor injectable oncolytic viruses, such as Reovirus, etc. in thermosensitive polymeric nanoparticles to release the medication after thermotherapy at a temperature of 39-43 degrees C. or more to damage the tumor cells but control the inflammatory process and prevent a cytokine storm formation or autoimmune response.

Although the antibodies are found on the regular tumor cells, finding them on the cancer stem cells (CSC) may not be easy so that these cells can be targeted. In one embodiment, the antibody coated nanoparticles are conjugated with a slow release polymer, such as polylactic or glycolic acid or porous silicon that can stay around the tumor cells or the CSC and their vasculature to damage the genetic components of the cancer stem cells and their vascular supply, while carrying VLPs or intratumor injectable oncolytic viruses, checkpoint inhibitors, and Rock inhibitors inhibiting Rho-kinases, GSK 269962, potent and selective ROCK inhibitor GSK 429286, Selective Rho-kinase (ROCK) inhibitor H1152 dihydrochloride, Selective Rho-kinase (ROCK) inhibitor Glycyl H 1152 dihydrochloride, Selective Rho-kinase (ROCK) inhibitor; more selective analogue of H1152, cell-permeable, selective Rho-kinase inhibitor OXA 06 dihydrochloride, potent ROCK inhibitor PKI1447 dihydrochloride, potent and selective ROCK inhibitor; antitumor SB 772077B, potent Rho-kinase inhibitor; vasodilator SR 3677 dihydrochloride, potent, selective Rho-kinase (ROCK) inhibitor TC-S7001, potent and highly selective ROCK inhibitor; orally active Y-27632 dihydrochloride or Wnt inhibitors such as FH535, IWP-2, PNU-74654, IWR-Tendo, IWR-exo, Demethoxycurcumin, CCT036477, KY02111, WAY-316606, SFRP, IWP, LGK974, C59, Ant1.4Br/Ant 1.4C1, ivermectin, niclosamide, apicularen and bafilomycin, XAV939, XAV939, G007-LK and G244-LM, NSC668036, SB-216763, gemtuzumab, or GSK inhibitors GSK 429286, and Avastin, an anti-VEGF to induce an immune stimulation locally but control the inflammation and further damage or killing the tumor by stimulation of an immune response while the existence of the radioactive coating, such as functionalized radioactive gold or combination of gold and ferric oxide nanoparticles with activatable cell-penetrating peptides (ACPPs) or alpha or a beta radiator, of the nanoparticles helps with the continuous radiation even if they are picked up by the cytotoxic lymphocytes, macrophages, or killer cells, preventing them from becoming either a victim of the tumor cell or to be converted to a hybrid tumor cell, while continually locally radiating the CSC or tumor cells from inside them and killing the tumor cells, along with the patient's immune cells by the effect of the localized radiation and killing them so as to be removed by either cellular immune response or plasmapheresis or dielectrophoresis, and removing the released toxins from the circulation.

In one embodiment, viruses are causes of the development of the cancer, such as Merkel-cell carcinoma, papilloma virus, Herpes virus, Creutzfeldt-Jakob disease, or Epstein-Barr virus, that can produce both skin cancer or brain cancer, or may lead to neurodegenerative processes, such as Alzheimer's disease, similarly the prions can cause inflammation in the brain, the inflammatory process caused by the viruses, or prions activates p38 MAPK synaptotoxic signaling pathway and causes neuronal damage, it is known that the Rock inhibitors control the inflammatory process and prevent cell death and particularly contribute to neuronal regeneration and branching of the axons, similarly GSK-3 beta inhibition by Lithium causes spreading the of cerebellar neurons and has implication in treatment of Alzheimer's disease, or Parkinson disease. In one embodiment, the Rock inhibitors or GSK inhibitors are conjugated with polymeric nanoparticles such as polylactic or glycolic acid, and can be administered intranasally to travel to the brain via the olfactory nerve.

In one embodiment, the controlled thermotherapy is done to release any medication at the desired temperature of 37-42 degrees C. at any desired location in the body for other applications beyond cancer therapy, such as treating chronic inflammation such as arthritis, uveitis, pain, muscle spasm, peripheral or central nerves, or generalized pain, etc. bacterial or viral, fungal, or protozoal infections or the complication of previous therapy such as radiotherapy or chemotherapy affecting the heart, vascular system, central nervous system (CNS), respiratory system, gastrointestinal system, genitourinary system, in general, or locally where the organ or the tissue is affected using thermal therapy.

In one embodiment, the temperature controlled thermotherapy includes all sources of energy under the control of photoacoustic, thermoacoustic, or simple thermocouple or fiber optics that can be placed close to the lesion, such as the skin, mucosa, or any place in the body or can be brought via a fiber optic using the source of energy externally or internally, light, laser, electromagnetic radiation, focused ultrasound, microwave, radiofrequency, alternating magnetic field at low or high frequency or electrically induced increased thermal effect, under the control of the unit and software, etc. to measure and image the temperatures in one, two, or three dimensional fashion and control the temperature at the desired level at desired temperatures ranging from 37 degrees C. to 40 degrees C. to 42 degrees C. to 45 degrees C. or 50 degrees C. or more and for the desired time.

In one embodiment, the system can be used to measure the increased temperature in the tissue, or joint, etc., beyond 37 degrees C., and can be measured using photoacoustic technology, etc. indicating an inflammatory response in the body that can be treated with medication and reduction of the temperature after therapy that indicates effectiveness of the therapy for the lesion located internally or externally to the body.

In one embodiment, one can use the combination of thermal energy, under a control of software, to heat up locally outside or inside the body precisely and control the temperature at a desired level of 37-43 degrees C. or more and to release medication from the antibody coated nanoparticles, such as liposomes/micelles, either injected inside a lesion or in the circulating the tumor.

In one embodiment, when a lesion is a tumor, the precision thermotherapy can either kill the tumor cells by increasing the temperature beyond 43 C degrees to damage the tumor cell non-invasively which are more sensitive to the temperatures beyond 40 C degrees than the normal cells, because of their natural high metabolism and their rapid multiplication. At lower temperatures of 40-43 C degrees, the thermal energy sensitizes the tumor and makes their cell membranes more vulnerable to chemotherapy inducing cell death and makes it less likely for the tumor cells to modify the genetic composition and become chemotherapy resistance. Benign lesions do not show increased temperatures, but still can be damaged or eliminated by repeated precision thermotherapy and precise temperature increase in the tumor.

In one embodiment, the tumor cell becomes less likely to protect themselves by producing checkpoint proteins to protect themselves from cellular immune therapy.

In one embodiment, the increased temperature causes the leaky tumor capillaries to relax and become leaky, increasing the temperature to 45-47 C degrees or more to initiate protein denaturation if applied for a period of 3-6 minutes or more, and tumor cell death.

In one embodiment, increasing the temperature to 43 C degrees can melt the lipid polymeric composition of certain nanoparticles, such as liposomes and micelles or polymeric nanoparticles, lactic and polyglycolic acid or anhydrides and solid lipids, etc. and these nanoparticles carry medication which are released slowly.

In one embodiment, the liposomes, micelles or nanoparticles are conjugates with antibody or aptamers that when injected inside the tumor or in the circulation, the nanoparticles attach to the cell membrane of the tumor.

In one embodiment, if the nanoparticles are conjugated with cell penetrating proteins, the nanoparticles or liposomes/micelles penetrate the cell membrane and release the medication in the membrane.

In one embodiment, the nanoparticles can absorb the thermal energy, such as light or laser at wavelengths of 200 nm-8000 nm and heat up, transferring the heat to the tumor cells or damaging the tumor cell membranes.

In one embodiment, the thermal energy is a focused ultrasound, a microwave, radiofrequency (RF), or an alternating magnetic field (AMF), etc.

In one embodiment, the metallic nanoparticles (NPs) absorb more radiation energy than the surrounding normal tissue and the temperature of the tumor cells to which the NPs are attached rises even more than the normal cells.

In one embodiment, the absorption of energy by the nanoparticles causes the nanoparticles to expand and create an ultrasonic sound that can be recorded by a transducer, or the focused ultrasound can initiate a back scatter energy that changes its frequency depending on the temperature at the focal point.

In one embodiment, the focused ultrasound can create a harmonic wave in the tissue that changes depending on the temperature and can be received by a transducer and recorded by a computer.

In one embodiment, the focused ultrasound produces an RF wave in the heated tissue that can be recorded by an ultrasound receiver, to be converted to an image.

In one embodiment, the focused ultrasound waves can produce an image that can be received by an ultrasound transducer and converted to an image of the area through which the ultrasound has passed.

In one embodiment, the thermotherapy can damage the tumor cells either by the thermal injury or by a combination of thermotherapy and chemotherapy, and the tumor cells release more antigenic not only from their cell membrane proteins but also their cytoplasm enhancing the humoral and cellular response to the tumor.

In one embodiment, the focused ultrasound can damage the cell without significantly increasing the temperature if applied in pulses with high intensity for a short period of time, creating a mechanical energy (e.g., break the wall of liposomes or micelles, etc.).

In one embodiment, thermotherapy can be combined with administration of culture grown modified CAR-T cells administered inside the tumor or in the arteries close to the tumor or intravenously to fight the remaining tumor cells, whether malignant or benign, and also their exosomes or extracellular vesicles, not only locally, but also systemically by attacking the metastatic lesions and circulation tumor cells inducing a remote killing effect away from the original tumor.

In one embodiment, thermotherapy uses antibody or aptamers-coated liposomes or micelles filled with medications and either with metallic or non-metallic nanoparticles or thermosensitive polymers which deliver medication and melting in temperatures of 40-43 C degrees using focused ultrasound, or laser, or alternating magnetic field, microwaves, or radiofrequency (RF) under the control of the thermal energy using proportional-integral-derivative (PID) software for maintaining the temperature at a desired level to release medication(s), etc. (e.g., by melting or breaking the liposomes for a period of time of 0.5 seconds to 10 minutes or longer without damaging the normal tissue while affecting survival of malignant tumor cells, exosomes or circulating tumor cells).

In one embodiment, the lesion can be imaged simultaneously with its temperature, using photoacoustic, thermoacoustic or ultrasound imaging, recording the backscatter or change of the backscatter, or harmonic waves or the RF waves or combination of them in one or two dimensional fashion or using a combination of ultrasound and photoacoustic imaging simultaneously to measure the temperature of the heated tissue and transmit the information to PID to control the thermal energy by software.

In one embodiment, the thermal energy source can be a laser, an ultrasound, an alternating magnetic field, a microwave, or a radiofrequency (RF) unit, all controlled by a software that controls thermal energy at the desired temperature level.

In one embodiment, the antibody-coated liposomes can be loaded antibody/aptamer-coated nanoparticles, and then when released by thermal energy, the NPs attach to the desired cells or tumor cells.

In one embodiment, the pluralities of liposomes or micelles carry in addition to medication, the nanoparticles or multiple other medications that can modify the response of the tumor to the cellular immune therapy, such as one or more checkpoint inhibitors.

In one embodiment, pluralities of liposomes or micelles carry various medications and or immune modifiers, such as Wnt inhibitors, Rock inhibitors, GSK inhibitors, integrin inhibitors, IL-6 inhibitors, or low molecular weight heparin.

In one embodiment, the pluralities of liposomes/micelles carry immune stimulators, such as viral like particles (VLP), oncolytic viruses, venoms, etc. One mechanism is by releasing the VLP, immune stimulation with IL-2, IL-6, IL-17, toll-like receptor 7/8 or interferons with antibody-coated polymeric nanoparticles or dendrimers as slow release compounds with cell penetrating peptides (CPPs) and/or activatable CPP (ACPP).

In one embodiment, the temperature is maintained at about 43-44 C degrees or more for a desired period of time of 2-4 minutes release using PID or another software to control the temperature at desired level of the medication which is measured.

### Example 1

The thermosensitive liposomes consisting of DPPC: At a composition of 96:4 mol %. The liposomes are formed using the thin film hydration method. 100 mg of lipids are dissolved in 10 mL of isopropanol the medication such as doxorubicin is added to the desired concentration. The solution is aliquoted to 1 mL batches in glass vials and the solution is dried by leaving the vial under a stream of nitrogen gas, followed by overnight desiccation in vacuum. The lipid film is hydrated with (1) HEPES buffer or (2) doxorubicin and/or antibody-coated nanoparticles in the antibody-coated liposomes as needed.

In one embodiment, the liposomes form as known in the art after application of sufficient thermal energy to disperse phospholipids using a polar solvent such as water, etc. and the multilamellar liposomes are broken into unipolar bilayer vesicles by sonication.

In one embodiment, the other ingredients for drug delivery, etc. can be incorporated inside the liposome during the liposome formation such as, metallic NP, gold, zinc, magnetic nanoparticles, iron oxide NPs, or paramagnetic nanoparticles or antibody-coated polymeric slow release medication, or polymeric nanoparticles such as polylactic acid (PLA), polyglycolic acid, polylactic and glycolic acid (PLGA), polycaprolactone, polyanhydride, micelles or solid lipids. Nanoparticles that may be formulated as antibody/aptamers-coated microspheres or antibody-coated dendrimers or antibody-coated nanoparticles, including antibody-coated quantum dots, nanotubes, nanowires, antibody-coated nanoparticles, polymer-drug conjugate, antibody-coated polymeric-drug nanoparticle, antibody/aptamers-coated dendrimer-medication, and antibody coated iron oxide, or other magnetic NPs, or gold nanoparticles with or without medications. In this embodiment, one can use liposomes or micelles filled with the desired medication or medications.

In one embodiment, antibody/aptamer-coated liposomes, micelles, nanoparticles, and/or microparticles or with (alpha)-cyclodextrin, or (beta)-cyclodextrin, or (gamma)-cyclodextrin, hydroxypropyl-b-cyclodextrin (bHPCD)/medication can be given, intravenously or intra-arterially, nasally through olfactory nerve or by inhalation to reach the brain or lung tumors, or via a catheter, by local injection, orally, or inside the tumor prior to precision thermotherapy or can be delivered as slow release compounds implanted or injected inside the tissue with any polymeric drug compound, such as, for example, polycaprolactone, poly(glycolic) acid, poly(lactic) acid, etc.

In one embodiment, alternatively the antibody-conjugated liposomes/micelles and nanoparticles can be conjugated with aptamers to deliver medication, etc. with the liposome/micelles to specific targets.

In one embodiment, since aptamers can be easily removed from the circulation, the best approach to targeting a tumor is to inject them either inside a tumor or intra-arterially or intravenously shortly before thermotherapy using low intensity focused ultrasound (LIFU) for 2-6 minutes or more or pulsed high intensity focused ultrasound (HIFU) for precise drug delivery.

In one embodiment, an alternative thermal energy source, such as laser of any wavelength, microwave, or radiofrequency waves or an alternating magnetic field and magnetic nanoparticles are used and combined with liposomes filled with magnetic nanoparticles to break the liposomes and release their contents locally at a predetermined temperature under the control of thermal energy using a software at 40-43 degrees C. or more to break up the liposomes and release the medication or other nanoparticles from the liposomes, micelles, or vesicles.

In one embodiment, the LIFU can be applied to multiple areas of the tumor after the administration of medications inside the tumor, intravenously or in the feeding artery to release the medication along with the release of immune stimulators, VLP, oncolytic viruses, or toll-like receptor 4, etc. with simultaneous cellular therapy. In one embodiment, this methodology can repeatedly administered every 3 months, etc. as needed after thermotherapy to stimulate the immune response as an anti-tumor vaccination with or without administration of natural killer (NK) cells and affect potential metastatic lesions, tumor cells, and/or their extracellular vesicles.

In one embodiment, aptamers conjugated with liposomes, or other nanoparticles or other slow release delivery polymers, such as polyethylene glycol (PEG), polylactic acid or polyglycolic acid or combining them with temperature sensitive lipids permitting the release of the medication for a longer time at the desired location.

In one embodiment, the aptamers conjugated with nanoparticles, liposomes, and/or micelles for an initial therapeutic effect and can be followed after a period of time with an antidote to prevent its continuous action if needed. This methodology is specifically desired when using toxins such as venom or botulinum toxin at pictogram to nanogram concentrations with aptamers-conjugated nanoparticles to target a tumor with bee venoms, etc. to enhance an immune response locally at the site of a tumor while the aptamer antidote is injected subsequently to dampen the excessive cellular immune response to prevent excessive damage to the normal tissue or prevent cytokine response.

In one embodiment, pluralities of liposomes and/or micelles can carry any combination of medication including anti-cancer medication such as doxorubicin, etc. or immune stimulators, anti-VEGF, Diamox, sodium bicarbonate, etc., cellular pathway inhibitors, such as Rock inhibitors, such as Fasudil or Botox, or Wnt inhibitors, such as niclosamide, ivermectin, and Selamectin and alpha lipoidic acid (ALA), statins or small molecule Wnt inhibitors with controllable temperature release or other anti-inflammatory medication including statins, NSAIDs, cyclosporine A or mycophenolic acid, etc.

In one embodiment, the antibody/aptamers conjugated liposomes can also carry polymeric nanoparticles that are conjugated with either an aptamer or monoclonal or polyclonal antibody while simultaneously can carry various medications such as anti-cancers, such as doxorubicin, etc. or cell pathway inhibitors, such as Rock inhibitors, Wnt inhibitors, GSK-3 inhibitors, IL-6 inhibitors, low molecular weight heparin, GSK 429286 or anti-integrin, abciximab, Eptifibatide, Tirofiban, αIIbβ3 antagonists, Natalizumab, 3 mg to ±52 μg/mL, MLN-00002, Firategrast, IVL745, antagonists of αvβ3 and/or αvβ5 integrins, to be released at the desired temperature and site (e.g., at a tumor site) or to treat a degenerative disease, such as Alzheimer's or Parkinson disease.

In one embodiment, the antibody-conjugated aptamers, conjugated liposomes, or polymeric nanoparticles can carry a variety of medications such as anti-cancers, such as doxorubicin, etc. or cell pathway inhibitors, such as Rock inhibitors, Wnt inhibitors, GSK-3 inhibitors, or anti-integrins, IL-6 inhibitors, or low molecular weight heparin, and injected locally inside the tumor or intra-arterially, for example, in the carotid artery of an artery feeding an organ such as stomach, intestine, lung, nose, and throat, breast, CNS, spinal cord, or ocular tumors, prostates, breast cancer, ovarian tumor, nose cancer, throat cancer, skin cancer, mucosa cancer, intestinal or lung cancer, genitourinary system cancer, bone cancer, cerebrospinal cancer, or a benign compressive tumor, e.g., of the brain or prostate, etc. or close to a desired site to be attached to specific tumor cells first, before its distribution in the rest of the body. This applies also to a localized inflammatory process or degenerative process.

In one embodiment, at least two anticancer medications are combined in antibody-coated or aptamer-conjugated liposomes that can be released simultaneously after local application of thermotherapy for therapy of chemotherapy resistant neoplasms or a benign compressive tumor.

In one embodiment, at least two medications are incorporated in slow release polymers such as PLA or GA or PGLA, anhydrides, polycaprolactone, or chitosans and incorporated in the antibody or aptamer conjugated liposomes/micelles or solid lipids and injected inside the tumor or intravenously or intra-arterially with or without subsequent localized thermotherapy at the site of the at least one tumor lesion.

In one embodiment, multiple areas of the tumor are treated with localized precision thermotherapy to induce an immune response to the damaged tumor cells.

In one embodiment, the pluralities of antibody or aptamer-conjugated liposomes are loaded with checkpoint inhibitors, such as the checkpoint inhibitors: PD-1 PD-L1, CTLA-4, Jagged 1 inhibitor 15D11, etc. and immune stimulators, such as VLP, IL-2, IL-4 TLR7, etc. and can be combined with anticancer medications with the antibody-coated liposomes or antibody-coated polymeric nanoparticles, such as polylactic acid (LA), polyglycolic acid, polylactic and glycolic (PLGA) polymer acid, polycaprolactone, polyanhydride, micelles or lipids nanoparticles that may be formulated as antibody-coated microspheres or antibody-coated dendrimers or antibody-coated nanoparticles to be injected inside the tumor or at least one lesion with a fine needle or inside a feeding artery or systemically to be attached to the tumor cells surface. In one embodiment, the tumor cells are irradiated immediately after administration of the medication with either a laser with a wavelength of 400-1500 nm or infrared laser, or microwave or focused ultrasound or radiofrequency beam or alternating magnetic field and magnetic antibody-coated nanoparticle to locally increase the tumor cells' temperature, while controlling the thermal energy using a proportional integral derivative (PID) system that controls the energy delivery unit to maintain the temperature locally at about 43 C degrees or more to make the tumor cell vulnerable to the medication, while the slow release medications can be released from the polymeric nanoparticles for a desired time.

In one embodiment, in therapy of malignant and benign lesions, the liposome/micelles carry piezoelectric nanoparticles that can be heated with focused ultrasound and simultaneously create an electric charge in the tumor depolarizing the tumor cells and affecting their physiological function leading to cell death.

In one embodiment, one can conjugate the liposomes or the nanoparticles with anionic aptamers and inject them either in the tumor or intra-arterially or intravenously and after a desired time one can neutralize the anionic aptamers with cationic aptamers at the desired site by injecting them locally or systemically.

In one embodiment, in treating cancer or benign lesions, the liposomes are conjugated with an aptamer or a monoclonal or polyclonal antibody filled with polymeric nanoparticles/liposomes conjugated with viral-like particles or other immune stimulators to enhance immune response locally after the injection inside a tumor or intra-arterially close to a lesion to attach to the tumor cells preferentially before getting in the circulation. In one embodiment the liposomal delivery is combined afterward with administration of controlled local thermotherapy using a focused ultrasound as low intensity focused ultrasound or pulsed hi-intensity focused ultrasound to release the medication from the liposome at temperature of 39-43 C degrees and subsequently the medication locally while heating the tumor cells locally. In one embodiment, the release of the medication or along with immune stimulators, such as VLP, or antibody-coated nanoparticles carrying IL-2, IL-4, IL 6, TLR7, etc. can enhance cellular immune response that can systemically affect other metastatic lesions in the body, thus treating one or two or more lesions in the body or circulating tumor cells/exosomes having the same biomarkers as the tumor antigen, but also the visible and invisible lesions in other parts of the body.

In one embodiment, the thermotherapy using focused ultrasound, LIFU, or pulsed high intensity focused ultrasound (PHIFU) is applied locally for release of locally or intravenously, etc. liposomes/micelles filled with medications to treat, e.g., a joint arthritis, or reduce the pain or using LIFU to increase the temperature for release of nerve pain, migraine, or using LIFU for nerve stimulation, local tissue regeneration in brain, or in cosmetic facial rejuvenation, etc. under the control of the thermal energy software for a desired time or one can stop the pain of peripheral nerves with LIFU administered locally for a desired time with or without medication release or enhanced oxygenation.

In one embodiment, the immune response or cytokine storm can be damped by local or intravenous application of the medication, such as anti-VEGF, Rock inhibitors, acetazolamide, and/or IL6 inhibitors, and by performing a kidney dialysis or electrophoresis to clean up the damaged tumor cells before reinfusing the blood cells back in the patient along with other anti-inflammatory agents including Rock inhibitors, IL-6 inhibitors, NSAIDs, statins, etc.

In one embodiment, the antibody or aptamer-conjugated liposomes/micelles filled with medication are simultaneously administered with culture grown natural killers or genetically modified NK cells or CAR-T cells, along with pathway inhibitors or checkpoint inhibitors, medication or other immune stimulators such as VLP or IL2, IL-4, IL-6, TLR7 conjugated with pluralities of antibody-coated polymeric nanoparticles, etc. locally inside the tumor or intravenously but preferably intra-arterially to reach the tumor at desired location before their distribution systemically. This treatment can be combined with thermotherapy of the tumor with simultaneous control of the temperature at 43 C degrees and imaging.

In one embodiment, cell penetrating peptides (CPP), activatable CPP (ACPP), biotin, and/or streptavidin and antibody-coated nanoparticles or liposomes conjugated with (alpha)-cyclodextrin, (beta)-cyclodextrin, (gamma)-cyclodextrin, hydroxypropyl-b-cyclodextrin (bHPCD) conjugated with thermosensitive polymers, such as PLA, PGA, chitosan, and polyanhydride or porous silicon nanoparticles and/or microparticles conjugated with siRNS, DNA, RNAi, CRISPR-Cas9, medications, such as Wnt inhibitors, checkpoint inhibitors, or Rock inhibitors, integrin inhibitors or along with CAR-T cells grown in cell culture and sensitized to the tumor antigens along with immune stimulation with VLP, adjuvants, sodium bicarbonate and/or Anti-VEGFs to modify the acidic tumor cell environment, toll-like receptors 6/7/8 or interferons. In one embodiment, the antibody-coated nanoparticles/liposomes can be injected in the circulation locally in the tumor systemically, but preferably intra-arterially with subsequent dielectrophoresis and or kidney dialysis and administration of anti-inflammatory agents to prevent cytokine storm in the patient.

### Example 2

In an example, the lipid solution was extruded 9 times through a polycarbonate disk and two stacked 0.1 μm filter supports at a temperature above the main phase transition temperature, the doxorubicin with or without gold or magnetic nanoparticles of 1-500 nm or more are added, and the synthesized liposomes were used within 48 hours.

The nanoparticles, liposomes, and/or micelles can be conjugated with monoclonal antibodies or polyclonal antibodies or aptamers (see e.g., the antibodies in Table 1 below) to seek desired normal cells or tumor cells after intravenous or intra-arterial administration or directly inside the tumor to release any medication which is attached to the nanoparticles such as doxorubicin, anti-cancer medication, anti-inflammatory medications, and/or cell pathway inhibitors at a desired temperature of 39-44 C degrees, preferably 43 C degrees.

In an example, the release of the doxorubicin can be measured using fluorescence de-quenching assay and the release is calculated by the formula:

$$R = \frac{{I}_{x} - {I}_{0}}{{I}_{100} - {I}_{0}}$$

### In Vitro Drug Release (Water-Bath Heating)

The loaded liposomes were exposed to increased temperature by placing the loaded-liposomes containing tubes in a temperature controlled water bath. The release of doxorubicin was determined using a fluorescence de-quenching assay. The fractional release of doxorubicin was calculated (see below). R is the fractional release, Ix is the fluorescence of treated sample, IO is the fluorescence of a non-treated sample, and I100 is the fluorescence of the sample treated with 1% Triton X-100 (vol/vol) which is a detergent that lyses the liposomes and therefore corresponds to the complete released drug fluorescence reference. The fluorescence (Ex 485 nm/Em 590 nm) was measured with a multimode plate reader.

### Example 3

The release of doxorubicin is evaluated in a water bath (WB) by increasing the temperature of the WB gradually from 37 C to 43 C. The experiment demonstrated that 100% liposomes release the medication at 43 C degrees temperature.

### Example 4

BT474 colone 5 cells of the breast cancer were grown and exposed in tissue culture to various concentration or doxorubicin 0.17 fM (0.6 μM), 0.5 fM (1.8 μM), 1.7 fM (6 μM), and 5 fM (18 μM). The two higher concentrations, 1.7 fM (6 μM), and 5 fM (18 μM) were performed for four days and the cells survival were counted. The majority of cells exposed to the highest concentration of doxorubicin of 1.8 μM died.

### Example 5

Gold nanoparticles and doxorubicin loaded liposomes were filled in a sample holder and placed inside a pork loin. The samples were then exposed either to low intensity focused ultrasound (LIFU) producing a controlled thermal effect of 43 C degrees for five minutes 50% duty cycle and output power of 2.1 W. The control sample was water bath of 37 C degrees. The maximum release of liposomes was found with LIFU for five minutes of exposure causing 5 C degrees rise of the temperature.

### Example 6

Ultrasound thermometry: Ultrasound is an attractive thermometry modality due to its relatively high sensitivity to change in temperature and fast data acquisition and processing capabilities. A temperature sensitive acoustic parameter is required for ultrasound thermometry in order to track the changes in that parameter during the treatment. Currently, the main ultrasound thermometry methods are based on variation in the attenuation coefficient, the change in backscattered energy of the signal (CBE), the backscattered radio-frequency (RF) echo-shift due to change in the speed of sound and thermal expansion of the medium, and change in the amplitudes of the acoustic harmonics. An ultrasound thermometry method was implemented based on a recently developed method, the change in CBE generated by nonlinear propagation of the ultrasound beam.

### Example 7

A couple of experiments were conducted with an ultrasound scanner and LIFU transducer. The central frequency of the transducer was increased from 4.2 MHz to 11.3 MHz. This was done to increase the sensitivity of ultrasound thermometry methods. The focal temperature was maintained at 43° C. for 4 minutes based on thermocouple measurement and backscattered RF signals were acquired to generate temperature change. The plot of temperature measurement from the calibrated thermocouple was generated. Maps of temperature change were produced using the change in backscattered energy (CBE). The average percentage change in CBE at the focal region of the LIFU was estimated, the heated region at the focus of LIFU expanded during the heating phase and it shrank during the cooling phase. The percentage change in CBE remained relatively constant during the heating phase where the temperature was controlled and maintained at 43° C. for 4 minutes and it gradually decreased during the cooling phase. The results show that the higher frequency is more sensitive to temperature change.

### Example 8

Ultrasound thermometry: New experiments were performed using an ultrasound scanner and LIFU to control the temperature at the focal region of the LIFU. The focal temperature was maintained at 43° C. for 4 minutes based on thermocouple measurement and backscattered RF signals were acquired to generate temperature maps. The plot of the average focal temperature using the linear (fundamental) change in backscattered energy (CBE) technique was obtained. The results showed a good correlation between the thermocouple measurement and ultrasound backscattered methods.

### Example 9

Temperature estimation from temperature-controlled system using a calibrated needle thermocouple was done using software. The temperature was controlled for 4 minutes to a target temperature of 43° C. and then it was let to naturally cool down. A temperature map was created over the entire period of time.

In the experiments conducted, the thermal mode increased cell death compared to the controls.

In the one or more experiments conducted, the temperature could be maintained for 4-5 minutes at 43 C degree for a complete breakdown of the liposomes and it took 15 minutes for the tissue to return to the normal 37 C degrees. It is expected that this cooling effect will be a lot shorter in vivo in presence of the blood circulation. This technology has application for treating the tumors as small as 5 mm in diameter to larger ones (e.g., 1-2 cm or larger) particularly if combined with medication and other modalities to stimulate the immune response, such as VLP or tumoricidal viruses or Toll like receptor 4, in the tissue after administration of local or intra-arterial administration or inhalation of antibody or aptamer-conjugated loaded liposomes and the tumor is exposed to the medication before its distribution in the body to stimulate immune response. In addition, since the liposomes can break at temperature below 43 degrees C., the heating of the tumor induces release of the medication from the liposomes before their uptake by the tumor cellular elements. Simultaneously, an excessive immune response can be prevented by administration of pluralities of antibody/aptamer-coated liposomes loaded with pathway inhibitors such as Rock inhibitors, Wnt inhibitors, anti-integrins, GSK-3 inhibitors, IL-6 inhibitors, or low molecular weight heparin with or without ant-VEGFs. However this technology can be used for localized delivery of any medication as needed with simultaneous imaging with or without if the medication is not harmful to the body and the ultrasound unit is modified accordingly for such use.

### Example 10

PA thermometry: noninvasively controlling laser thermal therapy. Photoacoustic (PA) imaging is an emerging hybrid modality that has the contrast of optical imaging and the high resolution of ultrasound imaging. The ability of photoacoustic imaging to provide structural and functional imaging leads to its investigation in diverse application areas, including temperature monitoring in tissues. Real-time and non-invasive thermometry techniques are essential in thermal therapies to monitor and control the treatment. In this example, the use of PA thermometry in controlling thermal therapies was investigated. Specifically, the focus was on laser-induced thermal therapy with gold nano rods (GNR) deposited in the targeted region. The use of GNR both enhanced the laser heating process and improved the PA signal-to-noise (SNR) of the PA signal.

In one embodiment, alternating magnetic field (AMF) thermal therapy may be combined with magnetic nanoparticles (MNPs). External AMFs are not altered by tissue and therefore can be applied to any depth. This is a significant advantage over lasers and ultrasound. AMF treatment is advocated for advanced stage cancers or a benign compressive tumor that can no longer be removed surgically. The development of automatic real-time control of alternating magnetic fields (AMFs) combined with nanoparticle systems composed of magnetic nanoparticles (MNPs) can be useful in the future.

In one embodiment of a low dose of thermotherapy using laser or focused ultrasound, AMF and magnetic nanoparticles or microwaves at temperatures of 39-43 degrees C. for a period of time with proportional integral derivative control using software alone or in combination with an adjuvant administration such as toll-like receptor 4 or VLP, etc. or a dead tumor cell vaccine administered locally or intravenously or intra-arterially close to the lesion, e.g., breast cancer, prostate cancer, etc. enhances the humoral and cellular immune response to the tumor cells by increasing the T-cell and killer cell response to the localized tumor or its metastatic lesions or circulating tumor cells of the patient.

In another embodiment of low dose of thermotherapy using laser or focused ultrasound, AMF and magnetic nanoparticles or microwaves at temperatures of 39-43 degrees C. for a period of time with proportional integral derivative control using software alone or in combination with an adjuvant administration, such as toll-like receptor 4 or VLP, etc., or a dead tumor cell vaccine, or natural killer (NK) cells grown in the cell culture, along with one or more pathway inhibitors, such as a Rock inhibitor, Wnt inhibitor, GSK inhibitor, or integrin inhibitor administered locally or intravenously, or intra-arterially close to the lesion, e.g., breast cancer, prostate cancer, etc. enhances the humoral and cellular immune response to the tumor cells by increasing the T-cell and killer cell response to the localized tumor or its metastatic lesions or circulating tumor cells of the patient. The effect of the therapy can be evaluated by one of the imaging modalities showing shrinkage of the tumor and reduced circulating tumor cells or enhance humoral antibody in the blood.

In one embodiment of a low dose of thermotherapy using laser or focused ultrasound, AMF and magnetic nanoparticles or microwaves at temperatures of 39-43 degrees C. for a period of time with proportional integral derivative control using software alone or in combination with antibody-coated nanoparticles (NPs), magnetic or non-magnetic, gold or other metallic NPs, or rod-shaped or combined with micelles or liposomes filled with medication or an adjuvant administration, such as toll-like receptor 4, or VLP, etc. or a dead tumor cell vaccine is administered locally or intravenously or intra-arterially close to the lesion, e.g., breast cancer, prostate cancer, etc. enhances the humoral and cellular immune response to the tumor cells by increasing the T-cell and killer cell response to the localized tumor or its metastatic lesions or circulating tumor cells of the patient.

In one embodiment of a low dose of thermotherapy using laser or focused ultrasound, AMF and magnetic nanoparticles (NPs) or microwaves at temperatures of 39-43 degrees C. for a period of time with proportional integral derivative control using software alone or in combination with an adjuvant, such as toll-like receptor 4 or VLP, etc. and antibody-coated radioactive gold NPs or other antibody-coated nanoparticles filled in liposomes that break up during the thermotherapy releasing the radioactive particles, or in combination with a dead tumor cell vaccine with or without anti-VEGF administered locally or intravenously or intra-arterially close to the lesion, e.g., breast cancer, prostate cancer, or cancer in any other place in the body, etc. enhances the humoral and cellular immune response to the tumor cells by increasing the T-cell and killer cell response to the localized tumor or its metastatic lesions or circulating tumor cells of the patient eliminating the tumor.

In one embodiment of a low dose of thermotherapy using laser or focused ultrasound, AMF and magnetic nanoparticles or microwaves at temperatures of 39-43 degrees C. for a period of time with proportional integral derivative control using software alone or in combination with an adjuvant administration, such as toll-like receptor 4 or VLP, etc. or a dead tumor cell vaccine with antibody-coated gold nanoparticles of 1-20 nm in diameter or administered locally or intravenously or intra-arterially close to the lesion, e.g. a breast cancer, prostate cancer, etc. where subsequent low dose radiation is applied locally or by a stereotactic low dose radiation using x-ray, proton beam or helium beam where the effect is enhanced by the gold nanoparticles, and is useful for simultaneous imaging and enhances the humoral and cellular immune response to the tumor cells by increasing the T-cell and killer cell response to the localized tumor or its metastatic lesions or circulating tumor cells of the patient. The results are evaluated by CT-scan, MRI, or ultrasound imaging of the lesion or the treatment is repeated as needed.

In another embodiment, the functionalized nanoparticles are conjugated with one of the recombinant, monoclonal, or polyclonal antibodies or aptamers known in the art and administered along with either one or more toxin(s) or antibodies, along with a medication that is provided at a much lower dose systemically to kill the already compromised tumor cells. Monoclonal antibodies that may be used include, but are not limited to, those shown in Table 1 below, e.g., rituximab, obinuzumab, oftumumab, etc.

Any of the features or attributes of the above described embodiments and variations can be used in combination with any of the other features and attributes of the above described embodiments and variations as desired.

Although the invention has been shown and described with respect to a certain embodiment or embodiments, it is apparent that this invention can be embodied in many different forms and that many other modifications and variations are possible without departing from the spirit and scope of this invention.

Moreover, while exemplary embodiments have been described herein, one of ordinary skill in the art will readily appreciate that the exemplary embodiments set forth above are merely illustrative in nature and should not be construed as to limit the claims in any manner. Rather, the scope of the invention is defined only by the appended claims and their equivalents, and not, by the preceding description.

TABLE 1

| Name | Trade name | Type | Source | Target | Use |
|---|---|---|---|---|---|
| 3F8 | | mab | mouse | GD2 | neuroblastoma |
| 8H9 | | mab | mouse | B7-H3 | neuroblastoma, |
| | | | | | sarcoma, metastatic |
| | | | | | brain cancers |
| Abagovomab | | mab | mouse | CA-125 (imitation) | ovarian cancer |
| Abciximab | ReoPro | Fab | chimeric | CD41 (integrin | platelet aggregation |
| | | | | alpha-IIb) | inhibitor |
| Abituzumab | | mab | humanized | CD51 | cancer |
| Abrilumab | | mab | human | integrin α4β7 | inflammatory bowel |
| | | | | | disease, ulcerative |
| | | | | | colitis, Crohn's disease |
| Actoxumab | | mab | human | Clostridium difficile | Clostridium difficile |
| | | | | | infection |
| Adalimumab | Humira | mab | human | TNF-α | Rheumatoid arthritis, |
| | | | | | Crohn's Disease, |
| | | | | | Plaque Psoriasis, |
| | | | | | Psoriatic Arthritis, |
| | | | | | Ankylosing Spondylitis, |
| | | | | | Juvenile Idiopathic |
| | | | | | Arthritis, Hemolytic |
| | | | | | disease of the newborn |
| Adecatumumab | | mab | human | EpCAM | prostate and breast |
| | | | | | cancer |
| Aducanumab | | mab | human | beta-amyloid | Alzheimer's disease |
| Afelimomab | | F(ab')₂ | mouse | TNF-α | sepsis |
| Afutuzumab | | mab | humanized | CD20 | lymphoma |
| Alacizumab pegol | | F(ab')₂ | humanized | VEGFR2 | cancer |
| ALD518 | | ? | humanized | IL-6 | rheumatoid arthritis |
| Alemtuzumab | Campath, | mab | humanized | CD52 | Multiple sclerosis |
| | MabCampath | | | | |
| Alirocumab | | mab | human | NARP-1 | hypercholesterolemia |
| Altumomab pentetate | Hybri-ceaker | mab | mouse | CEA | colorectal cancer |
| | | | | | (diagnosis) |
| Amatuximab | | mab | chimeric | mesothelin | cancer |
| Anatumomab | | Fab | mouse | TAG-72 | non-small cell lung |
| mafenatox | | | | | carcinoma |
| Anetumab ravtansine | | mab | human | MSLN | cancer |
| Anifrolumab | | mab | human | interferon α/β | systemic lupus |
| | | | | receptor | erythematosus |
| Anrukinzumab (=IMA- | | mab | humanized | IL-13 | ? |
| 638⁾ | | | | | |
| Apolizumab | | mab | humanized | HLA-DR ? | hematological cancers |
| Arcitumomab | CEA-Scan | Fab' | mouse | CEA | gastrointestinal |
| | | | | | cancers (diagnosis) |
| Ascrinvacumab | | mab | human | activin receptor-like | cancer |
| | | | | kinase 1 | |
| Aselizumab | | mab | humanized | L-selectin (CD62L) | severely injured |
| | | | | | patients |
| Atezolizumab | | mab | humanized | CD274 | cancer |
| Atinumab | | mab | human | RTN4 | ? |
| Atlizumab | Actemra, | mab | humanized | IL-6 receptor | rheumatoid arthritis |
| (=tocilizumab) | RoActemra | | | | |
| Atorolimumab | | mab | human | Rhesus factor | hemolytic disease of |
| | | | | | the newborn[citation |
| | | | | | needed] |
| Bapineuzumab | | mab | humanized | beta amyloid | Alzheimer's disease |
| Basiliximab | Simulect | mab | chimeric | CD25 (α chain of IL- | prevention of organ |
| | | | | 2 receptor) | transplant rejections |
| Bavituximab | | mab | chimeric | phosphatidylserine | cancer, viral infections |
| Bectumomab | LymphoScan | Fab' | mouse | CD22 | non-Hodgkin's |
| | | | | | lymphoma (detection) |
| Begelomab | | mab | mouse | DPP4 | ? |
| Belimumab | Benlysta, | mab | human | BAFF | non-Hodgkin |
| | LymphoStat-B | | | | lymphoma etc. |
| Benralizumab | | mab | humanized | CD125 | asthma |
| Bertilimumab | | mab | human | CCL11 (eotaxin-1) | severe allergic |
| | | | | | disorders |
| Besilesomab | Scintimun | mab | mouse | CEA-related antigen | inflammatory lesions |
| | | | | | and metastases |
| | | | | | (detection) |
| Bevacizumab | Avastin | mab | humanized | VEGF-A | metastatic cancer, |
| | | | | | retinopathy of |
| | | | | | prematurity |
| Bezlotoxumab | | mab | human | Clostridium difficile | Clostridium difficile |
| | | | | | infection |
| Biciromab | FibriScint | Fab' | mouse | fibrin II, beta chain | thromboembolism |
| | | | | | (diagnosis) |
| Bimagrumab | | mab | human | ACVR2B | myostatin inhibitor |
| Bimekizumab | | mab | humanized | IL17A and IL17F | ? |
| Bivatuzumab | | mab | humanized | CD44 v6 | squamous cell |
| mertansine | | | | | carcinoma |
| Blinatumomab | | BiTE | mouse | CD19 | cancer |
| Blosozumab | | mab | humanized | SOST | osteoporosis |
| Bococizumab | | mab | humanized | neural apoptosis- | dyslipidemia |
| | | | | regulated proteinase | |
| | | | | 1 | |
| Brentuximab vedotin | | mab | chimeric | CD30 (TNFRSF8) | hematologic cancers |
| Briakinumab | | mab | human | IL-12, IL-23 | psoriasis, rheumatoid |
| | | | | | arthritis, inflammatory |
| | | | | | bowel diseases, |
| | | | | | multiple sclerosis |
| Brodalumab | | mab | human | IL-17 | inflammatory diseases |
| Brolucizumab | | mab | humanized | VEGFA | ? |
| Brontictuzumab | | mab | Notch 1 | cancer | |
| Canakinumab | Ilaris | mab | human | IL-1? | rheumatoid arthritis |
| Cantuzumab | | mab | humanized | mucin CanAg | colorectal cancer etc. |
| mertansine | | | | | |
| Cantuzumab | | mab | humanized | MUC1 | cancers |
| ravtansine | | | | | |
| Caplacizumab | | mab | humanized | VWF | thrombotic |
| | | | | | thrombocytopenic |
| | | | | | purpura, thrombosis |
| Capromab pendetide | Prostascint | mab | mouse | prostatic carcinoma | prostate cancer |
| | | | | cells | (detection) |
| Carlumab | | mab | human | MCP-1 | oncology/immune |
| | | | | | indications |
| Catumaxomab | Removab | 3funct | rat/mouse hybrid | EpCAM, CD3 | ovarian cancer, |
| | | | | | malignant ascites, |
| | | | | | gastric cancer |
| cBR96-doxorubicin | | mab | humanized | Lewis-Y antigen | cancer |
| immunoconjugate | | | | | |
| Cedelizumab | | mab | humanized | CD4 | prevention of organ |
| | | | | | transplant rejections, |
| | | | | | treatment of |
| | | | | | autoimmune diseases |
| Certolizumab pegol | Cimzia | Fab' | humanized | TNF-α | Crohn's disease |
| Cetuximab | Erbitux | mab | chimeric | EGFR | metastatic colorectal |
| | | | | | cancer and head and |
| | | | | | neck cancer |
| Ch.14.18 | | mab | chimeric | ??? | neuroblastoma |
| Citatuzumab bogatox | | Fab | humanized | EpCAM | ovarian cancer and |
| | | | | | other solid tumors |
| Cixutumumab | | mab | human | IGF-1 receptor | solid tumors |
| Clazakizumab | | mab | humanized | Oryctolagus | rheumatoid arthritis |
| | | | | cuniculus | |
| Clenoliximab | | mab | chimeric | CD4 | rheumatoid arthritis |
| Clivatuzumab | hPAM4-Cide | mab | humanized | MUC1 | pancreatic cancer |
| tetraxetan | | | | | |
| Codrituzumab | | mab | humanized | glypican 3 | cancer |
| Coltuximab ravtansine | | mab | chimeric | CD19 | cancer |
| Conatumumab | | mab | human | TRAIL-R2 | cancer |
| Concizumab | | mab | humanized | TFPI | bleeding |
| Crenezumab | | mab | humanized | 1-40-β-amyloid | Alzheimer's disease |
| CR6261 | | mab | human | Influenza A | infectious |
| | | | | hemagglutinin | disease/influenza A |
| Dacetuzumab | | mab | humanized | CD40 | hematologic cancers |
| Daclizumab | Zenapax | mab | humanized | CD25 (α chain of IL- | prevention of organ |
| | | | | 2 receptor) | transplant rejections |
| Dalotuzumalo^{[39]} | | mab | humanized | insulin-like growth | cancer etc. |
| | | | | factor I receptor | |
| Dapirolizumab pegol | | mab | humanized | CD40 ligand | ? |
| Daratumumab | | mab | human | CD38 (cyclic ADP | cancer |
| | | | | ribose hydrolase) | |
| Dectrekumab | | mab | human | IL-13 | ? |
| Demcizumab | | mab | humanized | DLL4 | cancer |
| Denintuzumab | | mab | humanized | CD19 | cancer |
| mafodotin | | | | | |
| Denosumab | Prolia | mab | human | RANKL | osteoporosis, bone |
| | | | | | metastases etc. |
| Derlotuximab biotin | | mab | chimeric | histone complex | recurrent glioblastoma |
| | | | | | multiforme |
| Detumomab | | mab | mouse | B-lymphoma cell | lymphoma |
| Dinutuximab | | mab | chimeric | ganglioside GD2 | neuroblastoma |
| Diridavumab | | mab | human | hemagglutinin | influenza A |
| Dorlimomab aritox | | F(ab')₂ | mouse | ? | ? |
| Drozitumab | | mab | human | DR5 | cancer etc. |
| Duligotumab | | mab | human | HER3 | ? |
| Dupilumab | | mab | human | IL4 | atopic diseases |
| Durvalumab | | mab | human | CD274 | cancer |
| Dusigitumab | | mab | human | ILGF2 | cancer |
| Ecromeximab | | mab | chimeric | GD3 ganglioside | malignant melanoma |
| Eculizumab | Soliris | mab | humanized | C5 | paroxysmal nocturnal |
| | | | | | hemoglobinuria |
| Edobacomab | | mab | mouse | endotoxin | sepsis caused by |
| | | | | | Gram-negative |
| | | | | | bacteria |
| Edrecolomab | Panorex | mab | mouse | EpCAM | colorectal carcinoma |
| Efalizumab | Raptiva | mab | humanized | LFA-1 (CD11a) | psoriasis (blocks T-cell |
| | | | | | migration) |
| Efungumab | Mycograb | scFv | human | Hsp90 | invasive Candida |
| | | | | | infection |
| Eldelumab | | mab | human | interferon gamma- | Crohn's disease, |
| | | | | induced protein | ulcerative colitis |
| Elgemtumab | | mab | human | ERBB3 | cancer |
| Elotuzumab | | mab | humanized | SLAMF7 | multiple myeloma |
| Elsilimomab | | mab | mouse | IL-6 | ? |
| Emactuzumab | | mab | humanized | CSF1R | cancer |
| Emibetuzumab | | mab | humanized | HHGFR | cancer |
| Enavatuzumab | | mab | humanized | TWEAK receptor | cancer etc. |
| Enfortumab vedotin | | mab | human | AGS-22M6 | cancer expressing |
| | | | | | Nectin-4 |
| Enlimomab pegol | | mab | mouse | ICAM-1 (CD54) | ? |
| Enoblituzumab | | mab | humanized | B7-H3 | cancer |
| Enokizumab | | mab | humanized | IL9 | asthma |
| Enoticumab | | mab | human | DLL4 | ? |
| Ensituximab | | mab | chimeric | 5AC | cancer |
| Epitumomab cituxetan | | mab | mouse | episialin | ? |
| Epratuzumab | | mab | humanized | CD22 | cancer, SLE |
| Erlizumab | | F(ab')₂ | humanized | ITGB2 (CD18) | heart attack, stroke, |
| | | | | | traumatic shock |
| Ertumaxomab | Rexomun | 3funct | rat/mouse hybrid | HER2/neu, CD3 | breast cancer etc. |
| Etaracizumab | Abegrin | mab | humanized | integrin αvβ3 | melanoma, prostate |
| | | | | | cancer, ovarian cancer |
| | | | | | etc. |
| Etrolizumab | | mab | humanized | integrin α7 β7 | inflammatory bowel |
| | | | | | disease |
| Evinacumab | | mab | human | angiopoietin 3 | dyslipidemia |
| Evolocumab | | mab | human | PCSK9 | hypercholesterolemia |
| Exbivirumab | | mab | human | hepatitis B surface | hepatitis B |
| | | | | antigen | |
| Fanolesomab | NeutroSpec | mab | mouse | CD15 | appendicitis |
| | | | | | (diagnosis) |
| Faralimomab | | mab | mouse | interferon receptor | ? |
| Farletuzumab | | mab | humanized | folate receptor 1 | ovarian cancer |
| Fasinumab | | mab | human | HNGF | acute sciatic pain |
| FBTA05 | Lymphomun | 3funct | rat/mouse hybrid | CD20 | chronic lymphocytic |
| | | | | | leukaemia |
| Felvizumab | | mab | humanized | respiratory syncytial | respiratory syncytial |
| | | | | virus | virus infection |
| Fezakinumab | | mab | human | IL-22 | rheumatoid arthritis, |
| | | | | | psoriasis |
| Ficlatuzumab | | mab | humanized | HGF | cancer etc. |
| Figitumumab | | mab | human | IGF-1 receptor | adrenocortical |
| | | | | | carcinoma, non-small |
| | | | | | cell lung carcinoma |
| | | | | | etc. |
| Firivumab | | mab | human | influenza A virus | ? |
| | | | | hemagglutinin | |
| Flanvotumab | | mab | human | TYRP1(glycoprotein | melanoma |
| | | | | 75) | |
| Fletikumab | | mab | human | IL 20 | rheumatoid arthritis |
| Fontolizumab | HuZAF | mab | humanized | IFN-γ | Crohn's disease etc. |
| Foralumab | | mab | human | CD3 epsilon | ? |
| Foravirumab | | mab | human | rabies virus | rabies (prophylaxis) |
| | | | | glycoprotein | |
| Fresolimumab | | mab | human | TGF-β | idiopathic pulmonary |
| | | | | | fibrosis, focal |
| | | | | | segmental |
| | | | | | glomerulosclerosis, |
| | | | | | cancer |
| Fulranumab | | mab | human | NGF | pain |
| Futuximab | | mab | chimeric | EGFR | ? |
| Galiximab | | mab | chimeric | CD80 | B-cell lymphoma |
| Ganitumab | | mab | human | IGF-I | cancer |
| Gantenerumab | | mab | human | beta amyloid | Alzheimer's disease |
| Gavilimomab | | mab | mouse | CD147 (basigin) | graft versus host |
| | | | | | disease |
| Gemtuzumab | Mylotarg | mab | humanized | CD33 | acute myelogenous |
| ozogamicin | | | | | leukemia |
| Gevokizumab | | mab | humanized | IL-1β | diabetes etc. |
| Girentuximab | Rencarex | mab | chimeric | carbonic anhydrase | clear cell renal cell |
| | | | | 9 (CA-IX) | carcinoma[81] |
| Glembatumumab | | mab | human | GPNMB | melanoma, breast |
| vedotin | | | | | cancer |
| Golimumab | Simponi | mab | human | TNF-α | rheumatoid arthritis, |
| | | | | | psoriatic arthritis, |
| | | | | | ankylosing spondylitis |
| Gomiliximab | | mab | chimeric | CD23 (IgE receptor) | allergic asthma |
| Guselkumab | | mab | human | IL23 | psoriasis |
| lbalizumab | | mab | humanized | CD4 | HIV infection |
| Ibritumomab tiuxetan | Zevalin | mab | mouse | CD20 | non-Hodgkin's |
| | | | | | lymphoma |
| Icrucumab | | mab | human | VEGFR-1 | cancer etc. |
| Idarucizumab | | mab | humanized | dabigatran | reversal of |
| | | | | | anticoagulant effects of |
| | | | | | dabigatran |
| Igovomab | Indimacis-125 | F(ab')₂ | mouse | CA-125 | ovarian cancer |
| | | | | | (diagnosis) |
| IMAB362 | | mab | human | CLDN18.2 | gastrointestinal |
| | | | | | adenocarcinomas and |
| | | | | | pancreatic tumor |
| Imalumab | | mab | human | MIF | cancer |
| Imciromab | Myoscint | mab | mouse | cardiac myosin | cardiac imaging |
| Imgatuzumab | | mab | humanized | EGFR | cancer |
| Inclacumab | | mab | human | selectin P | ? |
| Indatuximab ravtansine | | mab | chimeric | SDC1 | cancer |
| Indusatumab vedotin | | mab | human | GUCY2C | cancer |
| Infliximab | Remicade | mab | chimeric | TNF-α | rheumatoid arthritis, |
| | | | | | ankylosing spondylitis, |
| | | | | | psoriatic arthritis, |
| | | | | | psoriasis, Crohn's |
| | | | | | disease, ulcerative |
| | | | | | colitis |
| Intetumumab | | mab | human | CD51 | solid tumors (prostate |
| | | | | | cancer, melanoma) |
| Inolimomab | | mab | mouse | CD25 (α chain of IL- | graft versus host |
| | | | | 2 receptor) | disease |
| Inotuzumab | | mab | humanized | CD22 | cancer |
| ozogamicin | | | | | |
| Ipilimumab | Yervoy | mab | human | CD152 | melanoma |
| Iratumumab | | mab | human | CD30 (TNFRSF8) | Hodgkin's lymphoma |
| Isatuximab | | mab | chimeric | CD38 | cancer |
| Itolizumab | | mab | humanized | CD6 | ? |
| Ixekizumab | | mab | humanized | IL-17A | autoimmune diseases |
| Keliximab | | mab | chimeric | CD4 | chronic asthma |
| Labetuzumab | CEA-Cide | mab | humanized | CEA | colorectal cancer |
| Lambrolizumab | | mab | humanized | PDCD1 | antineoplastic agent |
| Lampalizumab | | mab | humanized | CFD | ? |
| Lebrikizumab | | mab | humanized | IL-13 | asthma |
| Lemalesomab | | mab | mouse | NCA-90 | diagnostic agent |
| | | | | (granulocyte | |
| | | | | antigen) | |
| Lenzilumab | | mab | human | CSF2 | ? |
| Lerdelimumab | | mab | human | TGF beta 2 | reduction of scarring |
| | | | | | after glaucoma surgery |
| Lexatumumab | | mab | human | TRAIL-R2 | cancer |
| Libivirumab | | mab | human | hepatitis B surface | hepatitis B |
| | | | | antigen | |
| Lifastuzumab vedotin | | mab | humanized | phosphate-sodium | cancer |
| | | | | co-transporter | |
| Ligelizumab | | mab | humanized | IGHE | severe asthma and |
| | | | | | chronic spontaneous |
| | | | | | urticarial |
| Lilotomab satetraxetan | | mab | mouse | CD37 | cancer |
| Lintuzumab | | mab | humanized | CD33 | cancer |
| Lirilumab | | mab | human | KIR2D | ? |
| Lodelcizumab | | mab | humanized | PCSK9 | hypercholesterolemia |
| Lokivetmab | | mab | veterinary | Canis lupus | ? |
| | | | | familiaris IL31 | |
| Lorvotuzumab | | mab | humanized | CD56 | cancer |
| mertansine | | | | | |
| Lucatumumab | | mab | human | CD40 | multiple myeloma, non- |
| | | | | | Hodgkin's lymphoma, |
| | | | | | Hodgkin's lymphoma |
| Lulizumab pegol | | mab | humanized | CD28 | autoimmune diseases |
| Lumiliximab | | mab | chimeric | CD23 (IgE receptor) | chronic lymphocytic |
| | | | | | leukemia |
| Lumretuzumab | | mab | humanized | ERBB3 | cancer |
| Mapatumumab | | mab | human | TRAIL-R1 | cancer |
| Margetuximab | | mab | humanized | ch4D5 | cancer |
| Maslimomab | | ? | mouse | T-cell receptor | ? |
| Mavrilimumab | | mab | human | GMCSF receptor α- | rheumatoid arthritis |
| | | | | chain | |
| Matuzumab | | mab | humanized | EGFR | colorectal, lung and |
| | | | | | stomach cancer |
| Mepolizumab | Bosatria | mab | humanized | IL-5 | asthma and white |
| | | | | | blood cell diseases |
| Metelimumab | | mab | human | TGF beta 1 | systemic scleroderma |
| Milatuzumab | | mab | humanized | CD74 | multiple myeloma and |
| | | | | | other hematological |
| | | | | | malignancies |
| Minretumomab | | mab | mouse | TAG-72 | tumor detection (and |
| | | | | | therapy?) |
| Mirvetuximab | | mab | chimeric | folate receptor alpha | cancer |
| soravtansine | | | | | |
| Mitumomab | | mab | mouse | GD3 ganglioside | small cell lung |
| | | | | | carcinoma |
| Mogamulizumab | | mab | humanized | CCR4 | cancer |
| Morolimumab | | mab | human | Rhesus factor | ? |
| Motavizumab | Numax | mab | humanized | respiratory syncytial | respiratory syncytial |
| | | | | virus | virus (prevention) |
| Moxetumomab | | mab | mouse | CD22 | cancer |
| pasudotox | | | | | |
| Muromonab-CD3 | Orthoclone | mab | mouse | CD3 | prevention of organ |
| | OKT3 | | | | transplant rejections |
| Nacolomab tafenatox | | Fab | mouse | C242 antigen | colorectal cancer |
| Namilumab | | mab | human | CSF2 | ? |
| Naptumomab | | Fab | mouse | 5T4 | non-small cell lung |
| estafenatox | | | | | carcinoma, renal cell |
| | | | | | carcinoma |
| Narnatumab | | mab | human | RON | cancer |
| Natalizumab | Tysabri | mab | humanized | integrin α4 | multiple sclerosis, |
| | | | | | Crohn's disease |
| Nebacumab | | mab | human | endotoxin | sepsis |
| Necitumumab | | mab | human | EGFR | non-small cell lung |
| | | | | | carcinoma |
| Nemolizumab | | mab | humanized | IL31RA | ? |
| Nerelimomab | | mab | mouse | TNF-α | ? |
| Nesvacumab | | mab | human | angiopoietin 2 | cancer |
| Nimotuzumab | Theracim, | mab | humanized | EGFR | squamous cell |
| | Theraloc | | | | carcinoma, head and |
| | | | | | neck cancer, |
| | | | | | nasopharyngeal |
| | | | | | cancer, glioma |
| Nivolumab | | mab | human | PD-1 | cancer |
| Nofetumomab | Verluma | Fab | mouse | ? | cancer (diagnosis) |
| mercentan | | | | | |
| Obiltoxaximab | | mab | chimeric | Bacillus anthracis | Bacillus anthracis |
| | | | | anthrax | spores |
| Obinutuzumab | Gazyva | mab | humanized | CD20 | Chronic lymphatic |
| | | | | | leukemia |
| Ocaratuzumab | | mab | humanized | CD20 | cancer |
| Ocrelizumab | | mab | humanized | CD20 | rheumatoid arthritis, |
| | | | | | lupus erythematosus |
| | | | | | etc. |
| Odulimomab | | mab | mouse | LFA-1 (CD11a) | prevention of organ |
| | | | | | transplant rejections, |
| | | | | | immunological |
| | | | | | diseases |
| Ofatumumab | Arzerra | mab | human | CD20 | chronic lymphocytic |
| | | | | | leukemia etc. |
| Olaratumab | | mab | human | PDGF-R α | cancer |
| Olokizumab | | mab | humanized | IL6 | ? |
| Omalizumab | Xolair | mab | humanized | IgE Fc region | allergic asthma |
| Onartuzumab | | mab | humanized | human scatter factor | cancer |
| | | | | receptor kinase | |
| Ontuxizumab | | mab | chimeric/humanized | TEM1 | cancer |
| Opicinumab^{l} | | mab | human | LINGO-1 | multiple sclerosis |
| Oportuzumab monatox | | scFv | humanized | EpCAM | cancer |
| Oregovomab | OvaRex | mab | mouse | CA-125 | ovarian cancer |
| Orticumab | | mab | human | oxLDL | ? |
| Otelixizumab | | mab | chimeric/humanized | CD3 | diabetes mellitus type |
| | | | | | 1 |
| Otlertuzumab | | mab | humanized | CD37 | cancer |
| Oxelumab | | mab | human | OX-40 | asthma |
| Ozanezumab | | mab | humanized | NOGO-A | ALS and multiple |
| | | | | | sclerosis |
| Ozoralizumab | | mab | humanized | TNF-α | inflammation |
| Pagibaximab | | mab | chimeric | lipoteichoic acid | sepsis |
| | | | | | (Staphylococcus) |
| Palivizumab | Synagis, | mab | humanized | F protein of | respiratory syncytial |
| | Abbosynagis | | | respiratory syncytial | virus (prevention) |
| | | | | virus | |
| Panitumumab | Vectibix | mab | human | EGFR | colorectal cancer |
| Pankomab | | mab | humanized | tumor specific | ovarian cancer |
| | | | | glycosylation of | |
| | | | | MUC1 | |
| Panobacumab | | mab | human | Pseudomonas | Pseudomonas |
| | | | | aeruginosa | aeruginosa infection |
| Parsatuzumab | | mab | human | EGFL7 | cancer |
| Pascolizumab | | mab | humanized | IL-4 | asthma |
| Pasotuxizumab | | mab | chimeric/humanized | folate hydrolase | cancer |
| Pateclizumab | | mab | humanized | LTA | TNF |
| Patritumab | | mab | human | HER3 | cancer |
| Pembrolizumab | | mab | humanized | PDCD1 | cancer etc. |
| Pemtumomab | Theragyn | ? | mouse | MUC1 | cancer |
| Perakizumab | | mab | humanized | IL17A | arthritis |
| Pertuzumab | Omnitarg | mab | humanized | HER2/neu | cancer |
| Pexelizumab | | scFv | humanized | C5 | reduction of side |
| | | | | | effects of cardiac |
| | | | | | surgery |
| Pidilizumab | | mab | humanized | PD-1 | cancer and infectious |
| | | | | | diseases |
| Pinatuzumab vedotin | | mab | humanized | CD22 | cancer |
| Pintumomab | | mab | mouse | adenocarcinoma | adenocarcinoma |
| | | | | antigen | (imaging) |
| Placulumab | | mab | human | human TNF | ? |
| Polatuzumab vedotin | | mab | humanized | CD79B | cancer |
| Ponezumab | | mab | humanized | human beta-amyloid | Alzheimer's disease |
| Priliximab | | mab | chimeric | CD4 | Crohn's disease, |
| | | | | | multiple sclerosis |
| Pritoxaximab | | mab | chimeric | E. coli shiga toxin | ? |
| | | | | type-1 | |
| Pritumumab | | mab | human | vimentin | brain cancer |
| PRO 140 | | ? | humanized | CCR5 | HIV infection |
| Quilizumab | | mab | humanized | IGHE | asthma |
| Racotumomab | | mab | mouse | N-glycolylneuraminic | cancer |
| | | | | acid | |
| Radretumab | | mab | human | fibronectin extra | cancer |
| | | | | domain-B | |
| Rafivirumab | | mab | human | rabies virus | rabies (prophylaxis) |
| | | | | glycoprotein | |
| Ralpancizumab | | mab | humanized | neural apoptosis- | dyslipidemia |
| | | | | regulated proteinase | |
| | | | | 1 | |
| Ramucirumab | Cyramza | mab | human | VEGFR2 | solid tumors |
| Ranibizumab | Lucentis | Fab | humanized | VEGF-A | macular degeneration |
| | | | | | (wet form) |
| Raxibacumab | | mab | human | anthrax toxin, | anthrax (prophylaxis |
| | | | | protective antigen | and treatment) |
| Refanezumab | | mab | humanized | myelin-associated | recovery of motor |
| | | | | glycoprotein | function after stroke |
| Regavirumab | | mab | human | cytomegalovirus | cytomegalovirus |
| | | | | glycoprotein B | infection |
| Reslizumab | | mab | humanized | IL-5 | inflammations of the |
| | | | | | airways, skin and |
| | | | | | gastrointestinal tract |
| Rilotumumab | | mab | human | HGF | solid tumors |
| Rinucumab | | mab | human | platelet-derived | neovascular age- |
| | | | | growth factor | related macular |
| | | | | receptor beta | degeneration |
| Rituximab | MabThera, | mab | chimeric | CD20 | lymphomas, |
| | Rituxan | | | | leukemias, some |
| | | | | | autoimmune disorders |
| Robatumumab | | mab | human | IGF-1 receptor | cancer |
| Roledumab | | mab | human | RHD | ? |
| Romosozumab | | mab | humanized | sclerostin | osteoporosis |
| Rontalizumab | | mab | humanized | IFN-α | systemic lupus |
| | | | | | erythematosus |
| Rovelizumab | LeukArrest | mab | humanized | CD11, CD18 | haemorrhagic shock |
| | | | | | etc. |
| Ruplizumab | Antova | mab | humanized | CD154 (CD40L) | rheumatic diseases |
| Sacituzumab govitecan | | mab | humanized | tumor-associated | cancer |
| | | | | calcium signal | |
| | | | | transducer 2 | |
| Samalizumab | | mab | humanized | CD200 | cancer |
| Sarilumab | | mab | human | IL6 | rheumatoid arthritis, |
| | | | | | ankylosing spondylitis |
| Satumomab pendetide | | mab | mouse | TAG-72 | cancer (diagnosis) |
| Secukinumab | | mab | human | IL-17A | uveitis, rheumatoid |
| | | | | | arthritis psoriasis |
| Seribantumab | | mab | human | ERBB3 | cancer |
| Setoxaximab | | mab | chimeric | E. coli shiga toxin | ? |
| | | | | type-2 | |
| Sevirumab | | ? | human | cytomegalovirus | cytomegalovirus |
| | | | | | infection |
| Sibrotuzumab | | mab | humanized | FAP | cancer |
| SGN-CD19A | | mab | humanized | CD19 | acute lymphoblastic |
| | | | | | leukemia and B-cell |
| | | | | | non-Hodgkin |
| | | | | | lymphoma |
| SGN-CD33A | | mab | humanized | CD33 | Acute myeloid |
| | | | | | leukemia |
| Sifalimumab | | mab | humanized | IFN-α | SLE, dermatomyositis, |
| | | | | | polymyositis |
| Siltuximab | | mab | chimeric | IL-6 | cancer |
| Simtuzumab | | mab | humanized | LOXL2 | fibrosis |
| Siplizumab | | mab | humanized | CD2 | psoriasis, graft-versus- |
| | | | | | host disease |
| | | | | | (prevention) |
| Sirukumab | | mab | human | IL-6 | rheumatoid arthritis |
| Sofituzumab vedotin | | mab | humanized | CA 125 | ovarian cancer |
| Solanezumab | | mab | humanized | beta amyloid | Alzheimer's disease |
| Solitomab | | mab | mouse | EpCAM | ? |
| Sonepcizumab | | ? | humanized | sphingosine-1- | choroidal and retinal |
| | | | | phosphate | neovascularization |
| Sontuzumab | | mab | humanized | episialin | ? |
| Stamulumab | | mab | human | myostatin | muscular dystrophy |
| Sulesomab | LeukoScan | Fab' | mouse | NCA-90 | osteomyelitis (imaging) |
| | | | | (granulocyte | |
| | | | | antigen) | |
| Suvizumab | | mab | humanized | HIV-1 | viral infections |
| Tabalumab | | mab | human | BAFF | B-cell cancers |
| Tacatuzumab | AFP-Cide | mab | humanized | alpha-fetoprotein | cancer |
| tetraxetan | | | | | |
| Tadocizumab | | Fab | humanized | integrin αIIbβ3 | percutaneous coronary |
| | | | | | intervention |
| Talizumab | | mab | humanized | IgE | allergic reaction |
| Tanezumab | | mab | humanized | NGF | pain |
| Taplitumomab paptox | | mab | mouse | CD19 | cancer[citation needed] |
| Tarextumab | | mab | human | Notch receptor | cancer |
| Tefibazumab | Aurexis | mab | humanized | clumping factor A | Staphylococcus aureus |
| | | | | | infection |
| Telimomab aritox | | Fab | mouse | ? | ? |
| Tenatumomab | | mab | mouse | tenascin C | cancer |
| Teneliximab | | mab | chimeric | CD40 | ? |
| Teplizumab | | mab | humanized | CD3 | diabetes mellitus type |
| | | | | | 1 |
| Teprotumumab | | mab | human | CD221 | hematologic tumors |
| Tesidolumab | | mab | human | C5 | ? |
| TGN1412 | | ? | humanized | CD28 | chronic lymphocytic |
| | | | | | leukemia, rheumatoid |
| | | | | | arthritis |
| Ticilimumab | | mab | human | CTLA-4 | cancer |
| (=tremelimumab) | | | | | |
| Tildrakizumab | | mab | humanized | IL23 | immunologically |
| | | | | | mediated inflammatory |
| | | | | | disorders |
| Tigatuzumab | | mab | humanized | TRAIL-R2 | cancer |
| TNX-650 | | ? | humanized | IL-13 | Hodgkin's lymphoma |
| Tocilizumab[6] | Actemra, | mab | humanized | IL-6 receptor | rheumatoid arthritis |
| (=atlizumab) | RoActemra | | | | |
| Toralizumab | | mab | humanized | CD154 (CD40L) | rheumatoid arthritis, |
| | | | | | lupus nephritis etc. |
| Tosatoxumab | | mab | human | Staphylococcus | ? |
| | | | | aureus | |
| Tositumomab | Bexxar | ? | mouse | CD20 | follicular lymphoma |
| Tovetumab | | mab | human | CD140a | cancer |
| Tralokinumab | | mab | human | IL-13 | asthma etc. |
| Trastuzumab | Herceptin | mab | humanized | HER2/neu | breast cancer |
| TRBS07 | Ektomab | 3funct | ? | GD2 | melanoma |
| Tregalizumab | | mab | humanized | CD4 | ? |
| Tremelimumab | | mab | human | CTLA-4 | cancer |
| Trevogrumab | | mab | human | growth | muscle atrophy due to |
| | | | | differentiation factor | orthopedic disuse and |
| | | | | 8 | sarcopenia |
| Tucotuzumab | | mab | humanized | EpCAM | cancer |
| celmoleukin | | | | | |
| Tuvirumab | | ? | human | hepatitis B virus | chronic hepatitis B |
| Ublituximab | | mab | chimeric | MS4A1 | cancer |
| Ulocuplumab | | mab | human | C-X-C chemokine | hematologic |
| | | | | receptor type 4 | malignancies |
| Urelumab | | mab | human | 4-1BB | cancer etc. |
| Urtoxazumab | | mab | humanized | Escherichia coli | diarrhoea caused by E. |
| | | | | | coli |
| Ustekinumab | Stelara | mab | human | IL-12, IL-23 | multiple sclerosis, |
| | | | | | psoriasis, psoriatic |
| | | | | | arthritis |
| Vandortuzumab | | mab | humanized | STEAP1 | cancer |
| vedotin | | | | | |
| Vantictumab | | mab | human | Frizzled receptor | cancer |
| Vanucizumab | | mab | humanized | angiopoietin 2 | cancer |
| Vapaliximab | | mab | chimeric | AOC3 (VAP-1) | ? |
| Varlilumab | | mab | human | CD27 | ? |
| Vatelizumab | | mab | humanized | ITGA2 | ? |
| Vedolizumab | | mab | humanized | integrin α4β7 | Crohn's disease, |
| | | | | | ulcerative colitis |
| Veltuzumab | | mab | humanized | CD20 | non-Hodgkin's |
| | | | | | lymphoma |
| Vepalimomab | | mab | mouse | AOC3 (VAP-1) | inflammation |
| Vesencumab | | mab | human | NRP1 | ? |
| Visilizumab | Nuvion | mab | humanized | CD3 | Crohn's disease, |
| | | | | | ulcerative colitis |
| Volociximab | | mab | chimeric | integrin α5β1 | solid tumors |
| Vorsetuzumab | | mab | humanized | CD70 | cancer |
| mafodotin | | | | | |
| Votumumab | HumaSPECT | mab | human | tumor antigen | colorectal tumors |
| | | | | CTAA16.88 | |
| Zalutumumab | HuMax-EGFr | mab | human | EGFR | squamous cell |
| | | | | | carcinoma of the head |
| | | | | | and neck |
| Zanolimumab | HuMax-CD4 | mab | human | CD4 | rheumatoid arthritis, |
| | | | | | psoriasis, T-cell |
| | | | | | lymphoma |
| Zatuximab | | mab | chimeric | HER1 | cancer |
| Ziralimumab | | mab | human | CD147 (basigin) | ? |
| Zolimomab | aritox | mab | mouse | CD5 | systemic lupus |
| | | | | | erythematosus, graft- |
| | | | | | versus-host disease |

The invention claimed is:

1. A cancer treatment and imaging method using compressive, non-thermal low power focused ultrasound, the method comprising the steps of:
administering a plurality of antibody or aptamer-conjugated nanoparticles, liposomes, and/or micelles to a patient in need thereof so as to target a tumor in the patient, the administered antibody or aptamer-conjugated nanoparticles, liposomes, and/or micelles being coated with cell penetrating peptides (CPPs) and/or a polymer on the nanoparticles, and the administered antibody or aptamer-conjugated nanoparticles, liposomes, and/or micelles containing a medication and/or gene, and a dye or indicator in the polymer coating, at least some of the antibody or aptamer-conjugated nanoparticles, liposomes, and/or micelles attaching to surface antigens of tumor cells of the tumor so as to form a tumor cell/nanoparticle/liposome/micelle complex;
exciting the antibody or aptamer-conjugated nanoparticles, liposomes, and/or micelles in a first compressive, non-thermal, low power focused mode using an ultrasound source that generates a focused compressive ultrasonic wave so as to peel off the polymer coating of the nanoparticles and/or break the liposomes and/or micelles by the focused vibrational force of the ultrasonic wave, thereby releasing the dye or indicator into the circulation of the patient and the medication and/or gene at the tumor site; and
imaging a body region of the patient so as to detect the dye or indicator released into the circulation of the patient.

2. The cancer treatment and imaging method according to claim 1, where, in the first compressive, non-thermal, low power focused mode, the ultrasonic wave generated by the ultrasound source has a frequency between about 10 kilohertz and about 100 kilohertz, and a power of about 1 Watt per cm².

3. The cancer treatment and imaging method according to claim 1, wherein the method further comprises the steps of:
heating the nanoparticles in a second thermal high power focused ultrasound mode using a high power focused ultrasound source that raises the temperature of the tumor cell/nanoparticle complex to a temperature of about 41° C. to about 43° C. so as to damage one or more tumor cell membranes at the tumor site and melt the polymer coating of the nanoparticles, thereby releasing the dye or indicator into the circulation of the patient and the medication and/or gene at the tumor site; and
alternating the heating of the nanoparticles in the second thermal high power focused ultrasound mode with the focused compressive low power ultrasonic wave in the first compressive non-thermal mode with the focused ultrasound under the control of a processor controlling the thermal energy intensity and duration of the ultrasound source.

4. The cancer treatment and imaging method according to claim 3, where, in the second thermal mode, the ultrasonic wave generated by the ultrasound source has a frequency between about 150 kilohertz and about 300 kilohertz, and a power of 1 Watt/cm² to 50 Watts/cm², or greater than 50 Watts/cm².

5. The cancer treatment and imaging method according to claim 3, where the nanoparticles comprise piezoelectric nanoparticles, and where the focused compressive ultrasonic wave in the first compressive non-thermal mode or second thermal high power focused ultrasound mode is delivered in a pulsed manner so as to generate an electric pulse from the piezoelectric nanoparticles that depolarizes the one or more tumor cell membranes so as to damage the tumor cells and makes the one or more tumor cell membranes accessible to the medication.

6. The cancer treatment and imaging method according to claim 1, where the antibody or aptamer-conjugated nanoparticles, liposomes, and/or micelles contain the medication in the polymer coating, and the medication is selected from the group consisting of Wnt inhibitors, Rock inhibitors, metformin, buformin, syrosingopine, phenformin, anti-vascular endothelial growth factors (anti-VEGFs), checkpoint inhibitors, macrolides, glycogen synthase kinase (GSK) inhibitors, an antineoplastic medication, and combinations thereof.

7. The cancer treatment and imaging method according to claim 6, wherein the patient comprises exhausted cytotoxic lymphocytes in the microenvironment of the tumor, and the method further comprises the steps of:
administering at least one of Rock inhibitors, Wnt inhibitors, glycogen synthase kinase inhibitors, IL-6 inhibitors, an anti-VEGF, and low molecular weight heparin to the microenvironment of the tumor in the patient;
removing toxins and dead cells from the blood of the patient after the administration of the at least one of the Rock inhibitors, Wnt inhibitors, glycogen synthase kinase inhibitors, IL-6 inhibitors, anti-VEGF, and low molecular weight heparin by electrophoresis or plasmapheresis so w to prevent a cytokine storm; and
reinfusing the blood back into the patient after the blood has been cleaned by the electrophoresis or the plasmapheresis.

8. The cancer treatment and imaging method according to claim 1, wherein the dye or indicator released is fluorescein or methylene blue.

9. The cancer treatment and imaging method according to claim 1, where the nanoparticles comprise gold nanoparticles, magnetic nanoparticles, or non-magnetic nanoparticles.

10. The cancer treatment and imaging method according to claim 1, wherein the nanoparticles further contain oncolytic viruses in the polymer coating; and
wherein the exciting of the nanoparticles using the ultrasound source further releases the oncolytic viruses from the polymer coating, the oncolytic viruses preferentially infecting and killing the tumor cells of the tumor, the circulating tumor cells, and/or the tumor exosomes.

11. The cancer treatment and imaging method according to claim 1, wherein the antibody or aptamer-conjugated nanoparticles further contain an adjuvant in the polymer coating, the adjuvant being selected from the group consisting of a dead tumor cell vaccine, bee venom, scorpion venom, toll-like receptor 4, viral-like particles (VLPs), and combinations thereof; and
wherein, when the adjuvant is released from the polymer coating of the nanoparticles, liposomes, and/or micelles, the adjuvant provides a humoral response and/or a signaling function for attracting T-cells and killer cells of the patient that attack the tumor cells of the tumor, the circulating tumor cells, and/or the tumor exosomes.

12. The cancer treatment and imaging method according to claim 11, where the nanoparticles comprise gold nanoparticles, and the method further comprising the step of:
subsequently applying local low dose radiation or stereotactic low dose radiation using x-ray, a proton beam, or a helium beam where an effect is enhanced by the gold nanoparticles, and the low dose radiation is useful for simultaneous imaging and enhances the humoral and cellular immune response to the tumor cells by increasing the T-cell and killer cell response to the tumor cells of the tumor, the circulating tumor cells, and/or the tumor exosomes.

13. The cancer treatment and imaging method according to claim 1, wherein the nanoparticles contain the medication in the polymer coating, and the nanoparticles with the medication are administered by means of the nasal mucosa so as to travel through the olfactory nerves and/or trigeminal nerves to the brain.

14. The cancer treatment and imaging method according to claim 13, further comprising the step of:
administering stem cells by means of the nasal mucosa so as to travel through the olfactory nerves to the brain.

15. A cancer or benign tumor treatment and imaging method, the method comprising the steps of:
systemically administering antibody-coated piezoelectric or pyroelectric nanoparticles to a patient in need thereof so as to target a tumor in the patient, the piezoelectric or pyroelectric nanoparticles being further coated with a polymer, and a medication being conjugated with the polymer coating of the piezoelectric or pyroelectric nanoparticles, the piezoelectric or pyroelectric nanoparticles attaching to surface antigens of tumor cells of the tumor so as to form a tumor cell/nanoparticle complex;
applying a pulsed electrical current to the piezoelectric or pyroelectric nanoparticles using an electrical source at the site of the tumor so as to create an electroacoustic sound from the piezoelectric or pyroelectric nanoparticles;
recording the electroacoustic sound generated by the piezoelectric or pyroelectric nanoparticles using a transducer to convert the electroacoustic sound to an electrical signal; and
amplifying and transmitting the electrical signal to a processor so that a 1-dimensional, 2-dimensional, or 3-dimensional image of the tumor structure is able to be generated in the form of electroacoustic computed tomogram.

16. The cancer or benign tumor treatment and imaging method according to claim 15, wherein the method further comprises the step of:
increasing the permeability of one or more tumor cell membranes of the tumor using the pulsed electrical current or a thermal energy source, thereby facilitating the entry of the medication into the tumor cells of the tumor.

17. The cancer or benign tumor treatment and imaging method according to claim 15, wherein the method further comprises the step of:
heating antibody or aptamer-conjugated liposomes or micelles, and/or the piezoelectric or pyroelectric nanoparticles and the medication using a high power focused ultrasound source operating in a thermal mode so as to raise the temperature of the tumor cell/nanoparticle/liposome/micelle complex to a temperature of about 41° C. to about 43° C., thereby damaging one or more tumor cell membranes at the tumor site and melting the polymer coating of the nanoparticles to release the medication at the tumor site.

18. The cancer or benign tumor treatment and imaging method according to claim 17, where the electrical source comprises a battery device with an anode located on a first side of the body of the patient and a cathode being located on a second side of the body of the patient, the pulsed electrical current passing through the body of the patient from the anode to the cathode of the battery device, and where the pulsed electrical current further damages the one or more tumor cell membranes at the tumor site and drives the released medication into the tumor cells at the tumor site.

19. The cancer or benign tumor treatment and imaging method according to claim 15, wherein the method further comprises the steps of:
administering antibody or aptamer-conjugated magnetic or paramagnetic nanoparticles and/or medication-filled liposomes to the patient; and
exciting the magnetic or paramagnetic nanoparticles and/or the medication-filled liposomes using an energy source producing an alternating magnetic field operating in a thermal mode so as to raise the temperature of the tumor cell/nanoparticle complex and/or liposome complex to a temperature of about 41° C. to about 43° C. or more, thereby releasing one or more medications from the liposomes, damaging one or more tumor cell membranes at the tumor site, and melting the polymer coating of the nanoparticles to release the medication at the tumor site.

20. The cancer or benign tumor treatment and imaging method according to claim 19, wherein the alternating magnetic field operating in the thermal mode has a frequency greater than 300 kilohertz.

21. The cancer or benign tumor treatment and imaging method according to claim 15, wherein the tumor in the patient being targeted comprises a benign compressive tumor selected from the group consisting of a fibroma, schwannoma, neurofibroma, meningioma, leiomyoma, angioma craniopharyngioma, papilloma, osteoma, osteoblastoma, Ewing sarcoma, osteosarcoma, eosinophilic granuloma, aneurysm, mesenchymal chondrosarcoma, fibrous dysplasia, angioma, angiosarcoma, pituitary adenoma, hemangioblastoma, glioma, and combinations thereof.

22. A cancer or benign tumor treatment method, the method comprising the steps of:
administering a plurality of antibody or aptamer-conjugated nanoparticles, liposomes, and/or micelles to a patient in need thereof so as to target a tumor in the patient, the administered antibody or aptamer-conjugated nanoparticles, liposomes, and/or micelles being coated with cell penetrating peptides (CPPs) and/or a polymer on the nanoparticles, and the administered antibody or aptamer-conjugated nanoparticles, liposomes, and/or micelles containing a medication and/or gene, and a dye or indicator in the polymer coating, at least some of the antibody or aptamer-conjugated nanoparticles, liposomes, and/or micelles attaching to surface antigens of is tumor cells of the tumor so as to form a tumor cell/nanoparticle/liposome/micelle complex; and
heating the antibody or aptamer-conjugated nanoparticles, liposomes, and/or micelles using an energy source so as to raise the temperature of the tumor cell/nanoparticle complex, micelle complex, and/or liposome complex to a temperature of about 41° C. to about 43° C. or more, thereby releasing one or more medications from the antibody or aptamer-conjugated nanoparticles, liposomes, and/or micelles, damaging one or more tumor cell membranes at the tumor site, and/or melting the polymer coating of the nanoparticles to release the medication at the tumor site.

23. The cancer or benign tumor treatment method according to claim 22, wherein the energy source for heating the antibody or aptamer-conjugated nanoparticles, liposomes, and/or micelles is selected from the group consisting of focused ultrasound laser, an alternating magnetic field, microwave radiation, and radiofrequency (RF) energy.
